(19) United States
(12) Patent Application Publication
(10) Pub. No.: US 2024/0301420 A1
(43) Pub. Date: Sep. 12, 2024

(54) METHOD FOR MODULATING UNPRODUCTIVE ALTERNATIVE SPLICING

(71) Applicant: The Trustees of Columbia University in the City of New York, New York, NY (US)

(72) Inventors: Paola RINCHETTI, New York, NY (US); Georgia NTERMENTZAKI, New York, NY (US); Dmytro USTIANENKO, New York, NY (US); Yocelyn RECINOS, New York, NY (US); Xiaojian WANG, Fort Lee, NJ (US); Francesco LOTTI, New York, NY (US); Chaolin ZHANG, Scarsdale, NY (US)

(21) Appl. No.: 18/622,162

(22) Filed: Mar. 29, 2024

Related U.S. Application Data
(63) Continuation of application No. PCT/US2022/077302, filed on Sep. 29, 2022.
(60) Provisional application No. 63/249,659, filed on Sep. 29, 2021.

(51) Int. Cl. C12N 15/113

(52) U.S. Cl. CPC C12N 15/113; C12N 2310/13; C12N 2320/33

(57) ABSTRACT

A method of increasing or decreasing expression of a target mRNA and protein for treatment of certain disease conditions by cells having a pre-mRNA that comprises a poison exon and encodes the target protein, and can include contacting the cells with an antisense oligomer (ASO) complementary to a targeted portion of the pre-mRNA.

### CROSS-REFERENCE TO RELATED APPLICATIONS

This application is a continuation of PCT patent application PCT/US2022/077302, filed on Sep. 22, 2024, entitled “Method For Modulating Unproductive Alternative Splicing”, which claims priority to U.S. Provisional Application No. 63/249,659 filed on Sep. 29, 2021, the contents of which are incorporated by reference in their entireties.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 178,614 bytes .xml file named “44010.096US-PAT” created on May 28, 2024.

### FIELD

The present subject matter relates to a method for modulating alternative splicing, and particularly, to a method for upregulating or downregulating functional mRNA and protein production and treating monogenic disorders or indications by modulating unproductive alternative splicing.

### BACKGROUND

It has been estimated that 10% of the world's population are affected by monogenic conditions, which can be caused by mutations that result in deficiency of functional proteins or aberrant expression of toxic proteins. The protein deficiency can include haploinsufficiency, in which heterozygous loss-of-function (LoF) mutations result in unproductive transcripts that do not produce functional proteins, or hypomorphic alleles that produce mutant or truncated proteins with reduced activity, thus reducing the amount or activity of functional protein products. While currently many of such conditions do not have effective treatment options, therapeutic approaches that can restore the level of functional mRNA and proteins are promising.

KBG syndrome is a rare genetic disorder characterized by developmental delay, intellectual disability, short stature, and multiple dysmorphic features (Herrmann et al., 1975; Morel Swols et al., 2017). In most cases, KBG syndrome is caused by heterozygous LoF mutations in ANKRD11 (Sirmaci et al., 2011) or microdeletions of the 16924.3 region harboring the ANKRD11 gene (Sacharow et al., 2012), which encodes a protein that functions as a chromatin coregulator (Zhang et al., 2004; Zhang et al., 2007; Neilsen et al., 2008).

Sotos syndrome is a developmental disorder characterized by learning disability, overgrowth, as well as distinct facial features. Over 90% of Sotos syndrome patients are haploinsufficient for NSD1 gene encoding nuclear receptor-binding Su(var)3-9, Enhancer-of-zesteand Trithorax domain-containing protein 1.

Currently, treatment options for KBG syndrome and Sotos syndrome are limited, with a focus on symptom management on a case-by-case basis (Morel Swols, D., et al. 2017. “KBG syndrome.” Orphanet J Rare Dis 12: 183; Baujat, G. and V. Cormier-Daire. 2007. “Sotos syndrome.” Orphanet J Rare Dis 2: 36).

In addition to neurodevelopmental and morphological phenotypes, Sotos syndrome patients with NSD1 haploinsufficiency show an accelerated epigenetic clock, a pattern of DNA methylation in the individual genome that can be used to predict biological age (Horvath, S. 2013), as well as advanced bone age, as compared to their chronological ages (Martin-Herranz et al. 2019; Jeffries, A. R., et al. 2019). On the other hand, overexpression of NSD1 due to genomic duplications causes ‘reverse Sotos syndrome’, which is characterized by short stature, developmental, microcephaly, delayed bone age (Zhang, H., et al. 2011). These observations suggest that upregulation of NSD1 may provide a means of slow down or reverse the epigenetic clock, while downregulation of NSD1 can accelerate the epigenetic clock, with an impact on the aging process. Furthermore, somatic mutations in NSD1 can cause a range of tumors (Papillon-Cavanagh et al., 2017; Shiba et al., 2013). Normalization of NSD1 expression and function can potentially provide an approach to control tumor development.

Alternative splicing (AS) is a molecular mechanism to produce multiple transcript and protein variants (isoforms) from single genes. Alternative splicing is ubiquitous, occurring in >90% of multi-exon human genes (Pan et al., 2008; Wang et al., 2008). About two-thirds of alternative splicing events produce a mix of protein-coding transcripts and unproductive transcripts due to introduction of in-frame premature termination codons (PTCs) by inclusion or exclusion of the alternative exon. The PTC-containing transcripts are either eliminated by the cell (e.g., through non-sense mediated decay, NMD, or other RNA degradation pathways) without translation, or they are translated into truncated proteins, with no or reduced function (FIG. 1A). In this disclosure, these exons are referred to as “poison exons”.

In principle, the expression of the functional mRNA and the protein product can be increased by modulating splicing of the poison exons, thereby suppressing the unproductive transcript isoform and restoring the production of the functional protein. However, in practice, to increase the protein level to an extent that is clinically meaningful, the relative abundance of the unproductive transcripts (i.e., percent inclusion of a poison exon) has to be sufficiently high. For example, in the case of haploinsufficency, inclusion of a poison exon has to be >50% to achieve two-fold upregulation of the protein from the intact allele to restore the physiological level, assuming that efficient suppression of the poison exon can be achieved by a therapeutic agent.

The identification of relatively abundant poison exons is a major challenge in the field for many reasons. First, since the unproductive transcripts containing poison exons are degraded by the cell, their true abundance level is difficult to measure. Second, conventional genetic or pharmaceutical approaches commonly used to suppress RNA degradation is not completely efficient. Third, although there are tens of thousands of potential poison exons in the human genome and thousands of those are in genes implicated in genetic diseases, in the vast majority of cases, the unproductive isoform appears to have a very low level (e.g., a criterion of 3% exon inclusion used in Lim, K H., et al. (2020), “Antisense Oligonucleotide Modulation of Non-Productive Alternative Splicing Upregulates Gene Expression” Nat Commun 11:3501). This raised the concern that many of the poison exons are unlikely viable drug targets. For example, using RNA-seq data derived from human brains of different ages, over 40,000 poison exons were identified. Among them, only in ˜1300 cases (3%), the unproductive isoform is expected to be sufficiently abundant (i.e., between 30% and 70%) in neonatal brain (Yan, Q., et al. 2015. “Systematic discovery of regulated and conserved alternative exons in the mammalian brain reveals NMD modulating chromatin regulators” Proc Natl Acad Sci USA 112: 3445-3350.). Importantly, the level of the vast majority of the poison exons is intrinsically low even before degradation (Pan, Q., et al. 2006. “Quantitative microarray profiling provides evidence against widespread coupling of alternative splicing with nonsense-mediated mRNA decay to control gene expression” Genes Dev. 20: 153-158). Therefore, a limited number of relatively abundant poisonous exons with therapeutic potential are hidden in tens of thousands of low abundance exons (a needle in the haystack situation).

Once an abundant poison exon is identified, an antisense oligomer (ASO) can be used as a therapeutic agent to bind to a target region by Watson-Crick base complementarity (Havens and Hastings, 2016; Lim et al., 2020) (FIG. 1B). The target region can be within the exon, or in the upstream/downstream regions that contain regulatory sequences normally recognized by endogenous splicing factors for controlling the exon inclusion level. These sequences can be several hundred nucleotides away from the alternative exon, but sometimes they can be more distal. The ASO binding interferes with splicing factor binding, thereby modulating splicing of the poison exon. This results in modulating production of the functional mRNA and protein. The gene targeted by the ASO can be the same gene that is mutated in the disease or indication, or a gene that can be upregulated to functionally compensate for the disruption of the disease-causing gene. One successful example of this strategy is treatment of spinal muscular atrophy (caused by disruption of SMN1 gene) using ASOs targeting a paralogous gene SMN2 to produce the functionally intact protein (Hua, Y., et al. 2008. “Antisense masking of an hnRNP A1/A2 intronic splicing silencer corrects SMN2 splicing in transgenic mice” Am J Hum Genet 82: 834-848). Experiments replicating these results in human cells are shown in FIGS. 2A-2B.

Therefore, identifying an abundant poison exon and modulating its alternative splicing to increase or decrease functional mRNA and protein levels is highly desired for treatment of monogenic disorders, such as KBG syndrome, Sotos syndrome, reverse Sotos syndrome, and other disease conditions, such as aging and cancer. While a major focus of this invention is upregulation of gene and protein expression, the method and compositions we developed can be also used to downregulate gene and protein expression, in certain conditions, such as reverse Sotos syndrome, when such modulation is beneficial.

### SUMMARY

A method of increasing or decreasing expression of a target functional mRNA or protein by cells having a precursor mRNA (pre-mRNA) that can be spliced into an unproductive RNA containing a poison exon or functional mRNA that can be translated into the target protein, can include contacting the cells with an antisense oligomer (ASO) complementary to a targeted portion of the precursor mRNA to generate functional mRNA encoding the target protein. The target protein can be selected from the group consisting of ANKRD11 and NSD1. The antisense oligomer (ASO) can bind to a targeted portion of the pre-mRNA encoding the target protein and modulate binding of a factor involved in splicing of the poison exon. The poison exon can be selected from exon 3× in the ANKRD11 gene, exon 4× in the ANKRD11 gene, and exon 11× in the NSD1 gene.

A method of treating a monogenic disorder and other related disease conditions in a subject in need thereof by increasing or decreasing expression of a target functional mRNA or protein by cells of the subject, wherein the cells have a pre-mRNA that comprises a poison exon and encodes the target protein when splicing of the poison exon is suppressed, can include contacting the cells with an antisense oligomer (ASO) complementary to a targeted portion of the pre-mRNA. The target protein can be selected from the group consisting of ANKRD11 and NSD1. The antisense oligomer (ASO) can bind to a targeted portion of the pre-mRNA and modulate binding of a factor involved in splicing of the poison exon. The poison exon can be selected from exon 3× in the ANKRD11 gene, exon 4× in the ANKRD11 gene, and exon 11× in the NSD1 gene. The disease conditions can be selected from KBG syndrome, Sotos syndrome, reverse Sotos syndrome, normal and pathological aging, and cancer.

### BRIEF DESCRIPTION OF DRAWINGS

Various embodiments will now be described in detail with reference to the accompanying drawings.

FIG. 1A is a diagram showing how inclusion of a poison exon by alternative splicing limits the production of functional mRNA and proteins.

FIG. 1B is a diagram showing suppression of the poison exon by antisense oligomers (ASOs) to increase functional mRNA and protein production and to treat disease caused by protein deficiency, which can include, but not limited to, haploinsufficiency.

FIG. 2A is a schematic illustration of SMN2 minigene splicing reporter encompassing exon 6 to exon 8 (the position of a downstream intronic splicing silencer ISS-N1, targeted by the FDA approved ASO drug nusinersen, sold under the SPINRAZA® brand, is highlighted).

FIG. 2B is a gel image of RT-PCR analysis of SMN2 exon 7 inclusion after treatment of ASO at different concentrations (HEK293 cells were co-transfected with the SMN2 minigene and ASO at different concentrations, followed by RT-PCR and agarose gel electrophoresis to analyze exon 7 inclusion level). The quantification of exon inclusion is indicated below the image.

FIG. 3A depicts UCSC genome browser view of ANKRD11 splicing isoforms. The positions of the two poison exons (exon 3× and 4×) we identified are indicated.

FIGS. 3B-3C depict zoom-in view highlighting poison exon 3× (FIG. 3B) and poison exon 4× (FIG. 3C) concerned in this invention. Note exon 4× has two alternative 3′ splice sites, which can result in 22 nucleotide difference in the size of the exon. The genomic coordinates of each exon (UCSC human genome assembly hg19) are provided.

FIG. 4A depicts UCSC genome browser view of NSD1 gene structure including the position of a poison exon concerned in this invention.

FIG. 4B depicts zoom-in view highlighting poison exon 11×. The genomic coordinates of the exon (UCSC human genome assembly hg19) are provided.

FIGS. 5A-5C depict validation of ANKRD11 mRNA upregulation using 2′ oMe-PS ASOs (Seq. NO 7-9) targeting splice sites of poison exon 4×. HEK293 cells transfected with individual ASOs at different concentrations, followed by RT-PCR and q-PCR to analyze exon inclusion and ANKRD11 mRNA levels. (5A) UCSC genome browser view depicting the position of ASOs we tested; (5B) is a graph depicting dosage dependent skipping of the poison exon targeted by ASOs (a representative gel image of RT-PCR analysis, together with the quantification of exon inclusion is shown above the graph); and (5C) is a graph showing results of RT-qPCR analysis quantifying relative expression level of ANKRD11 with/without ASO treatment. Mean and standard error of the mean (SEM) are shown (n=2). Statistical significance of upregulation upon ASO treatment was evaluated using single sided t-test (* p<0.1; ** p<0.05).

FIG. 6A-6C depicts splicing modulation and upregulation of Ankrd11 expression in the mouse brain using a 2′ MOE-PS ASO (ASO 5′-2 in FIG. 5A; Seq. NO 8) targeting the 3′ splice site of the poison exon 4×. (6A) is a schematic illustration showing the position of the ASO as well as intracerebroventricular (ICV) injection of ASO at 50 μg to neonatal mice at postnatal day 2 (P2). Injection of saline was used for control. Cortex tissues were collected and analyzed for Ankrd11 mRNA abundance at P9. (6B) is a gel image showing results of RT-PCR analysis (top) and a bar plot showing quantification of exon inclusion level (bottom) with/without ASO treatment. (6C) is a bar plot showing results of RT-qPCR analysis quantifying relative expression level of Ankrd11 with/without ASO treatment. Mean and standard error of the mean (SEM) are shown (n=2). Statistical significance of upregulation upon ASO treatment was evaluated using single sided t-test (* p<0.05).

FIGS. 7A-7C depict validation of NSD1 mRNA upregulation using 2′ oMe-PS ASOs targeting splice sites of poison exon 11× (Seq. NO 10-11). (7A) UCSC genome browser view depicting the position of ASOs we tested; (7B) is a graph depicting dosage dependent skipping of the poison exon targeted by ASOs (a representative gel image of RT-PCR analysis, together with the quantification of exon inclusion is shown above the graph; and (7C) is a graph showing results of RT-qPCR analysis quantifying relative expression level of NSD1 with/without ASO treatment. Mean and standard error of the mean (SEM) are shown (n≥3). Statistical significance of upregulation upon ASO treatment was evaluated using single sided t-test.

FIG. 8A-8F depicts ASO-mediated upregulation of Nsd1 mRNA and protein in the mouse brain (8A-8D) and NSD1 mRNA in hiPSC-derived brain organoid (8E,8F). (8A) is a cartoon showing wild type P2 mice treated with 25 μg of 2′ MOE-PS ASO targeting the 5′ splice site (Seq. NO 11) or saline by ICV injection. Cortex tissues were harvested 7 days after treatment. (8B) is a bar plot showing RT-qPCR analysis that quantifies relative expression level of Nsd1 mRNA upon ASO treatment. (8C,8D) depict western blots (8C) and quantification (8D) of Nsd1 protein after ASO treatment. (8E) is a cartoon showing brain organoids differentiated from human iPSCs. Organoids were treated with ASO by free uptake (20 μM). After 72 hrs, cells were collected for analysis. (8F) RT-qPCR analysis quantifying relative expression level of NSD1 mRNA upon ASO treatment. Mean and SEM are shown in bar plots (n=3). * p<0.05, ** p<0.01; single-sided t-test.

FIGS. 9A-9B is a schematic illustration of the design of a 10-nt step ASO walk (9A) and 1-nt step microwalk (9B) to screen splicing-modulating ASOs targeting the alternative exon or flanking intronic sequences.

FIG. 10 depicts schematic illustration of ASO screening for ANKRD11 by targeting exon 4× (Seq. NO 12-69). The UCSC genome browser view depicts the positions of ASOs we screened by ASO walk with 15-nt 2′ MOE-PS ASOs at 5 nucleotide steps.

FIG. 11A-11B depicts results of ASO screening targeting ANKRD11 exon 4× in cell line BEK 293T. Cells transfected with individual ASOs at 80 nM with mock transfection (no ASO) as control. RNA was extracted from treated cells for RT-PCR analysis to quantify exon inclusion level. (11A) is a representative image of agarose gel electrophoresis of PCR-amplified products from each ASO tested. (11B) shows quantification of exon inclusion for each ASO tested Statistical analysis was performed using one-way ANOVA (*p<0.05; **p<0.01; ***p<0.001; *****p<0.0001 with Dunnett multiple test correction). ASOs that decrease (ASO 29-33, 37, 41; corresponding to Seq. NO 40-44, 48, 52) or increase (ASOs 4-8, 43-44; corresponding to Seq. NO 15-19, 54-55) exon inclusion most effectively are highlighted in red and blue boxes, respectively.

FIG. 12A-12C depicts additional validation of four ANKRD11 ASO candidates (ASOs 29, 31, 33, 41; corresponding to Seq. NO 40, 42, 44, 52) identified by ASO walk. The ASO targeting the 5′ end of the exon (ASO 5′, denoted ASO 5′-2 in FIG. 5A; Seq. NO 8) was included as a positive control. (12A) is a representative image of agarose gel electrophoresis of PCR-amplified products from each ASO tested. (12B) is quantification of exon inclusion for each ASO tested. (12C) is RT-q-PCR analysis quantifying upregulation of ANKRD11 mRNA level after treatment with ASO 31 (Seq. NO 42). Statistical analysis was performed using one-way ANOVA, *p<0.05; **p<0.01; ***p<0.001; *****p<0.0001 with Dunnett multiple test correction.

FIG. 13 depicts two regions important for inclusion of ANKRD11 exon 4× identified through ASO screening (sequence targeted by ASO 29-33 and sequence targeted by ASO 41). Three additional 2′ MOE-PS ASOs were designed and tested based on screening and cross-species conservation of targeted sequences (ASO S1-S3, corresponding to Seq. NO 70-72). Note that the RNA sequence targeted by each ASO is shown at the bottom and the actual ASO sequence is the reverse complementary to the sequence shown.

FIG. 14A-14C depicts additional validation of four ANKRD11 ASO candidates (ASOs 37, S1, S2, S3; corresponding to Seq. NO 48, 70-72) determined based on ASO walk. (14A) is a representative image of agarose gel electrophoresis of PCR-amplified products from each ASO tested. (14B) is quantification of exon inclusion for each ASO tested. (14C) is RT q-PCR analysis quantifying upregulation of ANKRD11 mRNA after treatment with ASO S1 (Seq. NO 70). Statistical analysis was performed using one-way ANOVA, *p<0.05; **p<0.01; ***p<0.001; *****p<0.0001 with Dunnett multiple test correction.

FIG. 15 depicts schematics of ASO screening for NSD1 targeting exon 11× (Seq. NO 73-128). UCSC genome browser view depicting the position of ASOs we screened by ASO walk with 15-nt 2′ MOE-PS ASOs at 5 nucleotide steps.

FIG. 16A-16B depicts results of ASO screening targeting NSD1 exon 11× in cell line BEK 293T. Cells transfected with individual ASOs at 80 nM with mock transfection (no ASO) as control. Cells were then treated with emetine to inhibit translation and NMD 5 hrs before collection. No ASO, no emetine treatment (MOCK-) was included as an additional control. RNA was extracted from treated cells for RT-PCR analysis to quantify exon inclusion level. (16A) is a representative image of agarose gel electrophoresis of PCR-amplified products from each ASO tested. (16B) Quantification of exon inclusion for each ASO tested. Statistical analysis was performed to compare cells with/without ASO treatment in the presence of emetine using one-way ANOVA, *p<0.05; **p<0.01; ***p<0.001; *****p<0.0001 with Dunnett multiple test correction. ASOs that decrease (ASOs 23-25, 46-48; corresponding to Seq. NO 95-97, 104-106) or increase (ASOs 55-56; corresponding to Seq. NO 113,114) exon inclusion most effectively are highlighted in red and blue boxes, respectively.

FIG. 17 depicts two regions (sequence targeted by ASO 23-25 and sequence targeted by ASOs 46-48; corresponding to Seq. NO 95-97, 104-106) important for exon inclusion and one region (sequence targeted by ASOs 55-56; corresponding to Seq. NO 113,114) important for exon skipping.

### DETAILED DESCRIPTION

### Definitions

The following definitions are provided for the purpose of understanding the present subject matter and for constructing the appended patent claims.

It is noted that, as used in this specification and the appended claims, the singular forms “a”, “an”, and “the” include plural references unless the context clearly dictates otherwise.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”. As used herein, the term “comprise” or variations thereof such as “comprises” or “comprising” are to be read to indicate the inclusion of any recited feature (e.g., in the case of an antisense oligomer, a defined nucleobase sequence) but not the exclusion of any other features. Thus, as used herein, the term “comprising” is inclusive and does not exclude additional, unrecited features (e.g., in the case of an antisense oligomer, the presence of additional, unrecited nucleobases).

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The present disclosure provides compositions and methods for modulating alternative splicing of genes known to cause monogenic diseases (especially disorders with autosomal dominant inheritance) that can be clearly targeted by an antisense oligonucleotide (ASO) to effectively restore functional mRNA and protein production, including ANKRD11 for KBG syndrome and NSD1 for Sotos syndrome, reverse Sotos syndrome, normal and pathological aging and cancer.

One of the alternative splicing events in the targeted genes that can lead to unproductive alternative splicing or unproductive mRNA transcripts is the inclusion of an extra exon in the mRNA transcript that can induce retention of the transcript in the nucleus and mRNA decay, which could be due to different mechanisms including nonsense mediated mRNA decay (NMD). Herein, these exons are referred to as “poison exon”. An embodiment of the present disclosure provides a method of increasing or decreasing expression of a target mRNA or protein by cells having a pre-mRNA that comprises one or more poison exons; when the poison exon is skipped, mRNA will be produced by the cell to encode the target protein. The method can include contacting the cells with an antisense oligomer (ASO) complementary to a targeted portion of the pre-mRNA encoding the target mRNA and protein.

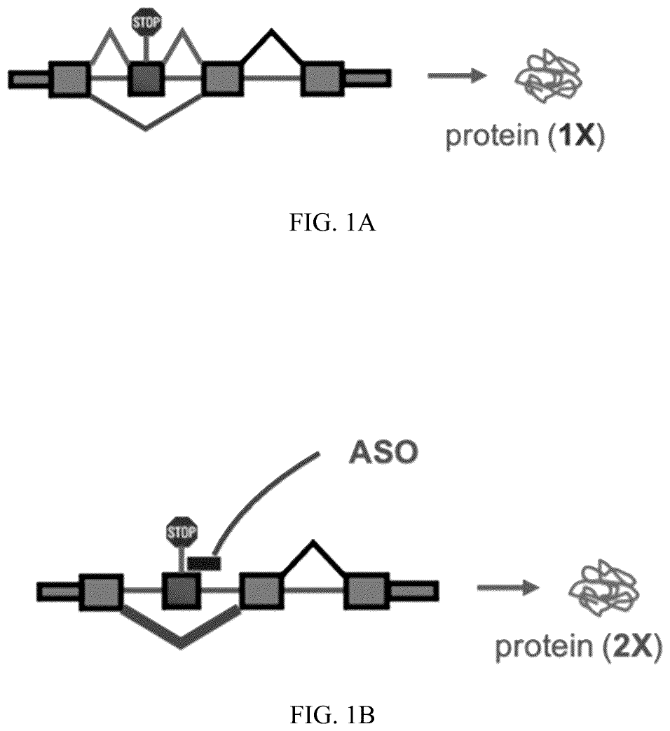

A poison exon is an exon that contains a premature termination codon (PTC) either in the exon or in the downstream mRNA sequence that can activate RNA decay pathways (for example, the NMD pathway) if included in a mature RNA transcript (FIG. 1A). Mature mRNA transcripts containing such a poison exon may be unproductive or they can be translated to generate truncated proteins with reduced or altered activity. Inclusion of a poison exon in mature RNA transcripts may downregulate gene expression.

The relationship between an antisense oligonucleotide (ASO) and its reverse complementary nucleic acid target, to which it hybridizes, is commonly referred to as “antisense”. “Targeting” a therapeutic agent to a target region or targeted portion of a chosen nucleic acid target can include identifying a nucleic acid sequence whose function is to be modulated. The target region can be within a poison exon or in the upstream/downstream regions that are normally recognized by endogenous splicing factors for controlling exon inclusion level. In an embodiment, an ASO can be used as the therapeutic agent to bind to the target region by Waston-Crick base complementarity. The ASO binding interferes with splicing factor binding, thereby modulating splicing of the poison exon. This results in modulating production of the functional mRNA and protein (FIG. 1).

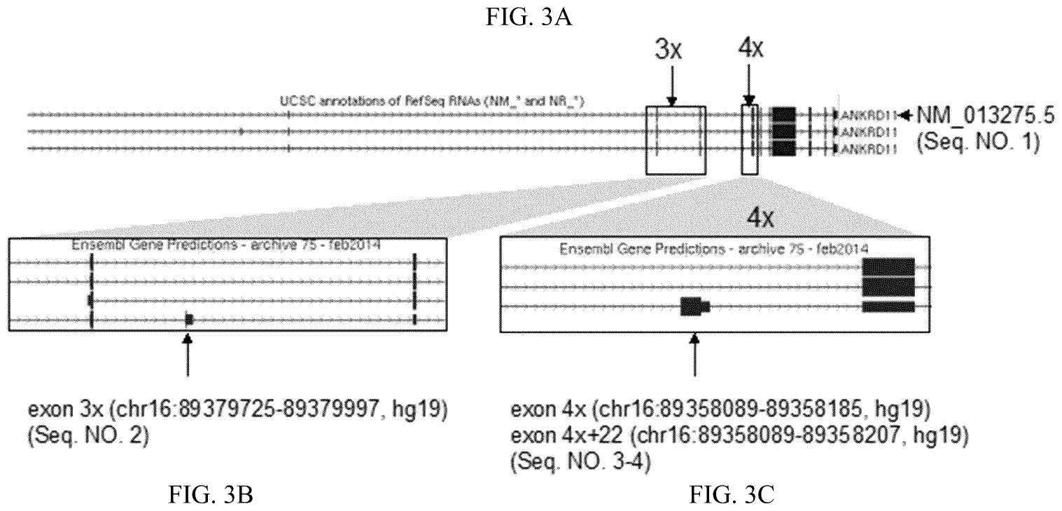

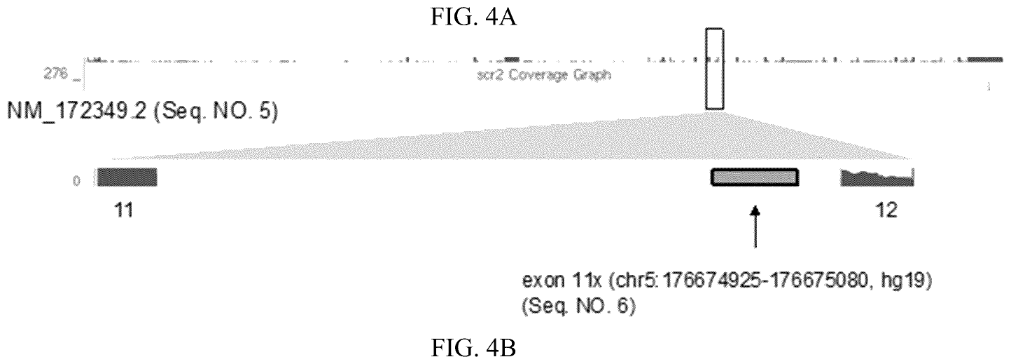

In order to effectively modulate splicing to suppress the unproductive transcript isoform and to increase the functional mRNA and protein level, or to enhance the unproductive transcript isoform and to decrease the functional mRNA and protein level, to an extent that is clinically meaningful, the level of the unproductive transcripts (i.e., percent inclusion of a poison exon) has to be abundant or relatively abundant (for example, >10%, >30% or >50%). As provided herein, the present inventor has identified abundant poison exons in genes known to cause monogenic diseases (especially developmental disorders with autosomal dominant inheritance) that can be clearly targeted by ASOs to effectively restore functional protein production, including ANKRD11 for KBG syndrome (FIG. 3) and NSD1 for Sotos syndrome, reverse Sotos syndrome, normal and pathological aging, and cancer (FIG. 4).

In various embodiments, the present disclosure provides an ASO which can target ANKRD11 or NSD1 pre-mRNA transcripts to effectively modulate splicing and thereby upregulate or downregulate functional mRNA and protein expression level. Various regions or sequences on the ANKRD11 or NSD1 pre-mRNA can be targeted by the ASO. In some embodiments, the ASO targets a sequence within an abundant poison exon of an ANKRD11 or NSD1 pre-mRNA transcript. In some embodiments, the ASO targets a sequence upstream (or 5′) from the 5′ end of the poison exon (3′ splice site) of an ANKRD11 or NSD1 pre-mRNA transcript. In some embodiments, the ASO targets a sequence downstream (or 3′) from the 3′ end of the poison exon (5′ splice site) of an ANKRD11 or NSD1 pre-mRNA transcript. In some embodiments, the ASO targets a sequence that is within an intron flanking on the 5′ end of the poison exon of an ANKRD11 or NSD1 pre-mRNA transcript. In some embodiments, the ASO targets a sequence that is within an intron flanking the 3′ end of the poison exon of an ANKRD11 or NSD1 pre-mRNA transcript. In some embodiments, the ASO targets a sequence comprising the poison exon-intron boundary of an ANKRD11 or NSD1 pre-mRNA transcript. A poison exon-intron boundary can refer to the junction of an intron sequence and the poison exon region. The intron sequence can flank the 5′ end of the poison exon, or the 3′ end of the poison exon. In some embodiments, the ASO targets a sequence within the exon of an ANKRD11 or NSD1 pre-mRNA transcript. In some embodiments, the ASO targets a sequence within an intron of an ANKRD11 or NSD1 pre-mRNA transcript. In some embodiments, the ASO targets a sequence comprising both a portion of an intron and a portion of the exon of an ANKRD11 or NSD1 pre-mRNA transcript.

In an embodiment, an abundant poison exon is selected from exon 3× of ANKRD11, exon 4× of ANKRD11, and exon 11× of NSD1 (FIGS. 3A-3C and 4A-4B). In some embodiments, the ASO targets a sequence from about 1 to about 1500 nucleotides upstream (or 5′) from the 5′ end of the poison exon. In some embodiments, the ASO targets a sequence from about 1 to about 50 nucleotides, about 50 to about 100 nucleotides, about 100 to about 200 nucleotides, about 200 to about 500 nucleotides, about 500 to about 1000 nucleotides, or about 1000 to about 1500 nucleotides upstream (or 5′) from the 5′ end of the poison exon region. In some embodiments, the ASO targets a sequence more than 1500 nucleotides upstream (or 5′) from the 5′ end of the poison exon. In some embodiments, the ASO targets a sequence from about 1 to about 1500 nucleotides downstream (or 3′) from the 3′ end of the poison exon. In some embodiments, the ASO targets a sequence from about 1 to about 50 nucleotides, about 50 to about 100 nucleotides, about 100 to about 200 nucleotides, about 200 to about 500 nucleotides, about 500 to about 1000 nucleotides, or about 1000 to about 1500 nucleotides downstream from the 3′ end of the poison exon. In some embodiments, the ASO targets a sequence more than 1500 nucleotides downstream from the 3′ end of the poison exon.

In some embodiments, the ANKRD11 poison exon containing pre-mRNA transcript is encoded by a genetic sequence with at least about 80%, 85%, 90%, 95%, 96%, 97%, 98%, 99% or 100% sequence identity to SEQ ID NO. 1.

In some embodiments, the NSD1 poison exon containing pre-mRNA transcript is encoded by a genetic sequence with at least about 80%, 85%, 90%, 95%, 96%, 97%, 98%, 99% or 100% sequence identity to SEQ ID NO. 5.

In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream (or 5′) from the 5′ end of exon 3× of ANKRD11, exon 4× of ANKRD11, or exon 11× of NSD1. In some embodiments, the ASO targets a sequence about 1500 nucleotides, about 1000 nucleotides, about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream (or 3′) from the 3′ end of exon 3× of ANKRD11, exon 4× of ANKRD11, or exon 11× of NSD1.

In some embodiments, the ASO has a sequence complementary to the targeted portion of the poison exon-containing pre-mRNA according to any one of SEQ ID nOs: 2, 3, 4, and 6.

In some embodiments, the ASO targets a sequence upstream from the 5′ end of the poison exon. For example, the ASO targeting a sequence upstream from the 5′ end of exon 3× of ANKRD11, exon 4× of ANKRD11, or exon 11× of NSD1 comprises a sequence that is at least about 80%, 85%, 90%, 95%, 97%, or 100% complimentary to at least 8 contiguous nucleic acids of any one of SEQ ID nOs: 2, 3, 4, and 6. In some embodiments, the ASO targets a sequence downstream from the 3′ end of an poison exon. For example, the ASO targeting a sequence downstream from the 3′ end of exon 3× of ANKRD11, exon 4× of ANKRD11, or exon 11× of NSD1 comprises a sequence that is at least about 80%, 85%, 90%, 95%, 97%, or 100% complimentary to at least 8 contiguous nucleic acids of any one of SEQ ID nOs: 2, 3, 4, and 6.

In some embodiments, the ASO targets a sequence within a poison exon.

In some embodiments, the methods described herein are used to increase or decrease the production of a functional NSD1 or ANKRD11 mRNA or protein. As used herein, the term “functional” refers to the amount of activity or function of a NSD1 or ANKRD11 mRNA or protein that is necessary to eliminate any one or more symptoms of a monogenic disorder or other disease conditions, such as KBG syndrome, Sotos syndrome, reverse Sotos syndrome, normal and pathological aging, and cancer. Embodiments of the methods described herein can modulate splicing of poison exons using the ASO and, thereby, reduce the level of the unproductive transcript isoforms and upregulate functional mRNA and protein products. The ASO can target particular exons in alternatively spliced pre-mRNAs to suppress poison exons and, thereby, increase functional mRNA and protein production for treatment of disease conditions caused by protein deficiency including haploinsufficiency. The ASO can also target particular exons in alternatively spliced pre-mRNAs to enhance poison exons and, thereby, decrease functional mRNA and protein production for treatment of disease conditions caused by protein overexpression or gain of toxic function.

In an embodiment, the present disclosure provides compositions and methods for modulating alternative splicing of ANKRD11 or NSD1, to increase or decrease the production of protein-coding mature mRNA, and thus, translated functional ANKRD11 or NSD1 protein. In an embodiment, the compositions and methods can be useful for treating a disease condition. The disease condition can be caused by deficiency of protein function, such as haplo-insufficiency, or gain of toxic function.

In an embodiment, a method of treating a monogenic disorder can include administering a pharmaceutically effective amount of a therapeutic agent for modulating unproductive alternative splicing to a patient in need thereof. In an embodiment, the disease condition is selected from KBG syndrome, Sotos syndrome, reverse Sotos syndrome, normal and pathological aging, and cancer. The therapeutic agent can target an exon selected from exon 3× of ANKRD11 (e.g., between canonical exons 3 and 4), exon 4× (e.g., between canonical exons 4 and 5) of ANKRD11, and exon 11× of NSD1 (e.g., between canonical exons 11 and 12). In an embodiment, the monogenic disorder is KBG syndrome, and the therapeutic agent targets an exon selected from exon 3× and exon 4× of ANKRD11. In an embodiment, the monogenic disorder is Sotos syndrome and the therapeutic agent targets exon 11× of NSDL. The exon numbering is based on the ANKRD11 isoform sequence in reference to NM_013275.5 and NSD isoform sequence in reference to NM_172349.2. It is understood that the exon numbering may change in reference to a different ANKRD11 or NSD1 isoform sequence. One of skill in the art can determine the corresponding exon number in any isoform based on the exon sequences provided herein or using the number provided in reference to the mRNA sequence at NM_013275.5 for ANKRD11 or NM_172349.2 for NSD1. One of skill in the art also can determine the sequences of flanking introns in any ANKRD11 or NSD1 isoform for targeting using the methods described herein, based on an exon sequence provided herein or using the exon number provided in reference to the mRNA sequence at NM_013275.5 for ANKRD11 or NM_172349.2 for NSD1. In an embodiment, the therapeutic agent includes an antisense oligomer (ASO) to modulate splicing of the poison exon of choice, or multiple ASOs to modulate splicing of one or more poison exons of choice. The therapeutic agent can reduce the level of unproductive transcript isoforms and upregulate functional mRNA and protein products.

An embodiment of the present disclosure provides a method of increasing or decreasing expression of a target protein by cells having a pre-mRNA that comprises a poison exon and encodes the target protein. The method can include contacting the cells with an antisense oligomer (ASO) complementary to a targeted portion of the pre-mRNA. In an embodiment, the target protein is selected from the group consisting of ANKRD11 and NSD1. In an embodiment, the targeted portion of the pre-mRNA is selected from exon 3× of ANKRD11 (between canonical exons 3 and 4) and exon 4× (between canonical exons 4 and 5) of ANKRD11. In an embodiment, the targeted portion of the pre-mRNA includes exon 11× (between canonical exons 11 and 12) of NSD1.

According to an embodiment, a method of treating a disease condition in a subject in need thereof can include increasing expression of a target protein by cells of the subject that have a pre-mRNA that comprises a poison exon and encodes the target protein. The cells of the subject can be contacted with an antisense oligomer (ASO) complementary to a targeted portion of the pre-mRNA encoding the target protein. In an embodiment, the target protein is selected from the group consisting of ANKRD11 and NSD1. In an embodiment, the targeted portion of the pre-mRNA includes exon 11× (between canonical exons 11 and 12) of NSD1. In an embodiment, the targeted portion of the mRNA is selected from exon 3× of ANKRD11 (between canonical exons 3 and 4) and exon 4× (between canonical exons 4 and 5) of ANKRD11. In an embodiment, the targeted portion of the mRNA is selected from exon 3× of ANKRD11 (between canonical exons 3 and 4) and exon 4× (between canonical exons 4 and 5) of ANKRD11 and the monogenic disorder is KBG syndrome. In an embodiment, the targeted portion of the mRNA includes exon 11× of NSD1 (between canonical exons 11 and 12) and the monogenic disorder is Sotos syndrome.

The present inventor identified abundant poison exons in genes known to cause monogenic diseases and additional disease conditions that can be targeted by ASOs to effectively restore functional mRNA and protein production, including ANKRD11 for KBG syndrome and NSD1 for Sotos syndrome, reverse Sotos syndrome, normal and pathological aging, and cancer.

Through systematic analysis using RNA sequencing in a large panel of human tissues and cells across different conditions using bioinformatics algorithms (Yan, Q., et al. 2015. “Systematic discovery of regulated and conserved alternative exons in the mammalian brain reveals NMD modulating chromatin regulators.” Proc Natl Acad Sci USA 112: 3445-3350), in combination with additional validation by RT-PCR and RT-qPCR, the present inventor has identified two abundant poison exons, exons 3× and 4× in the ANKRD11 gene (FIGS. 3A-3B), and one abundant poison exon 11× in the NSD1 gene (FIG. 4A-4B), as druggable candidates (exon numbering follows Refseq NM_013275 or ANKRD11 and NM_172349 for NSD1). For the ANKRD11 gene, the apparent exon 4× inclusion level is estimated to be up to 43% and the apparent exon 3× inclusion level is up to 24%. The NSD1 poison exon has an estimated inclusion level up to 65%. The highest level of poison exon inclusion is frequently observed upon inhibition of the RNA degradation pathway in certain conditions. Since inhibition of RNA degradation pathways is not complete, the actual abundance of the unproductive isoform is likely higher, making them promising candidates to be targeted by ASOs to restore functional ANKRD11 or NSD1 protein production. We note for each of these two genes, there are multiple poison exons which were estimated to have low abundance (<10%) (Yan, Q., et al. 2015. “Systematic discovery of regulated and conserved alternative exons in the mammalian brain reveals NMD modulating chromatin regulators.” Proc Natl Acad Sci USA 112: 3445-3350), and they are unlikely drug target to bring clinically meaningful upregulation of the targeted mRNA and protein.

In some embodiments, the antisense oligomer is at least 80%, at least 85%, at least 90%, at least 95%, at least 98%, at least 99%), or 100%, complementary to the targeted portion of the pre-mRNA.

In some embodiments, the subject is a human. In some embodiments, the subject is a non-human animal. In some embodiments, the subject is a fetus, an embryo, or a child. In some embodiments, the cell is in a subject. In some embodiments, the cells are ex vivo. In some embodiments, the cell is in vitro (e.g., in cell culture).

Provided herein is a composition comprising an antisense oligomer (ASO) that induces exon skipping or inclusion by binding to a targeted portion of the ANKRD11 or NSD1 pre-mRNA containing a poison exon. As used herein, the terms “ASO”, “antisense oligonucleotide” and “antisense oligomer” are used interchangeably and refer to an oligomer such as a polynucleotide, comprising nucleobases that hybridizes to a target nucleic acid (e.g., poison exon containing pre-mRNA) sequence by Watson-Crick base pairing or wobble base pairing (G-U). The ASO may have exact sequence complementary to the target sequence or near complementarity (e.g., sufficient complementarity to bind the target sequence and modulating splicing). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited.

In some embodiments, ASOs “specifically hybridize” to or are “specific” to a target nucleic acid or a targeted portion of a pre-mRNA containing a poison exon. Typically, such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) needs not be 100% complementary to that of its targeted portion of the nucleic acid to hybridize. In certain embodiments, ASOs can comprise at least 70%, at least 75%, at least 80%, at least 85%, at least 90%, at least 95%, at least 96%), at least 97%, at least 98%, or at least 99% sequence complementarity to a targeted portion within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using sequence alignment programs, such as BLAST (basic local alignment search tools) and PowerBLAST, known in the art (Altschul, et al, J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

An ASO described herein may comprise nucleobases of RNA or DNA moieties in which only a portion of its nucleobases hybridize to the target sequence. For example, the ASO can be in the form of a circular DNA or RNA.

The ASOs described herein comprise nucleobases that are complementary to nucleobases present in a target portion of a poison exon-containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art.

One or more nucleobases of an ASO may be any naturally occurring, unmodified nucleobase such as adenine, guanine, cytosine, thymine, and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoyl cytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3-5′ phosphodiester linkage connecting sugar moieties of the oligomer. The backbone structure or oligomer linkages of the ASOs described herein may include (but are not limited to) phosphorothioate (PS), phosphorodithioate, phosphoroselenoate, phosphorodiselenoate, phosphoroanilothioate, phosphoraniladate, phosphoramidate, and the like. In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphorothioate linkage. In some embodiments, the backbone modification is a phosphoramidate linkage.

In some embodiments, the stereochemistry at each of the phosphorus internucleotide linkages of the ASO backbone is random. In some embodiments, the stereochemistry at each of the phosphorus internucleotide linkages of the ASO backbone is controlled and is not random. In some embodiments, a composition used in the methods of the disclosure comprises an ASO that has diastereomeric purity of at least about 90%, at least about 91%, at least about 92%, at least about 93%), at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability. In some embodiments, an ASO used in the methods of the disclosure, including, but not limited to, any of the ASOs, comprises about 5-100%>Rp, at least about 5%>Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-0 ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.” In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA).

Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides (2′MOE-PS). ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein.

Methods of synthesizing ASOs will be known to one of skill in the art. Alternatively or in addition, ASOs may be obtained from a commercial source. Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., while a nucleotide that is directly adjacent and downstream of the reference point is designated “plus one.”

In some embodiments, two or more ASOs with different chemistries but complementary to the same targeted portion of the poison exon-containing pre-mRNA are used. In some embodiments, two or more ASOs that are complementary to different targeted portions of the poison exon-containing pre-mRNA are used.

In some embodiments, the antisense oligonucleotides of the disclosure are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine, or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptide, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base, or phosphate group, as understood in the art and described in the literature, e.g., using a linker. Linkers can include a bivalent or trivalent branched linker. In embodiments, the conjugate is attached to the 3′ end of the antisense oligonucleotide.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the poison exon (e.g., a portion of sequence located upstream of the target/included exon) to approximately 100 nucleotides downstream of the 5′ splice site of the target/included exon (e.g., a portion of sequence of the exon located downstream of the target/included exon). For example, a first ASO of 20 nucleotides in length may be designed to specifically hybridize to nucleotides −100 to −81 relative to the 3′splice site of the target/included exon. A second ASO may be designed to specifically hybridize to nucleotides −95 to −76 relative to the 3′splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site. In some embodiments, the ASO can target a sequence within the poison exon. In some embodiments, the ASO can target a sequence can span the exon-intron boundaries. In some embodiments, the ASOs can be tiled from about 500 nucleotides upstream of the 3′splice site of the exon, to about 500 nucleotides downstream of the 5′splice site of the exon. In some embodiments, the ASOs can be tiled from about 1000 nucleotides upstream of the 3′splice site of the exon, to about 1000 nucleotides downstream of the 5′ splice site of the exon.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping.

ASOs that when hybridized to a region of a pre-mRNA result in exon skipping and increased mRNA and protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (e.g., efficiency, rate, extent) and protein production by methods known in the art and described herein. The animal models may also be any phenotypic or behavioral indication of the disease or disease severity.

The ASOs described herein can encompass any pharmaceutically acceptable salts, esters, or salts of such esters, or any other compound which, upon administration to an animal including a human, is capable of providing (directly or indirectly) the biologically active metabolite or residue thereof. The ASOs may also be admixed, encapsulated, conjugated, or otherwise associated with other molecules, molecule structures or mixtures of compounds, as for example, liposomes, receptor targeted molecules, oral, rectal, topical or other formulations, for assisting in uptake, distribution and/or absorption.

A pharmaceutical composition for treating monogenic disorders can include the ASO and a pharmaceutically acceptable carrier. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives known in the art may be included, for non-limiting examples, starches, sugars, diluents, granulating agents, lubricants, binders, disintegrating agents, and the like.

The pharmaceutical compositions may be administered in any number of ways depending upon whether local or systemic treatment is desired and upon the area to be treated. In some embodiments, the pharmaceutical composition is administered by intrathecal injection, intracerebroventricular injection, intraperitoneal injection, intramuscular injection, subcutaneous injection, intravitreal, or intravenous injection of the subject.

The composition can be presented in a form suitable for administration with a frequency as needed depending on the disease (for example, daily, weekly, monthly, or once every four months). The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the therapeutic agent or an amount effective to treat a disease, such as monogenic disease caused by haploinsufficiency, may be determined initially using standard approaches known to the art, and adjusted for specific targeted diseases in specific patients.

The present teachings are illustrated by the following examples.

### Example 1

Increase of SMN2 Exon 7 Inclusion Using ASOs Targeting Intronic Splicing Regulatory Element to Increase Full-Length SMN2 mRNA Level

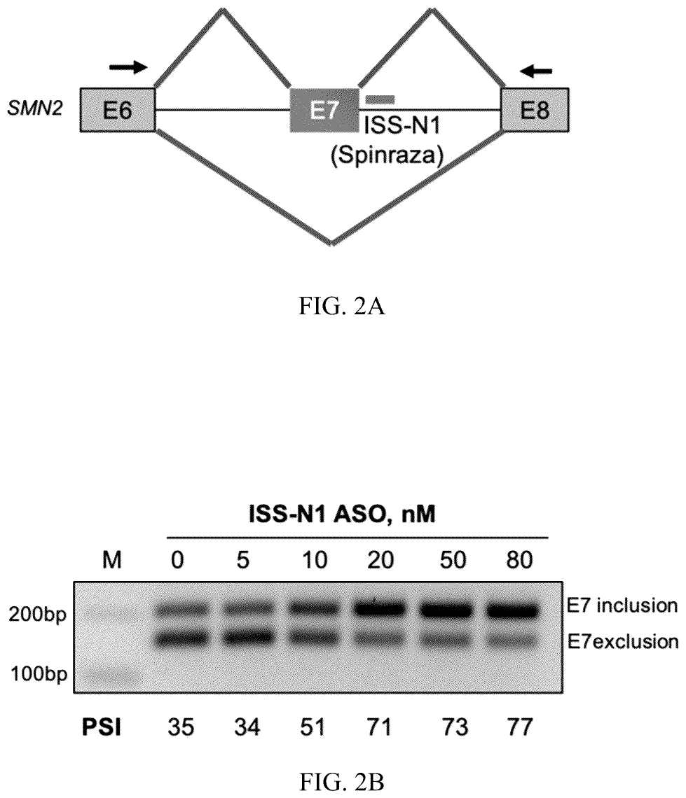

To illustrate the current art of using ASO to modulate pre-mRNA splicing and increase the production of functional mRNA and protein, we used ASO to target a sequence within intron 7 of SMN2 (the same ASO sequence as Spinraza® brand nusinersen, an FDA approved drug to treat spinal muscular atrophy) to increase exon 7 inclusion (FIGS. 2A-2B). Dose-dependent increase of exon inclusion was observed as measured by RT-PCR, which was similar to observations reported in the literature (Hua Y., Vickers T. A., Okunola H. L., Bennett C. F., Krainer A. R. 2008. “Antisense masking of an hnRNP A1/A2 intronic splicing silencer corrects SMN2 splicing in transgenic mice.” Am J Hum Genet 82: 834-848).

### Example 2

### Identification of Abundant Poison Exons in ANKRD11 and NSD1

Through systematic analysis of RNA-seq data in a large panel of human tissues and cells across different conditions using bioinformatics algorithms (Yan, Q., et al. 2015. “Systematic discovery of regulated and conserved alternative exons in the mammalian brain reveals NMD modulating chromatin regulators.” Proc Natl Acad Sci USA 112: 3445-3350), in combination with validation by RT-PCR, the present inventor has identified two abundant poison exons, exons 3× and 4× in the ANKRD11 gene (FIGS. 3A-3B), and one abundant poison exon 11× in the NSD1 gene (FIG. 4A-4B), as druggable candidates (exon numbering follows Refseq NM_013275). For the ANKRD11 gene, the apparent exon 4× inclusion level is estimated to be up to 43% and the apparent exon 3× inclusion level is up to 24%. The NSD1 poison exon has an estimated inclusion level up to 65%. The highest level of poison exon inclusion is frequently observed upon inhibition of the RNA degradation pathway in certain conditions. Since inhibition of RNA degradation pathways is not complete, the actual abundance of the unproductive isoform is likely higher, making them promising candidates to be targeted by ASOs to restore functional ANKRD11 or NSD1 protein production. We note for each of these two genes, there are multiple poison exons which were estimated to have low abundance (<10%) (Yan, Q., et al. 2015. “Systematic discovery of regulated and conserved alternative exons in the mammalian brain reveals NMD modulating chromatin regulators.” Proc Natl Acad Sci USA 112: 3445-3350), and they are unlikely drug target to bring clinically meaningful upregulation of the targeted mRNA and protein.

### Example 3

Inhibition of the Poison Exon Increases Protein-Coding mRNA Level

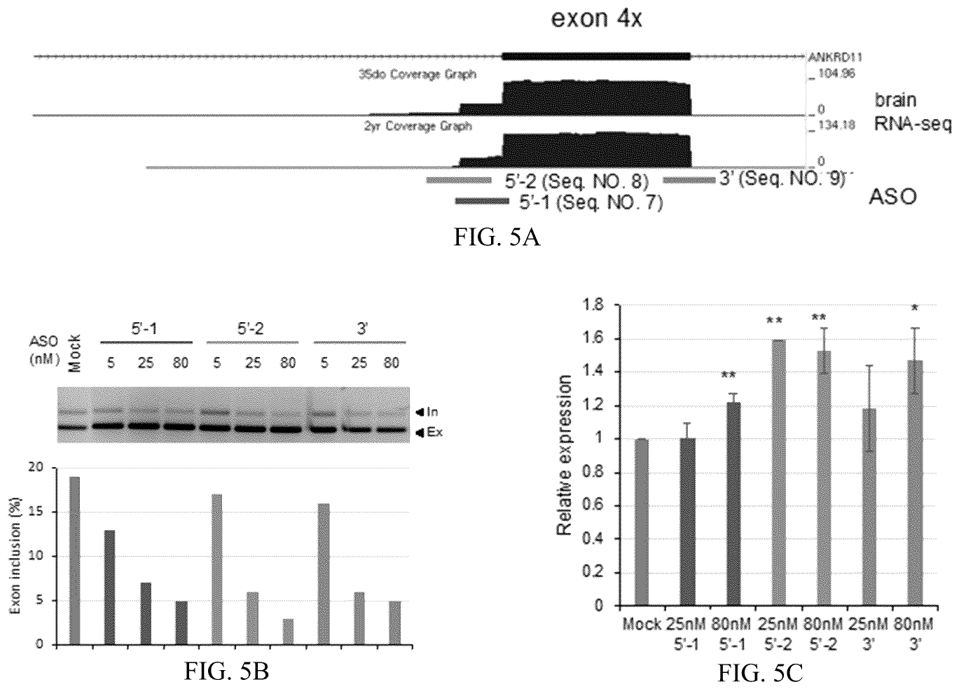

Whether confirm the abundance of the poison exons we identified and test whether they can be inhibited by ASOs, we tested ANKRD11 exon 4× using ASOs targeting the splice sites. Since this exon has two alternative 3′ splice sites, three ASOs with 2′ oMe-PS modifications (IDT) were used, one for each splice site (FIG. 5A; Seq. nOs 7-9). Each ASO was transfected individually at different concentrations into HEK293 cells. After 24 hrs, cells were harvested to examine changes in ANKRD11 splicing and mRNA expression level. For each ASO, three concentrations (5 nM, 25 nM and 80 nM) were tested. All three ASOs inhibited exon inclusion based on RT-PCR analysis (FIG. 5B) and lead to increase of the steady state mRNA level, as measured by RT-qPCR (FIG. 5C), in a dosage-dependent manner. The ASO that overlapped with both 3′ splice sites (Seq. NO. 8) achieved the best performance and resulted in 1.6-fold increase in mRNA level (FIG. 5C), confirming the promise of the disclosed method.

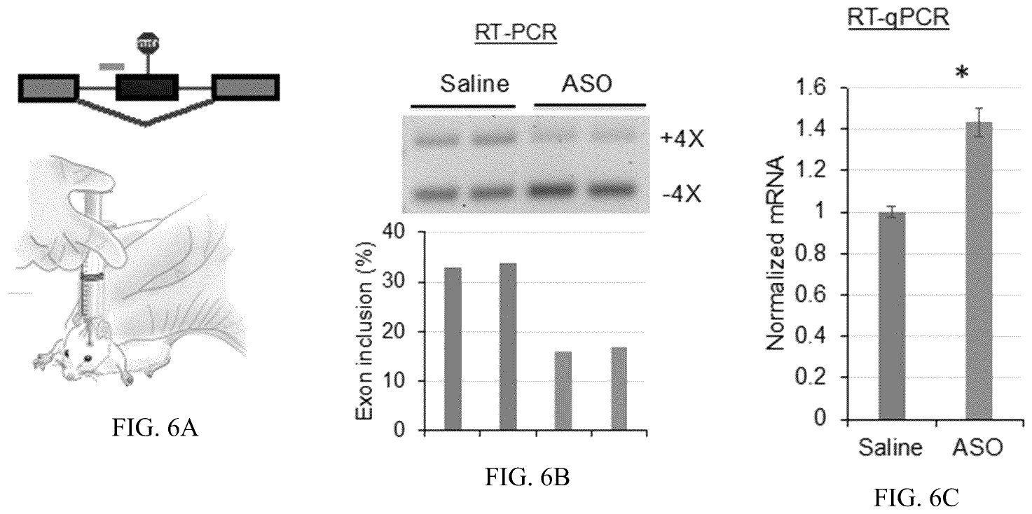

To further validate the identified ASO in upregulating ANKRD11 expression in vivo, we performed intracerebroventricular (ICV) injection of the ASO that overlapped with both 3′ splice sites (ASO sequence: 5′-GCATCTAAAGGCATCAACACAGAGCACTAA-3; with 2′MOE-PS chemistry; this sequence is one nucleotide different at position 7 from the human version Seq. NO 8) with 2′ MOE-PS chemistry at 50 g into neonatal (P2) mouse brain (FIG. 6A). Injection of saline was used for control. Cortex tissues were collected and analyzed for Ankrd11 RNA and protein abundance at P9. Compared to saline control, ASO treatment resulted in significant reduction of exon 4× from 33% to 17% and increase of Ankrd11 protein-coding mRNA for 1.4-fold (FIG. 6C).

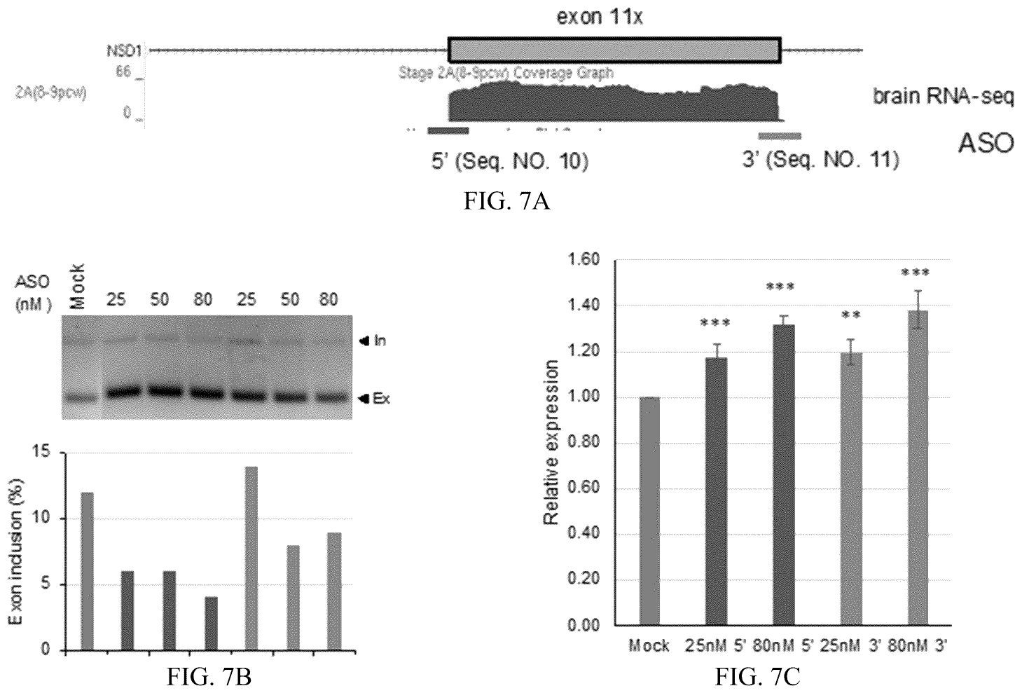

Similarly, two 2′oMe-PS ASOs targeting the splice sites of NSD1 poison exon 11× were tested to inhibit inclusion of the poison exon in NSD1 pre-mRNA (FIG. 7A; Seq. NO. 10-11). We also observed dose-dependent skipping of the poison exon (FIG. 7B), and consistent increase of NSD1 mRNA level up to ˜1.4 fold (FIG. 7C; Seq. NO. 11) upon ASO treatment.

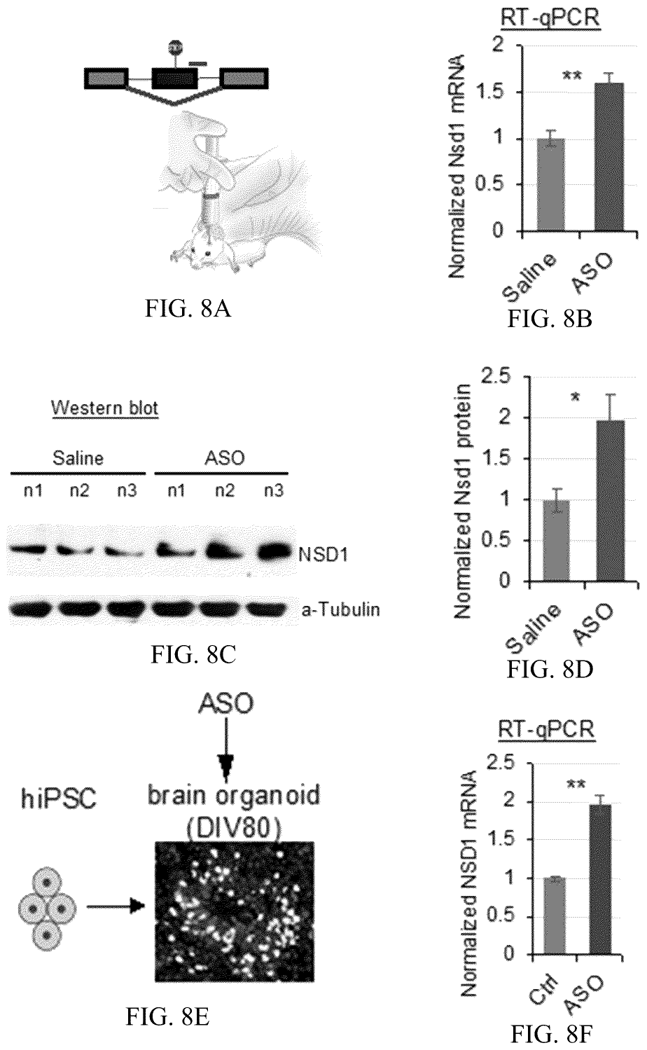

We performed in vivo validation of Nsd1 mRNA and protein upregulation by injecting the ASO targeting the 5′ splice site (Seq. NO 11; 2′MOE-PS chemistry) into neonatal (P2) mouse brain. For this experiment, we took advantage of the fact that the ASO target sequence is conserved between human and mouse. Wild type mice at postnatal day 2 (P2) were treated with a single dose of 25 μg ASO or saline through ICV injection (FIG. 8A). Seven days after injection, cortex tissues were harvested for biochemical analysis. We observed 1.6-fold increase in Nsd1 mRNA upon ASO injection by RT-qPCR (FIG. 8B), and 2-fold increase in NSD1 protein by Western blots (FIG. 8C,D).

We also tested ASO-mediated NSD1 upregulation in brain organoids differentiated from human iPSCs (FIG. 8E). In this experiment, organoids treated with ASO (Seq. NO 11; 2′MOE-PS chemistry) by free uptake at 20 μM resulted in robust upregulation of NSD1 mRNA up to 2-fold (FIG. 8F).

### Example 4

### Design of ASO Walk and Microwalk to Screen Candidate ASOs

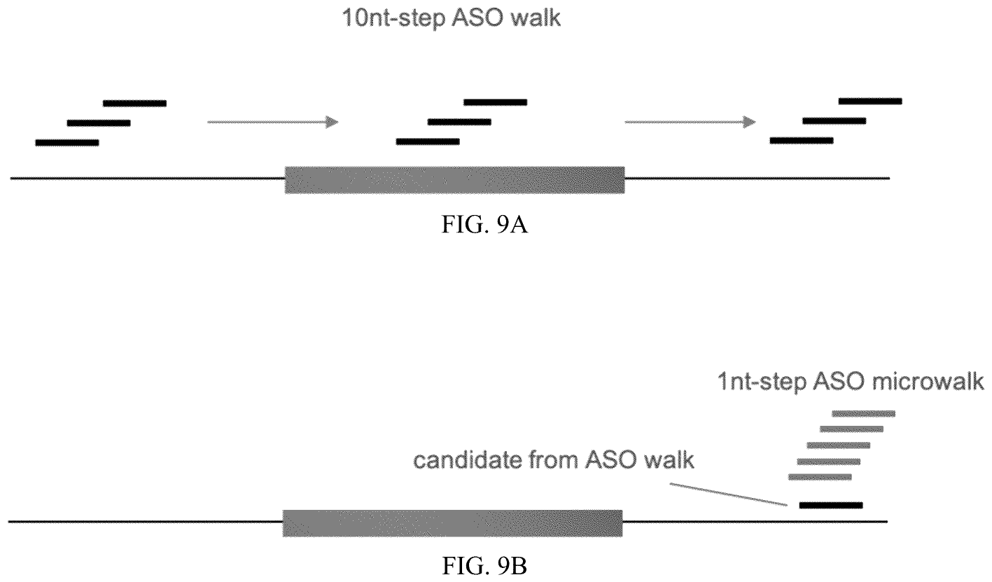

Similar to previous studies (Hua et al., 2007; Hua et al., 2008), an ASO walk strategy may be used to identify additional ASOs that can inhibit the inclusion and determine the optimal ASOs for further clinical development. Specifically, for each poison exon, a panel of 20-nt ASOs will be designed to target the alternative exon and flanking intronic sequences (for example, from −100 nt upstream of the 3′ splice site of the poison exon to 100 nt downstream of the 5′ splice site of the poison exon) at 10 nt steps (FIG. 9A). Once regulatory regions are identified, a second “microwalk” of 1-nt step, as well as ASOs of different sizes, can be performed (FIG. 9B). Following a standard approach, each ASO can be introduced into 3-6×10⁵ HEK293T cells (embryonic kidney origin; by transfection at 80 nM) or by gymnotic (free) uptake; 20 μM) (Han et al., 2020; Lim et al., 2020). Non-targeting (scrambled) or no ASO controls will also be included as controls. Cells treated with ASOs for 24 hrs will be harvested for RT-PCR/SDS-PAGE to quantify splicing and qPCR to quantify mRNA abundance; protein levels will be confirmed for representative ASOs by Western blots using specific antibodies.

### Example 5

### ASO Walk to Screen Candidate ASOs for ANKRD11 Upregulation or Down-Regulation

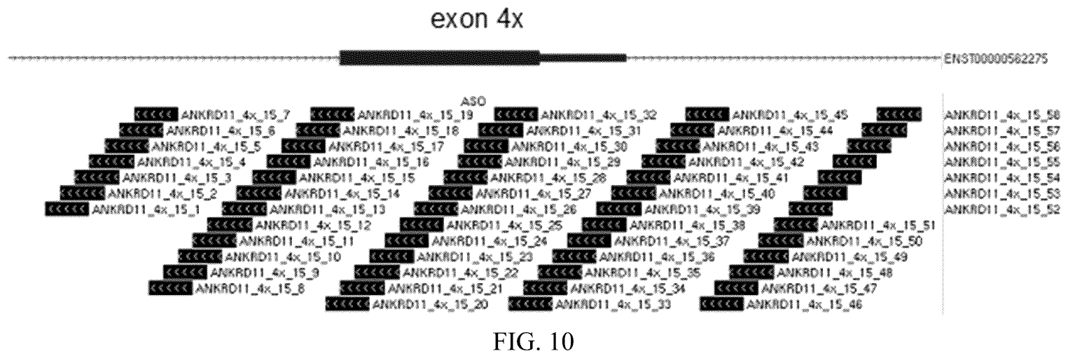

Following the general guidelines as described in Example 4, we performed ASO walk to systematically screen ASOs that are most effective in modulating ANKRD11 exon 4× splicing and ANKRD11 expression. We designed and synthesized a panel of 15 nt ASOs with 2′ MOE-PS chemistry that target exon 4× or flanking intronic sequences (FIG. 10; Seq. NO 12-69). Cell line HEK 293T was used to screen ASOs. Cells were transfected with individual ASOs at day 0 with Lipofectamine. Treated cells were harvested after 48 h and RNA was extracted. RT-PCR was performed to quantify ANKRD11 exon 4× inclusion.

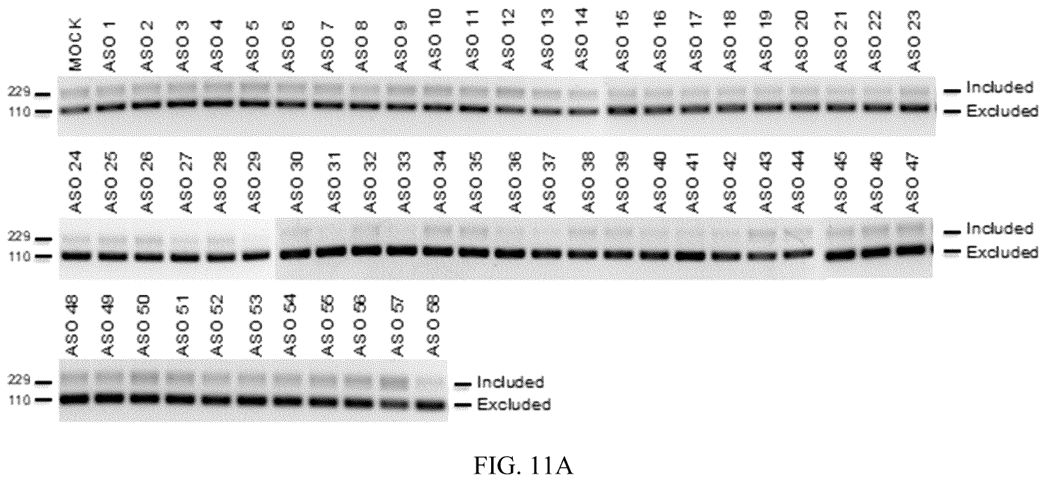

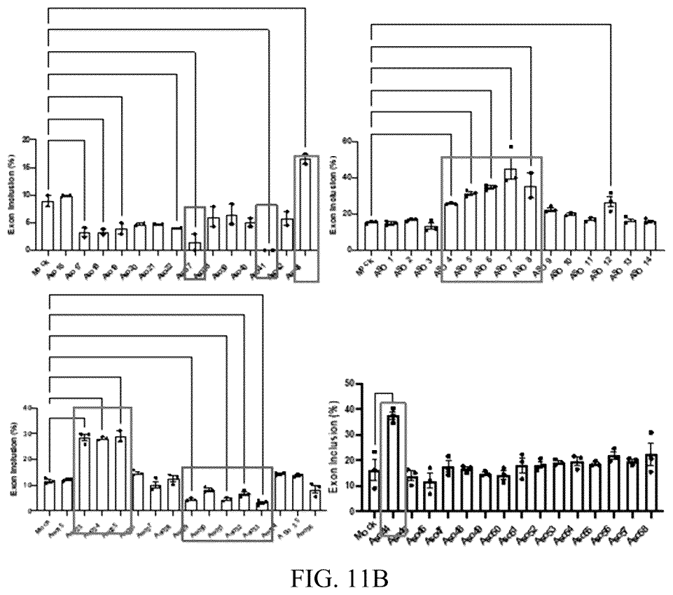

As shown in FIGS. 11A and 11B, ASOs 29-33, 37 and 41, corresponding to Seq. NO 40-44, 48 and 52, are most effective in decreasing exon 4× inclusion (and thus upregulation of ANKRD11 mRNA and protein). ASOs 4-8 and 43-44, corresponding to Seq. NO 15-19 and 54-55 are most effective in increasing exon 4× inclusion (and thus down-regulation of ANKRD11 mRNA and protein).

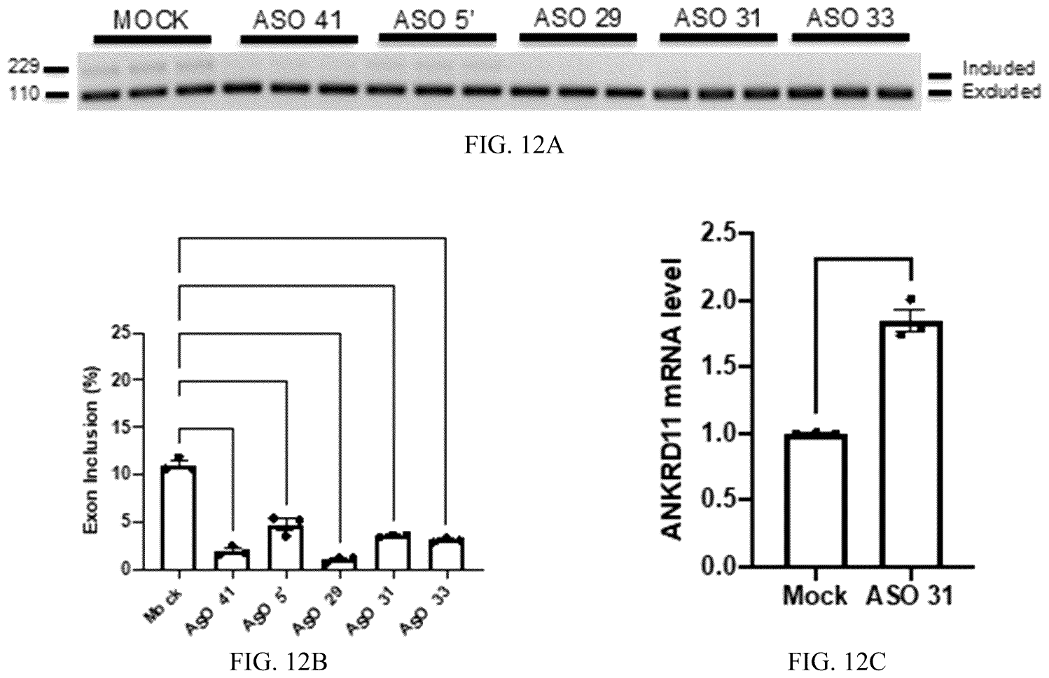

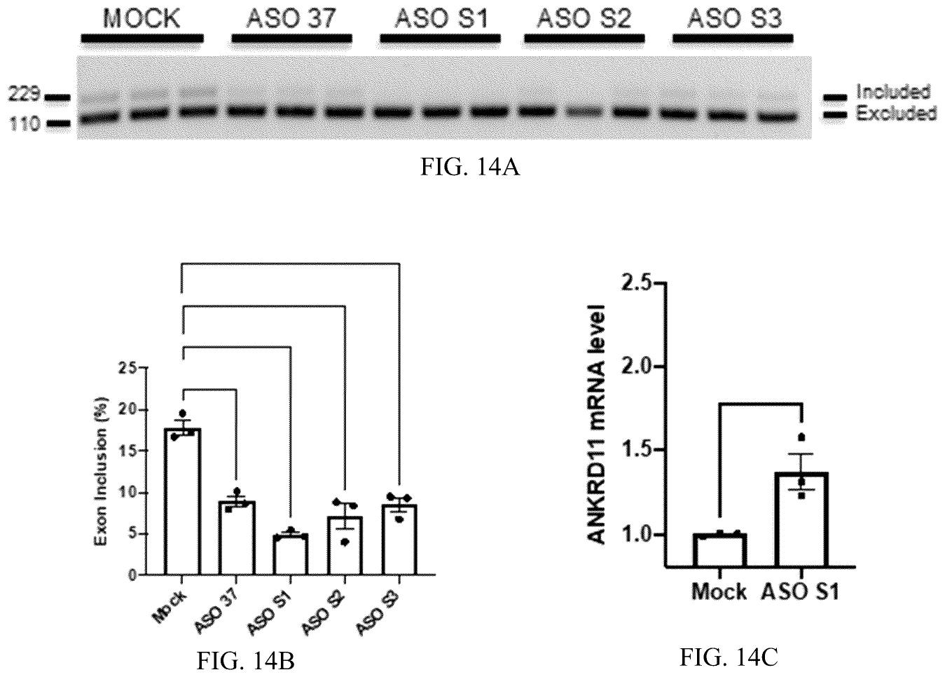

Skipping of ANKRD11 exon 4× by ASOs 29, 31, 33, 37 and 41, corresponding to Seq. NO 40, 42, 44, 48 and 52 was further validated by additional independent experiments (FIGS. 12A, 12B, 14A and 14B.

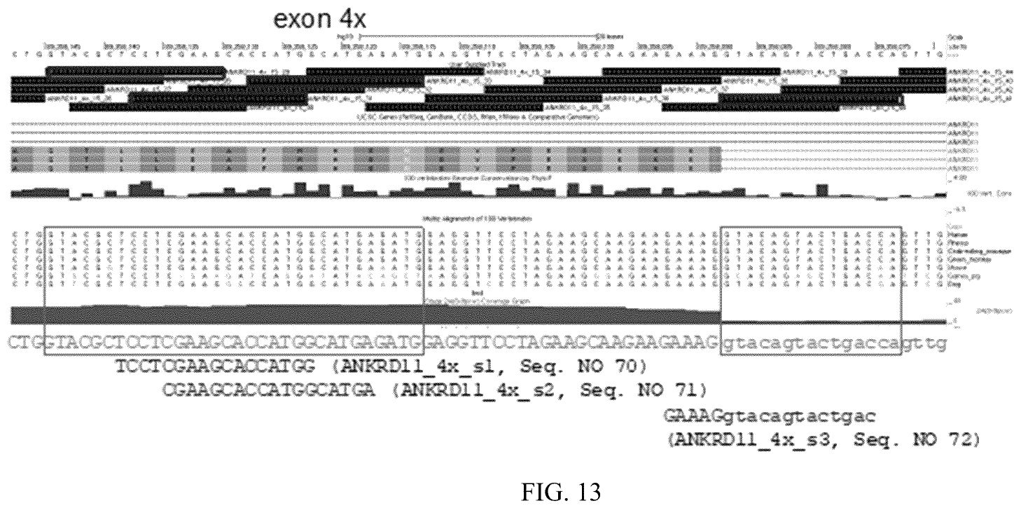

Based on the screening results, we identified two major regions that contain splicing-regulatory sequences that are important for exon 4× inclusion: one overlapped with ASO sequences 29-33 and the other targeted by ASO 41. To facilitate pre-clinical studies using model organisms, we designed three additional ANKRD11 ASOs S1-53, corresponding to Seq. NO 70-72 (FIG. 13). These ASOs were also able to skip exon 4× (FIGS. 14A and 14B).

Finally, we tested upregulation of ANKRD11 mRNA by qPCR. We confirmed that cells treated with ANKRD11 ASO 31 (Seq. NO 42) and S1 (Seq. NO 70) are able to upregulate ANKRD11 mRNA for 1.8- and 1.4-fold, respectively (FIGS. 12C and 14C).

### Example 6

### ASO Walk to Screen Candidate ASOs for NSD1 Upregulation or Down-Regulation

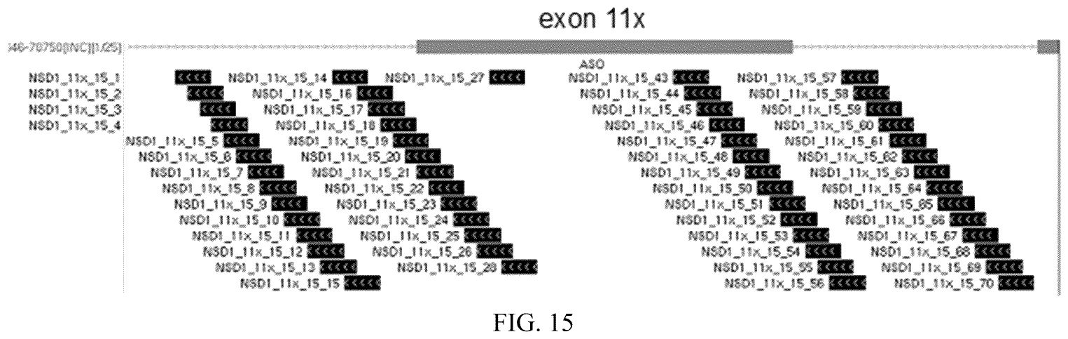

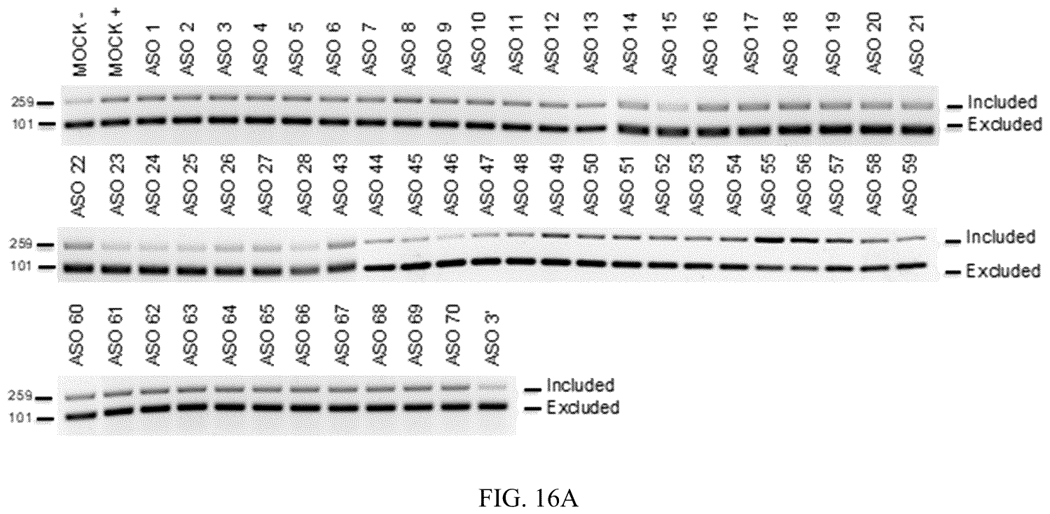

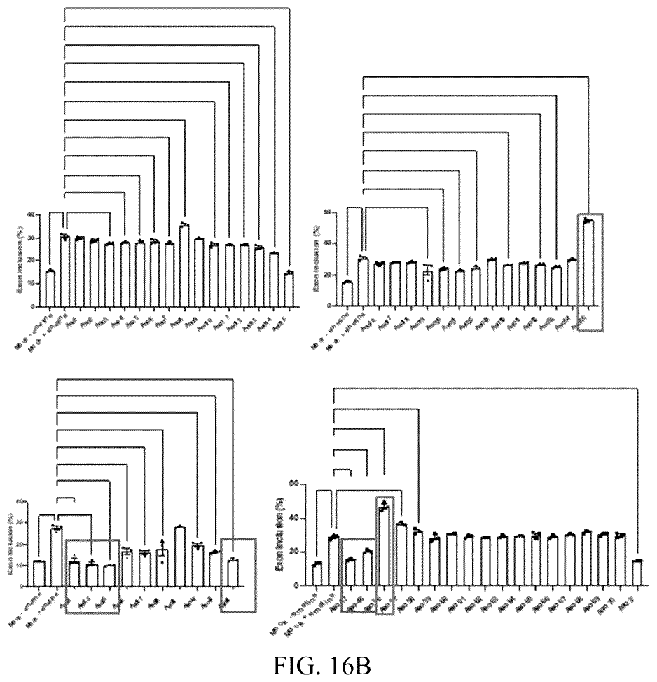

Following the general guidelines as described in Example 4, we performed ASO walk to systematically screen ASOs that are most effective in modulating NSD1 exon 11× splicing and NSD1 expression. We designed and synthesized a panel of 15 nt ASOs with 2′ MOE-PS chemistry that target exon 4× or flanking intronic sequences (FIG. 15; Seq. NO 73-128). Cell line BEK 293T was used to screen ASOs. Cells were transfected with individual ASOs at day 0 with Lipofectamine. 43 hrs after transfection, cells were treated with emetine to inhibit translation and NMD. After another 5 hrs (or 48 hrs after ASO transfection), treated cells were harvested and RNA was extracted. RT-PCR was performed to quantify NSD1 exon 11× inclusion. As shown in FIGS. 16A and 16B, ASOs 23-25 and 46-48, corresponding to Seq. NO 95-97 and 104-106, are most effective in decreasing exon 11× inclusion (and thus upregulation of NSD1 mRNA and protein). ASOs 55-56, corresponding to Seq. NO 113-114 are most effective in increasing exon 11× inclusion (and thus down-regulation of NSD1 mRNA and protein).

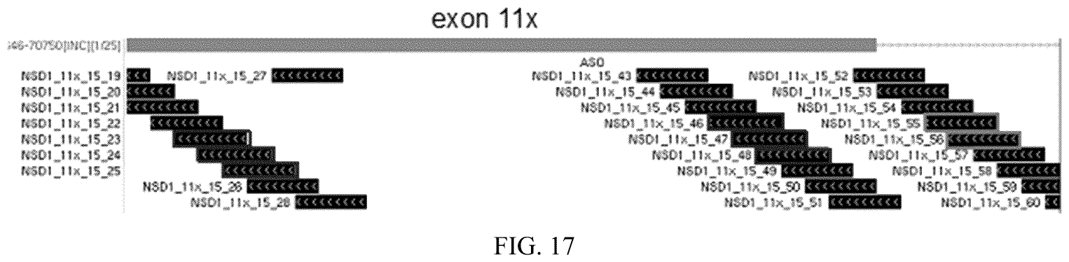

Based on the screening results, we identified two major regions that contain splicing-regulatory sequences that are important for exon 11× inclusion: one overlapped with ASO 23-25 and the other targeted by ASOs 46-48 (FIG. 17).

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

TABLE 1
ANKRD11 pre-mRNA and poison exon sequences.

| Seq. | | Genomic | | |
|---|---|---|---|---|
| ID | Seq Name | Coordinates (hg19) | Strand | Sequence |
| 1 | ANKRD11 pre-mRNA | chr16:89334029- | − | AGAGGCCGCCCTGAGACGGTGCGCGATGGA |
| | (based on | 89556969 | | CCGAGGGCCCCAGCCGGGGAGGCGCCGCCG |
| | NM 013275.5) | | | CCGAGCCCGCGGCCAGACGCCCCATCAGTA |
| | | | | GCGTCCGCACCGGGAGCCGCGGCTCTCGCCC |
| | | | | GAGCCGTGGGCGCGCCCGAGGGGGGGGCTC |
| | | | | GCCTCCCGCCGTCCCTCGCAGCTCTGCCGGG |
| | | | | CCCGAGCCCGCGCCGCCGCCGCCGCCGCCTT |
| | | | | GCCGCTCGGGCCGCGCGGCCCGGGAAACGC |
| | | | | GGCCGCGGGCTGCATGGGCAGCGCCCGCGC |
| | | | | CCCGCCGCTGAGCCGTCGCGGAGCCGCGCA |
| | | | | GCCCTCGGAGCACGGTGAGAGGCGCCGCTG |
| | | | | GTCTGGGGGCGGTGGTCGGGGCGGGCACGG |
| | | | | GGCATTCGCGCGGCCTTGCGGCCTGCAGGCC |
| | | | | TTCCCCGGCGACGGAGCTGCGCCGCGGGCCT |
| | | | | CCGGGCGGGCCTGGGGGGTCGGGGCCGGGT |
| | | | | GGGCGGGGGTCTTTGGGGGCCCGGGGCGAT |
| | | | | CGTGAGGGACCAATAATGGGTCCCGGAGCG |
| | | | | GGCCTACGGGTCCGGGTTCGGGGCAGCCGG |
| | | | | GGGTCTTTGGGGGCCCGGGGTGGCCGTGAG |
| | | | | GGGCCCATAGGGGGCTCCGGGGGGGGCCTG |
| | | | | GGAGGTCGGCGGAGCTTGGGTCGGCCGTAA |
| | | | | GGGGCGGACGGGGGCTCCGGGCGGGCCTGG |
| | | | | GTGGCCGGGGGCCCGCGGCGGCTCTGAGGG |
| | | | | GCCGATTGGGGGCTCCGGGGCGGGCCTGGG |
| | | | | GGCCGTGGGGGCCCGGGGCGGCCGTGAGAG |
| | | | | GCGGACAGGGGGCTCCGGGGCGGGCCTCGG |
| | | | | GGGCTCGGAGCGGCCGTGAGGGGCGGACGG |
| | | | | GGGGCTTCGGGGGGGGCCTGCAGGTCTTGG |
| | | | | GGACCTGAGGCTGCCGGGAGGGGCTGCCAG |
| | | | | GGGGCGCTGGCCGGGCGCCGGGTTCTGCGG |
| | | | | AGCTGGGGCGCCGACCTCTGACCCGCGAGA |
| | | | | GGGGCGCCTTCGCCGTGCTGGTCGTAGTTGT |
| | | | | TATTCTCAGCGTCCCTATTATTATCGCTGTTT |
| | | | | TGAAATGAGAGCAGGCGGCTCTCGGGCTCC |
| | | | | GAGCCGGAGGGGGAGGGCGAACTGGGGACC |
| | | | | TGGGGGCGTCGGGGTTGCAGGAGGCGCGCG |
| | | | | TAGGCCGAGGAGGGGCAGGAATGCGGGCAG |
| | | | | CCGTGTGGGGGGTGTTAGGGGGAGGGTAGG |
| | | | | CGGGCGGGTGTGGGGGGTGGCTGGGAGGAA |
| | | | | AGCGGTGGCGGTGGCGGCTGCAACAGCAGC |
| | | | | CCTTGGCCTCAAGGAACAATGTGAGACGTTG |
| | | | | CCTGAAATGTTAATTTCCGTTCCTCATTTCAT |
| | | | | CATCCCCTGGCAGGGCGGTAGCTGTGTGTGT |
| | | | | GGTGTATGTGTGTGCGCGCGCGCGCGCGCGC |
| | | | | GCGTGTGTGTGTGTGTGTAGGGTTGGCCCTG |
| | | | | CCACATTGATTCAGTCCCCTCTCAAAGAGGG |
| | | | | ATTGTACTGTTAACTCTTGTGTTTGTGTTATT |
| | | | | TGGGAAGGTTGGTCGGGGGGAGTCTTGATTT |
| | | | | TTCTCGAGGCTTGCTCTTTTCCTGGTGCCCCA |
| | | | | TTAAGATTTTCTGCTTCTGTTGTGTTTTTGGA |
| | | | | AGGTTAGTGTTATATATCAGCTTCCAAGAAG |
| | | | | TTTTGGAAGAGGCTTGGGAAGATGAAGCTG |
| | | | | GTCTAACAGCTCCCTATGCTTTGAAACTGTT |
| | | | | TTCCTTCTATGTAACATGCTTAGGATTCATC |
| | | | | GTTTTTGTAGATTATGAGTAGTTTTGTATCCT |
| | | | | TTTGCAAGAACAGGGTTTTATGGTAGAGAA |
| | | | | ATTAGATTTCAGGCTTTCTTACATGAGGAGA |
| | | | | GAGTTTTATGCAGTCATTAGAGCAATATCCT |
| | | | | TAACACTTGAAATGAGAAATAAAAGTGTGC |
| | | | | ACAGTTGTTGAACTGCAAAACTTAGAGAATC |
| | | | | TTTTGATCCTTTTTGGGATGTTGAGACTTAGC |
| | | | | CAAACATATAAGATGATATTACCTGTGGAA |
| | | | | AAAGTGCCATCGAAACAGTTACTGTTTTTGT |
| | | | | TGTGTGCAGACCTAGTCTATGGCATAACGTT |
| | | | | CAAAATCGGAGACCCTGAGGCTGTTTCTTTG |
| | | | | GTTTCTCTGAGACCTTGATTTCTCTTAGTAAG |
| | | | | TATTGGCTGCCCTGGAAGACTTCGAGTTCTG |
| | | | | TTAGAGAATGATTACAGAAGTCCTCTGATTT |
| | | | | TACTCTGCAACTGTAGTTAAACTAGAAAAAA |
| | | | | AGGGGGGGGGGGGGATGTCTTGCTCAGCCT |
| | | | | TCATTGTGACCCTTGCATTGGATCTCGTATA |
| | | | | GTTGGTGTGGCCTCCCCTACCCTCCATTCAG |
| | | | | CTGTTGTTCTCCTCTTTCTGTAGTTTCTCCCC |
| | | | | GAAGAGCAGGATCTCCTTCGATAGGCTGGC |
| | | | | CATCTGAATGGAACTGGATGGGAGGGAACA |
| | | | | AAGACCAAGTTGCTGTGACGTTTTCTCTGTT |
| | | | | CTTCGTGATGCCTCTTAAGTTTGTTGAAAAG |
| | | | | TTCCCCACAAGATAGTGCTAAATTTGACCTA |
| | | | | AACTTGAAACTTTCAGCAGTTTTTTTCTTCTG |
| | | | | TTCTATTTTTGCTGTAGTAAAATATACATAA |
| | | | | CACAATTTACCAGAATTTTGTTTTTAAAGGT |
| | | | | GTCACCTTTAAAACCTGAGTTCCTCTTTTGC |
| | | | | AGCTCTCATTTGCGTCAGCCTTCTGACTTGC |
| | | | | ATTGCCACCGCCAGATGCTTTTCTCCGGACT |
| | | | | GGCCATGCTGGGCACCCCATACCAGCAGTG |
| | | | | CCATCACACCCTCCCAGATGGGCTGTTGATG |
| | | | | ACGAGCGGCTGCCATGTTAGCGGTAATTACA |
| | | | | GTGTTGATACGGTGGCAAGTAGAACTCCCTA |
| | | | | CAAATAGAGTGAAGGAAACTATGCTGTTTGT |
| | | | | GTTGGACAGTATTTTTCCAAGAAGTTTTTGT |
| | | | | GCCCCTTTTTATTTTATTTTATTTTTATTTTTT |
| | | | | TGGAGTCTCGCTCTGTCGCCCAGGCTGGAGT |
| | | | | GCAGTGGCGCAATCTTGGCTCATTGCAACCT |
| | | | | CCGTCTCCTGGGGTTCAAGCAGTTCTCTGCC |
| | | | | TCAGCCTCCCGAGTAGCTGGGATTACAGGCG |
| | | | | CCCACCGCCACGCCTGGCTAATTTTTGTATT |
| | | | | TTTAGTAGAGATGGGGTTTCACCATGTTGGC |
| | | | | CACGCTGGTCTTGAACTCCTAACCTTGTGAT |
| | | | | CCACCTGCCTTGGCCTCCCAAAGTGCTGGGA |
| | | | | TTACAGGCGTGAGCCACCGCGCCCGGCCTCT |
| | | | | TTTTGTGCCTTTTTAAGTAGATTTGACATAG |
| | | | | AAAGGTTATGCTTCATCAAATATAAGAGGA |
| | | | | GTCTTATTTTGCATATGGGCAGAGAGCCCAT |
| | | | | CATCAATTAAATAACATAATTAGAGAGTATC |
| | | | | AGTAAATGCTGGCTCAGAAAATAATCTGCAT |
| | | | | TTTGTTGCCAAAATAAGTGTTTTGATCTGTC |
| | | | | CATTACTCTGTGTAACTCCCTTCATCCTCAA |
| | | | | ATCGTAAGTGTAATGGGTGAGTGTCTCTTAA |
| | | | | TAGCAAGGTATTTGTAGTTAATCGGTGAAAA |
| | | | | TGATGGTGCATCCCTTGTCTGGTGGCTGCGT |
| | | | | CAGTTTGTGACTCTTGTGTAGTCAGTGCTCT |
| | | | | GTGGGAATTCAGTGTGGCCCGTTTGATAAAC |
| | | | | TTTATAGAAAAATGGAATGAATTCAAATAA |
| | | | | AAGTTATTGTCTTGAAATTTTGAAATGTTTTC |
| | | | | AGTGTGTGTTCTCTTAATTTGAAATTGTTTTT |
| | | | | TTCTTTTTTTTTTTTTTGAGACGGAGTCTCGC |
| | | | | TTTGTTGCCCAGGCTGGAGTGCAGTGGCATG |
| | | | | ATCTCGACTCACTGCAACCTCCACCTCCTGG |
| | | | | GTTTAAGCAATTCTCCTGCCTCAGCCTCCCA |
| | | | | AGTAGCTGGGATTACAGACACCTCCTACCAA |
| | | | | ACACGGCTAATTTTTTTTTATTGTTAGTAGA |
| | | | | GACTGGAGTTTCACCATGTTGGCCAGGCTGG |
| | | | | TTTCAAACTCCTGACCTCAAGTGATCCACCT |
| | | | | GCCTCGGCCTCCCAAAGTGCTGGGATTATAG |
| | | | | ACGTGAGCCACCATGCCTGGCCTCAAATGTT |
| | | | | ATTTTTATGATAACGTCCCAAATGGTGACTG |
| | | | | TGGCCTAACTCTGATACCATGCCCCATTCTT |
| | | | | GACCCTGCCCCAAGCTTCCAGTCATTCTGGC |
| | | | | TGCGTTGGCCTTGCCTTTCCTCAGTTGGGCC |
| | | | | ATGTGCCATAGTTTGTGGCATGGCCTCGACA |
| | | | | TACCTCGCTGCCATCTCATGCAGATCCCCCC |
| | | | | AAACTCCAAGATAACTCCTACTCAGCCTTTG |
| | | | | CGTCTTAGTGTAATATCACTTCCTTTAGTAA |
| | | | | GTCTTTTTTTTTTTTTTTTGAGGCAGGGTCTC |
| | | | | GGTGTGTTGCTCAGGCTGGAGGGCAGCGGC |
| | | | | CAGTTGCGTGAACGTGGCTCTCTTCAGTCTC |
| | | | | GACTCCCTGAGCTGAAGCGATCCTCCCACCT |
| | | | | CAGACTCCTGAGTAGCTGGGACCACAGGCA |
| | | | | TGTGCCGCCGTGCCCAGCTAATTTATTTTAC |
| | | | | TTTTTGTAGAAACAGGGTCTTCCTGTGTTGT |
| | | | | GTAGGCCTCAGCCTTCTGGGGTTGGCTTACC |
| | | | | TCAGTCTCCTAAAGTGTTGGGATTATAGGCA |
| | | | | TGAGCCACTGTGCTCAGCCCCCTTTTGTTTTC |
| | | | | ATTACAAGTTTGCTTTTATAACTTAGATAAA |
| | | | | GGTGGGAAGACAGTTTTCACAATTAAGGCA |
| | | | | GAACAAGAGATAAAGAAACATAAAGGGAG |
| | | | | AGTTGTCTTTGTGGGAAGAAAGTGCTTTCTG |
| | | | | TGAAAGCACTTTTAGCAGGACTGCTGTTTTC |
| | | | | AGGCCAGAGTAGAAGGGCCAGAAGCAGTTT |
| | | | | TTATTCAGATTTCCTCATCGTTTCCATCAAGG |
| | | | | TCTCAGTGACTGTAAAGGATGGGTTGAAATC |
| | | | | AGTCTGGACTAAGTGTGGTTTGCTGCTGCTG |
| | | | | CTTATGAGGATTAAGTGTCTCGTTTGTCCGG |
| | | | | AGGCCCCTCTGGTCACATGTAGCTGAGCAGG |
| | | | | TGGAGTGATATGAAAGTTTTAGCATTTTAAT |
| | | | | GAAGAAAAAATGATCTCTTTGGAGGGAAAG |
| | | | | ACAACGAAATGGAGGTGTGCCCTATTGATTT |
| | | | | TTTTCTGTATGTTTAGGTTTTCAAAGTTTCTT |
| | | | | TGTAAATGAAACTGTTATGTAATTGGCAAAA |
| | | | | GTTTGCTTTTTAAAGACTAATTGTGGGTATA |
| | | | | TTTGGGAGATTGCGACTTGTTTTAGATTTTTG |
| | | | | GGTTTACAGTAAGGGGCTAGGGTATTTGGTG |
| | | | | GAGGGGAACTGGTTGATTGTGGTTGAAAAC |
| | | | | ATTCAGATACTATTCTAGCTCTAAGAAACCT |
| | | | | CACAGTTTTGTTTTGTTTTGAGACAGAGTCT |
| | | | | CTGTCACCTAGGCTGGAGTGCAGTGGCACG |
| | | | | ATCTTGGGTCATAGCAACCTCTGCCTCCCGG |
| | | | | ATTCAAGTGATTCTTCTGCCTCAGCCTCCCG |
| | | | | AGTAGCTGGGACTACAGGCCCATACCACCA |
| | | | | CTCCCAGTTAGTGTTTTTTTTTTTTTTTTTTTA |
| | | | | AAGTAGAGATGGGGTTTCCCCATGTTGGTCA |
| | | | | GGCTGGTCTGGAACCCCTGACCTCAGGTGAT |
| | | | | CCAGCCGTCTTGGCCTCCCAAAGTGCTGGGA |
| | | | | TTACAGGTGTGAACTACCGCGCCTAGCCAGA |
| | | | | AACCCCACAGTGCTAAAGCTGGCTCTGTGAA |
| | | | | TAAAGGGTTAAGAGGGTTTTCAGTGTGAAA |
| | | | | ATAAAAGTAAGCCGTCCTGTCTGTATGGCTG |
| | | | | AGATTTTTCAGGGGCCTAGAGGACCGAATCC |
| | | | | TGTTGAGTGAAAATCCAGTCTTCTGGGCTTC |
| | | | | CCCACACCCGCTTTCTCTCTTCCTCTTGGAGG |
| | | | | TCAAGGTTGAGACAGCTGTCAGGGTGCTGCT |
| | | | | GTCTCAGTTACAGCGTCCTTGTGGCTGGTGG |
| | | | | AGGGTGAGACCTGTTTCCAGGCCTGTCCTTC |
| | | | | GCATCTGTGCAGTGAATTATTTATTATCCTG |
| | | | | GAAATCTGTAGGGCTCTTTTTTCCACGTGTA |
| | | | | AAGCAACCAATGTGTACCTGTTCATTATTTG |
| | | | | AAAATGTTGGCTTTTTCTCAACAAACCATTT |
| | | | | TTATACCATATGAAAATTTCTTTTGTCATGGT |
| | | | | AAAATTCAGAAGACTTTCCCCTTCATTACTT |
| | | | | AACTCTAAAAAATGTGACTTTTAAGAATGGC |
| | | | | TGAAATTGAAATGTTATTTGTAAATGCCTAA |
| | | | | TAACTTTATGATATCAGAGGTTATTTTTTATG |
| | | | | TAAAATTAGCAAATAAACCTCTTTTCTTGGT |
| | | | | GCATTGATAGTAAGTTGCCCTTCCTCGACTC |
| | | | | CCTATGGCTCTTTTCAGACTTAGGATGCTAA |
| | | | | TGGCTTAGAATAAATTTTGGAATGCAGTATT |
| | | | | TCCAAGTAAAGGGAAGATTGCGTAGGCTGC |
| | | | | TTGGTTCCTGAGGTTTTACTAGAGTTAGGAT |
| | | | | TAGCTTACTGCTGCATTCATATCTGACACAA |
| | | | | TCAAAGAAGACTTTGGTTTAATTCTGGATGA |
| | | | | TGATGTAAACTTGAAATCATTATGGCTGTAC |
| | | | | TGTTTAAACTTATCTAAAATAGAGAAGGTAA |
| | | | | GACGCAGTGAAGGACCTTATTTTTCTCTTAA |
| | | | | GAAATCAAGCTTTGTTAGTATCCACCATGTT |
| | | | | TCTAGATGTGGTTTTACATCTTGCAAAACAG |
| | | | | GAAATAAAAGTAAAAAACCAAAAAAACCCC |
| | | | | AGAGCACCACTCTTGAAAGGATTAAGTTTTT |
| | | | | AAAAATGATTTTGACTAAGATGTCTGGCTGA |
| | | | | TTAAAGGATGTGCAGAGCACTGAATAACCTT |
| | | | | TGCCTTTTCTGATGGTGACAAAGAAGAAATC |
| | | | | CAGCTTTCAGGCAGCCGAAGAGCGTCTCGA |
| | | | | GAGCTTGTAGTGTTAGTATTCCACAGCCCCA |
| | | | | CAGTTGATTCGGATTTCAAGGAATTTTTAGA |
| | | | | CTTTGTGGATTTTTTCTTCACTATAATTGTAT |
| | | | | GTTTGGCTCCTAATTTATTTAAATTACATAC |
| | | | | ATAGATATTTTTGTTACTTTGAGAATAGTCT |
| | | | | ATCTGAAATTTGAAGTTCTTTAGAGCTTAAT |
| | | | | ATATTAAATATGCTAACACTCAAAACATTTT |
| | | | | CTTTCTTTTTTTTTTTTTTTGAGATGGAGTTTC |
| | | | | CCTCTTGTTGCCCAGGCATGATCTTGGCTCA |
| | | | | CCGCAACCTCCGCCTCCCGGGTTCAGGCGAT |
| | | | | TCTCCTACCTCATCTTCCCGAGTAGCTGGGA |
| | | | | TTACAGGCACGCGCCACCACTCCTGGCTAAT |
| | | | | TTTGTATTTTTCGTAGAGACGGGGTTTCTCC |
| | | | | GTGTTGGTCAGGCTGGTCTCAAACTCCCGAC |
| | | | | CTCAGGTGATCTGCCTGCCTTGGCCTCCCAA |
| | | | | AGGGCTGGGATTACAGGCGTGAGCCACCAT |
| | | | | GCCCGGCCTAAACATTTTCTCACAGGCATTT |
| | | | | TTCCCCTGACACATGCGAGAGGTATCTTTGA |
| | | | | ATTGTATCCTTTATCTTTTAGTGTGAAACTCA |
| | | | | GAAAAGTGATGCACGCTTGCACTTACAGTTC |
| | | | | AGGTAAAATGTTAAGCATATTCAATGAGATT |
| | | | | TACATTCATGCTTGATTTTTCTTTGGCAAAGT |
| | | | | CTTTAGATCTGATTCTGCTAAACTTGGGTTCT |
| | | | | CACCAGATGACTGGCTTTTAAAAGAAGATG |
| | | | | ATGTTGAAACTGACTCTTGTAAAAAAGGAC |
| | | | | ATTTAGTAGAAGCTAATGGTACGGTGAAGTT |
| | | | | TTAGAGAGTTGAGGAGAAAATCTGTCTTAG |
| | | | | AACTTAATCTGTGCCTTTTCCTTAATAGCTTT |
| | | | | CTTCTAAGCCCATAAATATATTGGTTCAGGG |
| | | | | GGATGAGTGAAGGCAAATGGAGGGTGTGGA |
| | | | | AGGGAGACAGAGAACAGCTCTTGGGCGTTG |
| | | | | GAGAAGTGCTGGGACTTGTGTCAGTGCTGCC |
| | | | | CGTGTTGGTTTTCCAGCGCTGCTGTAACTAA |
| | | | | CCACCATAACCTTAGATTTATTGTTTTATAGT |
| | | | | TCTGTTGATTAGAAGCCTAACATGATCTGCC |
| | | | | TGGGCTAAAATCTGGGTAGGTAGGGCTGGTT |
| | | | | CCTTCTGGAGGCTCCAGGGGAGAATCTGTGT |
| | | | | TCTTGTCTTTTCCGTGTTCCAGAGGCTGCCTG |
| | | | | CATTCGGCCTGTAGTGCTTTCCTCAGTCTTCA |
| | | | | AAGCCAGCCTCATGGCATCTCTCTGACTCCT |
| | | | | GCTGCCCACCCGCTTTTTTGGAGAGTCTCGG |
| | | | | CTCTGTTGCCCAGGCTGGAGTGCAGTGGCAC |
| | | | | AATCAAGGCTCACTGCAGCGTCGACCTCCTG |
| | | | | GGCTCAATCAATCCTCCCGCCTCAGCCCCCC |
| | | | | AGCAGTGCTGGAATTACAGGCATGGAGCCA |
| | | | | CTGTGCCTGGCCCTTGTTTCACTTTTGGATCC |
| | | | | TTGTGGTTACAAATTGGGCCCACCCGAGCAC |
| | | | | TGCGAGGTCATCTCCCTCTCTGCTGATTAGT |
| | | | | AACCTCAGTTCCCTTTTCCACGTAACCTAAG |
| | | | | GCATTCACAGGTTCCAGGGGTGAGCAAGCT |
| | | | | GGAGGTTTTTCCAGTAATGTTCCCTTGCTGT |
| | | | | CCTCAGATGCCATCCGGGATCCCACATTGCA |
| | | | | TTTAGTTGTCATGTCTCCTTAATCTCGTCTGC |
| | | | | AACCATTTCTCAGCGTTTCCCTGTTTTTCATG |
| | | | | ACCTTGGCAGTTTTGATGAGGGCAGTCATCT |
| | | | | TTTTTTATGTCTTTGTGACTGACCGTTCACAC |
| | | | | ATTGGCATGTTTGTAAAGCACGGTGTGGCTG |
| | | | | ACGTGTGGCTCACCTGACTGCTTTTCCAAAT |
| | | | | GGGGTGCAGTGTGCTCTACAGTGGGAGAAC |
| | | | | AGCTTTGTGTCTTCTTTTAGCTGGAAGGAGC |
| | | | | TACATGTTTTATAGAAGGGACTTCTGAAACT |
| | | | | AGACAAACTCTGCTTTTTCTGATGTTTCACT |
| | | | | GATTTCCTTCACAGATGTTCATTGGCTGCTC |
| | | | | ACTGGGTGCAGTGCTGTGGCCTCCTGTTCTG |
| | | | | AGATCTGGGAGAAAGATGCATTTAGTTGACT |
| | | | | AAAGCTTGCATTAAATATTGGTTCTTATGAA |
| | | | | AAGAGAGATCATAGATCAGAGGAGGGAAAG |
| | | | | GCTCTGTGACCTGGGAGATTTAAGGGAAGA |
| | | | | AGTGCTATTTTTTTTTTTTTTAACTGTAAGCT |
| | | | | ATGTTTCTGTTTTAAAAAAAGAATCAGTAGA |
| | | | | ATGTCACTGCAGAAATCAGAGTAAGGGCAT |
| | | | | CTTCGACTTCGGGAAGGCTGGGGACAAAGG |
| | | | | CTGGGAAGGCCGCATGTAACGTGAGAATGT |
| | | | | CAGCCAGACGTGTCTGAACACGAAAGCCCA |
| | | | | TCCAAGAAGATGGAAGATGACAGCAGATTA |
| | | | | ACCTGGAAGCATGACTGGAGTTGGGTTGTGT |
| | | | | AGGGTTTGCAATGATAGACCTGAATTTCATT |
| | | | | TGCTTACTTGTTTTGAGGCAGAAGATGATGT |
| | | | | GTCAGAGTTTGCAGGCGGTGGATGCCTAGAT |
| | | | | GAAAGCACTGGCTTTGCAACACCATGGTGCC |
| | | | | TAAATGGTTCTTTTTTTGAGACAAGGTCTTG |
| | | | | CTCTGTCACTCAGGGTGGAGTGCAGTGGCGT |
| | | | | GACCATATCTCACTGCATCCTTGAGCTTCTG |
| | | | | GGCTCAAGTGAGCCTCCTGCGTCAACGTCAT |
| | | | | GAGTGGCTAGAACTACAGACGTGCGCTAAT |
| | | | | TTTTAAAAATTCTTTGTAGAAATGGGGTTTC |
| | | | | GCTATGTTTCTTAGGCTGATGTTGAACTCCT |
| | | | | GGCCTCAAGTAGTCCTCCTGCCTCAGCCTCC |
| | | | | CAAAGTGCTGGGATGACAGGCACAAGACAT |
| | | | | CACATGCAGCCTTAAGAAGATCCCTACAAG |
| | | | | AGAACCTGGAGAAACTTGAAATAGGGAAGG |
| | | | | GGACTCGAAAGCACACTGTGTAGGTGAATT |
| | | | | GTGAAGACTTCATGGTGTTAACTGGTATTCA |
| | | | | TTAAAAAGACTTGGAAAAAGAGAAGATGAA |
| | | | | TTGATTCCCAGTGGAGGGAATGGGGCAAAC |
| | | | | ATTAGTTAGGAGAGTGGCTTTGTGAAACGAT |
| | | | | AGAGCAAAGGAGACAGAAAAGAAAGCAGG |
| | | | | AATGGGGAACGGGGTGGTGGGGCAGAAGCT |
| | | | | GGGTGCTGTTTATTTTGTGCCTACTGTGTGTC |
| | | | | ACTCTCCATTCTCTGCCCTGGGGACATTTGA |
| | | | | CAGATGTGGACGTCACTGGTGTGATTTGCCC |
| | | | | ACATTCTGGAGGGAGAAGTGGGCATCTCAG |
| | | | | TTGAGGAAAGGAAATGGCTATCTGGGGAAT |
| | | | | TCTTGAGGGGCACAGCCCAGGACGGAGCCT |
| | | | | TGTGTTAGATGGGTACCTGCTGGCTCTGCTC |
| | | | | AGCTCGGCAGATGTGTTGCCTAAATGACTTT |
| | | | | AATGGCCAAGGCAGACTGGTATTTGCCCTCA |
| | | | | GATATTTTGTCAGGACAGTGTAAAATGTGGG |
| | | | | CCAAGATAGTGGTTTTGGGTCCAAACAGAA |
| | | | | GGGGGTTAGAGATTTTGGAGGTTGAAGCCT |
| | | | | GCTGCTAGAAGGAGTATAGAAAGAAGAACG |
| | | | | GGGATTCTGTTTGCCCAAATACATGGTTAGG |
| | | | | CTCATCTTGGACTTGTTTTTGTTTTATATCTT |
| | | | | CTGTTACTTCTATGCACATATTAATAGATAC |
| | | | | CTAATTAATATTTGGTTGGAAATCTTAAAAG |
| | | | | TTAGGATTTTTTTTTTCTTTTTTTTTGAGATG |
| | | | | GAGTCTCACTCTGTCGCCCAGGCTGGAGTGC |
| | | | | AGTGGTGTGATCTCGGCTCACTGCAACCTCC |
| | | | | GCCTCCTGGGTTCACGCGATTCTCCTGCCTC |
| | | | | AGCCTCCCGAGTAGCTGGGACTACAGGCAC |
| | | | | CCACCACCAGGCTCGGCTAATTTTTTTGTAT |
| | | | | TTTTAATAGAGTCAGGGTTTCGCCATGTTAG |
| | | | | CCAGAATGGTCTCAATCTCCTGACCTTGTGA |
| | | | | TCCACCCGCCTTGGCCTCTCAAAGTGCTGGG |
| | | | | ATTACAGGCGTCAGCCATCACGCCTGGCCTA |
| | | | | AAAGTTAGGATTTTTAAAAAGGCTTGTGGTC |
| | | | | TGAGAAGGAAGGCAATAATACTTGGCAGGA |
| | | | | AAGACTTAATTTTTTTTTTTTGAGACTGAGTC |
| | | | | TTGCTCTGTTGCCCAGGCTGGAGTGCAATGA |
| | | | | CACGATCTCAGCTCACTGCAACCTCTGCCTC |
| | | | | TCGGGTTCAAGCATTTCTCCTGCCTCAGCCT |
| | | | | CCCGAGTAGCTGGGATTAGAGGTGTGTGCC |
| | | | | ATCACGCCCGGCTAGTTTTTTTTTTTTTTTTT |
| | | | | TTGAGATGGAGTCTCGCTCTGTCACCCAGGC |
| | | | | TGGAGTGCAGTGGCACAATCTCGGCTCACTG |
| | | | | CAAGCTCCGACTCCCAGGTTCATGCCATTCT |
| | | | | CCTGCCTCAGCCTCCCGAGTGGTTGGGACTA |
| | | | | CAGGCACCTGCCACCACCCCCGGCTAATTTT |
| | | | | TTGTATTTTTAGTGGAGATGGGGTCTCACCG |
| | | | | TGTTAGCCAGGATGGTCTCAATCTCCTGAAC |
| | | | | TCGTGATCCGCCCCCTCGGCCTCCCAAAGTG |
| | | | | CTGGGATTACAGGTGTGAGCCACTGCGTCTG |
| | | | | GCCTTTTTAAAATTTAATTTAATTTTTTTTGG |
| | | | | GGGCGGAGTTTCGCTCTGTTGCCCAGGCTGG |
| | | | | AGTGCAGTGGTGTGATCTCAGCTCACTGCAA |
| | | | | CCTCCGCCTCCTGGGTTCAAATGATTCTCTT |
| | | | | GCCTCAGCCTCCAGAATAGCTGGGATTACAG |
| | | | | GCATGTGCTACCAAGCCCAGCTAATTTTTTT |
| | | | | GTATTTTTAGTAGACATGGGGTTTCTCCGTG |
| | | | | TTGGTCAGGCTGGTCTCAAACTCCCGACCTC |
| | | | | AGATGATCCGCCTGCCTCGGCCTCCTTAAGT |
| | | | | GCTGGGATTGCAGGTGTGAGCCACTGCGCC |
| | | | | AGGCCTAGATATTTTTTATATCAGGTCACAT |
| | | | | GTGTTTTATCTAGGAGGCAAACTTTCCTTGT |
| | | | | AATTAGTGTTTTTTTTTTCCTTTTTTTGTTTTT |
| | | | | GTTTGAGTGGAGTCTCGCCACTGCGTCTGGC |
| | | | | CGTCATTAATGTTTTTCTAGTGATGTTGCACT |
| | | | | CTCTTGACGTCAGTTAACTGTCACCTGAATC |
| | | | | TTCAGGGACTGGGTTGGGTTGTGGGGTAGGT |
| | | | | GGTGGCGTGACTTGCTGTTGATGGGACTGCT |
| | | | | GTCCACAGGAACAGGAGGACCCACTAGTTA |
| | | | | CACAGCTGCCAAAGGTGTGGGTCTTGAGGC |
| | | | | CCGAAGGTTGAGGTTGCAGTGAGCTGAGAT |
| | | | | CATGCCACTGGTCTCTAGCCCCGGTGACAGA |
| | | | | GTGAGAACATCTCAAAAAAGAAAAGAAAAA |
| | | | | GTGTTGGGTCATTATTTACACACCCAACAAG |
| | | | | TGAAAGGTCTGGAGGGCCCAAGTGAGGACA |
| | | | | GCTCAGTTCAGGCATGTTTAACCTCCAGATT |
| | | | | ACCTCATCCTTGAGAAATTTAGGAATAATCC |
| | | | | CAGTGTCAGTCTTTGGAGGTGTGGTTCTTTG |
| | | | | ATGTTAGTCATGGGAATAAGGAATTTGCATT |
| | | | | AAGCCTCAAGAATTGTGGAAAGTCCTAATG |
| | | | | AGAAATGTCACGGGCAGAAACTGCCACCCG |
| | | | | CTTTAGTGGTGCAGGTGATGGCTGTGCCGGA |
| | | | | GAGCTGCGCGTAGCCGGCTGCTGGAGGAGA |
| | | | | GGACGTGGCCTGTACAGGCAGCTTCGCTCTG |
| | | | | GGGGGTGACTGTTGGAGGGCAGCGGGGACG |
| | | | | AAAGGGACACATTTGTATGATGCTCAGTTGT |
| | | | | CAGCAGGATGTCCAGGTTGCTTTTTTGGAGG |
| | | | | GCAGTGGGTCCTTGAGACCTTGAGACCCATT |
| | | | | CTCTTTGGAAAGTAATGAGTTACCGGGTGGG |
| | | | | GGCTGTAGGAGGATGTTCAGTTGTGGTGTGG |
| | | | | AGGCGTCGGGTTGCTTAACCTCAGAGGGATC |
| | | | | TTTTTTCCTATGAGTTGTGTAGAAGAGGATG |
| | | | | TCTTCCATAGATTTGAAGGACGTTAAAAAAA |
| | | | | AAACCAACCCCAATGTGGCTTTCCTTCTTTC |
| | | | | TCACTAGGGTGATAGTCCGACGTGCATGTCT |
| | | | | GTTTCCTGGACCCTGGATGTGAAAATGGGGT |
| | | | | TCTTGAAGGCACTGGGAGTGTTTCTCTGCTG |
| | | | | GCCCCAGAGGCCTGTGTGTGCTGCTGGCGAG |
| | | | | GCAGCTGGGCCACTGTCACTGTGCCCTGACA |
| | | | | ACAACGTTGGGAACTGTTCCTGAAAGTGTTA |
| | | | | AACAAAATTTCAGTTTATTAAGCGCTCCTCT |
| | | | | CATTAAGCCATTGTTTTATTTTTCTTTTTTGA |
| | | | | TTTTTTGGGCTTCTTTTGAAGCCATAATAAAT |
| | | | | TGGAATAGAAAGAATACATACCAAGAAACA |
| | | | | AATATTAGGTCTGATTTTTTAATTTTTTGTTA |
| | | | | TTATTTTTGAGATGGAGTCTTGCTCTGTCCCC |
| | | | | AGACTGGAGTGCAGTGGCGTGATCTTGGCTC |
| | | | | ACTGAAACCTTCGCCTCCTGCGTTCAAGCAG |
| | | | | TTCTCCTGCATTCAAGCAGTTCTCCTGCCTGC |
| | | | | CACCACACCTGGCTGATTTTTGAATTTTTAG |
| | | | | TAGAGTTGGGGTTTCACCATGTTGGCCAGGC |
| | | | | TGGTCTTGAACTCCTGACCTCAGGTGATGTA |
| | | | | CCTGCTCAGCCTCCCAAAGTGCTGGGATTAC |
| | | | | AGGCATGAGCCACCGCGCCCGGCCTCTCCA |
| | | | | ATATTTTTATTATTAAAATGCTAAATACTGC |
| | | | | CGGGCCCAGTGGCTCACACCTGTAATCCCAG |
| | | | | CACTTTGGGAGGCTGAAGCGGGTGGATCAC |
| | | | | CTGAGGTCAGGAGTTCGCGACCAGCCTGGG |
| | | | | CCACATGGTGAAACCCTGTCTCTTCTAAAAA |
| | | | | TACAAAAAATTAGCTGGGCGTGGTGGCAGG |
| | | | | CGCCTGTAATCTCAGCTACTCGGGAGGGTGA |
| | | | | GGCAGGAGAATCGGTTGAACCCAGGAGGTG |
| | | | | GAGGTTGCAGTGAGCCAGAATCGCACCACT |
| | | | | GCACTCCAGCCTGGGTGACAGAGTGAGACG |
| | | | | CCATCTCAGAGAGAAAAAAAAGAAATTACG |
| | | | | CGTGGTGGCCCATGCCTGTCATCCCAGCTAC |
| | | | | TGAGGAGGCTGAGGCAGGAGAATCGCTTGA |
| | | | | ACATGGGAGGTGGAGGTTGCAGTGAGCCGA |
| | | | | GATGGCGCCACTGCACTCCAGCTTGGGCACC |
| | | | | AGAGTGAGACTCTGTCTCCAAAAAAAAAAA |
| | | | | AAAAGGGGAAATGACTTAAAGGTGATGGCT |
| | | | | TTTATACTTCTATTGTGCCTGTTTTCTGAGAT |
| | | | | ATAAATTTAACTAGCTAATTCTCTCGTGTTTT |
| | | | | AAATAGTAGACAAAGAAAGACAAGACCAAA |
| | | | | GGAGAACCTTTTTCTCTGTTTCTTACTCCGTC |
| | | | | TGCTTTTATTAATAGATGCTCACGGTGTGGT |
| | | | | CTTCCACTCACTTCCCCTTTCATCTCTGAGCT |
| | | | | TAACGAGCTCCTCGTATTATAGATTGTTACC |
| | | | | ATATCATGTGTTCCAGTCTGTGCCCGTGTAT |
| | | | | AAACGTGTGTTGTGTGTTACGCGATACTGTG |
| | | | | AGATGAGTCTGCCCAGAGGGACTCTGAAGT |
| | | | | CAGGACTGTGTCTTTTCCACACCTCTCCACC |
| | | | | CCAGCTCTCATCATGCCTCTGAGAGAACCAG |
| | | | | ATTCAGAGTGTGGTGAGGGGAGGATGAAGT |
| | | | | GGTTTGGGGTGGGCCTTGGGCCCCCATCTCT |
| | | | | TTGCTGGAAGTGTAGTATACCTCTAGGATAT |
| | | | | GTGTCCAAACTGTTGGCTGTGAGACCAAGG |
| | | | | AGGAGAAGTCTTTTTTGGCAGGCTAGTGCCT |
| | | | | GCGGCTTGAGGTCTCAGTGTCTGTAACTGCC |
| | | | | AGGCTGCAGAGCCCCACCTGGCTGAGTCAG |
| | | | | GAGTGTGTTGTAACCTGCCCACCTGCCCAGG |
| | | | | CTGGTTAGAAGCAAGCGTAGGCGTTGGGTCT |
| | | | | GCCTGTCCTGGTCCAGGCACCTCTCCTGGTT |
| | | | | TGGCCAGGTTTTGGTTTGTATTTATTCCTGAT |
| | | | | GTTGATGTGTAAATGATATCGTTACAAAGCA |
| | | | | GGTAGTTTGCTTTGCTATTCTACGAATAACC |
| | | | | CAAGAACCTGAGGATAATAGGACACGTTAA |
| | | | | CAGTCTGCTAGTTGAGAGTTCTGTTTCTGTG |
| | | | | ACTTCAGGGGACATATGACCATCCCGATTGT |
| | | | | GGTGGGTTATTAAGGCTGTGACAAGTCACA |
| | | | | GGTGGCTTTAGGGATGTCAAAGATAGGCAA |
| | | | | AGATAGGTTCATTTGAATTTGATTTCATCTTT |
| | | | | TGAGAATGGGTTGGTATACCTGAAATTGGCT |
| | | | | TTGTAGTTTTGGTATTTTGATGTGAGAAGGC |
| | | | | ATTGGCTGAATTTTTTTTGTTCTCATAATTTG |
| | | | | CATATTTTCTGTGTTTTCTCCATTGTTTGGCT |
| | | | | CAGTTGTTTTCTTTTTCTTTTTTTTTTTTTTTG |
| | | | | AGACACAGTCTCGCTCTATCACTCAGGCTGG |
| | | | | AGTGCAGTGGCGTGATCTTGGCTCCCTGCAA |
| | | | | CCTCCACCTCCCGGTTTCAAGCACTTCATCT |
| | | | | GCCTCAGCCTCCCAAGTAGCTGGGACTACAG |
| | | | | GTGCCCACCACCACGCCTGGTTAATTTTTAA |
| | | | | ATTTTTTTAGTAGAGACAGGGTTTCACCATG |
| | | | | TTGGCCAGGCTGGTCTTGAACTCCTCACCTC |
| | | | | AATTGATCCACCCACCTTGGCTTCCCAAAAT |
| | | | | GCTGGGATTCCAGGTGTGAGCCCCCGCACCT |
| | | | | GGCCGGGGTCAGTTGTTTTCGTGTTCCTTAT |
| | | | | CCCTCTTTAAACTTGGGAGAGCATTTTGTGT |
| | | | | TTCGTGGAGATATCACAGCATAGAACAGAA |
| | | | | TTTTGATTGTAATTGTTTGTTGTTGACTTGCT |
| | | | | GTAGTACTGTTTTCAGACTTCCAGTGTGAAC |
| | | | | GATAAAAGATTATCTTAAAATTTTAGGAAAA |
| | | | | ATTATCTTTTGTGTGGGTAGTGAATAAATAA |
| | | | | TTAGCATTAATGTTACCGATGTCTTCTACCT |
| | | | | AGTTCTTTATCAAAATTCTTTTCCTGACTGCA |
| | | | | ATATTTCATTTTAATAGAGAATAATTTTCAT |
| | | | | GTGAAACTGCTTGCTTTATATTTTCTTTCTGG |
| | | | | TTGCTATAGAAACAGTGCAGCGTGGTGGGTT |
| | | | | ACGGGGCTTTCATGAGGGAGCTCTCTGGGA |
| | | | | GGCAGCAGTCACCTGGTGATGAGGGAGGGG |
| | | | | AATGGAATTGTCTTCTTTCACATCCCAGCTT |
| | | | | GGCCACGGAGCCTCAGGAGCTGCATCATCA |
| | | | | GAATGATTGGATTTTCTTGTCCCTCCAGCTT |
| | | | | AAATGCTGTGGGCTTTGTTCTCCAGTGGAGT |
| | | | | GGTCATTGCCTTTTTTCCTACCTGTGAGTTAG |
| | | | | TCCATCTCTCCAACCCACTGACGACCCAAGA |
| | | | | GCTCCCCAGCCTTTCTCTGCCCGTCACAGTC |
| | | | | AAGAGGGTCTGTTGGAAAATTACTGACTGA |
| | | | | ACAACAGATGCCAGAGATTGCCCTGAATTGT |
| | | | | GGAAGTCCCGGCTGCCACCCCCTTACTTGAG |
| | | | | GTCCTCATCTCACTGCACCAGCAGGGGTGAG |
| | | | | GAGAGGGCAGGGGTGGCCCTGCACAAAGCT |
| | | | | GGGGAAGAAAGGAGGCAGCTCCCGGCCAGG |
| | | | | CAGGGCCCAGCTTTCCTTCTAGATCATCAGC |
| | | | | ACAAATTTGCGTTTGAAGATTTAACTTACAT |
| | | | | TTTATTTTTTTATTTGGATGATACATGTGTAA |
| | | | | GTTTTGGGATGTTATGCAGTGTTCTGAGGAT |
| | | | | TGACGTTGGTGGCATATGTAACCTTGAAGGC |
| | | | | AGCATTTTGTTAATTATAAAAAACTTGGAAT |
| | | | | ATAGTGATTGAGTATGAAATAATTGAAGCTG |
| | | | | TGAGTTCAGTTATGAAGAACTTGGTCTCGGA |
| | | | | GTCTCTCTGAGCTTGGGAGGCTGCCTTGGTC |
| | | | | TCAGTGGGGTTGGCATCATGTGTGTTGGTGG |
| | | | | TAGGTTGGAAAGAGCATTTGGAGTTCTGAA |
| | | | | GAGTGACCTGTGCTGCCTTTCTGGCAACCAG |
| | | | | TCATTATTTGTGCATGGAAGGAAGTAGCATG |
| | | | | GATGAAAGCTTTTCTTTCAAATGGGATGATG |
| | | | | TGTGGGGGTATATTTCTGGTCTTAAATTTTTT |
| | | | | TTTTTTTTTTTACATAAGGTAGAGACAGGGT |
| | | | | CTCACCATGTTGCCCAGGCTGGTCTCAAGGT |
| | | | | CCTGGGCTCAGTCATTCCTTCCACCTTGGCC |
| | | | | TCCCATAGTGCTGGGATTACAGGCATGAGTC |
| | | | | ACCATGCCAGGCCAGTGTATTTATTTTCTAC |
| | | | | TGCTGTGTAACAAATTGACACACAACACATT |
| | | | | TATTATCTCACAGTTTCTGTGGGTTGGAAGT |
| | | | | CTGGGCACAGCTTAGTGGGATCCTCTGCTCA |
| | | | | GGGTTTCATCCGGTTGCAATCTAGGTGTTGG |
| | | | | GTGGGACTGTGATCTTATCAGAGGCTCGAGT |
| | | | | GGGGGATGGTGTGCTTTTGAGCTCTTTCAGG |
| | | | | TTGTGGCAGAAGTTATCTCTTGCTGATTGTG |
| | | | | GGACGCAAGTCTTTGTTTTCTTGTTGACTGTC |
| | | | | CCTTGAGGTCTCCTCTTGACTCTTAGAGGCC |
| | | | | CCCTGTACTTGCTTGGCGCATGGCCCCTTGC |
| | | | | AGCTTGGCAGCTCACTTCAAAACAGCAAAG |
| | | | | AGTCTTCTGCTTCAGTAGACTAAGTCTTACG |
| | | | | TAACATAATGTAGTCATGGGAGTGACAGCG |
| | | | | CAGCAACTTTGCCACCTAATGCAACCCAGTC |
| | | | | AAGGGAAAGCCATTCCATTGTTTTCACCACA |
| | | | | TTCTGTGGGTTAGAAGCACGTCACAGGTCCC |
| | | | | ACCCGCCTTGCACTCCGGGCAAGGTGGCTAC |
| | | | | AAGGAGGCATGGACACTGGGTGGGGAGATT |
| | | | | GCCAGGCTCACCTAGGGTCTGTCTGCTGGCC |
| | | | | TGGGGTGTTCTTCAGAACCCTCATTCCCTGG |
| | | | | ATACTGACATGTCAGTCTCTGGGAATTGGTT |
| | | | | GCATCTCTCTCTCCTCCATGACGTATCAGTCT |
| | | | | GTGGGAGAGCGGTGCGTCTCTTCTCTCTCCT |
| | | | | CCATTTGCATGCATTCCCGGACCCACTTGTA |
| | | | | GAACGGAAGTAGAATCAAGACTCGTGGCCA |
| | | | | CAGTTTGGGTGGATGGGACACTTGGGCTTGC |
| | | | | CCTGGTACTATCAGGCTGTTTGCCCTATGCC |
| | | | | TTGCATTTCCCACTGTCCTGCCTGCCTCTCAG |
| | | | | GCTTATGGTGAGGATGAGGTGAGATGAGAT |
| | | | | GATGGATGTGGTCAGTGAAGCAGCTGGTCA |
| | | | | GGGCCTGGTGGAATTCTACTCTAGTCCAGTC |
| | | | | TGCCTCTTTCTCCACCTCTATTCTCTAGGCAG |
| | | | | AGATTGGTTATTTTCAGTAGTAAGTATGGAC |
| | | | | TCATCAGAGTTCTTGAGGTCTTTGCTTCCTGC |
| | | | | CTCAAATTCCCTTCTCTCCTTTCCCTGACTGA |
| | | | | TGCCTACTCATCCTTCAGGCCTCCATGTTCCC |
| | | | | AATGATCCTTTTTTTTTTTTGAGGCAGGGTCT |
| | | | | TGCTTTCTTGCCTCGGCTGGAGGGCAGTGGT |
| | | | | GTGATCCTGACTTATTGCAGCCTTGACCTCC |
| | | | | TGGGCTCAAGCCAAGCTGTCCTGGGTAGCTG |
| | | | | GGACCACAGGTGTGTACCACCACACCTGGCT |
| | | | | AATGTTTGTACTTTTTGTAGAGTCGGGGTCT |
| | | | | CACTGTGTTGCCCAGGCTGGTCTCCCAACTT |
| | | | | CTGGGCTCAAGTGATCCTCCTGCCTCAGCCT |
| | | | | CCCACAGTGCTGGGATCACAGACATGAGCC |
| | | | | ACTATGTCTGGCCCCCAGTGAGCCTCTTGAT |
| | | | | CTCATGGTTGAGCTCTTTTTTCACTTGTGACT |
| | | | | TCTGCCATTGGTTAGATTCTCACCTTTGGTCT |
| | | | | TGCGGTGCTCTCTGCTCCCCTCTGTGGTAAT |
| | | | | AATTTATTGTTGTGTTGTAATTATCTTTTACT |
| | | | | TCCTTTCCACTAAGTGATATTCATTGGATCCT |
| | | | | CAAATAAAAGGCTTGGCACACTTAGGGCAG |
| | | | | ATACCTAATAAAACAGTAACACCTCTGTGGT |
| | | | | CAGTATTTAAACGGTGTGCACAGCCATTAGA |
| | | | | ATGGAATAAGCTGGGCGCAGTGGAATGGGA |
| | | | | GGTTGATGTTGCAGTGACACATGAATGTGCC |
| | | | | ACTGCCCTCCAGCCTGGGGGACAGAGCAAG |
| | | | | AGCTCATCTCAAATAAATCAATAAATGGGTC |
| | | | | AGAGGAGTGTGATGTGCTGGTGACTCGGGT |
| | | | | CCTGCTCTGCCGCCGGAGCTTTTCCAGCCTT |
| | | | | GCTGCTCCTTTGCTTTGGATCTTTTCATTCCT |
| | | | | AGGTTTATTGTTGAGTCTGAGACGGTTTTTTT |
| | | | | TCTCCCTTCCCCTTCCCCTTAGTAAATGTGCA |
| | | | | GATCTCCTGATATTGCACCTGACTTAGAGAT |
| | | | | CTTGGTACCGTCCCTACAGATCCACACAAAC |
| | | | | ACAAAAGCACAGGTGATACTCAGGTTGGAA |
| | | | | CATGAAGATGCAAAGTCAGCATCCCCATTTG |
| | | | | CCTCTCACTTTTCTGTCTTCCTTAGCTGTGTC |
| | | | | CTGCGAGTGTTTATTGGGCAGTGTTGACAGC |
| | | | | AGCTTCAGTCTCAGAAAAAGGATGGGAAGT |
| | | | | TGCTCTCAGACTCGGAACCCTAAAGCTTGGT |
| | | | | CGGAATTAGGTTTCTGCCCTGATCCTGATGC |
| | | | | TCTTTCTGCCCGGTGGAGAGCACCTTTAAAA |
| | | | | GTAGTCACCTGAGGGTCAAGGATGGGTACA |
| | | | | ACAGCTTCCCATTTTATTCTAGGAGATGTGT |
| | | | | CTGGAGATAAAATGAGCATGTGATGTTTGGC |
| | | | | AGGGCTGCATGCCTCGAGGGTCATAGCCATT |
| | | | | GTCCCTGATGTTCAGGACCTGGTAACTGGGG |
| | | | | GAGTAAGGACTTAAGGTACATGTTTTCCTGT |
| | | | | TTCCTCTTTGCTGTGAGTTGTATGTGAGTTGA |
| | | | | TTTGGGTGGTAAATGAAATCATATCTTTTTTT |
| | | | | TTTTTTTTTTTTTTTTTTTGAGACAGAGTCTC |
| | | | | ACTGTCGCCCAGGCTGGAGTGCAGTGGCATC |
| | | | | ATCTCAGCTCACTGCAACCTCTGCCTCCTGA |
| | | | | GTTCAAGCAACTCTCCTTCCTCATCCTCCCA |
| | | | | AGTAGCTGGGATTACAGGTGTCCGCCACCAC |
| | | | | GCCCGGATAATTTTTGTATTTTTAGTAGAGA |
| | | | | GGAGGTTTCATCATGTGGGCCAAGCTGGTCC |
| | | | | TGACCTCAGGTGATCTGCCCACCTCTACCTC |
| | | | | CCAGAGTGCTGGGATTATAGGCGTGAGGCA |
| | | | | CCACACCTGGTCATGAAATCATATCTTAAGT |
| | | | | GTCTCCATGGTGGCCTAATTTGTTACCTGAA |
| | | | | GCTTTTTCTCAGAGCAGCCTCTAGCAAAGAG |
| | | | | AATCACTTCCTGTGGACTCCTTCAGGGCTGC |
| | | | | AGGGTAACTTGATGAGTTCTTGCCGTCTCGT |
| | | | | GAATTCCTGAGTGGTGAGAGCACCACTCCAC |
| | | | | ACAGGACTTCGGGGCAGCAGGCTTTTAGGTT |
| | | | | TTGCACACAGTTCCTCGAAAGCTGTGATTTG |
| | | | | GAAATCGCTAGAATTTCCAGATAGTAACTAG |
| | | | | TTTGGAGGGTCAATAGTGCTTTAGTTTTATTT |
| | | | | ATTTATTTTTTTTTACTTTTAAAACAGAGGTG |
| | | | | GGGTCTCACTCTGCTGTCAGGCTGATCTCCA |
| | | | | GCTTCTGAGCTCAAATGATCCTCCTGCCTTG |
| | | | | GCCTCCCAGAGTGCTGGGATTACAGGAGTG |
| | | | | AGCCACTGCGCCCAGCCTCAGTCATGCTTTT |
| | | | | AAATTGAGGATGTAGGAAGGAAGGCTTTGG |
| | | | | CTCCCATGCTTTCATGAGATTTCCTTTTTTTT |
| | | | | CTGAGACAGAGTCTCGCTCTGTCGCCCAGGC |
| | | | | TGGAGTGCAGTGGCATGATCTCAGCTCACTG |
| | | | | CAAGCTCCGCCTCCCAGGTTCACACCACTCT |
| | | | | CCTGCCTCAGCCTCCCGAGTAACTGGGACTA |
| | | | | CAAATGTCCGCCACCGTGCCCAGCTAATTTT |
| | | | | TTTTTGTATTTTTTAGTAGAGACGGGGTTTCA |
| | | | | CCGTGTTAGCCAGGATAGTCTTGATCTCCTG |
| | | | | ACCTTGTGATCCACCCGCCTGGTACTACCAA |
| | | | | AGTGCTGGGATTACAGGCATGAGCCACCGC |
| | | | | GCCCGGCCACGCCCGGCTAATTTTTTGTATT |
| | | | | TTTAGTAGAGACTGGGTTTCGCCATGTTAGC |
| | | | | CAGGATGGTCTCGATCTCCTGACCTTGTGAT |
| | | | | CTACCTGCCTTTGCCTCCCAAAGTGCTGGGA |
| | | | | TTAGAGGCGTGAGCCATCGCACCTGGCTGCT |
| | | | | TTCATGAGATTTCTTAGAGACTAATACTTTA |
| | | | | GTATTTACCCTCCTTTCTCAGTCTATGGTGTT |
| | | | | AACCAGTATTCCCTACCTACGTTTAGTCTGT |
| | | | | ACACAAAACACCCATGGCTGCCTCTCCTCAG |
| | | | | ACTGACCTGCGTTGACCTGGACCTGGATAAG |
| | | | | CTCCTCACTGTCATCTGAGGGGTGTGTTTCC |
| | | | | CCTTGTGTGCCTGTCCTAATAGTGCATCCCA |
| | | | | TTTCAGCGCTTTTTCTACAGGGCAGGATTTG |
| | | | | TAGAAAGGGTTTGAATCTTAGTGATAAGCTA |
| | | | | TGACCATGAGTAAGTTACTTCATTTTTCCTC |
| | | | | GCTTTTGGTTTTCTTGTAAGAATTGGGATTAT |
| | | | | AGGCCGGTGACATTATAGGCATGGTGACTC |
| | | | | ACGCCTGTAATCCCAGCGCTTTGGGAGGCCG |
| | | | | GGGCAGGCAGATCACAAGTTCAGGACACGG |
| | | | | AGACCATCCTGGCTAACACGGTGAAACCCC |
| | | | | GTCTCTACTAAAAATACAAAAAAATTAGCC |
| | | | | GGGCGTGGTGGCGGGCGCCTGTAGTCCCAG |
| | | | | CTACTCGAGAGGCTGAGGTAGGAGAGTGGC |
| | | | | GTGAACCCGGGAAGTGGAGGTTGCAATGAG |
| | | | | CCGAGATCGCACCACTGCGCTCCAGCCTGAG |
| | | | | CGACAGAGTGAGCTCCGTCAAAAACAAAAG |
| | | | | AAAAGGAAAAAGTACAACTGACTTTGTTTTT |
| | | | | CTGAAACGGAGCCTCACTCTGTCTCCGGGCG |
| | | | | CGATCTTGGCTCCCTGCAACTGCCGCTCCCG |
| | | | | GGTTCACGCCATTCTCCTGCCTCAGCCTCCC |
| | | | | GAGTAGCTGGGACTACAGGCGCCCGCCACC |
| | | | | ACGCCCAGCTATTTTTTTTGCATTTTTAGTAG |
| | | | | AGACGGGGTTTCACCGTGTTAGCCAGGATG |
| | | | | GTCTCCATCTCCTGACCTCGTGATCCGCCCG |
| | | | | CCTCAGCCTCCCAAAGTGCTGGAATTACAGG |
| | | | | TGTGAGCCACCGCGCCTGGCCTACTTTTTCC |
| | | | | TTTCTTATTTGCGTACGTTTTATCTCCTTTCT |
| | | | | CTTGGACTAGAATCTCCAGTACGGTGTTCAA |
| | | | | AAGAAGTGATGAGTGGAGATCAACCAGGTG |
| | | | | CGGTGGCTCACGCCTGTAATTCTAGCACTTC |
| | | | | GGGAGGCCAAGGTGGGTGGATCACCTGAGG |
| | | | | TTAGGAGTTTGACACCATCCTGGGCAACACA |
| | | | | GTGAAACCCTATCTTTACTGAAATGCAAAAA |
| | | | | AATTAGCTGGACGTGGCAGTGTTTGCCTCTA |
| | | | | TTCCCAGCTGCTCAGGAGGCAGAGGCTGGA |
| | | | | GAATCTCTTGAACCTGGGAGGCAGAGGTTG |
| | | | | CAGTGAGCCAAGATTGCGCTACAGCACTCTA |
| | | | | GCCTGGGCGACAGAGTGAGACTCCATCTCA |
| | | | | AAAGAAAAAAAAGAGTGGATATCACAGGCT |
| | | | | TATTTCTTTTTTTTCCTCTTTTTTTTTTTGAAA |
| | | | | CAGAGCCTCGCCCTGTGGCCCAAGCTGAAGT |
| | | | | GCAGTGCAGTGGTGGCTCACTGCAGGCTCTG |
| | | | | ACACAGGCTTATTTCTGATGGTAATTGAAAA |
| | | | | GTGTCCACTTTTTCACCATTAACCATGATGTT |
| | | | | TGCTGTGGGATTTCATAAAGGCACTTTATGA |
| | | | | GGTTGAGGAAGTTCCCTTCTATTACAAGTTT |
| | | | | GCTAAGTATCAGGAATGGACATTGAATTTTA |
| | | | | TAGTTTTCTTTTACATTTATTTATCATTTGGT |
| | | | | TTTGTTTTTTGAACGTTTAACCAATCATGTAT |
| | | | | TCCTGGGTTAAACCCACTTGGTGACAGTGTA |
| | | | | TCATTCTTCCTGTAGGATACATTGGCTGGCA |
| | | | | GGGTGTCAGCTGAGCCCTGTACGTTTCAACA |
| | | | | TCCAGCAGGCTGGCCATTGGGGCTGCCAAG |
| | | | | AACAGTAGGGCAGCAGAAGCAGGGGCCACA |
| | | | | TGGCTTCACTTTTGCTTCGTCCTTCTTTTTTTT |
| | | | | TGAGATAGCGTCTTACTCTGTTGCCCAGGCT |
| | | | | GGAGTGCAGTGGCGCGATCTCGGCTCACTGC |
| | | | | AACCTCTGCCTCCCAGGTTCCCAAGCAATTC |
| | | | | TCCAGCCTCAGCCTCCTGAGTAGCTAACATT |
| | | | | ACAGGCCTGTGCCACCGCGCTCGGCTAATTT |
| | | | | TTGTATTTTTAGTAGAGACAGGATTTCGACA |
| | | | | TGTTGGCCAGGCCAGTCTTAAACTTCTGGCC |
| | | | | TCAAGTGATCCACCTGTCTCAGCCTCCCAAA |
| | | | | GTCCTGGGATTACAAGTGTGAGCCATTGCAC |
| | | | | CTGGCCAACTTTTGCTTCATTCTGTTGGTAAC |
| | | | | AGCAAATCGGTGAGTGAGACTGGGTTCAAG |
| | | | | GGGTGCAAAATAGACTTTCCCCCCGACCTCA |
| | | | | TGATTGGAGGAGCTGCACTCACGTTGCAGGT |
| | | | | GTGGGTGAGAAGGTGATAGAGTCTGTGCCA |
| | | | | TGTGGCATAGCTACTACAACAACTTAAACCC |
| | | | | AAATCCTCTTAGTTTTGCTGTAGTCATCCAA |
| | | | | ATAATTGTTTAGATTTCTGCTTTGGTTTTCCT |
| | | | | TTTCAAGTTAACACTAAGTTAATAGACCCTT |
| | | | | CTTTCCAAGTTCATGATTACAGTGTCATAAA |
| | | | | GTGATAAAGACTGCAGTCTGGGCGTGGTGG |
| | | | | CTCACACCTGTAATCCCAGCACTTTGGGAGG |
| | | | | CCGAGGTGGGCAGATCACTTGAGGCCAGGA |
| | | | | GTTCAAGACCAGCCTGGCCAACATAACCAA |
| | | | | ACCCCGTCTCTACTAAAAATACAAAAAAACT |
| | | | | TACCTGGGTATGGTGGTGCGCATCTGTAGTC |
| | | | | CCAGCTACTCGGGAGGCTGAGGCAAGAGAA |
| | | | | ACACTTGAACCCGGGAGGCAGAGGTTGCAG |
| | | | | TGAGCTGAGATCACGCCACTCCACTTGAGCC |
| | | | | CGGGCAACAGAGCGAGACATTGTCTCAACA |
| | | | | AACAAACAAAACAAAACACTGTGGGTAGCA |
| | | | | AGTCACCCAGTCGTCTTATCTGATTTTTAAA |
| | | | | AACATATGCAGTATGTCTTACGGTATCTTGA |
| | | | | TTAGATTCACAACAGCATTTGGGATCAGCTG |
| | | | | TGCAGTGCATCCTGCGTGTTGAAGAGTGATA |
| | | | | CAGGGTCAGATGCAACGCCTGTAGTCTCAGC |
| | | | | ACTTTGAGAGGCCAAGGCGGGGGATCACTT |
| | | | | GAGACCAGGAGCTCAAGACCAGCCTGGGCA |
| | | | | GCATAGCAAGACCCCGTCTCTACAAAAATA |
| | | | | AAAATAAAACTTAGCTGGGTGTGATAGCAT |
| | | | | GTGCCTGTAGTCCCAGCTACTTGGGAGGCTG |
| | | | | AGGCTGGATGGCCACTTGAGCCCAGGGTTTC |
| | | | | TTTTTAGAGACAAGGTCTTACTCTGTGGTCC |
| | | | | AGGCTGGAGTGCAGTGTTGCCATCACAGCTC |
| | | | | ACTGCAGCCTCAACTTCCTGGGCTCAGGCAG |
| | | | | TTTTCCCACCTCAGCCTCTCAAATAGCTGAG |
| | | | | ACTACAGGTGCGTGCCACCATACCCAGCCA |
| | | | | GCTAATCTTTCTGTTTTTTTTTTTTGTAGAGA |
| | | | | TGGGGTTTAGCCATGTTGCCCTGGCCCATCT |
| | | | | CAAACTCCTGGGCTCAAGCGATCCACCTGCT |
| | | | | GTGGCCTCCCCAGGTTCTGGGATTATAGACA |
| | | | | TGAGCCGCCGCTCCTGGCCTGATTGCAGCTT |
| | | | | GTGGGTTTGGCAACTTTGGTCAATAAAAGAT |
| | | | | TATGTGGTCTTTTTCTCCCTGCGCCTTCTCAC |
| | | | | TCCTGGCACAGAGTTCCTGCAACCATTGGAA |
| | | | | TTTCTTATATGATAGGATGTCATTTGTTATTC |
| | | | | ATAACTAGCTCCTTTCAGATCACACCAGAGT |
| | | | | TTAAGCTAATGAACTGACAGGGTAGGGGTG |
| | | | | GTCACCAGGAAGACCAGATGATTATTAGAG |
| | | | | GGCTAGGGCTTTCATCTCCGGCCACTGACCT |
| | | | | CCAGGGAAGGGAGCAGGAGCTGAAGATGGA |
| | | | | GCTCTAAAAACTCTCGAACATGCCCGAGTTG |
| | | | | CGGGAGGGCCCCTGCCGCCCACACTTGGCTC |
| | | | | TTTGCATCTTTTGTTTGCTGCTACCGAGTTGT |
| | | | | CTTTTTTATATTTTCCATGTCGAACATGTGGA |
| | | | | ATACAGTTCAACAGCTTTTAATGTCTTTGTCT |
| | | | | ACTAATTTTAACACCTGTTTCAATTCTATGTT |
| | | | | GGTTTATTTCTTTTAACCTCATTATGGTTTGT |
| | | | | ATTTGTGTTTTTTTGAGCACTAATCGTCGTTT |
| | | | | TCTGAGGCTTGTATTTTTTCCTTCTTTGCCTA |
| | | | | GTGATTTTGTTGGATGCCAGGCATTAGGAAT |
| | | | | TTTACCCCGTTGGGTGATGACTTTCTTTTGAA |
| | | | | AAATGAACGAGATCTGGTTCACAGAATACA |
| | | | | AAATTTACCATTTACAGCTCACAGTTTAGTG |
| | | | | ATTTTTAGTATATTCATTATATTGTGCACCCA |
| | | | | TCACCACTGTCACATTCCAGCAAGTTTCCAT |
| | | | | CCTCATGCGGTCCCCTGTGTCATCAGTCCTT |
| | | | | GTCCCCCTTCCCCCTTTCCCTGACAACTGGT |
| | | | | ACTCTCTTTTCTATCTCTCTGGATTTGCCTAT |
| | | | | TGTGAATGTTTCCCATAAATGAGAATTAAGT |
| | | | | CTTTTGGGTTTTAGATAAATAAGATCATCAT |
| | | | | TTCTTCTTCAGAGAATTGAACTGTCAGCAGG |
| | | | | TGGGGGCTCGTTGCTTGTAGGGGGTGGGCTG |
| | | | | CATGCGCTCTGGTGTTTACCTGGTGTGCCTG |
| | | | | AGCCCACGGCCAGTGCAGCAGGTTCTGCCA |
| | | | | GCATCTTTTTCTGGGCAGCTTGTTGAGTTTAT |
| | | | | GACACAATCTCCTTTTACTGGTCCCTGTTGT |
| | | | | GATTGGCACCCTGACCTTTTAGAAAGTGTGA |
| | | | | TGGTGGCCAGGCGCGGTGGCTCACGCCTGTA |
| | | | | ATTCCAGCACTTTGGGAGGCCGAGGCGGGC |
| | | | | AGATCATGAGGTCAGGAGATCAAGACCATC |
| | | | | CTGGCTAACACGGTGAAACCCCATCTCTACT |
| | | | | AAAAATACAAAAAATTAGCCGGGCATGGTG |
| | | | | GCGGGCGCCTGCAGTCCCAGCTACTCGGGA |
| | | | | GGCTGAGGCAGGAGAATGGCGTGAACTCAG |
| | | | | GAGGTGGAGCTTGCAGTTAGCTGAGATCGT |
| | | | | GTCACTGCACTCCAGCCTGGGCGACAGAGC |
| | | | | GAGACTCCGACTCCGTCTCAAAAAATAACA |
| | | | | AAAACAAGAAAGTGTGATGGTGGCTGTGGA |
| | | | | GCAGAGGAGTCTTCTTTAGCTCGTCCACGTG |
| | | | | ACGTGCAGGAGTACATGGGAGTCGTGTGCT |
| | | | | GGTGGACACACAGAAGCGACTTTTTCCTTTG |
| | | | | TCTCATGGTGGAATGCTGGAGGAGCAAGTG |
| | | | | TTCCCACCTGCTGAGCAGCTGAAAGCAGGCT |
| | | | | TTCCGCGGATGTGTGCGGGCGGGCGGTGTG |
| | | | | GCACAGAAAGGCCTCGATGGACTGTCTGCTC |
| | | | | TTCACGGCAAGAGTGTGCTGCCCCTGTTCTC |
| | | | | TGTCGTGGCCTCTGCTGTAAAGATGAGAGAA |
| | | | | GTCCAACGCCAGTCTAATTCATGATTCTTTTT |
| | | | | TAGTTAAGCTTTTTAGGATTTTGTGGAAACT |
| | | | | TGTAGGATTTTCTGTTGATATGTGGGAGTCT |
| | | | | GACATTTTGCTATTTTTAGGGTTTAGATTGTG |
| | | | | ACTTTTTTATATTGGTTAGCATCTTGAAGCTC |
| | | | | TTTTAATATGCAGACTTGTCTTTTTAACTCCA |
| | | | | TCTCAGTTTTTCTGTCAAGGGCCAGTTAGAC |
| | | | | CTTGTGGCCGCATGTTCTCTGACAGCTCTTC |
| | | | | AGCTCTGCTGTTGTATCATGGAAGGAACCGG |
| | | | | GGCCACACTTAAAACGAGCCTGGCTGTTTTC |
| | | | | CAGTTACAACTATTACTAACACTGAAATTAG |
| | | | | AATTTCATATAATTTTCACTTACCACAAAAT |
| | | | | GTTATTTTCTTTTTCAACCAGGTATAAATATA |
| | | | | AACCTCATTCTTAGCCTGTGGACTGTCTAAA |
| | | | | AAAGACATTGGGCCAGATTCGGCCCCTGAC |
| | | | | CCCCAGGTGGTTTCCCCATCATAGTAATCAT |
| | | | | AGTAGATGAACTTGGAACTGTGTAGCTTTAT |
| | | | | TGGTTTTTCATCCTTGAAGCTTGCTTTTCCTC |
| | | | | TGCAGTGGTCCTGACTTTCTGTAGAGTTTGA |
| | | | | TCTCTGCGGCTTCTTCCTTCTGTAGAGTTTGG |
| | | | | TCTCTGTTTTCTCTAAACTTAAGTTATTTGTC |
| | | | | TGATTTCATTTACTTTCTGTTAATTTAGGTGC |
| | | | | TTAATTCTGGTCAACTATTGACATGTCATTCT |
| | | | | TCTGTTTTCCAGTGCTGTTACAGATTTATTCT |
| | | | | TTTCTTTTTTTCTTTTTCTTTTCTTTTCTTTTTT |
| | | | | TTTTTTTTTTAAGATGGAGTCTCACTCTGTCG |
| | | | | CCCAGGCTGGAGTGCAGTGTTGCGATCTTGG |
| | | | | CTCACTACAGCCTCCACCTCCCAGGTTCAAG |
| | | | | CGATTCTCGTGCCTCAGCCTCCTGAGTAGCT |
| | | | | GGGATTACAGGTGTCTGACCCCACGCCCAGC |
| | | | | TAATTTTTTGTATTTTTAGTAGAGATGAGGTT |
| | | | | TCACCATGTTGGCCTGGCTGGTCTTGAACTC |
| | | | | CTGACCTCGTGATCTGCCTGCCTCGGCCTCC |
| | | | | CAAAGTGCTGGGATTACAGGCGTGAGCCAC |
| | | | | CACGCTCTGCCAGATTTATTCTTTTCAAAAT |
| | | | | GTTTGTTACTTTAAGAAATTTTAGATAAGAG |
| | | | | GGTTAGATTCCACCATTGTGATCTTTTTTTTT |
| | | | | CTTTTGAGATGGAGTCTCGTTCTGTCACCCA |
| | | | | GGCTGGAGTGCAGTGGTGCGATCTTCGCTCG |
| | | | | CTACAGCCTCCACCTCCCAGGTTCAAGTGAT |
| | | | | TCTCGTGTCTCAGCCTCCCGAGTAGCTGGGA |
| | | | | CTACAGGCGCCCGCCACCATGCCTGATTAAT |
| | | | | TTTTGTATTTTTAGTAGAGACGGGGTTTCAC |
| | | | | CATGTTGGCCAGGCTGGTCTTTAACTCCTGA |
| | | | | CCTCAAGTGATCCGCCCACCTCACCCTCCCA |
| | | | | AAATGTTGGGATTACAGGTGTGAACCACTGT |
| | | | | GCCTGGCGTTAGCGTTGTGGTCTAATAGTAC |
| | | | | TACAGTACTATTGTTTTTTGTAAATAGAATT |
| | | | | GCAGTCTGAACAGGAAGAACTTCCACCATA |
| | | | | GGTGTTTTGAAGAAGTTAATTTTTTGCATAA |
| | | | | GTAGAAAGCCATGGGAGCATTAAACTTAAC |
| | | | | AGTCTATTGCTTGTGTGGTAACGTAGGGAAT |
| | | | | TAATTTTGAATTAAATGTGAACTAGACAATT |
| | | | | TGCTGTGGAATACTACGTTGAAATTATTGAA |
| | | | | AAACACTTATCCAGTGTGAGGCTTTTTTTTTT |
| | | | | TTTAATTTATTTATTTTTTATTGATAATTCTT |
| | | | | GGGTGTTTCTCATAGAGGGGGATTTGGCAGG |
| | | | | GTCATAGGACAATAGTGGAGGGAAGGTCAG |
| | | | | CAAATAAACAAGTGAACAAAGGTCTCTGGT |
| | | | | TTTCCTAGGCAGAGGACCCTGCGGCCTTCCG |
| | | | | CAGTGTTTGTGTCCCTGGGTACTTGAGATTA |
| | | | | GGGAGTGGTGATGACTCTTAACGAGTATGCT |
| | | | | GCCTTCAACCGTCTGTTTAACAAAGCACATC |
| | | | | TTGCACCGCCCTTAATCCATTTAACCCTGAG |
| | | | | TGGACACAGCACATGTTTCAGAGAGCACAG |
| | | | | GGTTGGGGGCAAGGTCACAGATCAACAGGA |
| | | | | TCCCAAGGCAGAAGTTTTCTTAGTACAGAAC |
| | | | | AAAATGAAAAGTCTCCCATGTCTACTTCTTT |
| | | | | CTACACAGACACGGCAACCATCCGATTTCTC |
| | | | | AATCTTTTCCCCACCTTTCCCCGCTTTCTATT |
| | | | | CCACAAAACCGCCACTGTCATCATGGCCCGT |
| | | | | TCTCAATGAGCTGTTGGGCACACCTCCCAGA |
| | | | | CGGGGTGGTGGCCGGGCAGAGGGGCTCCTC |
| | | | | ACTTCCCAGTAGGGGCGGCCGGGCAGAGGC |
| | | | | GCCCCTCACCTCCCGGGCGGGGCGGCTGGCC |
| | | | | GGGCGGGGGGGCTGACCCCCCCACCTCCCTC |
| | | | | CCGGATGGGGCGGCTGGCCTGGCGGGGGGC |
| | | | | TGACCCCCCCACCTCCCTCCCGGACGGGGCG |
| | | | | GCTGGCCTGGCGGGGGGGCTGACCCCCCCC |
| | | | | ACCTCCACCTCCCTCCCGGACGGGGCAGCTG |
| | | | | GGCGGGGGGCTGACCCCCTCACCTCCCTCCC |
| | | | | GGATGGGGCGGCTGCTGGGCGGAGACGCTC |
| | | | | CTCACTTCCCAGACGGGGTGGCTGCCGGGCG |
| | | | | GAGGGGCTCCTCACTTCTCAGACGGGGTGGT |
| | | | | TGCCGGGCAGAGGGTCTTCTCACTTGTCAGA |
| | | | | CGGGGTGGCCGGGCAGAGGTGCTCCTCACA |
| | | | | TCCCAGACGGGGCGGCGGGGCAGAGGCGCT |
| | | | | CCCCACATCTCAGACGATGGGCGGCCGGGC |
| | | | | AGAGACGCTCCTCACTTCCTAGATGTGATGG |
| | | | | CGGCCGGGAAGAGGTGCTCCTCACTTCCTAA |
| | | | | GTGGGATGGCGGCTGGGCGGAGACGCTCCT |
| | | | | CACTTTCCAGACTGGGCAGCCAGGCAGAGG |
| | | | | GGCTCCTCACATCCCAGATGATGGGCGGCCA |
| | | | | GGCAGAGACGCTCCTCACTTCCCAGACGGG |
| | | | | GTGGCGGCCGGGCAGAGGTTGCAGTCTCGG |
| | | | | CACTTTGGGAGGCCAAGGCAGGCGGCTGGG |
| | | | | AGGTGGAGGTTGTAGCGAGCCGAGATCATG |
| | | | | CCACTGCACTCCAGCCTGGGCACCATTGAGC |
| | | | | ACTGAGTGAACGAGACTCCGTCTGCAATCCC |
| | | | | GGCACCTCGGGAGGCCGAGGCTGGCGGATC |
| | | | | ACTTGCGGTTAGGGGCTGGAGACCGGCCTG |
| | | | | GCCAACACAGCGAAACCCCGTCTCCACCAA |
| | | | | AACCAGTCAGGCGTGGCGGCGTGAGCCTGC |
| | | | | AATCGCAGGCACTCGGCAGGCTGAGTCAGG |
| | | | | AGAATCAGGCAGGGGGGTTGCAGTGAGCCG |
| | | | | AGATGGCAGCAGTACAGTCCAGCTTCGACTC |
| | | | | AGCATGAGAGGGAGACCGTGGAAAGAGAG |
| | | | | GGAGAGGGAGACCATGGGGAGAGGGAGAG |
| | | | | GGAGAGAGGGAGAGGGAGAGGGGGAGAGG |
| | | | | GAGAGGGGGAGGGAGAGGGAGCGTGACGC |
| | | | | TTTTTTTAAATGAAGCTCGTGACAGACGGAA |
| | | | | GTATACCAGTGATTAAGAAGATGCTGGGAT |
| | | | | GGGCTTTTTCAATAGATGCTCTGCAGGTTTC |
| | | | | CAAAATGAGTGCCAGAGGAGGGGAGAGGGC |
| | | | | AGTGTCAGAGTCTTCTGAACATTCTGAGAGC |
| | | | | TGAGCTGCTGTGAGACAGGCTTAAATGAGA |
| | | | | ACCCTAGTTTTCAAAACTTAATGTTTTAATG |
| | | | | GGAATGACCATAGTTATTAGTGTTAAAAGAT |
| | | | | ACATTTCTTCTTATTTATTTAGAAGATGAAG |
| | | | | TTCAGAGAATTTAGGTAGCCTAAATAAGATG |
| | | | | GCATAGTTAGTAATTCTATGAGCTTTTCCTT |
| | | | | GTTTAGTAAATCGGTATTAAAATGGAATTAT |
| | | | | TAAGTGGGGTGTGGTGGCTCACGCCTGTAGT |
| | | | | CTCAGCACTTTCGGAGGCCGAGGGAAGCAG |
| | | | | ATAACGTGAGCACAGGCGTTTGAGACCAGC |
| | | | | CTGGGCAAATGAAGATACCCTGTTTCTACAA |
| | | | | AAAATACAAAAGTTAGCCAGGCGTGGTGGC |
| | | | | AGGTGCCTGTGGTCCCAGCTCCTCAGGAGGC |
| | | | | TGAGGGGAAGGATTCCCTGAGCCCAGCAGT |
| | | | | ATAAAATTGACCATTTGTACCATTTTTGAGT |
| | | | | GTGCAGTTCTCTGGTATTAGATTCACACGGT |
| | | | | GCAAAGCCATCACCACCATCCCTCTCCAGAA |
| | | | | CTTGGTCTTCCCAGACGACACCGGCTACCCA |
| | | | | TTAACACTAACTCTTCATCCCTCTCCCCATA |
| | | | | ACCTCGACTCTCCCCATAACCCCTGACCACC |
| | | | | AATCTGCTTTCTCTTATGAATGTCACCACTC |
| | | | | AAGGCACCTCTCCTATAAGGGGGGGGGGGG |
| | | | | GGTCATACGATATTTGTCCTTTCTTCTCTTAT |
| | | | | GAATGTCACCACTCAAGGTGCGTCTCCTATG |
| | | | | GGAGGGGGTCATACGATATTTGTCCTTTCTT |
| | | | | CTCTTATGAATGTCACCACTCAAGGTGCGTC |
| | | | | TCCTGCGGGGGGGTGGTCATACGATATTTGT |
| | | | | CCTTTCTTCTGTTATGAATGTCACCACTCAA |
| | | | | GGTGCGTCTCCTGTTGGGGGGGGGGGTCATA |
| | | | | CGATATTTGTCCTTTCTTCTCTTATGAATGTC |
| | | | | ACTGTCCAAGGCACCTCTCCTGTAAGGGGTG |
| | | | | GGGGGTCATACAATATTTGTCCTTTCGTGAC |
| | | | | TGGCATATTTCCCTTTTGGATCAGTTTGTTCC |
| | | | | TGTGGGTCAGAATCTTCATTTGAACAGTTTG |
| | | | | CCCCACAGCTCAGATTCCTCAATTGTGACTA |
| | | | | CCCCCTGCAGGTCAAATTCAATTTTTGTTTA |
| | | | | CTTATTTTTGAGACAGAGTCTTGCTGTCTTGC |
| | | | | TCGGGCTGGAGTGCAGTGGTGTGATCGTGG |
| | | | | ATCACTATAGCCTCGACCTCCTGGGCTCAAA |
| | | | | CAACCCGCCTGCTTCAACCTTCCATAGTGCT |
| | | | | GAGATTACATGCGTGAGCTGCTGTGCCCAGT |
| | | | | GTCAAATTTAATTTTTGTTTTGTTTTGTTTTT |
| | | | | GAGACAGAATCTCGCTCTGTCACCAAGGCTG |
| | | | | GAGTGCAATGGCACTATCTTGGCTCACTGCA |
| | | | | TCCCCCACCTCCCAAGTTCAAGCAATTTTCC |
| | | | | TGCTTCAGCCTCCTGAGTAGCTGGGATTACA |
| | | | | GGCACCTGGCACCACGCCCGGCTAATTTTTT |
| | | | | GTATTTTTAGTGGAGACGGGGTTTTGCCATA |
| | | | | TTGGCCAGGCTGGTCTTGAACTCCTGACCTC |
| | | | | AGGACATTTATAGGATACACTTATTATTTTT |
| | | | | ATGACCAAAGCATGTGATTTTTATTTTTTAA |
| | | | | TTTTAATTTTATTTTTTAATGTTTTTTGTTCTT |
| | | | | GTTGTTTTTGAGACGGTGTCTTAGTCTGTTGC |
| | | | | CCAGGCTGGAGTGCAGTGGCGCAATCTCAG |
| | | | | CTCACTGCAATCTCGGCCTCCCAGGTTCAAA |
| | | | | TGATTCTCCTGCCTCAGCCTCCCGAGTAGCT |
| | | | | GGGATTACAGGCGCACACCACCACGCCCAG |
| | | | | CTAATTCTTTTGTATTTTTAGTAGAGACGGA |
| | | | | GTTTCACCATGTTGGGTAGGCTGGTCTCAAA |
| | | | | CTCCGGACCTCAAATGATCCACCTACCTCAG |
| | | | | CCTCTCAAAGTGCTGGGATTACAGGTGTGAG |
| | | | | CCACCGCACCTGGCCTTTTTTTTTTCTTTTTT |
| | | | | TTTGGATACAGGGTCTTGCTTGGTCACCCAG |
| | | | | GATGGAGTGCAGTGACACGAAATTGGCTCA |
| | | | | CTGCAATCTCGACTTCCTGGGCTCAAGCGAT |
| | | | | CCTCCAGGCTCAGCCTCCTGAATAGCTGGGA |
| | | | | CTGCAGGCACGACCACCATGCCCAGCTACTT |
| | | | | TTTTATTGTTTGTAGACATGGGGCTGGTCTC |
| | | | | AGACGCCTCAAGAAATCCTCTTGCCTAGGCT |
| | | | | TCCCAGTGTGCTGGGATTACAAGCATGGGCC |
| | | | | ACTGTTCCTGAATTTTATTTTTTTAAACCTTT |
| | | | | TTATAGAACACGATCAGTTGTTTGATAAATA |
| | | | | CTGAAACAGTACTAGGAATCAGTTTTTTAGT |
| | | | | TGTTTACCAAACATATTATGCAGGAACTGAA |
| | | | | TTCACAAAAAGTTGTTTTGAAATTTGGTCCA |
| | | | | CAAATTCACTTAAGGTTGGAAATAAAAAAC |
| | | | | TTGTAAGAGGCCGGGTGTCGTGGCTCAAAA |
| | | | | AGAAAGGAAAGGATACTTTCAGGCTTAGAG |
| | | | | TTAGTCTTTTTTGTTGGAAATTTTCACAACTT |
| | | | | CAGAAAAACTTCATCAACAGGTTTAGAAGC |
| | | | | ATCCGTTTTATTGACTTTCCCGATTTCTTCGT |
| | | | | ATGAGTCAGTAATTGTTTTTGTTAACTTGAA |
| | | | | GATGGGTCTGAATTCTCTTTTCCAAGCTCTCT |
| | | | | CTGCTGGCTTCACTCTTACCACTGTTCCCTCC |
| | | | | TCTCAAGACATCCTTTCATGTAGATCTCATT |
| | | | | ATTATGGTCAGAAAACAGAACCAGATGCAC |
| | | | | ATCTGCCTTTCCCATCCATGCTCTTGGCCCA |
| | | | | ACCTTGAAGGTTGTCTTGATTTCTTAGAAAC |
| | | | | TCATCAAGAATTTATTCTTAGCATTGGCAGC |
| | | | | CATTGTCTGATCCATTTCCCATGTGAATAAA |
| | | | | TGCATGTATGGTTATTTCATCTAGCACAGTC |
| | | | | TTCCCTTGTTTAGTATAGTTTTTGAAGAGTTC |
| | | | | ACCACTGTGAATCTGGGTTCTTTCATCACAG |
| | | | | AGAAGTTTTCCTGAAGACAGGTGTATGTGAC |
| | | | | CAGGGTCACGACGGTGTGGGTTTGCTGCGTC |
| | | | | CCCTTGCCAGTGCCAGGACCCCTGAGGAACC |
| | | | | ACAGGACCAGTGGGCAGCTTCATGAGGCCG |
| | | | | GGGCCGCAGAGGAGATGCAGGCAGCCAGTC |
| | | | | AGCAGCAGGAAGCTGAGGCCTAGGGCACGC |
| | | | | AGTGGCCAGCAGCAGGCTGGCTCCCTCTTTG |
| | | | | GGAGGTTAAATCAAGTTTTGGTTACCAAAGG |
| | | | | CAAAGCAGGCTTGGATTTTGTTTAAGGAGAT |
| | | | | GCTGTTGAATGAAACAGCTTCTTTGTAGCAA |
| | | | | GCAGCCTTGGAGATGATTAATGGAACATGTC |
| | | | | TGAACTTGCCAGAGTGATGTTCAGCCTTCCA |
| | | | | GTGGAACTGCAAAATCATGAGTGAAAGCGT |
| | | | | GGCAAAGTATTTTCTTTAATGTAAAATGTTA |
| | | | | TTCCTAAGAAATGAAATTACGTGGCCGGGC |
| | | | | ACGGTGGCTCACGCCTGTAATCCCAGCACTT |
| | | | | TGGGAGGCCGAGATGGGCGGATCACGAGGT |
| | | | | CAGGAGATCGAGACCATC |
| 2 | ANKRD11 exon 3x | chr16:89379725- | − | ATTTTTCCAAAAGAATCGTGATCTCAGTGAC |
| | | 89379997 | | ATATACGTGGAAGATGGAAATGGAGCCCAC |
| | | | | AACTCTGCAGTGCATCCTGATGCCGCGCTGA |
| | | | | CCTGACGGCTTGTGCGTGTCCCTTTGGCTGC |
| | | | | ACCAGTGAGCACAGTGGCAGGCGTGTCAGA |
| | | | | GAAAGGCCCCTTCTGCAGACGGTCTCTCACC |
| | | | | ATTGCCGACCACGGAATCCCAGAACCGCTG |
| | | | | AGCTGCCTCGGGAAGAACCAGCAGGTGTCT |
| | | | | GCATCGTTGAGTGTGTTCTGATCCAAAG |
| 3 | ANKRD11 exon 4x | chr16:89358089- | − | ATGCCTCCAGCCCAGTCCCTGTTGTGGTGCT |
| | | 89358185 | | GCAAGGCTGGTACGCTCCTCGAAGCACCAT |
| | | | | GGCATGAGATGGAGGTTCCTAGAAGCAAGA |
| | | | | AGAAAG |
| 4 | ANKRD11 exon | chr16:89358089- | − | tgctctgtgttgatgccttcagATGCCTCCAGCCCAGTCC |
| | 4x + 22 | 89358207 | | CTGTTGTGGTGCTGCAAGGCTGGTACGCTCC |
| | | | | TCGAAGCACCATGGCATGAGATGGAGGTTC |
| | | | | CTAGAAGCAAGAAGAAAG |

TABLE 2
NSD1 pre-mRNA and poison exon sequences.

| | | Genomic | | |
|---|---|---|---|---|
| Seq. ID | Seq Name | Coordinates (hg19) | Strand | Sequence |
| 5 | NSD1 pre-mRNA | chr5: | + | GACGCGGGGGGAGGGGGGTGCGGCGAGCG |
| | (based on | 176560833-176727214 | | GCCCCGCTCTCTCCCCACCGCTCCGCTCGC |
| | NM_172349.2) | | | ACCCCAGTGTAATGAGGGTCACCCCCTCCC |
| | | | | CCCAGCTGGCCCGGGAGGGGGCGCGGGGC |
| | | | | ACGGTAACTAGTGCGCTGGGGTGGGCGGC |
| | | | | GGGCAGGCGCGAGGAGAAGGGAGGGAGG |
| | | | | AGGGTGGCCGGGCGGGGAAGATGGTGGTG |
| | | | | GCCGTAAGGTGAGGGGCTCGGGGGAGGGC |
| | | | | CAGGCGCGATGCGGGGTTGGTGGCCGGCG |
| | | | | GCGCTGCAGCCGCCGGCCTCCTCCCCCTCC |
| | | | | CCCTCCTCCATCACTACCAGCCGGGCTCAG |
| | | | | GCCTAGCTGGCCGGGCTGCCGCGAACTTCC |
| | | | | TCCCGGCGCGGCCCGTGCCCCGCCGGCCGC |
| | | | | CTGCGAACACCTCGGCCTCCGCCTCCCCTC |
| | | | | AGGTAGCAGGCTGCGGGGCGCGGGGCCGG |
| | | | | CTGCCCTCCCGCAGCAAACTTTGCTTGCTG |
| | | | | CTGAATATTGATGAGAGCGATCGGCTCGGC |
| | | | | TGGGAGGTGCTGCCGCGGCTGCGGGAAGG |
| | | | | AGCGCGGCCCGGGCAGGCGGCGGCGGCGT |
| | | | | CGGCAGCAGCCATGTTTTTCGAGCTGTAGC |
| | | | | AGCTGCTGCTACCCTGACTGGGCTTCGCTG |
| | | | | GCCGCCTCGGTTTCTCCCTCTGCCGGGTCCA |
| | | | | GGCCTCTTCGCCCTGCAGCTGCGGATCCAG |
| | | | | CAGGCCTGCATTCAGGAAGGCGAGCTCTGG |
| | | | | GGTGCAGCCGCCTCGGCCGGCTCGCCTGCG |
| | | | | GCCTGCGCACCGCCGCTGCAAAGGCTCCGG |
| | | | | CGCTGGCTGGGCGCAGGGTGCAGCGCTATT |
| | | | | GTGACCGCTGCGCCCTAGCGAGCCAGGAA |
| | | | | GGGGGGGGTACCTTTTTGTGCAGGGTCCAG |
| | | | | GAGCCCCCCTCGGACCCCGCAGCCTTTTGC |
| | | | | TTTTGAGAGATCCAGCTGCTCGACCCCTGG |
| | | | | CGAGGGAGGGGGAGGACTAGTCCTGTTTG |
| | | | | AGAATTGGGAATTTTGACGGGCAGAGGGG |
| | | | | TTTTAATTTTAGTTCATCCCAAGTGTCCACC |
| | | | | AGTCTACAGAGGAGGAAAAAGAGACGGGC |
| | | | | TGTTTCTATGTAGCAGGATCGGCCCAGCTT |
| | | | | CGGGAAAATGGAGTTTTCAGAGGCTCATCG |
| | | | | AGGCCATTTTTTCATCTCCAGTCGGGGGAA |
| | | | | CTTTTTCTGCCCATGGAAGTGCAGCAGAAA |
| | | | | GGCATAGAGGCCACTAGGCCTTGAAGTGGC |
| | | | | TGCCATTTTAAAGAGTCGAGTCAGATGGCC |
| | | | | TATTAACTCAGATTAATTGCTGTGCTTTTGG |
| | | | | ATTCCAGGTTGATGCCGGCCCAGGATGGAT |
| | | | | CAGACCTGTGAACTACCCAGAAGAAATTGT |
| | | | | CTGCTGCCCTTTTCCAATCCAGTGAATTTAG |
| | | | | ATGCCCCTGAAGACAAGGACAGCCCTTTCG |
| | | | | GTAATGGTCAATCCAATTTTTCTGAGCCAC |
| | | | | TTAATGGGTGTACTATGCAGTTATCGACTG |
| | | | | TCAGTGGAACATCCCAAAATGCTTATGGAC |
| | | | | AAGATTCTCCATCTTGTTACATTCCACTGCG |
| | | | | GAGACTACAGGATTTGGCCTCCATGATCAA |
| | | | | TGTAGAGTATTTAAATGGGTCTGCTGATGG |
| | | | | ATCAGAATCCTTTCAAGACCCTGAAAAAAG |
| | | | | TGATTCAAGAGCTCAGACGCCAATTGTTTG |
| | | | | CACTTCCTTGAGTCCTGGTGGTCCTACAGC |
| | | | | ACTTGCTATGAAACAGGAACCCTCTTGTAA |
| | | | | TAACTCCCCTGAACTCCAGGTAAAAGTAAC |
| | | | | AAAGACTATCAAGAATGGCTTTCTGCACTT |
| | | | | TGAGAATTTTACTTGTGTGGACGATGCAGA |
| | | | | TGTAGATTCTGAAATGGACCCAGAACAGCC |
| | | | | AGTCACAGAGGATGAGAGTATAGAGGAGA |
| | | | | TCTTTGAGGAAACTCAGACCAATGCCACCT |
| | | | | GCAATTATGAGACTAAATCAGAGAATGGTG |
| | | | | TAAAAGTGGCCATGGGAAGTGAACAAGAC |
| | | | | AGCACACCAGAGAGTAGACACGGTGCAGT |
| | | | | CAAATCGCCATTCTTGCCATTAGCTCCTCA |
| | | | | GACTGAAACACAGAAAAATAAGCAAAGAA |
| | | | | ATGAAGTGGACGGCAGCAATGAAAAAGCA |
| | | | | GCCCTTCTCCCAGCCCCCTTTTCACTAGGAG |
| | | | | ACACAAACATTACAATAGAAGAGCAATTA |
| | | | | AACTCAATAAATTTATCTTTTCAGGATGAT |
| | | | | CCAGATTCCAGTACCAGTACATTAGGAAAC |
| | | | | ATGCTAGAATTACCTGGAACTTCATCATCA |
| | | | | TCTACTTCACAGGAATTGCCATTTGTAAGC |
| | | | | AGTTTTTGGTACAACTTAAATATATACATA |
| | | | | TATGTATATATACAGGCCACTTAAAGGGAA |
| | | | | ACTTGTAACAAATTTGTTTTTGGTTGCTTAT |
| | | | | CAGTTCACAGCTGAAATCCTATTGCTAATC |
| | | | | ATAAGCTTTGGGCAAAATTTTACTTTGATTT |
| | | | | TTAAATTTATCTCTGTTGTATGAATTTGGTT |
| | | | | GTTTTAAGCTTTTTCCAAATAACTCTTCATT |
| | | | | GAGAGTAGGCTAATGCTTTTAAAGGCATTT |
| | | | | GATTGAGTTCAGGTTTAATTTCTCAAGTTG |
| | | | | GAGGTATACATATATGATTAAAAAAAAAA |
| | | | | AAAAAAGATGGGTTTTGGCCTGCCAGCACC |
| | | | | ATGAGTGCAGGTGAACCAATTTAGTACTTG |
| | | | | GAGTCCTGTTGCTATATGTGGCAGATTATTT |
| | | | | TTTTACTTGATGACTTGACTCTTACTTCAGG |
| | | | | TTGAAGGGCATTTTGAACACAGATTAAAGT |
| | | | | GGCTAAGATGAAGTTTTCTTGGACATTGTC |
| | | | | AAAATCTAAATTAGGCTAGTTTTTCTGAAC |
| | | | | TACCTGTTTTGAAGGTATAGCATCCTGTGCT |
| | | | | TTTGATAACTGCCACCATTAGCTCTTTTTTT |
| | | | | TTTTTTTGAGGTGGAGTCTCACTCTGTTGCC |
| | | | | AGGCTGGAGTGCAGTGGTTGATCACTGCAA |
| | | | | CCTCTGCCTCTTGGGTTCAAGCAATTCTCCT |
| | | | | GCCTCACCCTCCCGAGTAGCTGGGATTACT |
| | | | | GGTACCCATCACCACGCCCGGCTAATTTTT |
| | | | | GTATCACCATTAGCTCTTGAAGTTTTTCTAG |
| | | | | TTTTGTTTTGTTTTATTTTATTTTATTTTAAC |
| | | | | AGAACCCTAACTAAGACAAAGTTTTATATT |
| | | | | TATTTATTGTTTAGAGACTGGCCTTGTCATG |
| | | | | TTGCCCAGGCTGGCGTCGGGACTCCTGGGC |
| | | | | TCATTCGATCCTCCTGCATCAGCTAGAACT |
| | | | | ACAGTAGTTTCAGATTTTGAAGTGTGTATG |
| | | | | TGTATGTGTGATATGTATATATTCCGTGTGT |
| | | | | ATAGAAATGGAGAGTATCTTATTTGAGTTG |
| | | | | TTGTTTTCAGTAATGCTGTCAAGTATTGTTA |
| | | | | GAGGGTGATAAATGATAACATTTGTTTTTA |
| | | | | TTTGAGCTTATGAAGAATTTCTTGACTTTCT |
| | | | | AGCTAAATGATCAGTTCACTTCTCTTAGCCT |
| | | | | CAATTTTATTGCGTCTAAATTCCAGAAGTTC |
| | | | | TTGATTGCTATAAGATTCCTTCAGCTTTAAA |
| | | | | TATTAATATTTGATATTGATTTTGTTTCTGC |
| | | | | CCAAACACATTGTTTGGTCACCGCCGGTAA |
| | | | | TGTTAGCAAAGAGAATTTTTTTTGGCCAAC |
| | | | | AAATGTCTCATACCACATTCAGTTTTTATAA |
| | | | | GAAAAACTTTTATGGTATGTTGTTATTCTGA |
| | | | | GTTCATTAAACATTCGCTTTACCTTATATCC |
| | | | | CTGCTGTTCTTTAAAGTTACAGAGGGAGAA |
| | | | | TGTGGGTGTGTCACTTTTGTTTCTGTTGATT |
| | | | | TGTATCTTAATTATGCCTTGGTACTCCTTGG |
| | | | | TTTCTTGGCAATTGCAGATTTAAAAAAATT |
| | | | | TGCTTTAGTGGTTATCTTGAGTCTGAATTGT |
| | | | | CCTACACATTAGGGTGGGTAGGCTGTTTTG |
| | | | | AAAACCTATTGGCAGCTCAGACAAATCCTT |
| | | | | TTTCTTGGGTTCACGTTGAAATTTATTTTAT |
| | | | | ATATATATCGTGTCTTTGTTTTTGCACATAA |
| | | | | ATTTAAATCTGAGAATGGAGATAGATGTTT |
| | | | | CTCTAGAAGCATACAAATAGAATTGTAAAC |
| | | | | CTGTTTCTCGTCAAAGAGATGTTAGTGGAG |
| | | | | TATTGGTTCTATTAAAAAAAAAATGAAGGC |
| | | | | TGAGTGTGGTGGCTCACACCTGTAGTCCCA |
| | | | | GCACTTTGGGAGGCTGAGGTGGACAGATCA |
| | | | | CCTGAGGTCAGGAGTTTGAGACCAGTCTGG |
| | | | | CCAACATGGTGAAACTCCGTCTCTACAAAA |
| | | | | ATTAGCCGGGCGTGATGGTGGGCAACTGTA |
| | | | | ATCCCAGCTACTCGAGAGGCTGAGGCAGG |
| | | | | AGAATCGCTTGAACCCAGGAGGCAGAGGT |
| | | | | TGCAGTGAGCCAAGATTGCGCCATTGCACT |
| | | | | CCATACTGGGAAATAAGAGTGAAACTCTGT |
| | | | | CTCAAAAAAAAAAACAACAAAAAAACAAA |
| | | | | CAAACAAACAAACAAAAAACTGAAAATAT |
| | | | | TGGAGCCTTTAGATAGTAGGTTACATGTCT |
| | | | | AAAATGGGAGTTAGCAAATGTATAAATGTA |
| | | | | GAAGTTTTTTTTTCAGGGAGAAATTGAAAT |
| | | | | TGCTCAAAGACTTTATCACCTTGAAGAAGC |
| | | | | AAGTATGTAGTTTATTTATTTTTTTGAGACA |
| | | | | CAGTCATGCTGTCACCCAGGCTGGAGTGTA |
| | | | | GTGGCGCGATCTCAGCTCACTTCAACCACC |
| | | | | TCCTCCTGGGTTCAAGCGATTCTCCCACCTC |
| | | | | AGCCTCCCGAGTAGCTGGGACTACAGGTGT |
| | | | | GCACCACCATGCCTGACTACTTTTTGTATTT |
| | | | | TTATTAGAGACGAGGTTTCACCATGTGGGC |
| | | | | CAGGCTGGTCTTGAACTCCTGACCTCAGGT |
| | | | | GATCCGCCCACCTTGGCCTCCCAAAGTGCT |
| | | | | GGGATTACAGGCGTGAGCCACCGTACCCAT |
| | | | | CCCCTAATTTATTATTTTAGGAATTTGGTTC |
| | | | | AAAGTTGTGATTGAAATCTATTGCCTTTATT |
| | | | | TTTGCCTTTGATATTTTTAAACTGAAGACAT |
| | | | | TTTTTTTTTTGAGACGAAGTTTCACTCTTGT |
| | | | | TGCCCAGGCTGGAGTGCAATGGCATGATCT |
| | | | | CGGCTCACTGCAATCTCCGCCTTCTGGGTTC |
| | | | | AAGCAGTTCTCCTGCCTCAGCCTTCTGAGT |
| | | | | AGCTGGGATTACAGGTGCGCACCACCACCC |
| | | | | CAGCTAATTTTTGTATTTTTAGTAGAGATGG |
| | | | | GGTTTTACCATGTTGGCCCAGCTGGTCTCG |
| | | | | AACTCCTGACCTCAGGTGATCCACCCGCCT |
| | | | | CAGCCTCCCAAAGTGCTGGGATTACAGGTG |
| | | | | TGAGCCACGGAGCCCGGCCTCAGACTGAG |
| | | | | GACTTAAAAAGTGAGGTCAGGGTGGGCAT |
| | | | | GGTGGCTCACGCCTGTAATCCCAGCACTTT |
| | | | | GGGAGGCTGAGGCGGGTGGATCACCTGAG |
| | | | | ATGAGGATTTCAAGACCAGCCTGGCCAACA |
| | | | | TGGCAAAACCCCGTCTCTACTAAAAATACA |
| | | | | AAAAATTAGCTAGGCATGGTGGCAGGAGC |
| | | | | CTGTAATCTCAGCTATTTGGGAGGCTGAGG |
| | | | | CAGGAGAATCACTTGAACCCGGGAGGCTG |
| | | | | AGGTTGCAGTGAGCTGAGATCGCCCCATTG |
| | | | | CACTCTAGCCTGGGCAACAAGAGCGAAACT |
| | | | | CCCTCTCAAGAAAAAAAAAAACCATCCTGG |
| | | | | CCGACATGGTGAAACCCCGTCTCTACTAAA |
| | | | | AATACAAAAATTAGCTGGGCGTGGTGGCA |
| | | | | GGCTCGGGAGGTTGAGGCAGGAGAATCAC |
| | | | | TTGAACCCGGGAGGCGGAGGTTGCAGTGA |
| | | | | GCCGAGATTGTGCCACTGCACTCCAGCCTT |
| | | | | GAGACAGAGGGAGACTCCATCTCAAAAAA |
| | | | | AAAAAAAAAAAGCGGTCAATCTTAGAATG |
| | | | | CAAAGTTAGGTAAGCAATACAGCTTGAGA |
| | | | | AAAGTGTAATTAAAAATAACTTTTCTATGT |
| | | | | AGTCATGTGATATTAATGTATTCAACTTGTT |
| | | | | CACAGTTGATTTAAGTTATTGATATAGTAG |
| | | | | GTATTGTTACTATGCTGGGAATTTTAGAAA |
| | | | | ATCCTTAGCAAATTGCTATTTGTCTCTTTTT |
| | | | | GTCTGTAATTTTGGCTGGGCTTGGTGGCTA |
| | | | | ACACCTGTAATTCTAGCAAGTTGGGAAGCC |
| | | | | GAGACAAAAGGATTGCTTGGGGCCCAGAG |
| | | | | TTTGAAACTAGACTGGGCAACATAGTGAGA |
| | | | | TCCTGTCTCTACACTCAGTTGGTTGTGGTGG |
| | | | | TATGCCTGTAGTCCCAGCTACTCAGGAGGC |
| | | | | TGAGGCAGTAGTAGGATCACTTGAGGCCAG |
| | | | | AAGTTTGAGACTGCAGTGAGCCATGATCAT |
| | | | | GCCACTGCATTCCAGCCTAGGCAACAGAGC |
| | | | | AAGATCCTGTCAAAAAAAAAAAAAAAGGA |
| | | | | GAAAATTCTCTTGGCAGTGGGTAAGAGTAG |
| | | | | TTATTAGGGTTGTAGATTTCCTGTCTGGAAT |
| | | | | TAGAGAAAGAAGGGTCATATTTTCTGTTAT |
| | | | | TTTGTGTATCTACCTCTAAGTGGACTGTTTG |
| | | | | CCTCTTGTCACGAATTAGTAGCCTCTTCAGT |
| | | | | TTACCATCATGTGCTCTTATTTTCTCTGCAT |
| | | | | ACAGTGAAGTGATTGTCATTACAATTTATA |
| | | | | ATCCTGACCTGGTACTTTTATATTTAATTGG |
| | | | | GCTGATATTTTCTAATTCTTCCCAGTGTACA |
| | | | | AAGGTTTTATGCTTTGTTGTTGTTGTTGAGA |
| | | | | CAGGCTAGGTGCTTTGGATGTGGAGAATTA |
| | | | | AATGAGCATGGCATTTTCAGAGGATACTTG |
| | | | | TTGGAGATTGCTTGGGTAGGATGGATGTAG |
| | | | | TCAGGTAATGGGGCCTAGAAATTCAGACTG |
| | | | | AAGCATTTGGTATTGATGTGATGGGAACTG |
| | | | | GCAGCCCTTGAGAGATTTTAGCTGAGAAGT |
| | | | | GATGTAAAATCTGTTTGGAAGACTTTGAGT |
| | | | | AGAGGAGATTAGAGGCAAGGTTAGGATGT |
| | | | | AGGGTATGTTGCAATAGTAATTAAGACTTA |
| | | | | AGAATCGGCCCAGTGGCATGTACCTGTAGT |
| | | | | CCTAGCTACTCTGGAGGCCGAGGCAGGAG |
| | | | | GATCACTTGAGGCTGCAATTAGCTGTGATT |
| | | | | GTGCCTGTGAATAGCCACTGCACTCCAACC |
| | | | | TAGGCAATATAATGAGATTCTGTCTCTTAA |
| | | | | AAAAAAAATGAGCACAGTGAGTACTCTAA |
| | | | | AGAAAGGGGGTAAATCTAAAAGATTATTTC |
| | | | | AAAGGGAGAAAATTGGCAGCTTTTTGGGG |
| | | | | GCTACCTGATCTGGAGGCAGATTGGAGTCT |
| | | | | GGATTTGAGGAATGGAGAGAGATGAGGCA |
| | | | | GATGATGTCTAAGGCTTATAGTTTTGCTGC |
| | | | | CTGAGACAAAAATGATTCCTCAGAGGTTCC |
| | | | | TTCCTCTTCTCTACCCATCATCCCACAATTT |
| | | | | TCTACTCCCTCCTTAGCTATCTTGGAAGAA |
| | | | | AATTGATCTCTTCACACCTGAGGTTCTGCTC |
| | | | | TCTCTCCGATTCCCTCCTGGCTGGGTGACCT |
| | | | | TTTTTGTTTGTTTTTGTTTTTGTTTTGAGACA |
| | | | | GAGTCTCACTCTGTCACCCAGGCTGGAGTG |
| | | | | CAGTGGGGCGATCTCGGCTCACTGCAACCT |
| | | | | CTGCCTCCCAGGTTCAAGCAATTCTCTGCCT |
| | | | | CAGCCTCTGGAGTAGCTGGGATTACAGGCG |
| | | | | CCCGCCACCGCAACCAGCTAATTTTTATAT |
| | | | | TTTTAGTAGAGACGGGGTTTCACCATCTTG |
| | | | | GCCAGGCTGGTCTTGAACTCCTGACCTCGT |
| | | | | GATCCACCCGCCTTGGCCTCCCAAAGTGCT |
| | | | | GGGATTACAGGCGTGAGCCACCGCGCGCA |
| | | | | GCCTTTTTTTTTTTTTTTTTTTTTTTTTTTTTA |
| | | | | AGATGAATTCTTGCTCTGTTGCCCAGGCTG |
| | | | | GAGTGCAGTGGTGTGACCTTAGCCCACGGC |
| | | | | AACCTCCATCTCCTGGGTTCAAGAGATTCT |
| | | | | TGTGCCTCAGCCTCCCAAGTAGCTGGGATT |
| | | | | GCAGGCGCCCTCCACCATGCTTGGCTAATT |
| | | | | TTTGTATTTTTAGCAGAGAGAGGTTTCACC |
| | | | | ATGTTGGCCAGGCTGGTCTCGAACCCCTAA |
| | | | | CCTCAAGTGATCCACCTGCTTCAGTCTTTCA |
| | | | | AAGTGCTGGAATTACAGGTGTGAGCCACCA |
| | | | | CGACCTGCATACCACTTCTCAAACAGTCCT |
| | | | | TTTTTGCGTCCTTGTTCTCTTTTTCTTCCTCT |
| | | | | TTCTCTGCAGTCTCATTCACTTTCATTGATT |
| | | | | CTGCTGCTACTCCACTCTATGAAACTCTCTT |
| | | | | CTGAACTGACTTCAAACCAACAAATTCTAC |
| | | | | TTGTCAACTAAGCTGCTCCTCTACCTTGTGT |
| | | | | TATATTCACCTAAAATGTAATATTATTTCCT |
| | | | | TTTTTATTTTTCCTTTGGACAGGGTCTTTCT |
| | | | | CTGTCACCCAGGCTGTAGTGCAGTGGTGCC |
| | | | | ATCTCGGCTCACTGCAACCTCTGCCTTCTGG |
| | | | | GTTCAAGTGATTCTCCTGCCTCAGCCTCCTA |
| | | | | AGTAGCTGGGACTACAGGCGCCCACCACCA |
| | | | | TGCCTGGCTAATTTTTGTATTTTTAGTAGAG |
| | | | | ACAGGGTTTTGCCATGTTGGCCATGCTGGT |
| | | | | CTCAAACTCCTGACCTCAAGTGATACGCCT |
| | | | | GCCTCTGCTTCCCAAAGTGCTGGGATTACA |
| | | | | GGCATGAGCCACTGCGCCCAGCCTATTATT |
| | | | | TTCATTTTGAACCCATCTCTTTTATTGCCAA |
| | | | | ACACGCATTTACTTCTGTGTTCATGATGAC |
| | | | | ATCATTATCCTATTCATCTCAAAGCTGGAA |
| | | | | ACCTTGCAGTCAATCATTTAAATGATTAAA |
| | | | | ATACATTTGAGTACCTCTTGAGCCAGGCAC |
| | | | | TGCCAGTATAATAAAAAATAAAAAAATTA |
| | | | | AAAAAAGGAAAGAGATAGTTTGCTTTTAAG |
| | | | | GAACTTCACTGTGTGGCAAAAACTAGTGTA |
| | | | | AACAATGACAATACAGAATACTAAGTGGTC |
| | | | | TGGTAGGTGTTATGTATGCAGTACTTTGGG |
| | | | | AGTGTGGAGGAAGGCATGCCTAGAATAAT |
| | | | | CAGGGAGGACTTCACAGAGTGGTTATTTAT |
| | | | | AGTTTAAGCAGAGACATACCAGTAAGAGG |
| | | | | GAATAGCATATGCAAGTGGCCAGAAATCCT |
| | | | | TGGCTAGCTATCTGGGAGGAGTGGGGTTGT |
| | | | | CAGGAGATAAAGGTATAAAGATAGGCTTA |
| | | | | TATGCCGTGCTGTATAGTTGAATGTTTTTAC |
| | | | | TATTACAAAATTTTACAGATGCCCTCAGTTT |
| | | | | CTCCCTTTATTCATTTTTCTATGACATCTTT |
| | | | | ATTGTTGGTCTTCATTTAGTCTTTCCTTCCA |
| | | | | GTCTATCCTGTGTAAAATTACTTCCTACTTC |
| | | | | CAAAATGAGAAATACTGGGTCTCTACTTAA |
| | | | | ATTTGTAACCTAAATGCCTCACACCTCATTT |
| | | | | TCTGAACAAATAAAGCCCAAATTCAGTGTC |
| | | | | CTTTTTGATAGGATCCTGTCCTGACCTTTCC |
| | | | | AAATCTGATGCTAGAGCCTTGTGTACCCTG |
| | | | | AGTTCAGCCAAACTGAACTCTTAATGGTCC |
| | | | | CTTGCTCCATACTCTCCCCTTGCTCATGCCT |
| | | | | TTATTCTCCTGGTCTGATTCATCTTTGCATC |
| | | | | TTAACAGTGTATAGCATGGTGCCTTCTTTTT |
| | | | | ACTGGGGACATATCGAGTTAATGAATGAAT |
| | | | | GATGCTATTACAGAGGTACAGTTTGGGAAG |
| | | | | GGGAGTGAGTACATTTTAGAAAGGTGATAA |
| | | | | GTGGATTGTCAGCCTTCATCATTTTCAATGG |
| | | | | ACCAAATTACTAAAACTTTACAGGTTGGTT |
| | | | | GGTTTTTTTTCTTTTTTCATTTCCTCATGTAC |
| | | | | TCAATTTCTAAGGCTTTTTGAATTTGAGCTT |
| | | | | CCTAATATCTCATGCATTAATTTTTTTCTCC |
| | | | | ATTCTCAACTTTCACTCTTTTAATTAAGGAT |
| | | | | AATAATTTTTTTTTTTGAGATGGAGTCTTGC |
| | | | | TCTGTTGCACAGGTTCGAGGGCAGTGGTGC |
| | | | | GATCTTGGCTCACTGCAATCTCCGTCTGCC |
| | | | | GTGTTCAAGCAATTCTCCTGCCTCAGCCTCC |
| | | | | TGAGTAGCTGGGATTACAGGTGCATGCCAC |
| | | | | CACGCCTGGCTAATTTTTGTATTTTTAGAAG |
| | | | | AGATGGGGTTTCACCACGTTGGTTAAGCTG |
| | | | | GTCTTGAACTCCTGACCTTATGGTCCGCCTG |
| | | | | CCTCAGCCTCCCAAAGTGCTGGGATTACAG |
| | | | | GCATGAGCCACTGAGCCTGGCCAAGGATA |
| | | | | ATAAATTATAATGGTTTTAGGTTGGACATC |
| | | | | TCTGACTGCATACTGCACTGTGTTTACTGG |
| | | | | AAGAAGTCCCTTAATGTCTCTAAGGCCCAT |
| | | | | TTCCTCAGTTCTAAATTACGGCTAGTACCTT |
| | | | | CATTGGAGGGTTGTTAAGTCTATGATACAA |
| | | | | GATAACTTTTTTTTTTTTTTTTTTTTTGAGAC |
| | | | | AGAGTCTCTATCGCCCAGGCTGGAGTGCAA |
| | | | | AATGGCACGATCTTGGCTCACTGCAACCTC |
| | | | | CACCTCATGGGTTCAAGTTGATTCTCCTGCC |
| | | | | TCAGCCTCCCAAGTAGCTTGGATTATAGGC |
| | | | | ATGCGCCACCATGCCCGACTAATTTTGTGT |
| | | | | TTTTAGTAGAGATGGGGTTCACCACGTTGG |
| | | | | CCAGGCTGGTCGAACTCCTGACCTCAGGTG |
| | | | | ATCGACCCACCTCGGCCTCCCAAAGTTGCT |
| | | | | AGGATTACAGGTGTGAGCCATCTCTCCTGG |
| | | | | CCATGATACAAGATAATTTATATGAAGTAA |
| | | | | TACACTGCTGGTTCTGAAGTAGGTGTGCAG |
| | | | | TAAGTGATGCCTACTGCTGCATGCCAAGAG |
| | | | | TCAAATGTATATTTGAAAGAGTTGTGAATT |
| | | | | TCAAGAAAGATATTTTTGAGTTTTTTTTTTT |
| | | | | TTCTTTCTGAGACAGGGTCTTGTACTGTTTC |
| | | | | CCAGGCTAGAGTGCAGTGGCCTGATCTTGG |
| | | | | CTCCTGGCTGGGCCCAAGTGATCCACCGCC |
| | | | | CTCAGCCTTCCAACGTATTGGGATTACGGG |
| | | | | AATGAGCCACTGCATTTGGCTAAGTTTTTG |
| | | | | TTTTTTTTTTTCTCTATTTTTCCAAACTTATT |
| | | | | TGATTAGTAAGATAAAGACATTAACTGCTG |
| | | | | TTGACAGTTTCCATTTTTAATTAGTAATCAG |
| | | | | GAGCATTTGTTGTATTTTTGTTTGATAATCA |
| | | | | GAATAATTTAATTTGTGCAATAGGATCAAT |
| | | | | AGCTTTCTGTATTCCAACTGTTAAGTGGTGT |
| | | | | AAGTTTATTACATTGTTGCTTTTTGCAGGTT |
| | | | | GTCCTTTGTTCTAGATAGAAATGTTTAATTT |
| | | | | ATTCTTCCTGGTTTTCAGGGGAGCCCATTG |
| | | | | AAAGGAGATCCAGTCTCTGAAATTTAGTGG |
| | | | | TAGGATAATAACAATTGAACAGTTACTTTT |
| | | | | GAATCTAATTTAAATAATCTCAATTGTAGC |
| | | | | CTTTTAAAGCAATTCCTATGAACCTTTTTGA |
| | | | | ATTTAGAAAAGTAATACTTGGCCGGGCGCG |
| | | | | GTGGTTCACATCTATAATCCCAGCACTTTG |
| | | | | GGAGGCTGAGGGGGTGGATTATCTGAGGTC |
| | | | | AGGAGTTCAAGACCAGCCTGGCCAACGTA |
| | | | | GTGAAACCCTGTCTCTACTGAAAATACAAA |
| | | | | AAAAAATTAGCTGGGTGTGGTGGCACGTGC |
| | | | | CTGTAGTCCCAGCTACTCAGGAGGCTGATG |
| | | | | CAGGAGGATCGCTTGAACCCAGGAGGCAG |
| | | | | AGGTTGCAGTAAGCTGGGATTGTGCCACTG |
| | | | | CACTCCAGCCTGGGTGACAGAGTGAGACTT |
| | | | | TGTCTCAAAAAAAAAAAAAAAAAAGTCAA |
| | | | | ACTTAAAAATGGAATATAAAAATCTCTTGA |
| | | | | TTTTTGTCAGTTTTCATATACTCCCTCATTT |
| | | | | ACACTCTTAATATTCTATTAGAAATTGTCTC |
| | | | | TTCTCTCTACACACCCCTTTTTTTCCCTTTTG |
| | | | | GTTAATATGTTAAGACATCTTTTCATATGA |
| | | | | GCATGTAACATGTAACAAGATTTTTTTTTTT |
| | | | | TTTTTGGACAGTGTCTCGCTCTGTTGCTCAG |
| | | | | GCTGGAGTCTAGTAGTATGATCACAACTCA |
| | | | | CTGCAGTTTAGACCTCCTGTGTTAAAGTGA |
| | | | | TTCTCCTACTTTAGCCTCATGAGTAGTTGGG |
| | | | | ACTACAGGCCCATGCCACCACGCCTGGCTA |
| | | | | ATTAAAGAAAAAATTATTTGGTAGAGACAG |
| | | | | GGTCTTGCTATGTTGCCCAGGCTGGTCTTG |
| | | | | AATTTCTGGCTTCAGGCAATTCTCCTACTCT |
| | | | | GCATGAGCCACCTCAGCCGCGAATATTTTC |
| | | | | TTATTATGAAATTTTTGTTTAGATAAATGTT |
| | | | | GATTCACATGCAGTTGTAACAAATTCCATG |
| | | | | GCCAGGCTGGGCGTGGTGGCTCACGCCTGT |
| | | | | AATCCCAGCACTTTGGGAGGCTGAGGTGGA |
| | | | | TCACCTGAGGTTGGGAGTCCAAGACCAGCC |
| | | | | TGACCAACATGGAGAAACCCCGTCTCTACT |
| | | | | AAAAATACAAAATTAGCCAGGCGTGATGG |
| | | | | TGCGTGCTTGTAATCCCAGCTACTTGGGAG |
| | | | | GCTGAGGCAGAAGAATCACTTGAACCCGG |
| | | | | GAGGCGGAGGTTGTAGTGAGCCAAGATCG |
| | | | | TGCCATTGCACTCCAGCCTGGGCTAGAAGA |
| | | | | GCGAAACTCCATCTCAAAAAAAAAAAAAA |
| | | | | AAAATCAGGAAATTCCATGGGCTAGGCAC |
| | | | | AGTGACTTATGCCTGTAATCCCAGCGTTTT |
| | | | | GGAAGGCTGAGGTTGGAGGATTGCTTGAGC |
| | | | | CCAGGAGTTTGAGGCTACAGTGAACACTGA |
| | | | | CTGTGCCACTGCACTCCAGCCTGGGTGACC |
| | | | | CTGTCTCTTAAAAAAAAAAAAGAATACAG |
| | | | | AGAGGTCCCTTGTATATTTTGCCTGGTTTTG |
| | | | | CAATGGTAATATTTTGCAAAAAATATCTAA |
| | | | | TACCACACAACCAGAATATTGATGTTGATG |
| | | | | TACTTCACCAATCGTTTTTTTTTTTTTTTTTT |
| | | | | GAGTCGGAGTCTCCATCTGATGCCCAGGCT |
| | | | | AGAGTGCAGTGGCTCAATCTCGGCTCACTG |
| | | | | CAACCTCCACCTCCTGGGTTCAAGCAATTC |
| | | | | TCCTGCCTCAGCCTCCTGAGTAGCTGGGAC |
| | | | | TACAGGCGTGTGCTATGACGCCCAGCTAGT |
| | | | | TTTTGTATTTTTAGTAGAGACGGTGTTTCAC |
| | | | | CGTGTTATCCAGGGTGGTCTCAATCTCCCG |
| | | | | ACCTTGTGATCCGCCCGCCTCAGCCTCCCA |
| | | | | AAGTGTTGGGATTACAGGCTTGAGCCACCG |
| | | | | CGTCCAGCCAGTCTTACTTAGGCATTGACG |
| | | | | TTCATGTAATTTATCCATCTTATTCAGATGT |
| | | | | CCTTAAATTTTATCTTTTTCCTTAAAAGAAA |
| | | | | TCTGTATTTCTATCAGGACATTCTGGATGTC |
| | | | | CCCAGTTTTACTGGTAGTCTTTCATTGTGTG |
| | | | | TATATTAAGTTCTTTGTTTTTATCACCTGTA |
| | | | | TAGGTTAGTATATCCATGACTCCCGTCAAC |
| | | | | TTTCTAAATGTTCGCTGGGTGCAGTGGCTC |
| | | | | ATGCCTGTAATCCCAGCACTTTGGGAGGCT |
| | | | | GAGGCGGCTGGATCACCTGAGGTCAGTAGT |
| | | | | TCGAGACCAGTCTGGCCAACATGGTGAAAC |
| | | | | CCCGTGTCTACTAAAAATAAAAAAAAAATT |
| | | | | AGCTGGATATGGTGGGTCATGCCTGTAATC |
| | | | | CTAGCTACTCGGGAGGCTGAGGTTGGAGAA |
| | | | | TCGCTTGAACCCAGGAGGCGGAAGTTGCAG |
| | | | | TGAGCTGAGATCGCGCCGCTGCACTCTAGC |
| | | | | CTGGGTGACAGAGTATGTCTCTGTCTCAAA |
| | | | | AAAAAAAAAAAAAGTTGCTAAACATTTCTA |
| | | | | ATACCATAAGGATCCCTGCTGTTGCCAGCC |
| | | | | GTTTTAAAACTACATCCATCGTCTTCTTGGC |
| | | | | AACCTTCCATCTCTTTTTCGTATGTGACAGC |
| | | | | GTCTTGCTCTGCCGCCCAGGCTGGAGTGCA |
| | | | | GTAGTTGCATCTCAGCTCACTGCACCCTCT |
| | | | | GTGTCCCAGGCTTAAGCGATCCTCCCACCT |
| | | | | CAGCCTCCTGATTAGCTGCGACTACAGGCA |
| | | | | CTTGCCACCATGCCCCACTAATTTTTGTATG |
| | | | | TTTTTGTAGAGATGGGGTTTTACCATGTTGC |
| | | | | TCAAGCTCGTCTTGAACTCGTGAGCTCAAG |
| | | | | CAATCCGCCTGCCTTGGCCTCCCAAATGGC |
| | | | | TGGGATTACAGGCAGGAGCCACCATGCCTG |
| | | | | GCCTAGCCCCTCCATCTCTAGCCTTTGTCAG |
| | | | | TTACTAAACTTTTTTTCCTGAAGTTTTGTCA |
| | | | | TTTCACAAATGTTAGATAAACATGAGTCAT |
| | | | | ACAGTATGCAGCCTTTTGGGATTGTCTTTTT |
| | | | | TTCCCTTAGCATAATTTCCAGGGGATTCATC |
| | | | | TAAGTTGTTGACTAAATCAATAGTTGTTTTT |
| | | | | TTTGTTTGTTTTTTTTTTGAGACGGAGTTTC |
| | | | | ACTCTTGTGGACCAGGCTGGAGTGCAATGG |
| | | | | CATGATCTTGGCTCACTGCAACCTCCGCCT |
| | | | | CCCAGGTTCAAGCGATTCTCCTGCCTCAGC |
| | | | | CTCCTGAGCAGTTGGGATTATAGGCCCCTG |
| | | | | CCACCACACCCAGCTAATTTTTGTATTTTTA |
| | | | | GTAGAGATGGGGTTTCACCATGTTGGTCAG |
| | | | | GGTAGTCTTGAACTCCTGGCCTCAAGTGAT |
| | | | | CTACCTGCATTGGCCTCCCAAAGTGCTGGG |
| | | | | ATTACAGGTGTGAGCCACTGCGCACGGCCC |
| | | | | TAGTTTTTTCCTTTTTATCACTAAGTAATAT |
| | | | | TCCATGATACAAATATACCATGGTTTGCTT |
| | | | | GACCGTTCACCTGTTGAAGGACATCTGGGG |
| | | | | CAATGCTAGCTTTTGGTAATTAAGGTAAAA |
| | | | | GTACTATTTATGTTCATTTATGGGGTTTTGT |
| | | | | GTGACTGTAAGTTTTCACTTCTCTGGGATA |
| | | | | AATACCAGTAGAACAATTGCAGTATTATAT |
| | | | | GGTAATGGCATGTTAAGTTTTTTTTTTTTCC |
| | | | | TGAGAGGGAGTTTCGATCTTGTTGCCCAGG |
| | | | | CTGGAGTGCAATTGCGCGATCTTGGCTCGC |
| | | | | TGCAACCTCTGCCTCCTGGGTTCAAGCGAT |
| | | | | TGTCCTTTCTCAGCCTCGCATGTAGCTGGG |
| | | | | ATTATAGGTGTCAACCACCACACCCAGCTC |
| | | | | ATTTTTGTATTTTTAGTAGAGATGGGGTTTC |
| | | | | ACTGTGTTTGCCAGGCTGGTCCCAAACTCT |
| | | | | TGACCCCAGGTGATCCACCCTCCTCAGCCT |
| | | | | CCCAAAGTGCTGGGATTACAGGCGTGAGCC |
| | | | | ACGGCGCCCCGCCAATGTTCAGTTGTTTTTT |
| | | | | TGTTTTTTTGAGACAATCTCTCTCTGTCACC |
| | | | | CAGGCTGGAGGGCAGTGGCGCGATCCTGG |
| | | | | CTCACTGCAACCTCTGCCTCCCGGATTCAA |
| | | | | GCGATTATCCCGCCTCAGGCTCCTGAGTAG |
| | | | | CTGGGACCACAGGTGCACACCACCACACCA |
| | | | | GGCTAATTTTTTTATTTTTAGTAGAGACGGG |
| | | | | GTTTCACCATGTTGGGTCAGGCTGGTCTCG |
| | | | | AACTCCTGACCTCAGGTGATCCACCCACCT |
| | | | | CGGCCTCCCGAAGTGCTGGGATTACAGGTG |
| | | | | TGAGCCACCACGCCTGGCCCAATGTTCAGT |
| | | | | TTTATAAGAAACTACCAAGCTGTTTTCCCT |
| | | | | AGTGTCTGTACCATTTACATTCTCACTAGCA |
| | | | | GTATATGAGTGATCCAGTTTCTTTTATTTTT |
| | | | | TGTTTTTTGAGACGGAGTCTCGCCCTGTTGC |
| | | | | CCAGGCTGAAGTGCAGTGGCACGATCTCGG |
| | | | | CTCACTGCAACCTCTGCTTCCCGGCTTCAA |
| | | | | GTGATTCTCCTGCATCAGCCTCCCAAGTAG |
| | | | | CTGGGATTACAGGCATGTGCACCATGCCTG |
| | | | | GCTAATTTTTTGTATTTTTAGTAGAGATAGG |
| | | | | GTTTCACCATGTTGGCCAGGCTGGTCTCGA |
| | | | | ACTCCTGACCTCAGGTAATCCACCCATCTT |
| | | | | GGCTTCCCAAAGTCCTGGGATTTCAGGCAT |
| | | | | GAGCCATTGCACCTGGCCGAGTGCTTCAGT |
| | | | | TTCTATGCATCCTCACCAGCATTTGGTGTGG |
| | | | | TCACTATTTTAATTTTAGCCATTCGTGTAGA |
| | | | | TATGTAGTAATGTCTCATCTCATTATGTTTT |
| | | | | GTTTTTTTTTTTGAGACGGAATGTTGCTCTT |
| | | | | GTTGCCCAGACTGGAGTGCAGTGATGCCAT |
| | | | | CTCGGTTCACTGCAACCTCCACCTGCTGAG |
| | | | | TTCAAGCAATTCTCGTGCGTCAGCCTCTGG |
| | | | | AGTAGCTGGGATTATAGGTGTGCATCACCA |
| | | | | CGCCTGGCTAATTTTTGTATTTTTTAGTAGA |
| | | | | CATGGGGTTTCACCACGTTGGCCAGGCTGT |
| | | | | TCTTGAACTCCTGACCTCAGGTGAGCTGCC |
| | | | | CACCTCGGCCTCCCAAAGTGCTGGGATTAC |
| | | | | AGTTTTGTATGGTGGATTCCATGCAGAGAG |
| | | | | AGTTTTTTCTGTAGTCTAGATTAGCAGTCCC |
| | | | | CAGCCTTTTTGGCACCAGGGACCAAATTCC |
| | | | | TGGGAAACAGTTTTTCCACAGGTGGGAGTG |
| | | | | GGATGGTTTGGGGATGAAACTTTTCCACCT |
| | | | | TAGATTATCACGCATTAGTTAGAATCTCAT |
| | | | | AAGAAGCGCGCAACCTAGATCCCTTGCATT |
| | | | | TGCAGTTCACAATAGGGTTCATGATCCTCT |
| | | | | GAGAATCTAATGCCACCCCTGATGTGACAG |
| | | | | GAGTGGGAGCTCAGGCGATAATGCTCCCTT |
| | | | | GTCTGCTGTTCACCTCCTGCTATGCAGCCCG |
| | | | | GTTCCTAACAGGCTGAGAGGACCAGTACCA |
| | | | | TTCTGTGGCCTGGGCGTTGGGGACCCCTGT |
| | | | | TCTAGATGATCCACATTCTTTTAAATGCCTA |
| | | | | TATACAAACCATACTTTCTTTATTTCTTTTC |
| | | | | TTTTTTTGAGACAGTCTTACTCTGTCACCCA |
| | | | | GGCTAGAGTGCAATTGCGTGATCTTGGCAC |
| | | | | ACTGCAACCTCTGCCTCCCAAGTTCAAGTG |
| | | | | ATTCTCCTGCCTCAGCCTCCCGAGTAGTTA |
| | | | | GGACTACAGGTGTGTCCCACCATGCCTGGC |
| | | | | TAATTTTTTATATTTGTATTTTTTAATTTTTA |
| | | | | TTTATTTATTTATTTTTTTGAGATGGAGTCT |
| | | | | CGCTCTGTCACGCAAGCTGGAATGCAATGG |
| | | | | CACGATCTCGGCTCACTGCAACCTCCGCCT |
| | | | | CCCGAGCTCAAGCGATTCTCCTGCCTCAGC |
| | | | | CTCCTGTGTAGCTGGGATTACAGGCACCCG |
| | | | | CCACGACGCCTGGCTTTTTTGTATTTTTGTA |
| | | | | GAGACAGGTTTTCACTGTGTTGTCCGTTCTG |
| | | | | GTCTCAAACTCCTGAGTTCAGGGAATCCAC |
| | | | | CGCCTTGGCCTCCCAAAGTGCTGGGATTAC |
| | | | | AGTCGTGAGCCACCGCGCCCTGCCACAAAC |
| | | | | CATACTTTGAAAACGTTGCTTCCATTTTTAG |
| | | | | ATAATTTGTTAGGAAACCAATAAAATCATA |
| | | | | CATACTTGTGATTTTCCCTTAGTAAAACAC |
| | | | | AAATTTTAGTGTTTTTTGCTGTTATTATTAA |
| | | | | TACTTCTAAAGTTCCTTTCACATTGCTAGTG |
| | | | | ACCTTATATAAAATACCATAATGCTCTTCT |
| | | | | AGCAATTGCTGGAAAGATAAAATCTATTTT |
| | | | | AGAGAATGAACAATTATATTTTCACATTAG |
| | | | | ATTAAATTAAAAGTAATTACTGGTTATGTG |
| | | | | ATATTCCCTCACATACCAGAGTGAGTCTGA |
| | | | | AGGTAGTCTTTCTTTGTAAATTATGAGGCT |
| | | | | ATATTTCCTGTGTTATCTCTGATTTCTCTTG |
| | | | | ATGCTGTAATTGGAGTTGTTGGGTCTCCCT |
| | | | | GGTGAAAGTAGGTGATGTGCAAGTTGTGTC |
| | | | | TATACCCAGTGAAAATAACAGACATTAATG |
| | | | | CTACACTAATTTGTCATTGGAATTTTACATT |
| | | | | CAAAAGCATTTCTTTTTAAAAATATGATTG |
| | | | | TAAATTGGTAATTTATAGTTGTATATACCA |
| | | | | AAGGCATTTCTTTAACGTTATAGTTGGTTCA |
| | | | | ACTGAAAATACGTTAAGTCTGTTTTTATAA |
| | | | | TTAGTATATTGAGGAACAGCACTTCCATCG |
| | | | | TGTCACAATATATTAAGAATTGCCAGCAGG |
| | | | | GCACGGTGGCTCACGCCTATAATCCCAGCA |
| | | | | CTTTGGGAGGCCTAGGCGGGAGGATCACCT |
| | | | | GAAGCCAGGAGTCGAGACCAGCCTGGCTA |
| | | | | ACGTGGCCAAACCCCTATCTACTAAAAATA |
| | | | | CAAAAATTAGCCAGGTGTGATGGCGGGTGC |
| | | | | CTGTAGTCCCAGCTACTCGGGAGGCTGAGG |
| | | | | CAGGAGAATCCAGAATTGAATTGAACCCA |
| | | | | GGAGACGGAGGTTGCAGTGAGCCAAGATT |
| | | | | GTGCCATTGCACTCCAGCCTGGACAACACA |
| | | | | GCGAGACTCAGTCTTTTTTATTTTTATTTTT |
| | | | | ATTTTTGAGACGGAGTTTCGCTCTTGTTGCC |
| | | | | CAGGCTGGAGTGCAATGGCACAGTCTCGGC |
| | | | | TCCCTGCAACTTCTGCCTCCCGGGTTCAAG |
| | | | | CGATTCACCTACCTCAGCCTCCCGACTAGC |
| | | | | TGGGATTACAGGCATGTGCCACCACGCCCG |
| | | | | GCTAATTTTTGTATTTTTAGTAGAGATGGG |
| | | | | ATTTCTCCATGTTGGTCAGACTTGTCTCGGA |
| | | | | CTCCCAACCTCTGGTGATCTGCCCGCCTCG |
| | | | | GCTTCCCAAAGTGCTGGGATTACAGGCATG |
| | | | | AGCCACCGTGCGTGTCCTTTTTTTTTTTTTT |
| | | | | ATCTTTTGAGACAGGGTCTCACTCTGTTGG |
| | | | | CTAGGCTGGAGTGCAGTGATGCAGTCACAA |
| | | | | CTCACTGCAGCCTCAACCTCCCAGTCTCAA |
| | | | | GCAATACCCCCACCTCTGCCCCTTTGAGTA |
| | | | | GGCTGGGACTACAGGTGTGTGCCTTCATAC |
| | | | | CTAGCTAATTTTTTTTGTTTTGTTTTTTGAG |
| | | | | ACAGTCTTGCCCCATCGCCCAGGCTGGAGT |
| | | | | GCAGTGGTGCCATCTCGGCTCACTGAAAGC |
| | | | | TCCGCCTCCCGGGTTCACGCCATTCTCCTGC |
| | | | | CTCAGCCTCCCGAGTAACTGGGACCACAGG |
| | | | | TGCCCGCCACCACACCCGGCTAATTTTTTGT |
| | | | | ATTTTTAGTAGAGACGGGGTTTCACCATGT |
| | | | | TAGCCAGGATAGTCTCGTTCTCCTGACCTC |
| | | | | ATGATCCGCCTGCCTTGGCCTCCCAAAGTG |
| | | | | CTGGGATTACAGGTGTGAGCCACTGCACCT |
| | | | | GGCCATGCCCAGCTAATTTTTGTATTTTTTT |
| | | | | GTAGGGATGGGATGGCACTATGTTCCCTAG |
| | | | | GCTAGTCTTTAATTCTTGGGTTCAAGTGGTC |
| | | | | CTCCTGCCTCGGCCTCCCAAAGTGTTGGGA |
| | | | | TTACAGGTGTGAGCCACTGTGCCGAGCCAG |
| | | | | GTTGTGTGTGTGTGTATGTATGTATGTATGT |
| | | | | ATGTATGTATGTATGTATGTATGTTTGTATA |
| | | | | TATTTATATTTATTTTTTTGGAACTGCATCT |
| | | | | CACTTTCATCCAGGCCCGAATGCAGTGACA |
| | | | | TGATCTCAGCTCACTGCAACTTCTGCCTCCT |
| | | | | GGGTTCAAGCGATTCTTTTTTTTTTTTTTTTT |
| | | | | TTTTGAGACGGAGTCTCCCTCTGTCGCCAG |
| | | | | GTTCACTGCAAGCTCTGGCTCCCGGGTTCA |
| | | | | CGCCATTCTCCTGCCTCAGCCTCCCAAGTA |
| | | | | GCTGGGACTACAGATGCCCACCAGCATGCC |
| | | | | TGGCTAATTTTTTGTATTTTTAGTAGAGATG |
| | | | | GGGTTTCACTGGGGTTTCACCATGTTAGCC |
| | | | | AGGATGGTCTTGATCTCCTGACCTTGTGAT |
| | | | | CCGCCCGCCTCTGCTTCCCAAAGTGCTGGG |
| | | | | ATTACAGGCGTGAGCCACTGCGCCTGGCCA |
| | | | | TTTCTTTTTTTTTTTTGGCAAGTGATTCTTGT |
| | | | | GCCTCAGCCTCCCGAGTAGCTGAAATTATA |
| | | | | GGCGTGTGCCCTCAACGCCTGGGTAATTTT |
| | | | | TGTATTTTTAGTAGAGACAGGGTTTCACCA |
| | | | | TGTTGGACAGGCTGGTCTCAAACTCCTGGC |
| | | | | CTCAAGTGATCCACCCTCCTCAGCCTCCCA |
| | | | | AAGTGCTGGGATAACAGCTGTGAGCCACCG |
| | | | | TGCCCTTCCCAGGTTTTATATTTATTCTTTTT |
| | | | | TCCTTTTAAATTATGTTTTTATTTAGGTATT |
| | | | | GTACGTAAAGTGCTTTTCTAACAGAGCTTT |
| | | | | GGGGCAGAAGTGTTAGGGCAGGTCATTAA |
| | | | | ACCACTGAAATTAGTTCTTTGGAGGAGAAG |
| | | | | ATAATTGTTAGAGTTGTAAGTGAAGTCTTG |
| | | | | ATAGATACCTTATCAATTTCATAGTAATGT |
| | | | | CTGTGGAATTTCTTTTTCTGTTTTTTTTTTTT |
| | | | | TTAATTATTTCTTGAGGATTAACTGCTGATA |
| | | | | GTGGAATATCATATATATAGTTGGCTCTTG |
| | | | | ATGTACTTATTTCTGGATGGCTTTCCAAAA |
| | | | | GGATTTTACCATTTTACACACAGTTCTAAAT |
| | | | | AGTATATGAATTTAGCATTTGTCCCACACTT |
| | | | | AGATAGCACTGATTTTTTTTTTTATTAAGTG |
| | | | | GGTGCAAAATGCTACTACAAGATTGCTTTA |
| | | | | ATTACTACAGTTTTATTGATGAAAATGATTT |
| | | | | CTACTTGTTTACTGTTTGTATTTTTTTCTAG |
| | | | | GAGTTTTGTGTCTATATTCTTTGCTGATGTA |
| | | | | TCTTTTTGGATTTAATGTTTTATACATATTA |
| | | | | AATTTCTGTCTCATTGGATATAAATATTTTC |
| | | | | CCAATCTGGTTTTCATTTTAGTTAATGATTT |
| | | | | TCTGTAGTTGTATAGTCAAAGTTTCATTTAT |
| | | | | TATATAGCTAGATCTGTGTTTTCGAGTGATT |
| | | | | TATTGATTCAAAGCTTATTGTGCTTCTAGAT |
| | | | | ATTTGATAAACTGACTTTAGACTCTTGTAA |
| | | | | AAATTTGAAGAACTCATATCTACTACAGTC |
| | | | | TTACTGATTTAATAGGGGTTTTAATATCCA |
| | | | | GTACTATGCTAATAATTTTTATAGTGTTTTT |
| | | | | ACGACAATTTTTTGAGAACATAAGTTTTTA |
| | | | | GAGCTGTGGATGGAATGTTTTCTGCTCTAT |
| | | | | CAGTTATCCCTTCTGCGTAACAGACCCCTA |
| | | | | AGTGTAGCAGCTTAGAGGAGTAAATATTTA |
| | | | | TTATCTCACATTTTGTAAGGAATCATGGAG |
| | | | | TGGCTTAGCTGGATGGTGCTGGCTCAGTCT |
| | | | | CTCTAATGAATTTACAGTCAAGATGTCTGC |
| | | | | CAGGGCTGCGGTCTCTGAAGGCTGTAGGAT |
| | | | | CCCTGTCCAAGACGGCTCACTCATATGGAT |
| | | | | GCTAGCTCTTTGTATGAGGCCTGTTCTTTCC |
| | | | | CACTTGCACTTCTCCATAGGCCTGCTTACTG |
| | | | | TATGGTAGCTGGCTTTTCCCGGAGTGAGTG |
| | | | | ATCCAAGAGACAGGGACAGACCAAGCAGG |
| | | | | AAGATGCAGTAACTTTTTATGATGTGTATT |
| | | | | CTATTGGCTGGCCACACATACCAAGCAGAT |
| | | | | AGGGAAGGGATTACACAAAGGCATGAATA |
| | | | | CCATCAGGCTGGGATAATTGGGGGCCAGCT |
| | | | | TGGAATCTGGCTACCATATCCAACCAAATA |
| | | | | AGAAATTAATAGTTTTAATTAAAGGAAAAG |
| | | | | GATTATATTAAATAGACATTCGTTAGTTTTT |
| | | | | ACTTTTAAGCTGACCCAATCATTTTTCAGAT |
| | | | | TGAAGTTTTGAATAGATATATGATTAAAAA |
| | | | | ATACATGAAAAGTTAACCAGTGAAGTGACC |
| | | | | TCTGTGCCATGTTTGCTCAGGTAACGCACC |
| | | | | TCCAATTCTTGTGCTTTCCCGGAGACCACCT |
| | | | | TTTTTAAGAGAAAGGTAGTGGACTGTGCAC |
| | | | | ACTTGGTCTTCCTTTTTCACATAATGGTGTA |
| | | | | TGTTGAAATCTTTCCATTTTAGAGCATAGCT |
| | | | | TTCCCTTTTTAATTTTATTATTATTATTATTT |
| | | | | TTGAGACAGAGTCTCCCTCTGTCGCCCCAG |
| | | | | CTGGAATGCAATGGTGCGATCTCGGCTCAC |
| | | | | TGCAACCTCCAGCTCCTGGGTTCAAGTGAT |
| | | | | TCTCCTGCCTCAGCCACCTGAGTAGCTGGG |
| | | | | ATTACAGTCGCCTGCCACCATGCTCGGCTA |
| | | | | ATTTTTGTATTTTTAGTAGCGACGGGGTTTC |
| | | | | ACCATGTTGGCCAGGCTGGTCTCGAACTCC |
| | | | | TGACCTCAGGTTATCCACCTACCTCAGCCT |
| | | | | CCCAAAGTGCTGGGATTACAGGCGTGAGGC |
| | | | | ACCGTGCCCGGCAATTTTTTTTTTTTGAGTC |
| | | | | AGAGTCTTGTTCTGTTGCCCAAGTTGGAGT |
| | | | | GCAGTGGTTTGATCTCGGCTCACTGCAACC |
| | | | | TGTACCTCCTGGGTTCAAGTGATTCTCCTGC |
| | | | | CTCAGCCTCCCGAGTAGCTGGGACTACAGG |
| | | | | CATGCCCCACCATGCTTGGCTAATTTTGTAT |
| | | | | TTTAGTAGAGACTAGGTTTCTCCATGTTGGT |
| | | | | CAGGCTCGTGTCAAACTCCCTACCTCAGGG |
| | | | | GATCCGCCCACCTTGGCCTCCCAAAGTGCT |
| | | | | GGGATTATAGACGTTAGCCACCGCGCCTGG |
| | | | | CCTAATTTTTGTATTTTCAGTAGAAATTTTT |
| | | | | GTATTTCACTGTATTGGTCAGGCTGGTCTG |
| | | | | GAACTCCTGAGCTCAGGTGATCCACCCGCC |
| | | | | TCGGCCTCCCAAAGTGCTGGGATAACAGGA |
| | | | | GTGAGCCACTAGGTGTGACCTAATTTTTGT |
| | | | | ATTTTTAGTAGAGATGGGATTTCACCATGT |
| | | | | CGGCTAAGCTGGTCTCGAACTCCTGACCTC |
| | | | | AGGTGATCTGCCTGCCTTGGCCTCCCAATG |
| | | | | TGCTGGGATTATAGGCATAAGCCACCGCAC |
| | | | | TGGCTTTTTTTTTTTTTTTTTTTTTAAACCTG |
| | | | | GATGGTTTTATTTTGCATGAATGTATAGAT |
| | | | | ATTTCCTGTTCATACATTCTGAAAGTGAAC |
| | | | | AACTGTATATATGCAATTTATTTTTATTCTT |
| | | | | ATTTATTTATTTGTTTATTTTTTGAGACCAG |
| | | | | AGTCTCACTCTGTCGCCCAGGCTAGAGTGC |
| | | | | AATGACACAATCTCGGTTCACTGCAACCTC |
| | | | | TGCCTCCTGGGTTAAGCAATTCTTCTGCCTC |
| | | | | AGCTTCCCCAGTAGCTGGGATTACAGGTGT |
| | | | | CCGCTAATTTTTGTATTTTTACAAAATACAC |
| | | | | CCAGGTAATTTTTTGTAATTTTGGTAGAGA |
| | | | | CAGGTTTCACCATGTCGGCCAGGCTGGTCT |
| | | | | CGAACTCCTGACCTCAGGTGATATGCCCGA |
| | | | | CTCAGCCTCCCAAAGTGCTGGGATTACAGG |
| | | | | TGTGAGCCACTGCGTCTGGCCTGCATGGGG |
| | | | | ATTCTTAATGAAGATTAATTATTGTAGTTG |
| | | | | AGGGGGAAAAGGAATAATAAATATTTATT |
| | | | | GGACCCTAAATACCTTCGAATATGGAATAC |
| | | | | CCTAGGTATTCTAGGGCATTTAGGGACCAA |
| | | | | TAAATATTTATTCCTCCGTACTCTTCCCTCG |
| | | | | CTCTTTTCAGATTTTTTTTTTTTTTTTTTTTTT |
| | | | | TTTGAGATGGAGTCTTGCTCTGTCTCCAGG |
| | | | | CTGGAGTGCAGTGGCGCGATCTTGGCTCAC |
| | | | | TGCAACCTCTGCCTCCTGGGTTGAAGTGAT |
| | | | | TCTCTTGCCTCAGCCTCCTGAGTGGCTGGG |
| | | | | ACTACAGGTGCATACCACTATGCCCAGCTA |
| | | | | ATTTTTGTATTTTTTGTAGAGACAGGCTTTC |
| | | | | ACCATGTTGGCCAGGATGGTCTCGTTCTTT |
| | | | | AGACCTCGTGATCTGTCTTCCTCAGCCTCCC |
| | | | | AAAGTGTTGGAATTACAGGCGTAAGCCTCC |
| | | | | GCCGGGCCTTTTTTAGATTTTTAAGAGAATT |
| | | | | TTTGTTAAAGCATGAACTTAAAAAATCAGA |
| | | | | CTTGGCTTGGAGCGGTGGCTCATGGCCTCT |
| | | | | AGTCCCAGGACTTTGGGTGGCTGAGGCAAG |
| | | | | TGGATTGCTTGAGCCCAGGAGTTCAAGACC |
| | | | | TGCCTTGGCAATAATATCAAGACCCCCTCT |
| | | | | TCATGAAAAACAATCAAGCTAATACTTGAT |
| | | | | ACTATTTTACATAAGAATTTTTTATAGTATG |
| | | | | TCATGTTTTAATGTATATTGGTTATATAGTT |
| | | | | GCAAATTTAAAGGCATGGTGGTGGCTCATA |
| | | | | CCTGTAATCCCAGCACTTTGGGAGGCTGGG |
| | | | | GCGGGCAGATCTTCTGAGGTCAGGAGTTCA |
| | | | | AGACCAGCCTGGCCAACATGGTGGAACCCC |
| | | | | GTCTTAGGCTGAGGCAGGAGAATAGCTTGT |
| | | | | GCCCAGGAGGCAGAGGTTGCTTTGAGCTGA |
| | | | | GATCGCACCACGGCATTCCAGCCTGGAGGA |
| | | | | CAGAGCGAGACTCTGTCTCTAAATAAATAA |
| | | | | ATAAATAAATAAATGTATACTAACTGCATT |
| | | | | AGCAAGACTCCGTCTCTAAATAAATAAGTG |
| | | | | AATAAATAAATGTATACTAATTGCATTTTA |
| | | | | AAAATCAAAGTATAGGCCGGGTACGGTGG |
| | | | | CTCACAACTGTAATCCTAGCACTTTTGGAG |
| | | | | GCTGAGGTGGATGGATCACCTGAGGTCAGG |
| | | | | AGTTTGAGACCAGCCTGACCAACATGGTGA |
| | | | | AACTTTGTCTCTACTAAAAATACAAAATTA |
| | | | | GCTGGTGTGGTGGCGCATGGCTGTAATCCC |
| | | | | AGCTACTCGGGAGGCTGAGGTAGGAGAAT |
| | | | | TGCTTGAACCTGAGAGGTGGAGGTTGTGGT |
| | | | | GAGCGGAGATCGTGCTGTTGCACTCCAGCC |
| | | | | TGGGCAACAAGAGCGAAACTTCGTCTCCAA |
| | | | | GAAAAAAAAAATATATAATTCACATAAGA |
| | | | | TAAAATTCACCCTCTTTGGCCAGGCGCAGT |
| | | | | GGCTCATGCCTGTAATCCCAGCACTTTGGG |
| | | | | AGGTAGAGGTGGGCAGATCACTTGAGGTC |
| | | | | AGGGAGTTTGAGACCAGCCTGGCCAACATG |
| | | | | GTGAAACCCCATCTCTACTAAAAATACAAA |
| | | | | AATTAGCCCGGTGTGGTGGCATACACCTGT |
| | | | | AATCCACCTACTCAGGACGCTGAGTCTGCA |
| | | | | CTCAGTCCCTGGGCTACAGGGTGAAACTGT |
| | | | | ATCTCAAAAATAAAGAATAAAATGCAGCT |
| | | | | ACTTAAAGGGTGTAGAGTTGAACAACTGTT |
| | | | | ACCACTGTCTAATTCCAGAACCTTTCATCA |
| | | | | CCCCAAAAGAAAACCCATTCCCAGCAGTCA |
| | | | | TTTCCCATTAAGTCTCCTCTAGCCCCTCACA |
| | | | | ACCACTAATCTAATTCATGTTTCTATGTATT |
| | | | | TGCCTATTCTAGGCGTTTCATACAAATACA |
| | | | | GTCATATAATTTGTGGCCTTTCGTGTCTGAC |
| | | | | TTGTTTAACTTAGCATAATGTTTTAAGGCCC |
| | | | | ATTTATGTTGTTGTATGTATGCATACTTCAT |
| | | | | TCCATTTTACTGCTGAATATTGCTTTGTACT |
| | | | | GATGCCACTTTTTGTTTGTCTTTTCATCACT |
| | | | | TGACGGACATTTTGTTTCTTCCACTTTGTGG |
| | | | | CTGTTACAGGCAGTGCTACTGTGAAAATTT |
| | | | | GTATTAAAGTTTTAGCGTGAATATATGTTTT |
| | | | | CAGTTCTCTTGGGAAAATACCTAGAAGTGG |
| | | | | TATTGTCGGATCATAGGGTCATTCTATGTTT |
| | | | | AGCATTTTGAGGAACAGCCAGACTGTTTTA |
| | | | | CATAGTGGTTGCACCGTTTTACAGTCCTACT |
| | | | | TTAGCCTATATGGGTTCTAATTTCTTTCTTT |
| | | | | CTTTCTTTCTTTCTTTCTTTCTTTCTTTCTTTC |
| | | | | TTTCTTTCTTTCTTTCTTTCTTTTCTTTCTTTT |
| | | | | CTTTCTTTTCTTTCTTTTCTTTCTTTCTTTCTT |
| | | | | TCTTTTTTTAGAACAGAGTCTCCCTCTGTAG |
| | | | | CCCAGGCTGGAGTGCAGTGGCATGGTCTTG |
| | | | | GCTCACTGCAGCCTCCGCCTCTCGGGTTCA |
| | | | | AGCAATTCTCTGCCTCAGCCTCCCAAGTAG |
| | | | | CTTGGACTACAGGCGCCCGCCACCACGCCT |
| | | | | GGCTAATGTTTGTATTTTTGGTAGTGACAG |
| | | | | GGTTTCACCACATTGGCCAGGTTGGTCTTG |
| | | | | AACTCCTGACCTCAGGTGATTCACCCACCT |
| | | | | CGGCCTCCCAAAGTGCCGAGATTACAGGCA |
| | | | | TGAGCCACTGCATCCGGGCGTGGGTTCTAA |
| | | | | ATTCTTAATATTCTCATCAACATTTATTGCT |
| | | | | GTCTTTTTAATTTTAGCCTGTAATCCCAGCT |
| | | | | ACTAGGGCGACTGAGGTGGTAGCATCGCTT |
| | | | | GAGCCCAGGAAGCTGAGGCTGCAGTGAGC |
| | | | | CAAGATTGCACCACTGCACTCCAGGCTAGG |
| | | | | TGATGAAGTGAGACTTCATCTCAAAAAAAA |
| | | | | AAAAAAGGAAGTAATGGCAAAAACTGGAA |
| | | | | TTATTTTGCACCAACTTAAATATTTAGATCT |
| | | | | TTAATACCTTTGGAAAGTTTTTTATATATAG |
| | | | | TTTGTGTGTGTGTGTGTGTATATATACACAC |
| | | | | ATATATATATACACACACATATATACACAC |
| | | | | ATATATATGAATGATTTTATATATATATATA |
| | | | | TATATATATATATGAATGATATATATATAT |
| | | | | ATATATGAATGAATGAATGAGATGGAGTCT |
| | | | | CACTCTGTCACCCAGGCAGGAGTGCAGTGG |
| | | | | TGCCATTTTGGCTTATGGCAGCCTCCGCCTC |
| | | | | CGGGGTTCAAGTGATTCTTGTACCTCAGCC |
| | | | | TCCCGAGTTGCTGGGATTACAGGCACTCGC |
| | | | | CACCATGCCCGGATTTTTTGTCTTAATTCAT |
| | | | | GAAGGATGAATTAAGTCTGCAGTTGTTCTT |
| | | | | TTTCCCTTTTTCTTTCCAGTTTTTTTTTTTGT |
| | | | | TTGTTTGTTTGTTTTTGAGACACAGTCTCAC |
| | | | | TCGGTTGTCCAGGCTGGAGTGCGGTGGCAG |
| | | | | TATCTTGGCTCCCTGTAACCCATCTCCCTGG |
| | | | | TTCAAGCGATTCCGGTGCCTCAGCTTCCCA |
| | | | | AGTAGCTAGGATTACAGGTGTGTGACACCA |
| | | | | CACCTGGTTAATTTTTGTATTTTTAGTAGAG |
| | | | | ACGAGGTTTCACCGCATTGGTTAGGTTGGT |
| | | | | CTCAAAACTCCTGACCTCAGGTGAACCGCC |
| | | | | CACCTAAGCCTTCCAAAGTGCTGAGATTAC |
| | | | | ATGCATGAGCCACCAAGTCTGGCCTAAGTC |
| | | | | TGAATTTTTTTTTTTTTTTTTTTGAGACGGA |
| | | | | GTTTCGCTCTTGTTGCCCAGGCTGGAGTGC |
| | | | | AATGGTGCGATCTTGGCTAACCGCAACCTC |
| | | | | CGCCTCCCACGTTCAAGCAATTCTGCCTCA |
| | | | | GCCTCCCGAGTAGCTGGGATTGCAGGCATA |
| | | | | TACCACCACGCCTGGCTAATTTTGTATTTTT |
| | | | | GTTAGAGATGGGGTTTCTCCGTGTTGAGAC |
| | | | | TGGTCTCGAACTCCTGACCTCAGGTGATCC |
| | | | | GCCTGCCTCGGCCTCCCAAAGTGCTGGGAT |
| | | | | TACAGGTGTGAACCACTGCACCCGGCCGAA |
| | | | | TATATTTTTTTTTTTTTAAATGGAGTCTCGC |
| | | | | TCTGTGGCCCAGGCTGGAATGCAGCGGTGT |
| | | | | GATCTTAGCTCACTGCAACCTCTGCCTCCCT |
| | | | | GGCTCAAGCGATTCTCCTGCTTCAGCCTCCT |
| | | | | GAGTACCTGGGACCACAGGTGTGCACCACC |
| | | | | ATGCCTGAATAATTTTTTTGTGTTTTTGTAG |
| | | | | AGATGGAGTTTCACCATGTTGGCCAGGCTG |
| | | | | ATCTTGAACTACTGACCTCAGGTGATGTGC |
| | | | | CTGCCTCCGCCTTCCCAAGTGCTGGGATTA |
| | | | | CAGGCATGAGCTACTGTACCCGGCTAAGTG |
| | | | | TACAGTGTTCTTGTGATGTCTTTGTCTGGTG |
| | | | | TTGGTATCAGGGTAATACTGTCTTCAAGAT |
| | | | | TACCCTTGAATGAGCTTTACTTCATTTTTTA |
| | | | | ATGTGTTTTTTTTTCTTTTCTTTTGTTTTTTG |
| | | | | TTTTTGAGACAGAGTTTCACTCTGTCGCAC |
| | | | | AGGCTGGAATCCACACTCTAGGCTCGCTGC |
| | | | | AGCCTCCACCTCCCAGGTTCAAGAGATTCT |
| | | | | CCTGTGTCAGCCTCTTGAGTAGCTGGGGTT |
| | | | | ACAGGCACGTGCCACGACGCCCGGCTGATT |
| | | | | TTTTTGTATTTTTAGTAGTGACGGGCTTTCA |
| | | | | CCATGTTGGCCAGGCTGGTCTCGAACTCCT |
| | | | | GACATCAAGTGACCTGCCTTCCTCAGCCTC |
| | | | | CCAAAGTGTTGGGATTACAGGAGTGAGCCA |
| | | | | CTGTGCCCCGCCTGCAATTACTTCTTAAGTT |
| | | | | CTCAATTAAAAGAGAGTTTATCAAGGACTT |
| | | | | TTTTTGGTAATTTTGCATTTTGAAAATTGCT |
| | | | | AACATTAACTGGGACAGCCCTTTTATTTATT |
| | | | | TATTTGTCACTCAGTTGTTTTTTTGAGTTGC |
| | | | | CTACTATGTCCCAGGCACTGGTAAGATAGG |
| | | | | AGTATCATTGTACCTGAGGCAGGGCAACAT |
| | | | | GTGCTTGCTTGAGAGGAGCATGATCTAGGA |
| | | | | TTATAAGGACTGCAACCTCCCCTTCCCAGG |
| | | | | TTGAAGCAGTTCTCATGCCTCAGCCTCCCA |
| | | | | AGTAGCTGGGACTACAGCCATGAGCCACCA |
| | | | | CGCCCAGCTAATTTTTGTGTTTTTAGTAGAG |
| | | | | ATGAGGTTTCCCCATGTTGGCCAGGCTAGT |
| | | | | CTCAACTTCTGGACCTCAGGTGATCTGCCC |
| | | | | ACTTCAGCCTCCCAAAGTGCTGAAATTACA |
| | | | | GGAGTAATTTTATTCTCCCAAAGCTGCTGC |
| | | | | TTTGGGAGAATAAAAAGTTGAGTATGGGCC |
| | | | | AGGCATGGGGGCTGATGCCTGTGATCGCAG |
| | | | | CACTTTAGGAGACTGAGGTGGGAGTCTAGC |
| | | | | TTGAGCCCAGTAGTTTGAGACAAGCCTGGG |
| | | | | GAACATAGGGAGATCCGGCCTCTACAAAA |
| | | | | AAAATAAATTAGCTGGGTGGAGTGGCATGT |
| | | | | GCCTGTGGTCCCAGCTACTTGGGTGGTTGA |
| | | | | GGTGGGAAGATATCTGAGCTCAGGAGTTCC |
| | | | | AGGCTGCAGTGAGCTCTGATTATGCACTCC |
| | | | | AGCCTGGGTGACAGAGTGAGATGCTGTCTC |
| | | | | AAAAAAAAAAATTCAGTGTGGCGTGATTA |
| | | | | GGCTGGGAGGGTGGGGCAGGAAGGGATGA |
| | | | | CATTGGAGGGGTAGGCAAGGTGTAGATAG |
| | | | | ACCTTTCCCTATATTCTCCTATTTTTAAAAA |
| | | | | ATTTTTTTCTAAATAGAGATAGGGTCTTACT |
| | | | | ATTTTGCCCAGGCTGGGTCTCAAACTCCTG |
| | | | | GGCTCAAGTAATCCTTCCATCTAGGCCTCT |
| | | | | ATTTTTTGTGCAAACGATTGAAATTATATTT |
| | | | | TTTTTACCTGAATTTTTCCTGTGAACATTGG |
| | | | | GTTATTTATAAACCTGTTTTCTGTTTCTTTCT |
| | | | | TTCTTTTTTTTTTTTTTTGTTTTTGTTTTTTGA |
| | | | | GATAGAGTCCAGCCTGGAGTGCTGTGGCAT |
| | | | | GATCTTGGCACACTTGCAACCTCTGCCTCCT |
| | | | | GGGTTCAGGTGATTCTCCTCCTCTAGCCTCC |
| | | | | TCCACGCCTGGCTAATATTTGTATTTTTAGT |
| | | | | AGAGATGGGGTTTCACCATGTTGGCCGGGC |
| | | | | TGTTCTTGAACTCCTGGTTTCAACAGATCCA |
| | | | | CCTGCCTCAGCCTGCCAAAGTGCTGAGATT |
| | | | | ACAGGTGTGAGCCACTGTTCTAGGCACTTG |
| | | | | TTTCTGTTTCTTAATTTTGGCTGCTACTCAG |
| | | | | TGGGAAAAAGCACAGATTGAATCTAATTGA |
| | | | | GGCCGGGCGCTGTGGCTCACTCCTGTAATT |
| | | | | TCAGCACTTTGGGAGGCTGAGGTGGGCAGA |
| | | | | TCACCTGAGATCCAGAGTTCGAGACTAGCC |
| | | | | TGGCCAACATGGGGAAACCTCATCTCTACT |
| | | | | AAAAACACAAAAATTAGTTGGGCGTGGTG |
| | | | | GCTCATGGCTGTAGTCCCAGCTACTCGGGA |
| | | | | GGCTGAGGCATGAGAATTGCTTCAACCCGG |
| | | | | GAGGTGGAGGTTGCAGTGAGCTGAGATCA |
| | | | | GGACACTGCCCTCCAGGTTGGGCAAGAGA |
| | | | | GTGAGACTCGGTCTTAAAAAAAAAAAAAA |
| | | | | ATCTAGTTGAAAAATGTCATCGGGTCTTTC |
| | | | | CAAATTTTTACTAGGAATTTGTTAAAATTA |
| | | | | ACCAGGCTGGAAGTCATTATAGTTTGTTTG |
| | | | | TTTGTTTGTTTGAGATGGGGGTCTCACTCTG |
| | | | | TCACGCAGGCTGGAGTTCAGTGGTAGGATC |
| | | | | TCGGCTCACTGCAACCTCTGCATCCCAGAT |
| | | | | TCAAGCGATCCTCTCACCTCTGCCTCATGA |
| | | | | GTAGTTGGAACCACAGGCATGTGTCACCAT |
| | | | | GCTTTTGTAGAGACAGGGTTTCTTTCGCCCT |
| | | | | ATTGGCTAGGCTGGTCTCAAACTTGTGAGC |
| | | | | TCAAGCGATCCGCCCACCTTGGCCTCCCAA |
| | | | | AGTGCTGGGATTACAGGCATGAGTTACCTT |
| | | | | GCCTTGCCCATTATAGCTTTTTTGAGGCTGG |
| | | | | GTCTTACTCTCTGTCATGCAGGCTGGACTG |
| | | | | CAGTGGTGTGATCTAAGCTCACTGCCTCCT |
| | | | | GGGCTCAAGCAGTCCTCCCACCTCAGCCTC |
| | | | | CTGAGTAGCTGGCACAGGCGCTACCTCACC |
| | | | | CATCTAATTTTTTATTTTTTTTAGAGATGGG |
| | | | | GTTTTGCCATGTTTGCCCAGGCTGGTCTAG |
| | | | | AATTCATGAGCTCAAGTGATCTACCTGCCT |
| | | | | CGGCCTCCCAATGTGCTGGGATTACAGACA |
| | | | | TGAGCCACTATGTTCAGCCATACCTGGCTA |
| | | | | ATTTTTAAAAAATGTTTTCAAGAGACAGGG |
| | | | | TCTCCCTGTGTTGCCCAGGTTGGTCTCAAGT |
| | | | | TCCTGGGATTACTGCTGGCCTTCAAAAGTA |
| | | | | AATGTGAAATAATTAGTTAATTTCTCCCTC |
| | | | | AGTTGACAAATAATGCCAAAAGTGATAAA |
| | | | | GATTAATGAAATGTCTCTTTTTTTTTTTTTTT |
| | | | | TTTGAGACGGAGTCTCGTTCTGTTGCCAAG |
| | | | | TCTGGAATGCAGTGGCACGATCTCGGCTCA |
| | | | | CTGCAACGTCCACCTACTGGGTTCAAGTGA |
| | | | | TTCTCCTGCCTCAGCCTCCCGAGTAGCTGG |
| | | | | GACTACAGGCACGCATCACCATGCCCGGCT |
| | | | | AATTTTTGTATTTTTAGTAGAGACGGGGTTT |
| | | | | CACTATGTTGGCCAGGCTGGTCTTGAACTC |
| | | | | CTGACCTCATGATCCACCCACCTTGGCCTC |
| | | | | CCAAAGTGCTGGGATTACAGGCATGAGCCA |
| | | | | CCGCGCCCAGCCATGAAATTTCTTACGTAG |
| | | | | AAAGGCAGCTTGGGATTGTAGAAAGAATG |
| | | | | TAGGCTTTGGAGTTGGACAGGCCTCCATTT |
| | | | | GAGACCATACTTGAGTCCCGTGCTTGCCTT |
| | | | | AGACAAAGAACCTCTCAACCTTAGTTTTTA |
| | | | | ATCTATAAGGTGTTTTGAAAATTAATTCCT |
| | | | | AGTTCAGTACATGGCACATGGTAGGTACCT |
| | | | | GCTGCTATCCATAATTCTCTTAGTTAATATA |
| | | | | TTCGGTGCCACATGCCAGGCAGCCAGGATC |
| | | | | TGTACTAAGCACCTAATAAGTATTATCTCA |
| | | | | TTTAATCCTCAAAAGAACCCCACCTGAGTT |
| | | | | GCTAGACAGCCATTATTTCAGGGTTACACA |
| | | | | TTAGGAAATTGAAGCTTAGAGAGATTTAAG |
| | | | | TGGTTAGCCAAGTGATGGTGCTGGTATTCC |
| | | | | AACTAAGGTCATCTGCTTTCAGAGCATTTA |
| | | | | CTTTCTGTTAGGCTGCCTCTCCTGTTGCAAA |
| | | | | GTACTAAGAACACAACTACATAATGTATTT |
| | | | | TTAGTGGATTCTTGTCTTTTTGTAAATAGAA |
| | | | | GGTTAAAATGAGAGGAATTTTTTTTTTGTTT |
| | | | | GGGAGACGTGGTCTCGCTCTGATGAGAGCT |
| | | | | AGAAATTTGATTACTTGTATTTCTGGTCTGC |
| | | | | ATAAAAATTTGGCCTAAAAACATCAATAGA |
| | | | | AAGGCAAGTGTCATCTGCAAATCTGTCCCA |
| | | | | TCCTGTTCTTCACAGGAAAATGTAACCTTTT |
| | | | | TTTTTTTTTTTTCTTTTTTTGAGATGGAGTCT |
| | | | | AGCTCTGTTGCCCAAGCTGGAGTGCAATGG |
| | | | | CATGGTTTCCCGCTCACTGCAACCTCTGCCT |
| | | | | TCTGGGTTCTAGCAGTTCTCCTGCCTCAGCC |
| | | | | TCCTGAGTAGCTGGGATTACAGGCGCCTGC |
| | | | | CACCATGCCTGGCTAATTTTTGTATTTTTAG |
| | | | | TAGAGACAGGGTTTCACCATGTTGGCCAGG |
| | | | | CTGGTCTTTAACTCCTGACCTCAGGTGATCC |
| | | | | GCCTGCCTCGGCCTCCCAAAGTGCTGGGAT |
| | | | | CACAGGTGTGAGCCACTGCGCCCGGGCTCA |
| | | | | AAATGTAACGTCTGTCTAGTATGAGGATTT |
| | | | | ATTTCCTTGTCTGACTTCTGAGTTGTAATCG |
| | | | | TTTATTAACAATCACATTGTAAGTTTATCTA |
| | | | | TGAAGTAATAAAATGTTCTTTCTGTATATTA |
| | | | | TACTGGAAATGAATGCTTCATTCAAAAAAT |
| | | | | AGTTTTATCTTGGGAAGGTAGCCACTTTTTA |
| | | | | AAAATTGAGGTAAAACGGCCAGGCACGGT |
| | | | | GGCTCACGCCCATAATTCCAGCACTTTGGG |
| | | | | AGGCCAAGGTGGGTGGAGATCACCTGAGG |
| | | | | TCAGAAGTTCAAGACCAGCCTGGCCAATAT |
| | | | | GGTGAAACTCCATCTCTACTAAAATACAAA |
| | | | | AATTAGACCGGCATGGTGGCAGGTGCCTGT |
| | | | | AATCCCAGCTACTCAGGAAGCTGAGGCAG |
| | | | | GAGAATCGCTTGAACCCAGGAGGTGGAGG |
| | | | | TTACAGTGAGCCGAGATCCTGCCGCTGCAT |
| | | | | TGAAGCCTGGGTGAGAAGAGCGAAACTCT |
| | | | | GTCTCATTAAAAAAAAAAAAAAAGAGGTA |
| | | | | AAATTTAAATAACTTAAGGCTGATTGTATT |
| | | | | GGCTTACACTTGTAATTCCAGCATTTTGGG |
| | | | | AGACCAAGGCAGGAGGATCACTTGAACTC |
| | | | | AGAAGTTTGAGACCAGCCTGGTCAACATAG |
| | | | | GGAAACCTCATCTCCACAAAAAATAAAAA |
| | | | | ATAAAATATAAAAACTTCAAAATTAAATAA |
| | | | | GTTACAGTTCACCATTGTAACCATTTTATTT |
| | | | | TATCCTATTTATTTTGAGACAGTCTTGTTTT |
| | | | | GTCACCCAGGCTGGAGTACAGTGGTGGGAT |
| | | | | CACAGCTCACTACAGCCTCCACCTTCCAGG |
| | | | | TTCAAGTGATTCTTCTGCCTCAGCCTCTGTA |
| | | | | ACTGGGATTACAGGTGCTTGCCACCACACC |
| | | | | CTGCTAATTTTTGTATTTTGATTAGAGACAG |
| | | | | GGTTTCACCATGTTGGCCCGATTGGTCTCG |
| | | | | AACTCCTGAGCTCAAGTGATCTGCCTGTCT |
| | | | | TGGCCTCCCAAAATGAGCCACCGTGCCTGT |
| | | | | CCCCTTAGTCTACTTTAAAATTCAATTTGCC |
| | | | | TTTTTTTTAAATTGTAAGAATTCCTTATATA |
| | | | | TTTTGGATATTAAATCCTTAACTAGGGATA |
| | | | | TGATTCGCAAATTTTTTTCCCCCATTCTGTT |
| | | | | TCTGTAGGCTCTTTGACATTCTTTTTCTTTCT |
| | | | | CTTTTTGAGACAAGGTCTTACTCTGTTGCCC |
| | | | | AGGCTAGAGTACAGTGGTGTGATCATAGCT |
| | | | | TACTACAGCCTCGACTTCCCTGGGCTGAAG |
| | | | | CAATCCTTACCTCCCACCTCAGCCTCCCAG |
| | | | | GTAACCAGGACTACAGGTGTACACCACCAT |
| | | | | GCCTGGCAAATCACTGTTGTTGTTGTTGTTG |
| | | | | TTGTTATAGCCATAGGCTCCCACTGTGTTGC |
| | | | | CCAGGCTGGGCTCAAGCAATCTTCTAGCCT |
| | | | | TCTAGCCTTGGCCTCCCGAAGTGGTGGGAT |
| | | | | TATGCGCATGATCGCTGCTCCCAGCCCACA |
| | | | | ATCTTTTTTTTTTTTTTTTTGAGATGGAGTCT |
| | | | | CGCTCTGTCACCCAGGCTGGAGTGCGGTGG |
| | | | | CGCAATCTCGGCTCATTGCAACCTCCGCCT |
| | | | | CCCGGGTTCAAGCGATTCTCCTGCCTCAGC |
| | | | | CTCCCGAGTGGCTGGGATTACAGGCACGTG |
| | | | | CTGCCACGCCCAGCTAATTTTTGTATTTTTT |
| | | | | TTTTAGAAGAGATGGGGTTTCACCATATTG |
| | | | | GCCAGGATGGTCTCGAACTCCTGACCTCAT |
| | | | | GATATGCCCACCTTGGCCTCCCAAAGTGCC |
| | | | | GGGATTACAGGCATGAGCCACCGCGCCTGG |
| | | | | CCTCCCAGCCCACATTCTTGATAATTTTCTT |
| | | | | TGCTTCTAAAAGTTTTGCTTTTAGGGTTGGG |
| | | | | CAAGGTGGCTTATGCCTATAATCCTAGCAC |
| | | | | TTTGGGAGGCTGAGGTGGGCGGATCTCTTG |
| | | | | AGCTCAGGAGTTCAAGAACACCCTGAGCA |
| | | | | ACATGGAAAAACCGTGTCTCTACAAAAAAT |
| | | | | GCAAAAATTAGCCAAGTGTGGTGGCATGCA |
| | | | | CCTGTAGTCTGAGCTACTGGGGAGGCTGTG |
| | | | | ACAGGAGGATCACTTGAATTGGACTGGAG |
| | | | | GCTGCAGTGAGTGAAAATGGTACCACTGCA |
| | | | | CTCCAGCCTGGGTAATAGAGTGAGATGCTG |
| | | | | TCTGAAAAAAAAAAAAAGTTTTAGTTTTTT |
| | | | | TGGGTGGGGGGATTTTAACTTCACCTAT |
| 6 | NSD1 exon 11x | chr5: | + | TGAAACCTTAAAATGGAACAGCTCAGAAA |
| | | 176674925-176675080 | | GTTCCAGTGGAACAAACAGCCTCAGAGCA |
| | | | | GTTAGTGGCAGGGCATGAGGCGCCCACTAC |
| | | | | CCGCCCAATCACAGCAGGGTTAGAACTAAC |
| | | | | ATTGCATGCAGTCCGCCCGAGTGATTGGCT |
| | | | | GAACATCT |

TABLE 3
ASO sequences targeting ANKRD11 exon 4x.

| Seq. | | ASO Sequence | Target sequence | Target Genomic | Target |
|---|---|---|---|---|---|
| ID | Seq. Name | (5′→3′) | (5′→3′) | Coordinate | Strand |
| 7 | ANKRD11 4x | CTGGAGGCATCTGAAGGCA | ATGCCTTCAGATGCCT | chr16:89358176- | − |
| | ASO 5'-1 | T | CCAG | 89358195 | |
| 8 | ANKRD11 4x | GCATCTGAAGGCATCAACA | TTAGTGCTCTGTGTTG | chr16:89358182- | − |
| | ASO 5'-2 | CAGAGCACTAA | ATGCCTTCAGATGC | 89358,211 | |
| 9 | ANKRD11_4x_ | CAGTACTGTACCTTTCTTCT | GCAAGAAGAAAGGTA | chr16:89358078- | − |
| | ASO 3' | TGC | CAGTACTG | 89358100 | |
| 12 | ANKRD11_4x_ | CTGCACTCATCTGAC | GTCAGATGAGTGCAG | chr16:89358271- | − |
| | 15_1 | | | 89358285 | |
| 13 | ANKRD11_4x_ | ACACCCTGCACTCAT | ATGAGTGCAGGGTGT | chr16:89358266- | − |
| | 15_2 | | | 89358280 | |
| 14 | ANKRD11_4x_ | CAAGCACACCCTGCA | TGCAGGGTGTGCTTG | chr16:89358261- | − |
| | 15_3 | | | 89358275 | |
| 15 | ANKRD11_4x_ | CGTAACAAGCACACC | GGTGTGCTTGTTACG | chr16:89358256- | − |
| | 15_4 | | | 89358270 | |
| 16 | ANKRD11_4x_ | CTCCTCGTAACAAGC | GCTTGTTACGAGGAG | chr16:89358251- | − |
| | 15_5 | | | 89358265 | |
| 17 | ANKRD11_4x_ | TCAGCCTCCTCGTAA | TTACGAGGAGGCTGA | chr16:89358246- | − |
| | 15_6 | | | 89358260 | |
| 18 | ANKRD11_4x_ | CCACCTCAGCCTCCT | AGGAGGCTGAGGTGG | chr16:89358241- | − |
| | 15_7 | | | 89358255 | |
| 19 | ANKRD11_4x_ | TGTTTCCACCTCAGC | GCTGAGGTGGAAACA | chr16:89358236- | − |
| | 15_8 | | | 89358250 | |
| 20 | ANKRD11_4x_ | TCGGCTGTTTCCACC | GGTGGAAACAGCCGA | chr16:89358231- | − |
| | 15_9 | | | 89358245 | |
| 21 | ANKRD11_4x_ | AGAGCTCGGCTGTTT | AAACAGCCGAGCTCT | chr16:89358226- | − |
| | 15_10 | | | 89358240 | |
| 22 | ANKRD11_4x_ | GTGTGAGAGCTCGGC | GCCGAGCTCTCACAC | chr16:89358221- | − |
| | 15_11 | | | 89358235 | |
| 23 | ANKRD11_4x_ | ACACGGTGTGAGAGC | GCTCTCACACCGTGT | chr16:89358216- | − |
| | 15_12 | | | 89358230 | |
| 24 | ANKRD11_4x_ | ACAAGACACGGTGTG | CACACCGTGTCTTGT | chr16:89358211- | − |
| | 15_13 | | | 89358225 | |
| 25 | ANKRD11_4x_ | CACTAACAAGACACG | CGTGTCTTGTTAGTG | chr16:89358206- | − |
| | 15_14 | | | 89358220 | |
| 26 | ANKRD11_4x_ | CAGAGCACTAACAAG | CTTGTTAGTGCTCTG | chr16:89358201- | − |
| | 15_15 | | | 89358215 | |
| 27 | ANKRD11_4x_ | CAACACAGAGCACTA | TAGTGCTCTGTGTTG | chr16:89358196- | − |
| | 15_16 | | | 89358210 | |
| 28 | ANKRD11_4x_ | GGCATCAACACAGAG | CTCTGTGTTGATGCC | chr16:89358191- | − |
| | 15_17 | | | 89358205 | |
| 29 | ANKRD11_4x_ | CTGAAGGCATCAACA | TGTTGATGCCTTCAG | chr16:89358186- | − |
| | 15_18 | | | 89358200 | |
| 30 | ANKRD11_4x_ | GGCATCTGAAGGCAT | ATGCCTTCAGATGCC | chr16:89358181- | − |
| | 15_19 | | | 89358195 | |
| 31 | ANKRD11_4x_ | CTGGAGGCATCTGAA | TTCAGATGCCTCCAG | chr16:89358176- | − |
| | 15_20 | | | 89358190 | |
| 32 | ANKRD11_4x_ | CTGGGCTGGAGGCAT | ATGCCTCCAGCCCAG | chr16:89358171- | − |
| | 15_21 | | | 89358185 | |
| 33 | ANKRD11_4x_ | AGGGACTGGGCTGGA | TCCAGCCCAGTCCCT | chr16:89358166- | − |
| | 15_22 | | | 89358180 | |
| 34 | ANKRD11_4x_ | ACAACAGGGACTGGG | CCCAGTCCCTGTTGT | chr16:89358161- | − |
| | 15_23 | | | 89358175 | |
| 35 | ANKRD11_4x_ | GCACCACAACAGGGA | TCCCTGTTGTGGTGC | chr16:89358156- | − |
| | 15_24 | | | 89358170 | |
| 36 | ANKRD11_4x_ | TTGCAGCACCACAAC | GTTGTGGTGCTGCAA | chr16:89358151- | − |
| | 15_25 | | | 89358165 | |
| 37 | ANKRD11_4x_ | CAGCCTTGCAGCACC | GGTGCTGCAAGGCTG | chr16:89358146- | − |
| | 15_26 | | | 89358160 | |
| 38 | ANKRD11_4x_ | CGTACCAGCCTTGCA | TGCAAGGCTGGTACG | chr16:89358141- | − |
| | 15_27 | | | 89358155 | |
| 39 | ANKRD11_4x_ | AGGAGCGTACCAGCC | GGCTGGTACGCTCCT | chr16:89358136- | − |
| | 15_28 | | | 89358150 | |
| 40 | ANKRD11_4x_ | CTTCGAGGAGCGTAC | GTACGCTCCTCGAAG | chr16:89358131- | − |
| | 15_29 | | | 89358145 | |
| 41 | ANKRD11_4x_ | TGCTTCGAGGAGCGT | ACGCTCCTCGAAGCA | chr16:89358129- | − |
| | 15_30 | | | 89358143 | |
| 42 | ANKRD11_4x_ | CATGGTGCTTCGAGG | CCTCGAAGCACCATG | chr16:89358124- | − |
| | 15_31 | | | 89358138 | |
| 43 | ANKRD11_4x_ | CATGCCATGGTGCTT | AAGCACCATGGCATG | chr16:89358119- | − |
| | 15_32 | | | 89358133 | |
| 44 | ANKRD11_4x_ | CATCTCATGCCATGG | CCATGGCATGAGATG | chr16:89358114- | − |
| | 15_33 | | | 89358128 | |
| 45 | ANKRD11_4x_ | ACCTCCATCTCATGC | GCATGAGATGGAGGT | chr16:89358109- | − |
| | 15_34 | | | 89358123 | |
| 46 | ANKRD11_4x_ | TAGGAACCTCCATCT | AGATGGAGGTTCCTA | chr16:89358104- | − |
| | 15_35 | | | 89358118 | |
| 47 | ANKRD11_4x_ | GCTTCTAGGAACCTC | GAGGTTCCTAGAAGC | chr16:89358099- | − |
| | 15_36 | | | 89358113 | |
| 48 | ANKRD11_4x_ | TTCTTGCTTCTAGGA | TCCTAGAAGCAAGAA | chr16:89358094- | − |
| | 15_37 | | | 89358108 | |
| 49 | ANKRD11_4x_ | CTTTCTTCTTGCTTC | GAAGCAAGAAGAAAG | chr16:89358089- | − |
| | 15_38 | | | 89358103 | |
| 50 | ANKRD11_4x_ | TGTACCTTTCTTCTT | AAGAAGAAAGGTACA | chr16:89358084- | − |
| | 15_39 | | | 89358098 | |
| 51 | ANKRD11_4x_ | AGTACTGTACCTTTC | GAAAGGTACAGTACT | chr16:89358079- | − |
| | 15_40 | | | 89358093 | |
| 52 | ANKRD11_4x_ | TGGTCAGTACTGTAC | GTACAGTACTGACCA | chr16:89358074- | − |
| | 15_41 | | | 89358088 | |
| 53 | ANKRD11_4x_ | CCAACTGGTCAGTAC | GTACTGACCAGTTGG | chr16:89358069- | − |
| | 15_42 | | | 89358083 | |
| 54 | ANKRD11_4x_ | CAAGGCCAACTGGTC | GACCAGTTGGCCTTG | chr16:89358064- | − |
| | 15_43 | | | 89358078 | |
| 55 | ANKRD11_4x_ | TAAATCAAGGCCAAC | GTTGGCCTTGATTTA | chr16:89358059- | − |
| | 15_44 | | | 89358073 | |
| 56 | ANKRD11_4x_ | ATCAGTAAATCAAGG | CCTTGATTTACTGAT | chr16:89358054- | − |
| | 15_45 | | | 89358068 | |
| 57 | ANKRD11_4x_ | AACACATCAGTAAAT | ATTTACTGATGTGTT | chr16:89358049- | − |
| | 15_46 | | | 89358063 | |
| 58 | ANKRD11_4x_ | TTTAAAACACATCAG | CTGATGTGTTTTAAA | chr16:89358044- | − |
| | 15_47 | | | 89358058 | |
| 59 | ANKRD11_4x_ | CACAGTTTAAAACAC | GTGTTTTAAACTGTG | chr16:89358039- | − |
| | 15_48 | | | 89358053 | |
| 60 | ANKRD11_4x_ | GCAGACACAGTTTAA | TTAAACTGTGTCTGC | chr16:89358034- | − |
| | 15_49 | | | 89358048 | |
| 61 | ANKRD11_4x_ | AAATGGCAGACACAG | CTGTGTCTGCCATTT | chr16:89358029- | − |
| | 15_50 | | | 89358043 | |
| 62 | ANKRD11_4x_ | ATATAAAATGGCAGA | TCTGCCATTTTATAT | chr16:89358024- | − |
| | 15_51 | | | 89358038 | |
| 63 | ANKRD11_4x_ | TGCAGATATAAAATG | CATTTTATATCTGCA | chr16:89358019- | − |
| | 15_52 | | | 89358033 | |
| 64 | ANKRD11_4x_ | AACAGTGCAGATATA | TATATCTGCACTGTT | chr16:89358014- | − |
| | 15_53 | | | 89358028 | |
| 65 | ANKRD11_4x_ | CTCCAAACAGTGCAG | CTGCACTGTTTGGAG | chr16:89358009- | − |
| | 15_54 | | | 89358023 | |
| 66 | ANKRD11_4x_ | CCCTCCTCCAAACAG | CTGTTTGGAGGAGGG | chr16:89358004- | − |
| | 15_55 | | | 89358018 | |
| 67 | ANKRD11_4x_ | CCCGTCCCTCCTCCA | TGGAGGAGGGACGGG | chr16:89357999- | − |
| | 15_56 | | | 89358013 | |
| 68 | ANKRD11_4x_ | CCTTCCCCGTCCCTC | GAGGGACGGGGAAGG | chr16:89357994- | − |
| | 15_57 | | | 89358008 | |
| 69 | ANKRD11_4x_ | TTCCACCTTCCCCGT | ACGGGGAAGGTGGAA | chr16:89357989- | − |
| | 15_58 | | | 89358003 | |
| 70 | ANKRD11_4x_ | CCATGGTGCTTCGAGGA | TCCTCGAAGCACCATG | chr16:89358123- | − |
| | s1 | | G | 89358139 | |
| 71 | ANKRD11_4x_ | TCATGCCATGGTGCTTCG | CGAAGCACCATGGCAT | chr16:89358118- | − |
| | s2 | | GA | 89358135 | |
| 72 | ANKRD11_4x_ | GTCAGTACTGTACCTTTC | GAAAGGTACAGTACTG | chr16:89358076- | − |
| | s3 | | AC | 89358093 | |

TABLE 4
ASO sequences targeting ANKRD11 exon 11x.

| Seq. | Seq. | ASO Sequence | Target sequence | Target Genomic | Target |
|---|---|---|---|---|---|
| ID | Name | (5′→3′) | (5′→3′) | Coordinate | Strand |
| 10 | NSD1 11x 5' | TAA GGT TTC ACT AAG | TCTCCCTTAGTGAAACC | chr5:176674915- | + |
| | | GGA GA | TTA | 176674934 | |
| 11 | NSD1 11x 3' | AAG CAC TTA CAG ATG | CTGAACATCTGTAAGTG | chr5:176675071- | + |
| | | TTC AG | CTT | 176675090 | |
| 73 | NSD1_11x_15_ | GGCATTCTATTCAAA | TTTGAATAGAATGCC | chr5:176674825- | + |
| | 1 | | | 176674839 | |
| 74 | NSD1_11x_15_ | TCTTGGGCATTCTAT | ATAGAATGCCCAAGA | chr5:176674830- | + |
| | 2 | | | 176674844 | |
| 75 | NSD1_11x_15_ | GCCCCTCTTGGGCAT | ATGCCCAAGAGGGGC | chr5:176674835- | + |
| | 3 | | | 176674849 | |
| 76 | NSD1_11x_15_ | ATAATGCCCCTCTTG | CAAGAGGGGCATTAT | chr5:176674840- | + |
| | 4 | | | 176674854 | |
| 77 | NSD1_11x_15_ | CCTAAATAATGCCCC | GGGGCATTATTTAGG | chr5:176674845- | + |
| | 5 | | | 176674859 | |
| 78 | NSD1_11x_15_ | TGTTTCCTAAATAAT | ATTATTTAGGAAACA | chr5:176674850- | + |
| | 6 | | | 176674864 | |
| 79 | NSD1_11x_15_ | ATCAGTGTTTCCTAA | TTAGGAAACACTGAT | chr5:176674855- | + |
| | 7 | | | 176674869 | |
| 80 | NSD1_11x_15_ | CCAAGATCAGTGTTT | AAACACTGATCTTGG | chr5:176674860- | + |
| | 8 | | | 176674874 | |
| 81 | NSD1_11x_15_ | TCCTTCCAAGATCAG | CTGATCTTGGAAGGA | chr5:176674865- | + |
| | 9 | | | 176674879 | |
| 82 | NSD1_11x_15_ | ATTTGTCCTTCCAAG | CTTGGAAGGACAAAT | chr5:176674870- | + |
| | 10 | | | 176674884 | |
| 83 | NSD1_11x_15_ | TACTTATTTGTCCTT | AAGGACAAATAAGTA | chr5:176674875- | + |
| | 11 | | | 176674889 | |
| 84 | NSD1_11x_15_ | TTGATTACTTATTTG | CAAATAAGTAATCAA | chr5:176674880- | + |
| | 12 | | | 176674894 | |
| 85 | NSD1_11x_15_ | TTTATTTGATTACTT | AAGTAATCAAATAAA | chr5:176674885- | + |
| | 13 | | | 176674899 | |
| 86 | NSD1_11x_15_ | TTAAGTTTATTTGAT | ATCAAATAAACTTAA | chr5:176674890- | + |
| | 14 | | | 176674904 | |
| 87 | NSD1_11x_15_ | CATTCTTAAGTTTAT | ATAAACTTAAGAATG | chr5:176674895- | + |
| | 15 | | | 176674909 | |
| 88 | NSD1_11x_15_ | GAAAACATTCTTAAG | CTTAAGAATGTTTTC | chr5:176674900- | + |
| | 16 | | | 176674914 | |
| 89 | NSD1_11x_15_ | GGAGAGAAAACATTC | GAATGTTTTCTCTCC | chr5:176674905- | + |
| | 17 | | | 176674919 | |
| 90 | NSD1_11x_15_ | CTAAGGGAGAGAAAA | TTTTCTCTCCCTTAG | chr5:176674910- | + |
| | 18 | | | 176674924 | |
| 91 | NSD1_11x_15_ | TTTCACTAAGGGAGA | TCTCCCTTAGTGAAA | chr5:176674915- | + |
| | 19 | | | 176674929 | |
| 92 | NSD1_11x_15_ | TAAGGTTTCACTAAG | CTTAGTGAAACCTTA | chr5:176674920- | + |
| | 20 | | | 176674934 | |
| 93 | NSD1_11x_15_ | CATTTTAAGGTTTCA | TGAAACCTTAAAATG | chr5:176674925- | + |
| | 21 | | | 176674939 | |
| 94 | NSD1_11x_15_ | TGTTCCATTTTAAGG | CCTTAAAATGGAACA | chr5:176674930- | + |
| | 22 | | | 176674944 | |
| 95 | NSD1_11x_15_ | TGAGCTGTTCCATTT | AAATGGAACAGCTCA | chr5:176674935- | + |
| | 23 | | | 176674949 | |
| 96 | NSD1_11x_15_ | CTTTCTGAGCTGTTC | GAACAGCTCAGAAAG | chr5:176674940- | + |
| | 24 | | | 176674954 | |
| 97 | NSD1_11x_15_ | TGGAACTTTCTGAGC | GCTCAGAAAGTTCCA | chr5:176674945- | + |
| | 25 | | | 176674959 | |
| 98 | NSD1_11x_15_ | TCCACTGGAACTTTC | GAAAGTTCCAGTGGA | chr5:176674950- | + |
| | 26 | | | 176674964 | |
| 99 | NSD1_11x_15_ | TTTGTTCCACTGGAA | TTCCAGTGGAACAAA | chr5:176674955- | + |
| | 27 | | | 176674969 | |
| 100 | NSD1_11x_15_ | GGCTGTTTGTTCCAC | GTGGAACAAACAGCC | chr5:176674960- | + |
| | 28 | | | 176674974 | |
| 101 | NSD1_11x_15_ | AATGTTAGTTCTAAC | GTTAGAACTAACATT | chr5:176675031- | + |
| | 43 | | | 176675045 | |
| 102 | NSD1_11x_15_ | CATGCAATGTTAGTT | AACTAACATTGCATG | chr5:176675036- | + |
| | 44 | | | 176675050 | |
| 103 | NSD1_11x_15_ | GACTGCATGCAATGT | ACATTGCATGCAGTC | chr5:176675041- | + |
| | 45 | | | 176675055 | |
| 104 | NSD1_11x_15_ | GGGCGGACTGCATGC | GCATGCAGTCCGCCC | chr5:176675046- | + |
| | 46 | | | 176675060 | |
| 105 | NSD1_11x_15_ | CACTCGGGCGGACTG | CAGTCCGCCCGAGTG | chr5:176675051- | + |
| | 47 | | | 176675065 | |
| 106 | NSD1_11x_15_ | CCAATCACTCGGGCG | CGCCCGAGTGATTGG | chr5:176675056- | + |
| | 48 | | | 176675070 | |
| 107 | NSD1_11x_15_ | TTCAGCCAATCACTC | GAGTGATTGGCTGAA | chr5:176675061- | + |
| | 49 | | | 176675075 | |
| 108 | NSD1_11x_15_ | AGATGTTCAGCCAAT | ATTGGCTGAACATCT | chr5:176675066- | + |
| | 50 | | | 176675080 | |
| 109 | NSD1_11x_15_ | CTTACAGATGTTCAG | CTGAACATCTGTAAG | chr5:176675071- | + |
| | 51 | | | 176675085 | |
| 110 | NSD1_11x_15_ | AAGCACTTACAGATG | CATCTGTAAGTGCTT | chr5:176675076- | + |
| | 52 | | | 176675090 | |
| 111 | NSD1_11x_15_ | CCATTAAGCACTTAC | GTAAGTGCTTAATGG | chr5:176675081- | + |
| | 53 | | | 176675095 | |
| 112 | NSD1_11x_15_ | TCTAGCCATTAAGCA | TGCTTAATGGCTAGA | chr5:176675086- | + |
| | 54 | | | 176675100 | |
| 113 | NSD1_11x_15_ | ATTTGTCTAGCCATT | AATGGCTAGACAAAT | chr5:176675091- | + |
| | 55 | | | 176675105 | |
| 114 | NSD1_11x_15_ | CTGCTATTTGTCTAG | CTAGACAAATAGCAG | chr5:176675096- | + |
| | 56 | | | 176675110 | |
| 115 | NSD1_11x_15_ | CTGGGCTGCTATTTG | CAAATAGCAGCCCAG | chr5:176675101- | + |
| | 57 | | | 176675115 | |
| 116 | NSD1_11x_15_ | TCCCTCTGGGCTGCT | AGCAGCCCAGAGGGA | chr5:176675106- | + |
| | 58 | | | 176675120 | |
| 117 | NSD1_11x_15_ | CCCCCTCCCTCTGGG | CCCAGAGGGAGGGGG | chr5:176675111- | + |
| | 59 | | | 176675125 | |
| 118 | NSD1_11x_15_ | TTTGACCCCCTCCCT | AGGGAGGGGGTCAAA | chr5:176675116- | + |
| | 60 | | | 176675130 | |
| 119 | NSD1_11x_15_ | TTCCATTTGACCCCC | GGGGGTCAAATGGAA | chr5:176675121- | + |
| | 61 | | | 176675135 | |
| 120 | NSD1_11x_15_ | GTCTCTTCCATTTGA | TCAAATGGAAGAGAC | chr5:176675126- | + |
| | 62 | | | 176675140 | |
| 121 | NSD1_11x_15_ | TTGATGTCTCTTCCA | TGGAAGAGACATCAA | chr5:176675131- | + |
| | 63 | | | 176675145 | |
| 122 | NSD1_11x_15_ | TATTATTGATGTCTC | GAGACATCAATAATA | chr5:176675136- | + |
| | 64 | | | 176675150 | |
| 123 | NSD1_11x_15_ | ATCTGTATTATTGAT | ATCAATAATACAGAT | chr5:176675141- | + |
| | 65 | | | 176675155 | |
| 124 | NSD1_11x_15_ | CCCACATCTGTATTA | TAATACAGATGTGGG | chr5:176675146- | + |
| | 66 | | | 176675160 | |
| 125 | NSD1_11x_15_ | AATGTCCCACATCTG | CAGATGTGGGACATT | chr5:176675151- | + |
| | 67 | | | 176675165 | |
| 126 | NSD1_11x_15_ | AAAATAATGTCCCAC | GTGGGACATTATTTT | chr5:176675156- | + |
| | 68 | | | 176675170 | |
| 127 | NSD1_11x_15_ | AAGAAAAAATAATGT | ACATTATTTTTTCTT | chr5:176675161- | + |
| | 69 | | | 176675175 | |
| 128 | NSD1_11x_15_ | TTGCAAAGAAAAAAT | ATTTTTTCTTTGCAA | chr5:176675166- | + |
| | 70 | | | 176675180 | |

### REFERENCES

Bershteyn M, Nowakowski T J, Pollen A A, Di Lullo E, Nene A, Wynshaw-Boris A, Kriegstein A R (2017) Human iPSC-derived cerebral organoids model cellular features of lissencephaly and reveal prolonged mitosis of outer radial glia. Cell Stem Cell 20:435-449 e434.
Birey F, Andersen J, Makinson C D, Islam S, Wei W, Huber N, Fan H C, Metzler K R C, Panagiotakos G, Thom N, O'Rourke N A, Steinmetz L M, Bernstein J A, Hallmayer J, Huguenard J R, Pasca S P (2017) Assembly of functionally integrated human forebrain spheroids. Nature 545:54-59.
Feng H, Moakley D F, Chen S, McKenzie M G, Menon V, Zhang C (2021) Complexity and graded regulation of neuronal cell type-specific alternative splicing revealed by single-cell RNA sequencing. Proc Natl Acad Sci USA 118:e2013056118.
Finak G, McDavid A, Yajima M, Deng J, Gersuk V, Shalek A K, Slichter C K, Miller H W, McElrath M J, Prlic M, Linsley P S, Gottardo R (2015) MAST: a flexible statistical framework for assessing transcriptional changes and characterizing heterogeneity in single-cell RNA sequencing data. Genome Biol 16:278.
Gallagher D, Voronova A, Zander M A, Cancino G I, Bramall A, Krause M P, Abad C, Tekin M, Neilsen P M, Callen D F, Scherer S W, Keller G M, Kaplan D R, Walz K, Miller F D (2015) Ankrd11 is a chromatin regulator involved in autism that is essential for neural development. Dev Cell 32:31-42.
Han Z, Chen C, Christiansen A, Ji S, Lin Q, Anumonwo C, Liu C, Leiser S C, Meena, Aznarez I, Liau G, Isom L L (2020) Antisense oligonucleotides increase Scnla expression and reduce seizures and SUDEP incidence in a mouse model of Dravet syndrome. Sci Transl Med 12:eaaz6100.
Havens M A, Hastings M L (2016) Splice-switching antisense oligonucleotides as therapeutic drugs. Nucleic Acids Res 44:6549-6563.
Herrmann J, Pallister P D, Tiddy W, Opitz J M (1975) The KBG syndrome-a syndrome of short stature, characteristic facies, mental retardation, macrodontia and skeletal anomalies. Birth Defects Orig Artic Ser 11:7-18.
Horvath, S. (2013). “DNA methylation age of human tissues and cell types.” Genome Biol 14(10): R115.
Hua Y, Krainer A (2012) Antisense-mediated exon inclusion. Methods Mol Biol 867:307-323.
Hua Y, Vickers T A, Baker B F, Bennett C F, Krainer A R (2007) Enhancement of SMN2 exon 7 inclusion by antisense oligonucleotides targeting the exon. pLoS Biol 5:e73.
Hua Y, Vickers T A, Okunola H L, Bennett C F, Krainer A R (2008) Antisense masking of an hnRNP A1/A2 intronic splicing silencer corrects SMN2 splicing in transgenic mice. Am J Hum Genet 82:834-848.
Jeffries, A. R., et al. (2019). ““Growth disrupting mutations in epigenetic regulatory molecules are associated with abnormalities of epigenetic aging”” Genome Res 29(7): 1057-1066.
Ka M, Kim W Y (2018) ANKRD11 associated with intellectual disability and autism regulates dendrite differentiation via the BDNF/TrkB signaling pathway. Neurobiol Dis 111:138-152. Kadoshima T, Sakaguchi H, Nakano T, Soen M, Ando S, Eiraku M, Sasai Y (2013) Self-organization of axial polarity, insideout layer pattern, and species-specific progenitor dynamics in human ES cell-derived neocortex. Proc Natl Acad Sci USA 110:20284-20289.
Kim J et al. (2019) Patient-customized oligonucleotide therapy for a rare genetic disease. N Engl J Med 381:1644-1652.
Lewis B P, Green R E, Brenner S E (2003) Evidence for the widespread coupling of alternative splicing and nonsense-mediated mRNA decay in humans. Proc Natl Acad Sci USA 100:189-192.
Licatalosi D D, Darnell R B (2010) RNA processing and its regulation: global insights into biological networks. Nat Rev Genet 11:75-87.
Lim K H, Han Z, Jeon H Y, Kach J, Jing E, Weyn-Vanhentenryck S, Downs M, Corrionero A, Oh R, Scharner J, Venkatesh A, Ji S, Liau G, Ticho B, Nash H, Aznarez I (2020) Antisense oligonucleotide modulation of non-productive alternative splicing upregulates gene expression. Nat Commun 11:3501.
Maquat L E (2004) Nonsense-mediated mRNA decay: splicing, translation and mRNP dynamics. Nat Rev Mol Cell Biol 5:89-99.
Martin-Herranz, D. E., et al. (2019). Screening for genes that accelerate the epigenetic aging clock in humans reveals a role for the H3K36 methyltransferase NSD1. Genome Biol 20(1): 146.
Morel Swols D, Foster J, 2^{nd}, Tekin M (2017) KBG syndrome. Orphanet J Rare Dis 12:183.
Neilsen P M, Cheney K M, Li C W, Chen J D, Cawrse J E, Schulz R B, Powell J A, Kumar R, Callen D F (2008) Identification of ANKRD11 as a p53 coactivator. J Cell Sci 121:3541-3552.
Nilsen T W, Graveley B R (2010) Expansion of the eukaryotic proteome by alternative splicing. Nature 463:457-463.
Pan Q, Shai O, Lee L J, Frey B J, Blencowe B J (2008) Deep surveying of alternative splicing complexity in the human transcriptome by high-throughput sequencing. Nature Genet 40:1413-1415.
Parfitt D A, Lane A, Ramsden C M, Carr A F, Munro P M, Jovanovic K, Schwarz N, Kanuga N, Muthiah M N, Hull S, Gallo J M, da Cruz L, Moore A T, Hardcastle A J, Coffey P J, Cheetham M E (2016) Identification and correction of mechanisms underlying inherited blindness in human iPSC-derived pptic cups. Cell Stem Cell 18:769-781.
Papillon-Cavanagh, S., et al. (2017). Impaired H3K36 methylation defines a subset of head and neck squamous cell carcinomas. Nat Genet 49(2): 180-185.
Shiba, N., et al. (2013). NUP98-NSD1 gene fusion and its related gene expression signature are strongly associated with a poor prognosis in pediatric acute myeloid leukemia. Genes Chromosomes Cancer 52(7): 683-693.
Pasca A M, Sloan S A, Clarke L E, Tian Y, Makinson C D, Huber N, Kim C H, Park J Y, O'Rourke N A, Nguyen K D, Smith S J, Huguenard J R, Geschwind D H, Barres B A, Pasca S P (2015) Functional cortical neurons and astrocytes from human pluripotent stem cells in 3D culture. Nat Methods 12:671-678.
Sacharow S, Li D, Fan Y S, Tekin M (2012) Familial 16q24.3 microdeletion involving ANKRD11 causes a KBG-like syndrome. Am J Med Genet A 158A:547-552.
Satterstrom F K et al. (2020) Large-scale exome sequencing study implicates both developmental and functional changes in the neurobiology of autism. Cell 180:568-584 e523.
Scarano E, Tassone M, Graziano C, Gibertoni D, Tamburrino F, Perri A, Gnazzo M, Severi G, Lepri F, Mazzanti L (2019) Novel mutations and unreported clinical features in KBG syndrome. Mol Syndromol 10:130-138.
Sheng L, Rigo F, Bennett C F, Krainer A R, Hua Y (2020) Comparison of the efficacy of MOE and PMO modifications of systemic antisense oligonucleotides in a severe SMA mouse model. Nucleic Acids Res 48:2853-2865.
Sirmaci A, Spiliopoulos M, Brancati F, Powell E, Duman D, Abrams A, Bademci G, Agolini E, Guo S, Konuk B, Kavaz A, Blanton S, Digilio M C, Dallapiccola B, Young J, Zuchner S, Tekin M (2011) Mutations in ANKRD11 cause KBG syndrome, characterized by intellectual disability, skeletal malformations, and macrodontia. Am J Hum Genet 89:289-294.
Van de Sande B, Flerin C, Davie K, De Waegeneer M, Hulselmans G, Aibar S, Seurinck R, Saelens W, Cannoodt R, Rouchon Q, Verbeiren T, De Maeyer D, Reumers J, Saeys Y, Aerts S (2020) A scalable SCENIC workflow for single-cell gene regulatory network analysis. Nat Protoc 15:2247-2276.
Velasco S, Kedaigle A J, Simmons S K, Nash A, Rocha M, Quadrato G, Paulsen B, Nguyen L, Adiconis X, Regev A, Levin J Z, Arlotta P (2019) Individual brain organoids reproducibly form cell diversity of the human cerebral cortex. Nature 570:523-527.
Walsh C A, Morrow E M, Rubenstein J L (2008) Autism and brain development. Cell 135:396-400.
Wang E T, Sandberg R, Luo S, Khrebtukova I, Zhang L, Mayr C, Kingsmore S F, Schroth G P, Burge C B (2008) Alternative isoform regulation in human tissue transcriptomes. Nature 456:470-476.
Weyn-Vanhentenryck S M, Feng H, Ustianenko D, Duffié R, Yan Q, Jacko M, Martinez J C, Goodwin M, Zhang X, Hengst U, Lomvardas S, Swanson M S, Zhang C (2018) Precise temporal regulation of alternative splicing during neural development. Nat Commun:2189.
Wilfert A B, Sulovari A, Turner T N, Coe B P, Eichler E E (2017) Recurrent de novo mutations in neurodevelopmental disorders: properties and clinical implications. Genome Med 9:101. Wolf F A, Angerer P, Theis F J (2018) SCANPY: large-scale single-cell gene expression data analysis. Genome Biol 19:15.
Wright C F et al. (2015) Genetic diagnosis of developmental disorders in the DDD study: a scalable analysis of genome-wide research data. Lancet 385:1305-1314.
Yan Q, Weyn-Vanhentenryck S M, Wu J, Sloan S A, Zhang Y, Chen K, Wu J Q, Barres B A, Zhang C (2015) Systematic discovery of regulated and conserved alternative exons in the mammalian brain reveals NMD modulating chromatin regulators. Proc Natl Acad Sci USA 112:3445-3350.
Zhang A, Li C W, Chen J D (2007) Characterization of transcriptional regulatory domains of ankyrin repeat cofactor-1. Biochem Biophys Res Commun 358:1034-1040.
Zhang A, Yeung P L, Li C W, Tsai S C, Dinh G K, Wu X, Li H, Chen J D (2004) Identification of a novel family of ankyrin repeats containing cofactors for p160 nuclear receptor coactivators. J Biol Chem 279:33799-33805.
Zhang, H., et al. (2011). Reversed clinical phenotype due to a microduplication of Sotos syndrome region detected by array CGH: microcephaly, developmental delay and delayed bone age. Am J Med Genet A 155A(6): 1374-1378.

We claim:

1. A method of increasing or decreasing expression of a target mRNA and protein by cells having a pre-mRNA that comprises a poison exon and encodes the target protein, the method comprising contacting the cells with an antisense oligomer (ASO) complementary to a targeted portion of the pre-mRNA encoding the target protein, wherein the target protein is selected from the group consisting of ANKRD11 and NSD1, wherein the antisense oligomer (ASO) binds to a targeted portion of the pre-mRNA and modulates binding of a factor involved in splicing of the poison exon, thereby modulating the level of the processed mRNA encoding the target protein and modulating the expression of the target protein in the cell, and wherein poison exon is selected from exon 3× in the ANKRD11 gene, exon 4× in the ANKRD11 gene, and exon 11× in the NSD1 gene.

2. The method of claim 1, wherein the targeted portion is proximal to the poison exon.

3. The method of claim 2, wherein the targeted portion is about 1 to about 1500 nucleotides upstream of 5′ end of the poison exon.

4. The method of claim 2, wherein the targeted portion is about 1 to about 1500 nucleotides downstream of 3′ end of the poison exon.

5. The method of claim 1, wherein the targeted portion is within the poison exon or wherein the targeted portion overlaps with the boundaries of the poison exon.

6. A method of treating a disease condition in a subject in need thereof by increasing or decreasing expression of a target protein by cells of the subject, according to the method of claim 1.

7. The method of claim 6, wherein the disease condition is KBG syndrome, the target protein is ANKRD11, and the poison exon is selected from exon 3× in the ANKRD11 gene, and exon 4× in the ANKRD11 gene.

8. The method of claim 6, wherein the disease condition is Sotos syndrome or reverse Sotos syndrome; the target protein is NSD1; and the poison exon is exon 11× in the NSD1 gene.

9. The method of claim 6, wherein the disease condition is normal or pathological aging; the target protein is NSD1; and the poison exon is exon 11× in the NSD1 gene.

10. The method of claim 6, wherein the disease condition is cancer; the target protein is NSD1; and the poison exon is exon 11× in the NSD1 gene.

11. The method of claim 6, wherein the targeted portion is proximal to poison exon.

12. The method of claim 11, wherein the targeted portion is about 1 to about 1500 nucleotides upstream of 5′ end of the poison exon.

13. The method of claim 11, wherein the targeted portion is about 1 to about 1500 nucleotides downstream of 3′ end of the poison exon.

14. The method of claim 6, wherein the targeted portion is within the poison exon, or wherein the targeted portion overlaps with the boundaries of the poison exon.

15. A method of increasing or decreasing expression of a target mRNA and protein by cells having an pre-mRNA that comprises a poison exon and encodes the target protein, the method comprising contacting the cells with an antisense oligomer (ASO) complementary to a targeted portion of the pre-mRNA, wherein the target protein is ANKRD11, wherein the antisense oligomer (ASO) binds to a targeted portion of the pre-mRNA and modulates binding of a factor involved in splicing of the poison exon.

16. The method of claim 15, wherein the poison exon is selected from exon 3× in the ANKRD11 gene and exon 4× in the ANKRD11 gene, and wherein the targeted portion is proximal to the poison exon or within the poison exon.

17. A method of increasing or decreasing expression of a target mRNA and protein by cells having an pre-mRNA that comprises a poison exon and encodes the target protein, the method comprising contacting the cells with an antisense oligomer (ASO) complementary to a targeted portion of the pre-mRNA, wherein the target protein is NSD1, wherein the antisense oligomer (ASO) binds to a targeted portion of the pre-mRNA and modulates binding of a factor involved in splicing of the poison exon.

18. The method of claim 17, wherein the poison exon is selected from exon 11× in the NSD1 gene, and wherein the targeted portion is proximal to the poison exon or within the poison exon.

19. The method of claim 1, wherein the target protein is ANKRD11 and the ASO is selected from the group consisting of ANKRD11 ASOs 5′-1 (Seq. NO 7), 5′-2 (Seq. NO 8), 5′-3 (Seq. NO 9), 4-8 (Seq. NO 15-19), 29-33 (Seq. NO 40-11), 37 (Seq. NO 48), 41 (Seq. NO 52), 43-44 (Seq. NO 54-55), and S1-S3 (Seq. NO 70-72).

20. The method of claim 1, wherein the target protein is NSD1 and the ASO is selected from the group consisting of NSD1 ASOs 5′ (Seq. NO 10), 3′ (Seq. NO 11), 23-25 (Seq. NO 95-97), 46-48 (Seq. NO 104-106), and 55-56 (Seq. NO 113-114).


SEQUENCE LISTING (128 sequences)

SEQ ID NO: 1 - DNA, 32125 bp, Homo sapiens
  source 1..32125: mol_type = genomic DNA
agaggccgccctgagacggtgcgcgatggaccgagggccccagccggggaggcgccgccgccgagcccgcggccagacgccccatcagtagcgtccgcaccgggagccgcggctctcgcccgagccgtgggcgcgcccgaggggcgggctcgcctcccgccgtccctcgcagctctgccgggcccgagcccgcgccgccgccgccgccgccttgccgctcgggccgcgcggcccgggaaacgcggccgcgggctgcatgggcagcgcccgcgccccgccgctgagccgtcgcggagccgcgcagccctcggagcacggtgagaggcgccgctggtctgggggcggtggtcggggcgggcacggggcattcgcgcggccttgcggcctgcaggccttccccggcgacggagctgcgccgcgggcctccgggcgggcctggggggtcggggccgggtgggcgggggtctttgggggcccggggcgatcgtgagggaccaataatgggtcccggagcgggcctacgggtccgggttcggggcagccgggggtctttgggggcccggggtggccgtgaggggcccatagggggctccggggggggcctgggaggtcggcggagcttgggtcggccgtaaggggcggacgggggctccgggcgggcctgggtggccgggggcccgcggcggctctgaggggccgattgggggctccggggcgggcctgggggccgtgggggcccggggcggccgtgagaggcggacagggggctccggggcgggcctcgggggctcggagcggccgtgaggggcggacggggggcttcggggcgggcctgcaggtcttggggacctgaggctgccgggaggggctgccagggggcgctggccgggcgccgggttctgcggagctggggcgccgacctctgacccgcgagaggggcgccttcgccgtgctggtcgtagttgttattctcagcgtccctattattatcgctgttttgaaatgagagcaggcggctctcgggctccgagccggagggggagggcgaactggggacctgggggcgtcggggttgcaggaggcgcgcgtaggccgaggaggggcaggaatgcgggcagccgtgtggggggtgttagggggagggtaggcgggcgggtgtggggggtggctgggaggaaagcggtggcggtggcggctgcaacagcagcccttggcctcaaggaacaatgtgagacgttgcctgaaatgttaatttccgttcctcatttcatcatcccctggcagggcggtagctgtgtgtgtggtgtatgtgtgtgcgcgcgcgcgcgcgcgcgcgtgtgtgtgtgtgtgtagggttggccctgccacattgattcagtcccctctcaaagagggattgtactgttaactcttgtgtttgtgttatttgggaaggttggtcggggggagtcttgatttttctcgaggcttgctcttttcctggtgccccattaagattttctgcttctgttgtgtttttggaaggttagtgttatatatcagcttccaagaagttttggaagaggcttgggaagatgaagctggtctaacagctccctatgctttgaaactgttttccttctatgtaacatgcttaggattcatcgtttttgtagattatgagtagttttgtatccttttgcaagaacagggttttatggtagagaaattagatttcaggctttcttacatgaggagagagttttatgcagtcattagagcaatatccttaacacttgaaatgagaaataaaagtgtgcacagttgttgaactgcaaaacttagagaatcttttgatcctttttgggatgttgagacttagccaaacatataagatgatattacctgtggaaaaagtgccatcgaaacagttactgtttttgttgtgtgcagacctagtctatggcataacgttcaaaatcggagaccctgaggctgtttctttggtttctctgagaccttgatttctcttagtaagtattggctgccctggaagacttcgagttctgttagagaatgattacagaagtcctctgattttactctgcaactgtagttaaactagaaaaaaaggggggggggcggatgtcttgctcagccttcattgtgacccttgcattggatctcgtatagttggtgtggcctcccctaccctccattcagctgttgttctcctctttctgtagtttctccccgaagagcaggatctccttcgataggctggccatctgaatggaactggatgggagggaacaaagaccaagttgctgtgacgttttctctgttcttcgtgatgcctcttaagtttgttgaaaagttccccacaagatagtgctaaatttgacctaaacttgaaactttcagcagtttttttcttctgttctatttttgctgtagtaaaatatacataacacaatttaccagaattttgtttttaaaggtgtcacctttaaaacctgagttcctcttttgcagctctcatttgcgtcagccttctgacttgcattgccaccgccagatgcttttctccggactggccatgctgggcaccccataccagcagtgccatcacaccctcccagatgggctgttgatgacgagcggctgccatgttagcggtaattacagtgttgatacggtggcaagtagaactccctacaaatagagtgaaggaaactatgctgtttgtgttggacagtatttttccaagaagtttttgtgcccctttttattttattttatttttatttttttggagtctcgctctgtcgcccaggctggagtgcagtggcgcaatcttggctcattgcaacctccgtctcctggggttcaagcagttctctgcctcagcctcccgagtagctgggattacaggcgcccaccgccacgcctggctaatttttgtatttttagtagagatggggtttcaccatgttggccacgctggtcttgaactcctaaccttgtgatccacctgccttggcctcccaaagtgctgggattacaggcgtgagccaccgcgcccggcctctttttgtgcctttttaagtagatttgacatagaaaggttatgcttcatcaaatataagaggagtcttattttgcatatgggcagagagcccatcatcaattaaataacataattagagagtatcagtaaatgctggctcagaaaataatctgcattttgttgccaaaataagtgttttgatctgtccattactctgtgtaactcccttcatcctcaaatcgtaagtgtaatgggtgagtgtctcttaatagcaaggtatttgtagttaatcggtgaaaatgatggtgcatcccttgtctggtggctgcgtcagtttgtgactcttgtgtagtcagtgctctgtgggaattcagtgtggcccgtttgataaactttatagaaaaatggaatgaattcaaataaaagttattgtcttgaaattttgaaatgttttcagtgtgtgttctcttaatttgaaattgtttttttcttttttttttttttgagacggagtctcgctttgttgcccaggctggagtgcagtggcatgatctcgactcactgcaacctccacctcctgggtttaagcaattctcctgcctcagcctcccaagtagctgggattacagacacctcctaccaaacacggctaatttttttttattgttagtagagactggagtttcaccatgttggccaggctggtttcaaactcctgacctcaagtgatccacctgcctcggcctcccaaagtgctgggattatagacgtgagccaccatgcctggcctcaaatgttatttttatgataacgtcccaaatggtgactgtggcctaactctgataccatgccccattcttgaccctgccccaagcttccagtcattctggctgcgttggccttgcctttcctcagttgggccatgtgccatagtttgtggcatggcctcgacatacctcgctgccatctcatgcagatccccccaaactccaagataactcctactcagcctttgcgtcttagtgtaatatcacttcctttagtaagtcttttttttttttttttgaggcagggtctcggtgtgttgctcaggctggagggcagcggccagttgcgtgaacgtggctctcttcagtctcgactccctgagctgaagcgatcctcccacctcagactcctgagtagctgggaccacaggcatgtgccgccgtgcccagctaatttattttactttttgtagaaacagggtcttcctgtgttgtgtaggcctcagccttctggggttggcttacctcagtctcctaaagtgttgggattataggcatgagccactgtgctcagcccccttttgttttcattacaagtttgcttttataacttagataaaggtgggaagacagttttcacaattaaggcagaacaagagataaagaaacataaagggagagttgtctttgtgggaagaaagtgctttctgtgaaagcacttttagcaggactgctgttttcaggccagagtagaagggccagaagcagtttttattcagatttcctcatcgtttccatcaaggtctcagtgactgtaaaggatgggttgaaatcagtctggactaagtgtggtttgctgctgctgcttatgaggattaagtgtctcgtttgtccggaggcccctctggtcacatgtagctgagcaggtggagtgatatgaaagttttagcattttaatgaagaaaaaatgatctctttggagggaaagacaacgaaatggaggtgtgccctattgatttttttctgtatgtttaggttttcaaagtttctttgtaaatgaaactgttatgtaattggcaaaagtttgctttttaaagactaattgtgggtatatttgggagattgcgacttgttttagatttttgggtttacagtaaggggctagggtatttggtggaggggaactggttgattgtggttgaaaacattcagatactattctagctctaagaaacctcacagttttgttttgttttgagacagagtctctgtcacctaggctggagtgcagtggcacgatcttgggtcatagcaacctctgcctcccggattcaagtgattcttctgcctcagcctcccgagtagctgggactacaggcccataccaccactcccagttagtgtttttttttttttttttttaaagtagagatggggtttccccatgttggtcaggctggtctggaacccctgacctcaggtgatccagccgtcttggcctcccaaagtgctgggattacaggtgtgaactaccgcgcctagccagaaaccccacagtgctaaagctggctctgtgaataaagggttaagagggttttcagtgtgaaaataaaagtaagccgtcctgtctgtatggctgagatttttcaggggcctagaggaccgaatcctgttgagtgaaaatccagtcttctgggcttccccacacccgctttctctcttcctcttggaggtcaaggttgagacagctgtcagggtgctgctgtctcagttacagcgtccttgtggctggtggagggtgagacctgtttccaggcctgtccttcgcatctgtgcagtgaattatttattatcctggaaatctgtagggctcttttttccacgtgtaaagcaaccaatgtgtacctgttcattatttgaaaatgttggctttttctcaacaaaccatttttataccatatgaaaatttcttttgtcatggtaaaattcagaagactttccccttcattacttaactctaaaaaatgtgacttttaagaatggctgaaattgaaatgttatttgtaaatgcctaataactttatgatatcagaggttattttttatgtaaaattagcaaataaacctcttttcttggtgcattgatagtaagttgcccttcctcgactccctatggctcttttcagacttaggatgctaatggcttagaataaattttggaatgcagtatttccaagtaaagggaagattgcgtaggctgcttggttcctgaggttttactagagttaggattagcttactgctgcattcatatctgacacaatcaaagaagactttggtttaattctggatgatgatgtaaacttgaaatcattatggctgtactgtttaaacttatctaaaatagagaaggtaagacgcagtgaaggaccttatttttctcttaagaaatcaagctttgttagtatccaccatgtttctagatgtggttttacatcttgcaaaacaggaaataaaagtaaaaaaccaaaaaaaccccagagcaccactcttgaaaggattaagtttttaaaaatgattttgactaagatgtctggctgattaaaggatgtgcagagcactgaataacctttgccttttctgatggtgacaaagaagaaatccagctttcaggcagccgaagagcgtctcgagagcttgtagtgttagtattccacagccccacagttgattcggatttcaaggaatttttagactttgtggattttttcttcactataattgtatgtttggctcctaatttatttaaattacatacatagatatttttgttactttgagaatagtctatctgaaatttgaagttctttagagcttaatatattaaatatgctaacactcaaaacattttctttctttttttttttttttgagatggagtttccctcttgttgcccaggcatgatcttggctcaccgcaacctccgcctcccgggttcaggcgattctcctacctcatcttcccgagtagctgggattacaggcacgcgccaccactcctggctaattttgtatttttcgtagagacggggtttctccgtgttggtcaggctggtctcaaactcccgacctcaggtgatctgcctgccttggcctcccaaagggctgggattacaggcgtgagccaccatgcccggcctaaacattttctcacaggcatttttcccctgacacatgcgagaggtatctttgaattgtatcctttatcttttagtgtgaaactcagaaaagtgatgcacgcttgcacttacagttcaggtaaaatgttaagcatattcaatgagatttacattcatgcttgatttttctttggcaaagtctttagatctgattctgctaaacttgggttctcaccagatgactggcttttaaaagaagatgatgttgaaactgactcttgtaaaaaaggacatttagtagaagctaatggtacggtgaagttttagagagttgaggagaaaatctgtcttagaacttaatctgtgccttttccttaatagctttcttctaagcccataaatatattggttcagggggatgagtgaaggcaaatggagggtgtggaagggagacagagaacagctcttgggcgttggagaagtgctgggacttgtgtcagtgctgcccgtgttggttttccagcgctgctgtaactaaccaccataaccttagatttattgttttatagttctgttgattagaagcctaacatgatctgcctgggctaaaatctgggtaggtagggctggttccttctggaggctccaggggagaatctgtgttcttgtcttttccgtgttccagaggctgcctgcattcggcctgtagtgctttcctcagtcttcaaagccagcctcatggcatctctctgactcctgctgcccacccgcttttttggagagtctcggctctgttgcccaggctggagtgcagtggcacaatcaaggctcactgcagcgtcgacctcctgggctcaatcaatcctcccgcctcagccccccagcagtgctggaattacaggcatggagccactgtgcctggcccttgtttcacttttggatccttgtggttacaaattgggcccacccgagcactgcgaggtcatctccctctctgctgattagtaacctcagttcccttttccacgtaacctaaggcattcacaggttccaggggtgagcaagctggaggtttttccagtaatgttcccttgctgtcctcagatgccatccgggatcccacattgcatttagttgtcatgtctccttaatctcgtctgcaaccatttctcagcgtttccctgtttttcatgaccttggcagttttgatgagggcagtcatctttttttatgtctttgtgactgaccgttcacacattggcatgtttgtaaagcacggtgtggctgacgtgtggctcacctgactgcttttccaaatggggtgcagtgtgctctacagtgggagaacagctttgtgtcttcttttagctggaaggagctacatgttttatagaagggacttctgaaactagacaaactctgctttttctgatgtttcactgatttccttcacagatgttcattggctgctcactgggtgcagtgctgtggcctcctgttctgagatctgggagaaagatgcatttagttgactaaagcttgcattaaatattggttcttatgaaaagagagatcatagatcagaggagggaaaggctctgtgacctgggagatttaagggaagaagtgctattttttttttttttaactgtaagctatgtttctgttttaaaaaaagaatcagtagaatgtcactgcagaaatcagagtaagggcatcttcgacttcgggaaggctggggacaaaggctgggaaggccgcatgtaacgtgagaatgtcagccagacgtgtctgaacacgaaagcccatccaagaagatggaagatgacagcagattaacctggaagcatgactggagttgggttgtgtagggtttgcaatgatagacctgaatttcatttgcttacttgttttgaggcagaagatgatgtgtcagagtttgcaggcggtggatgcctagatgaaagcactggctttgcaacaccatggtgcctaaatggttctttttttgagacaaggtcttgctctgtcactcagggtggagtgcagtggcgtgaccatatctcactgcatccttgagcttctgggctcaagtgagcctcctgcgtcaacgtcatgagtggctagaactacagacgtgcgctaatttttaaaaattctttgtagaaatggggtttcgctatgtttcttaggctgatgttgaactcctggcctcaagtagtcctcctgcctcagcctcccaaagtgctgggatgacaggcacaagacatcacatgcagccttaagaagatccctacaagagaacctggagaaacttgaaatagggaaggggactcgaaagcacactgtgtaggtgaattgtgaagacttcatggtgttaactggtattcattaaaaagacttggaaaaagagaagatgaattgattcccagtggagggaatggggcaaacattagttaggagagtggctttgtgaaacgatagagcaaaggagacagaaaagaaagcaggaatggggaacggggtggtggggcagaagctgggtgctgtttattttgtgcctactgtgtgtcactctccattctctgccctggggacatttgacagatgtggacgtcactggtgtgatttgcccacattctggagggagaagtgggcatctcagttgaggaaaggaaatggctatctggggaattcttgaggggcacagcccaggacggagccttgtgttagatgggtacctgctggctctgctcagctcggcagatgtgttgcctaaatgactttaatggccaaggcagactggtatttgccctcagatattttgtcaggacagtgtaaaatgtgggccaagatagtggttttgggtccaaacagaagggggttagagattttggaggttgaagcctgctgctagaaggagtatagaaagaagaacggggattctgtttgcccaaatacatggttaggctcatcttggacttgtttttgttttatatcttctgttacttctatgcacatattaatagatacctaattaatatttggttggaaatcttaaaagttaggattttttttttctttttttttgagatggagtctcactctgtcgcccaggctggagtgcagtggtgtgatctcggctcactgcaacctccgcctcctgggttcacgcgattctcctgcctcagcctcccgagtagctgggactacaggcacccaccaccaggctcggctaatttttttgtatttttaatagagtcagggtttcgccatgttagccagaatggtctcaatctcctgaccttgtgatccacccgccttggcctctcaaagtgctgggattacaggcgtcagccatcacgcctggcctaaaagttaggatttttaaaaaggcttgtggtctgagaaggaaggcaataatacttggcaggaaagacttaattttttttttttgagactgagtcttgctctgttgcccaggctggagtgcaatgacacgatctcagctcactgcaacctctgcctctcgggttcaagcatttctcctgcctcagcctcccgagtagctgggattagaggtgtgtgccatcacgcccggctagtttttttttttttttttttgagatggagtctcgctctgtcacccaggctggagtgcagtggcacaatctcggctcactgcaagctccgactcccaggttcatgccattctcctgcctcagcctcccgagtggttgggactacaggcacctgccaccacccccggctaattttttgtatttttagtggagatggggtctcaccgtgttagccaggatggtctcaatctcctgaactcgtgatccgccccctcggcctcccaaagtgctgggattacaggtgtgagccactgcgtctggcctttttaaaatttaatttaattttttttgggggcggagtttcgctctgttgcccaggctggagtgcagtggtgtgatctcagctcactgcaacctccgcctcctgggttcaaatgattctcttgcctcagcctccagaatagctgggattacaggcatgtgctaccaagcccagctaatttttttgtatttttagtagacatggggtttctccgtgttggtcaggctggtctcaaactcccgacctcagatgatccgcctgcctcggcctccttaagtgctgggattgcaggtgtgagccactgcgccaggcctagatattttttatatcaggtcacatgtgttttatctaggaggcaaactttccttgtaattagtgttttttttttcctttttttgtttttgtttgagtggagtctcgccactgcgtctggccgtcattaatgtttttctagtgatgttgcactctcttgacgtcagttaactgtcacctgaatcttcagggactgggttgggttgtggggtaggtggtggcgtgacttgctgttgatgggactgctgtccacaggaacaggaggacccactagttacacagctgccaaaggtgtgggtcttgaggcccgaaggttgaggttgcagtgagctgagatcatgccactggtctctagccccggtgacagagtgagaacatctcaaaaaagaaaagaaaaagtgttgggtcattatttacacacccaacaagtgaaaggtctggagggcccaagtgaggacagctcagttcaggcatgtttaacctccagattacctcatccttgagaaatttaggaataatcccagtgtcagtctttggaggtgtggttctttgatgttagtcatgggaataaggaatttgcattaagcctcaagaattgtggaaagtcctaatgagaaatgtcacgggcagaaactgccacccgctttagtggtgcaggtgatggctgtgccggagagctgcgcgtagccggctgctggaggagaggacgtggcctgtacaggcagcttcgctctggggggtgactgttggagggcagcggggacgaaagggacacatttgtatgatgctcagttgtcagcaggatgtccaggttgcttttttggagggcagtgggtccttgagaccttgagacccattctctttggaaagtaatgagttaccgggtgggggctgtaggaggatgttcagttgtggtgtggaggcgtcgggttgcttaacctcagagggatcttttttcctatgagttgtgtagaagaggatgtcttccatagatttgaaggacgttaaaaaaaaaaccaaccccaatgtggctttccttctttctcactagggtgatagtccgacgtgcatgtctgtttcctggaccctggatgtgaaaatggggttcttgaaggcactgggagtgtttctctgctggccccagaggcctgtgtgtgctgctggcgaggcagctgggccactgtcactgtgccctgacaacaacgttgggaactgttcctgaaagtgttaaacaaaatttcagtttattaagcgctcctctcattaagccattgttttatttttcttttttgattttttgggcttcttttgaagccataataaattggaatagaaagaatacataccaagaaacaaatattaggtctgattttttaattttttgttattatttttgagatggagtcttgctctgtccccagactggagtgcagtggcgtgatcttggctcactgaaaccttcgcctcctgcgttcaagcagttctcctgcattcaagcagttctcctgcctgccaccacacctggctgatttttgaatttttagtagagttggggtttcaccatgttggccaggctggtcttgaactcctgacctcaggtgatgtacctgctcagcctcccaaagtgctgggattacaggcatgagccaccgcgcccggcctctccaatatttttattattaaaatgctaaatactgccgggcccagtggctcacacctgtaatcccagcactttgggaggctgaagcgggtggatcacctgaggtcaggagttcgcgaccagcctgggccacatggtgaaaccctgtctcttctaaaaatacaaaaaattagctgggcgtggtggcaggcgcctgtaatctcagctactcgggagggtgaggcaggagaatcggttgaacccaggaggtggaggttgcagtgagccagaatcgcaccactgcactccagcctgggtgacagagtgagacgccatctcagagagaaaaaaaagaaattacgcgtggtggcccatgcctgtcatcccagctactgaggaggctgaggcaggagaatcgcttgaacatgggaggtggaggttgcagtgagccgagatggcgccactgcactccagcttgggcaccagagtgagactctgtctccaaaaaaaaaaaaaaaggggaaatgacttaaaggtgatggcttttatacttctattgtgcctgttttctgagatataaatttaactagctaattctctcgtgttttaaatagtagacaaagaaagacaagaccaaaggagaacctttttctctgtttcttactccgtctgcttttattaatagatgctcacggtgtggtcttccactcacttcccctttcatctctgagcttaacgagctcctcgtattatagattgttaccatatcatgtgttccagtctgtgcccgtgtataaacgtgtgttgtgtgttacgcgatactgtgagatgagtctgcccagagggactctgaagtcaggactgtgtcttttccacacctctccaccccagctctcatcatgcctctgagagaaccagattcagagtgtggtgaggggaggatgaagtggtttggggtgggccttgggcccccatctctttgctggaagtgtagtatacctctaggatatgtgtccaaactgttggctgtgagaccaaggaggagaagtcttttttggcaggctagtgcctgcggcttgaggtctcagtgtctgtaactgccaggctgcagagccccacctggctgagtcaggagtgtgttgtaacctgcccacctgcccaggctggttagaagcaagcgtaggcgttgggtctgcctgtcctggtccaggcacctctcctggtttggccaggttttggtttgtatttattcctgatgttgatgtgtaaatgatatcgttacaaagcaggtagtttgctttgctattctacgaataacccaagaacctgaggataataggacacgttaacagtctgctagttgagagttctgtttctgtgacttcaggggacatatgaccatcccgattgtggtgggttattaaggctgtgacaagtcacaggtggctttagggatgtcaaagataggcaaagataggttcatttgaatttgatttcatcttttgagaatgggttggtatacctgaaattggctttgtagttttggtattttgatgtgagaaggcattggctgaattttttttgttctcataatttgcatattttctgtgttttctccattgtttggctcagttgttttctttttctttttttttttttttgagacacagtctcgctctatcactcaggctggagtgcagtggcgtgatcttggctccctgcaacctccacctcccggtttcaagcacttcatctgcctcagcctcccaagtagctgggactacaggtgcccaccaccacgcctggttaatttttaaatttttttagtagagacagggtttcaccatgttggccaggctggtcttgaactcctcacctcaattgatccacccaccttggcttcccaaaatgctgggattccaggtgtgagcccccgcacctggccggggtcagttgttttcgtgttccttatccctctttaaacttgggagagcattttgtgtttcgtggagatatcacagcatagaacagaattttgattgtaattgtttgttgttgacttgctgtagtactgttttcagacttccagtgtgaacgataaaagattatcttaaaattttaggaaaaattatcttttgtgtgggtagtgaataaataattagcattaatgttaccgatgtcttctacctagttctttatcaaaattcttttcctgactgcaatatttcattttaatagagaataattttcatgtgaaactgcttgctttatattttctttctggttgctatagaaacagtgcagcgtggtgggttacggggctttcatgagggagctctctgggaggcagcagtcacctggtgatgagggaggggaatggaattgtcttctttcacatcccagcttggccacggagcctcaggagctgcatcatcagaatgattggattttcttgtccctccagcttaaatgctgtgggctttgttctccagtggagtggtcattgccttttttcctacctgtgagttagtccatctctccaacccactgacgacccaagagctccccagcctttctctgcccgtcacagtcaagagggtctgttggaaaattactgactgaacaacagatgccagagattgccctgaattgtggaagtcccggctgccacccccttacttgaggtcctcatctcactgcaccagcaggggtgaggagagggcaggggtggccctgcacaaagctggggaagaaaggaggcagctcccggccaggcagggcccagctttccttctagatcatcagcacaaatttgcgtttgaagatttaacttacattttatttttttatttggatgatacatgtgtaagttttgggatgttatgcagtgttctgaggattgacgttggtggcatatgtaaccttgaaggcagcattttgttaattataaaaaacttggaatatagtgattgagtatgaaataattgaagctgtgagttcagttatgaagaacttggtctcggagtctctctgagcttgggaggctgccttggtctcagtggggttggcatcatgtgtgttggtggtaggttggaaagagcatttggagttctgaagagtgacctgtgctgcctttctggcaaccagtcattatttgtgcatggaaggaagtagcatggatgaaagcttttctttcaaatgggatgatgtgtgggggtatatttctggtcttaaatttttttttttttttttacataaggtagagacagggtctcaccatgttgcccaggctggtctcaaggtcctgggctcagtcattccttccaccttggcctcccatagtgctgggattacaggcatgagtcaccatgccaggccagtgtatttattttctactgctgtgtaacaaattgacacacaacacatttattatctcacagtttctgtgggttggaagtctgggcacagcttagtgggatcctctgctcagggtttcatccggttgcaatctaggtgttgggtgggactgtgatcttatcagaggctcgagtgggggatggtgtgcttttgagctctttcaggttgtggcagaagttatctcttgctgattgtgggacgcaagtctttgttttcttgttgactgtcccttgaggtctcctcttgactcttagaggccccctgtacttgcttggcgcatggccccttgcagcttggcagctcacttcaaaacagcaaagagtcttctgcttcagtagactaagtcttacgtaacataatgtagtcatgggagtgacagcgcagcaactttgccacctaatgcaacccagtcaagggaaagccattccattgttttcaccacattctgtgggttagaagcacgtcacaggtcccacccgccttgcactccgggcaaggtggctacaaggaggcatggacactgggtggggagattgccaggctcacctagggtctgtctgctggcctggggtgttcttcagaaccctcattccctggatactgacatgtcagtctctgggaattggttgcatctctctctcctccatgacgtatcagtctgtgggagagcggtgcgtctcttctctctcctccatttgcatgcattcccggacccacttgtagaacggaagtagaatcaagactcgtggccacagtttgggtggatgggacacttgggcttgccctggtactatcaggctgtttgccctatgccttgcatttcccactgtcctgcctgcctctcaggcttatggtgaggatgaggtgagatgagatgatggatgtggtcagtgaagcagctggtcagggcctggtggaattctactctagtccagtctgcctctttctccacctctattctctaggcagagattggttattttcagtagtaagtatggactcatcagagttcttgaggtctttgcttcctgcctcaaattcccttctctcctttccctgactgatgcctactcatccttcaggcctccatgttcccaatgatccttttttttttttgaggcagggtcttgctttcttgcctcggctggagggcagtggtgtgatcctgacttattgcagccttgacctcctgggctcaagccaagctgtcctgggtagctgggaccacaggtgtgtaccaccacacctggctaatgtttgtactttttgtagagtcggggtctcactgtgttgcccaggctggtctcccaacttctgggctcaagtgatcctcctgcctcagcctcccacagtgctgggatcacagacatgagccactatgtctggcccccagtgagcctcttgatctcatggttgagctcttttttcacttgtgacttctgccattggttagattctcacctttggtcttgcggtgctctctgctcccctctgtggtaataatttattgttgtgttgtaattatcttttacttcctttccactaagtgatattcattggatcctcaaataaaaggcttggcacacttagggcagatacctaataaaacagtaacacctctgtggtcagtatttaaacggtgtgcacagccattagaatggaataagctgggcgcagtggaatgggaggttgatgttgcagtgacacatgaatgtgccactgccctccagcctgggggacagagcaagagctcatctcaaataaatcaataaatgggtcagaggagtgtgatgtgctggtgactcgggtcctgctctgccgccggagcttttccagccttgctgctcctttgctttggatcttttcattcctaggtttattgttgagtctgagacggttttttttctcccttccccttccccttagtaaatgtgcagatctcctgatattgcacctgacttagagatcttggtaccgtccctacagatccacacaaacacaaaagcacaggtgatactcaggttggaacatgaagatgcaaagtcagcatccccatttgcctctcacttttctgtcttccttagctgtgtcctgcgagtgtttattgggcagtgttgacagcagcttcagtctcagaaaaaggatgggaagttgctctcagactcggaaccctaaagcttggtcggaattaggtttctgccctgatcctgatgctctttctgcccggtggagagcacctttaaaagtagtcacctgagggtcaaggatgggtacaacagcttcccattttattctaggagatgtgtctggagataaaatgagcatgtgatgtttggcagggctgcatgcctcgagggtcatagccattgtccctgatgttcaggacctggtaactgggggagtaaggacttaaggtacatgttttcctgtttcctctttgctgtgagttgtatgtgagttgatttgggtggtaaatgaaatcatatcttttttttttttttttttttttttttgagacagagtctcactgtcgcccaggctggagtgcagtggcatcatctcagctcactgcaacctctgcctcctgagttcaagcaactctccttcctcatcctcccaagtagctgggattacaggtgtccgccaccacgcccggataatttttgtatttttagtagagaggaggtttcatcatgtgggccaagctggtcctgacctcaggtgatctgcccacctctacctcccagagtgctgggattataggcgtgaggcaccacacctggtcatgaaatcatatcttaagtgtctccatggtggcctaatttgttacctgaagctttttctcagagcagcctctagcaaagagaatcacttcctgtggactccttcagggctgcagggtaacttgatgagttcttgccgtctcgtgaattcctgagtggtgagagcaccactccacacaggacttcggggcagcaggcttttaggttttgcacacagttcctcgaaagctgtgatttggaaatcgctagaatttccagatagtaactagtttggagggtcaatagtgctttagttttatttatttatttttttttacttttaaaacagaggtggggtctcactctgctgtcaggctgatctccagcttctgagctcaaatgatcctcctgccttggcctcccagagtgctgggattacaggagtgagccactgcgcccagcctcagtcatgcttttaaattgaggatgtaggaaggaaggctttggctcccatgctttcatgagatttccttttttttctgagacagagtctcgctctgtcgcccaggctggagtgcagtggcatgatctcagctcactgcaagctccgcctcccaggttcacaccactctcctgcctcagcctcccgagtaactgggactacaaatgtccgccaccgtgcccagctaatttttttttgtattttttagtagagacggggtttcaccgtgttagccaggatagtcttgatctcctgaccttgtgatccacccgcctggtactaccaaagtgctgggattacaggcatgagccaccgcgcccggccacgcccggctaattttttgtatttttagtagagactgggtttcgccatgttagccaggatggtctcgatctcctgaccttgtgatctacctgcctttgcctcccaaagtgctgggattagaggcgtgagccatcgcacctggctgctttcatgagatttcttagagactaatactttagtatttaccctcctttctcagtctatggtgttaaccagtattccctacctacgtttagtctgtacacaaaacacccatggctgcctctcctcagactgacctgcgttgacctggacctggataagctcctcactgtcatctgaggggtgtgtttccccttgtgtgcctgtcctaatagtgcatcccatttcagcgctttttctacagggcaggatttgtagaaagggtttgaatcttagtgataagctatgaccatgagtaagttacttcatttttcctcgcttttggttttcttgtaagaattgggattataggccggtgacattataggcatggtgactcacgcctgtaatcccagcgctttgggaggccggggcaggcagatcacaagttcaggacacggagaccatcctggctaacacggtgaaaccccgtctctactaaaaatacaaaaaaattagccgggcgtggtggcgggcgcctgtagtcccagctactcgagaggctgaggtaggagagtggcgtgaacccgggaagtggaggttgcaatgagccgagatcgcaccactgcgctccagcctgagcgacagagtgagctccgtcaaaaacaaaagaaaaggaaaaagtacaactgactttgtttttctgaaacggagcctcactctgtctccgggcgcgatcttggctccctgcaactgccgctcccgggttcacgccattctcctgcctcagcctcccgagtagctgggactacaggcgcccgccaccacgcccagctattttttttgcatttttagtagagacggggtttcaccgtgttagccaggatggtctccatctcctgacctcgtgatccgcccgcctcagcctcccaaagtgctggaattacaggtgtgagccaccgcgcctggcctactttttcctttcttatttgcgtacgttttatctcctttctcttggactagaatctccagtacggtgttcaaaagaagtgatgagtggagatcaaccaggtgcggtggctcacgcctgtaattctagcacttcgggaggccaaggtgggtggatcacctgaggttaggagtttgacaccatcctgggcaacacagtgaaaccctatctttactgaaatgcaaaaaaattagctggacgtggcagtgtttgcctctattcccagctgctcaggaggcagaggctggagaatctcttgaacctgggaggcagaggttgcagtgagccaagattgcgctacagcactctagcctgggcgacagagtgagactccatctcaaaagaaaaaaaagagtggatatcacaggcttatttcttttttttcctctttttttttttgaaacagagcctcgccctgtggcccaagctgaagtgcagtgcagtggtggctcactgcaggctctgacacaggcttatttctgatggtaattgaaaagtgtccactttttcaccattaaccatgatgtttgctgtgggatttcataaaggcactttatgaggttgaggaagttcccttctattacaagtttgctaagtatcaggaatggacattgaattttatagttttcttttacatttatttatcatttggttttgttttttgaacgtttaaccaatcatgtattcctgggttaaacccacttggtgacagtgtatcattcttcctgtaggatacattggctggcagggtgtcagctgagccctgtacgtttcaacatccagcaggctggccattggggctgccaagaacagtagggcagcagaagcaggggccacatggcttcacttttgcttcgtccttctttttttttgagatagcgtcttactctgttgcccaggctggagtgcagtggcgcgatctcggctcactgcaacctctgcctcccaggttcccaagcaattctccagcctcagcctcctgagtagctaacattacaggcctgtgccaccgcgctcggctaatttttgtatttttagtagagacaggatttcgacatgttggccaggccagtcttaaacttctggcctcaagtgatccacctgtctcagcctcccaaagtcctgggattacaagtgtgagccattgcacctggccaacttttgcttcattctgttggtaacagcaaatcggtgagtgagactgggttcaaggggtgcaaaatagactttccccccgacctcatgattggaggagctgcactcacgttgcaggtgtgggtgagaaggtgatagagtctgtgccatgtggcatagctactacaacaacttaaacccaaatcctcttagttttgctgtagtcatccaaataattgtttagatttctgctttggttttccttttcaagttaacactaagttaatagacccttctttccaagttcatgattacagtgtcataaagtgataaagactgcagtctgggcgtggtggctcacacctgtaatcccagcactttgggaggccgaggtgggcagatcacttgaggccaggagttcaagaccagcctggccaacataaccaaaccccgtctctactaaaaatacaaaaaaacttacctgggtatggtggtgcgcatctgtagtcccagctactcgggaggctgaggcaagagaaacacttgaacccgggaggcagaggttgcagtgagctgagatcacgccactccacttgagcccgggcaacagagcgagacattgtctcaacaaacaaacaaaacaaaacactgtgggtagcaagtcacccagtcgtcttatctgatttttaaaaacatatgcagtatgtcttacggtatcttgattagattcacaacagcatttgggatcagctgtgcagtgcatcctgcgtgttgaagagtgatacagggtcagatgcaacgcctgtagtctcagcactttgagaggccaaggcgggggatcacttgagaccaggagctcaagaccagcctgggcagcatagcaagaccccgtctctacaaaaataaaaataaaacttagctgggtgtgatagcatgtgcctgtagtcccagctacttgggaggctgaggctggatggccacttgagcccagggtttctttttagagacaaggtcttactctgtggtccaggctggagtgcagtgttgccatcacagctcactgcagcctcaacttcctgggctcaggcagttttcccacctcagcctctcaaatagctgagactacaggtgcgtgccaccatacccagccagctaatctttctgttttttttttttgtagagatggggtttagccatgttgccctggcccatctcaaactcctgggctcaagcgatccacctgctgtggcctccccaggttctgggattatagacatgagccgccgctcctggcctgattgcagcttgtgggtttggcaactttggtcaataaaagattatgtggtctttttctccctgcgccttctcactcctggcacagagttcctgcaaccattggaatttcttatatgataggatgtcatttgttattcataactagctcctttcagatcacaccagagtttaagctaatgaactgacagggtaggggtggtcaccaggaagaccagatgattattagagggctagggctttcatctccggccactgacctccagggaagggagcaggagctgaagatggagctctaaaaactctcgaacatgcccgagttgcgggagggcccctgccgcccacacttggctctttgcatcttttgtttgctgctaccgagttgtcttttttatattttccatgtcgaacatgtggaatacagttcaacagcttttaatgtctttgtctactaattttaacacctgtttcaattctatgttggtttatttcttttaacctcattatggtttgtatttgtgtttttttgagcactaatcgtcgttttctgaggcttgtattttttccttctttgcctagtgattttgttggatgccaggcattaggaattttaccccgttgggtgatgactttcttttgaaaaatgaacgagatctggttcacagaatacaaaatttaccatttacagctcacagtttagtgatttttagtatattcattatattgtgcacccatcaccactgtcacattccagcaagtttccatcctcatgcggtcccctgtgtcatcagtccttgtcccccttccccctttccctgacaactggtactctcttttctatctctctggatttgcctattgtgaatgtttcccataaatgagaattaagtcttttgggttttagataaataagatcatcatttcttcttcagagaattgaactgtcagcaggtgggggctcgttgcttgtagggggtgggctgcatgcgctctggtgtttacctggtgtgcctgagcccacggccagtgcagcaggttctgccagcatctttttctgggcagcttgttgagtttatgacacaatctccttttactggtccctgttgtgattggcaccctgaccttttagaaagtgtgatggtggccaggcgcggtggctcacgcctgtaattccagcactttgggaggccgaggcgggcagatcatgaggtcaggagatcaagaccatcctggctaacacggtgaaaccccatctctactaaaaatacaaaaaattagccgggcatggtggcgggcgcctgcagtcccagctactcgggaggctgaggcaggagaatggcgtgaactcaggaggtggagcttgcagttagctgagatcgtgtcactgcactccagcctgggcgacagagcgagactccgactccgtctcaaaaaataacaaaaacaagaaagtgtgatggtggctgtggagcagaggagtcttctttagctcgtccacgtgacgtgcaggagtacatgggagtcgtgtgctggtggacacacagaagcgactttttcctttgtctcatggtggaatgctggaggagcaagtgttcccacctgctgagcagctgaaagcaggctttccgcggatgtgtgcgggcgggcggtgtggcacagaaaggcctcgatggactgtctgctcttcacggcaagagtgtgctgcccctgttctctgtcgtggcctctgctgtaaagatgagagaagtccaacgccagtctaattcatgattcttttttagttaagctttttaggattttgtggaaacttgtaggattttctgttgatatgtgggagtctgacattttgctatttttagggtttagattgtgacttttttatattggttagcatcttgaagctcttttaatatgcagacttgtctttttaactccatctcagtttttctgtcaagggccagttagaccttgtggccgcatgttctctgacagctcttcagctctgctgttgtatcatggaaggaaccggggccacacttaaaacgagcctggctgttttccagttacaactattactaacactgaaattagaatttcatataattttcacttaccacaaaatgttattttctttttcaaccaggtataaatataaacctcattcttagcctgtggactgtctaaaaaagacattgggccagattcggcccctgacccccaggtggtttccccatcatagtaatcatagtagatgaacttggaactgtgtagctttattggtttttcatccttgaagcttgcttttcctctgcagtggtcctgactttctgtagagtttgatctctgcggcttcttccttctgtagagtttggtctctgttttctctaaacttaagttatttgtctgatttcatttactttctgttaatttaggtgcttaattctggtcaactattgacatgtcattcttctgttttccagtgctgttacagatttattcttttctttttttctttttcttttcttttcttttttttttttttttaagatggagtctcactctgtcgcccaggctggagtgcagtgttgcgatcttggctcactacagcctccacctcccaggttcaagcgattctcgtgcctcagcctcctgagtagctgggattacaggtgtctgaccccacgcccagctaattttttgtatttttagtagagatgaggtttcaccatgttggcctggctggtcttgaactcctgacctcgtgatctgcctgcctcggcctcccaaagtgctgggattacaggcgtgagccaccacgctctgccagatttattcttttcaaaatgtttgttactttaagaaattttagataagagggttagattccaccattgtgatctttttttttcttttgagatggagtctcgttctgtcacccaggctggagtgcagtggtgcgatcttcgctcgctacagcctccacctcccaggttcaagtgattctcgtgtctcagcctcccgagtagctgggactacaggcgcccgccaccatgcctgattaatttttgtatttttagtagagacggggtttcaccatgttggccaggctggtctttaactcctgacctcaagtgatccgcccacctcaccctcccaaaatgttgggattacaggtgtgaaccactgtgcctggcgttagcgttgtggtctaatagtactacagtactattgttttttgtaaatagaattgcagtctgaacaggaagaacttccaccataggtgttttgaagaagttaattttttgcataagtagaaagccatgggagcattaaacttaacagtctattgcttgtgtggtaacgtagggaattaattttgaattaaatgtgaactagacaatttgctgtggaatactacgttgaaattattgaaaaacacttatccagtgtgaggctttttttttttttaatttatttattttttattgataattcttgggtgtttctcatagagggggatttggcagggtcataggacaatagtggagggaaggtcagcaaataaacaagtgaacaaaggtctctggttttcctaggcagaggaccctgcggccttccgcagtgtttgtgtccctgggtacttgagattagggagtggtgatgactcttaacgagtatgctgccttcaaccgtctgtttaacaaagcacatcttgcaccgcccttaatccatttaaccctgagtggacacagcacatgtttcagagagcacagggttgggggcaaggtcacagatcaacaggatcccaaggcagaagttttcttagtacagaacaaaatgaaaagtctcccatgtctacttctttctacacagacacggcaaccatccgatttctcaatcttttccccacctttccccgctttctattccacaaaaccgccactgtcatcatggcccgttctcaatgagctgttgggcacacctcccagacggggtggtggccgggcagaggggctcctcacttcccagtaggggcggccgggcagaggcgcccctcacctcccgggcggggcggctggccgggcgggggggctgacccccccacctccctcccggatggggcggctggcctggcggggggctgacccccccacctccctcccggacggggcggctggcctggcgggggggctgaccccccccacctccacctccctcccggacggggcagctgggcggggggctgaccccctcacctccctcccggatggggcggctgctgggcggagacgctcctcacttcccagacggggtggctgccgggcggaggggctcctcacttctcagacggggtggttgccgggcagagggtcttctcacttgtcagacggggtggccgggcagaggtgctcctcacatcccagacggggcggcggggcagaggcgctccccacatctcagacgatgggcggccgggcagagacgctcctcacttcctagatgtgatggcggccgggaagaggtgctcctcacttcctaagtgggatggcggctgggcggagacgctcctcactttccagactgggcagccaggcagaggggctcctcacatcccagatgatgggcggccaggcagagacgctcctcacttcccagacggggtggcggccgggcagaggttgcagtctcggcactttgggaggccaaggcaggcggctgggaggtggaggttgtagcgagccgagatcatgccactgcactccagcctgggcaccattgagcactgagtgaacgagactccgtctgcaatcccggcacctcgggaggccgaggctggcggatcacttgcggttaggggctggagaccggcctggccaacacagcgaaaccccgtctccaccaaaaccagtcaggcgtggcggcgtgagcctgcaatcgcaggcactcggcaggctgagtcaggagaatcaggcaggggggttgcagtgagccgagatggcagcagtacagtccagcttcgactcagcatgagagggagaccgtggaaagagagggagagggagaccatggggagagggagagggagagagggagagggagagggggagagggagagggggagggagagggagcgtgacgctttttttaaatgaagctcgtgacagacggaagtataccagtgattaagaagatgctgggatgggctttttcaatagatgctctgcaggtttccaaaatgagtgccagaggaggggagagggcagtgtcagagtcttctgaacattctgagagctgagctgctgtgagacaggcttaaatgagaaccctagttttcaaaacttaatgttttaatgggaatgaccatagttattagtgttaaaagatacatttcttcttatttatttagaagatgaagttcagagaatttaggtagcctaaataagatggcatagttagtaattctatgagcttttccttgtttagtaaatcggtattaaaatggaattattaagtggggtgtggtggctcacgcctgtagtctcagcactttcggaggccgagggaagcagataacgtgagcacaggcgtttgagaccagcctgggcaaatgaagataccctgtttctacaaaaaatacaaaagttagccaggcgtggtggcaggtgcctgtggtcccagctcctcaggaggctgaggggaaggattccctgagcccagcagtataaaattgaccatttgtaccatttttgagtgtgcagttctctggtattagattcacacggtgcaaagccatcaccaccatccctctccagaacttggtcttcccagacgacaccggctacccattaacactaactcttcatccctctccccataacctcgactctccccataacccctgaccaccaatctgctttctcttatgaatgtcaccactcaaggcacctctcctataaggggggggggggggtcatacgatatttgtcctttcttctcttatgaatgtcaccactcaaggtgcgtctcctatgggagggggtcatacgatatttgtcctttcttctcttatgaatgtcaccactcaaggtgcgtctcctgcgggggggtggtcatacgatatttgtcctttcttctgttatgaatgtcaccactcaaggtgcgtctcctgttgggggggggggtcatacgatatttgtcctttcttctcttatgaatgtcactgtccaaggcacctctcctgtaaggggtggggggtcatacaatatttgtcctttcgtgactggcatatttcccttttggatcagtttgttcctgtgggtcagaatcttcatttgaacagtttgccccacagctcagattcctcaattgtgactaccccctgcaggtcaaattcaatttttgtttacttatttttgagacagagtcttgctgtcttgctcgggctggagtgcagtggtgtgatcgtggatcactatagcctcgacctcctgggctcaaacaacccgcctgcttcaaccttccatagtgctgagattacatgcgtgagctgctgtgcccagtgtcaaatttaatttttgttttgttttgtttttgagacagaatctcgctctgtcaccaaggctggagtgcaatggcactatcttggctcactgcatcccccacctcccaagttcaagcaattttcctgcttcagcctcctgagtagctgggattacaggcacctggcaccacgcccggctaattttttgtatttttagtggagacggggttttgccatattggccaggctggtcttgaactcctgacctcaggacatttataggatacacttattatttttatgaccaaagcatgtgatttttattttttaattttaattttattttttaatgttttttgttcttgttgtttttgagacggtgtcttagtctgttgcccaggctggagtgcagtggcgcaatctcagctcactgcaatctcggcctcccaggttcaaatgattctcctgcctcagcctcccgagtagctgggattacaggcgcacaccaccacgcccagctaattcttttgtatttttagtagagacggagtttcaccatgttgggtaggctggtctcaaactccggacctcaaatgatccacctacctcagcctctcaaagtgctgggattacaggtgtgagccaccgcacctggccttttttttttctttttttttggatacagggtcttgcttggtcacccaggatggagtgcagtgacacgaaattggctcactgcaatctcgacttcctgggctcaagcgatcctccaggctcagcctcctgaatagctgggactgcaggcacgaccaccatgcccagctacttttttattgtttgtagacatggggctggtctcagacgcctcaagaaatcctcttgcctaggcttcccagtgtgctgggattacaagcatgggccactgttcctgaattttatttttttaaacctttttatagaacacgatcagttgtttgataaatactgaaacagtactaggaatcagttttttagttgtttaccaaacatattatgcaggaactgaattcacaaaaagttgttttgaaatttggtccacaaattcacttaaggttggaaataaaaaacttgtaagaggccgggtgtcgtggctcaaaaagaaaggaaaggatactttcaggcttagagttagtcttttttgttggaaattttcacaacttcagaaaaacttcatcaacaggtttagaagcatccgttttattgactttcccgatttcttcgtatgagtcagtaattgtttttgttaacttgaagatgggtctgaattctcttttccaagctctctctgctggcttcactcttaccactgttccctcctctcaagacatcctttcatgtagatctcattattatggtcagaaaacagaaccagatgcacatctgcctttcccatccatgctcttggcccaaccttgaaggttgtcttgatttcttagaaactcatcaagaatttattcttagcattggcagccattgtctgatccatttcccatgtgaataaatgcatgtatggttatttcatctagcacagtcttcccttgtttagtatagtttttgaagagttcaccactgtgaatctgggttctttcatcacagagaagttttcctgaagacaggtgtatgtgaccagggtcacgacggtgtgggtttgctgcgtccccttgccagtgccaggacccctgaggaaccacaggaccagtgggcagcttcatgaggccggggccgcagaggagatgcaggcagccagtcagcagcaggaagctgaggcctagggcacgcagtggccagcagcaggctggctccctctttgggaggttaaatcaagttttggttaccaaaggcaaagcaggcttggattttgtttaaggagatgctgttgaatgaaacagcttctttgtagcaagcagccttggagatgattaatggaacatgtctgaacttgccagagtgatgttcagccttccagtggaactgcaaaatcatgagtgaaagcgtggcaaagtattttctttaatgtaaaatgttattcctaagaaatgaaattacgtggccgggcacggtggctcacgcctgtaatcccagcactttgggaggccgagatgggcggatcacgaggtcaggagatcgagaccatc

SEQ ID NO: 2 - DNA, 272 bp, Homo sapiens
  source 1..272: mol_type = genomic DNA
atttttccaaaagaatcgtgatctcagtgacatatacgtggaagatggaaatggagcccacaactctgcagtgcatcctgatgccgcgctgacctgacggcttgtgcgtgtccctttggctgcaccagtgagcacagtggcaggcgtgtcagagaaaggccccttctgcagacggtctctcaccattgccgaccacggaatcccagaaccgctgagctgcctcgggaagaaccagcaggtgtctgcatcgttgagtgtgttctgatccaaag

SEQ ID NO: 3 - DNA, 97 bp, Homo sapiens
  source 1..97: mol_type = genomic DNA
atgcctccagcccagtccctgttgtggtgctgcaaggctggtacgctcctcgaagcaccatggcatgagatggaggttcctagaagcaagaagaaag

SEQ ID NO: 4 - DNA, 119 bp, Homo sapiens
  source 1..119: mol_type = genomic DNA
tgctctgtgttgatgccttcagatgcctccagcccagtccctgttgtggtgctgcaaggctggtacgctcctcgaagcaccatggcatgagatggaggttcctagaagcaagaagaaag

SEQ ID NO: 5 - DNA, 32767 bp, Homo sapiens
  source 1..32767: mol_type = genomic DNA
gacgcggggggaggggggtgcggcgagcggccccgctctctccccaccgctccgctcgcaccccagtgtaatgagggtcaccccctccccccagctggcccgggagggggcgcggggcacggtaactagtgcgctggggtgggcggcgggcaggcgcgaggagaagggagggaggagggtggccgggcggggaagatggtggtggccgtaaggtgaggggctcgggggagggccaggcgcgatgcggggttggtggccggcggcgctgcagccgccggcctcctccccctccccctcctccatcactaccagccgggctcaggcctagctggccgggctgccgcgaacttcctcccggcgcggcccgtgccccgccggccgcctgcgaacacctcggcctccgcctcccctcaggtagcaggctgcggggcgcggggccggctgccctcccgcagcaaactttgcttgctgctgaatattgatgagagcgatcggctcggctgggaggtgctgccgcggctgcgggaaggagcgcggcccgggcaggcggcggcggcgtcggcagcagccatgtttttcgagctgtagcagctgctgctaccctgactgggcttcgctggccgcctcggtttctccctctgccgggtccaggcctcttcgccctgcagctgcggatccagcaggcctgcattcaggaaggcgagctctggggtgcagccgcctcggccggctcgcctgcggcctgcgcaccgccgctgcaaaggctccggcgctggctgggcgcagggtgcagcgctattgtgaccgctgcgccctagcgagccaggaaggggggggtacctttttgtgcagggtccaggagcccccctcggaccccgcagccttttgcttttgagagatccagctgctcgacccctggcgagggagggggaggactagtcctgtttgagaattgggaattttgacgggcagaggggttttaattttagttcatcccaagtgtccaccagtctacagaggaggaaaaagagacgggctgtttctatgtagcaggatcggcccagcttcgggaaaatggagttttcagaggctcatcgaggccattttttcatctccagtcgggggaactttttctgcccatggaagtgcagcagaaaggcatagaggccactaggccttgaagtggctgccattttaaagagtcgagtcagatggcctattaactcagattaattgctgtgcttttggattccaggttgatgccggcccaggatggatcagacctgtgaactacccagaagaaattgtctgctgcccttttccaatccagtgaatttagatgcccctgaagacaaggacagccctttcggtaatggtcaatccaatttttctgagccacttaatgggtgtactatgcagttatcgactgtcagtggaacatcccaaaatgcttatggacaagattctccatcttgttacattccactgcggagactacaggatttggcctccatgatcaatgtagagtatttaaatgggtctgctgatggatcagaatcctttcaagaccctgaaaaaagtgattcaagagctcagacgccaattgtttgcacttccttgagtcctggtggtcctacagcacttgctatgaaacaggaaccctcttgtaataactcccctgaactccaggtaaaagtaacaaagactatcaagaatggctttctgcactttgagaattttacttgtgtggacgatgcagatgtagattctgaaatggacccagaacagccagtcacagaggatgagagtatagaggagatctttgaggaaactcagaccaatgccacctgcaattatgagactaaatcagagaatggtgtaaaagtggccatgggaagtgaacaagacagcacaccagagagtagacacggtgcagtcaaatcgccattcttgccattagctcctcagactgaaacacagaaaaataagcaaagaaatgaagtggacggcagcaatgaaaaagcagcccttctcccagcccccttttcactaggagacacaaacattacaatagaagagcaattaaactcaataaatttatcttttcaggatgatccagattccagtaccagtacattaggaaacatgctagaattacctggaacttcatcatcatctacttcacaggaattgccatttgtaagcagtttttggtacaacttaaatatatacatatatgtatatatacaggccacttaaagggaaacttgtaacaaatttgtttttggttgcttatcagttcacagctgaaatcctattgctaatcataagctttgggcaaaattttactttgatttttaaatttatctctgttgtatgaatttggttgttttaagctttttccaaataactcttcattgagagtaggctaatgcttttaaaggcatttgattgagttcaggtttaatttctcaagttggaggtatacatatatgattaaaaaaaaaaaaaaaagatgggttttggcctgccagcaccatgagtgcaggtgaaccaatttagtacttggagtcctgttgctatatgtggcagattatttttttacttgatgacttgactcttacttcaggttgaagggcattttgaacacagattaaagtggctaagatgaagttttcttggacattgtcaaaatctaaattaggctagtttttctgaactacctgttttgaaggtatagcatcctgtgcttttgataactgccaccattagctcttttttttttttttgaggtggagtctcactctgttgccaggctggagtgcagtggttgatcactgcaacctctgcctcttgggttcaagcaattctcctgcctcaccctcccgagtagctgggattactggtacccatcaccacgcccggctaatttttgtatcaccattagctcttgaagtttttctagttttgttttgttttattttattttattttaacagaaccctaactaagacaaagttttatatttatttattgtttagagactggccttgtcatgttgcccaggctggcgtcgggactcctgggctcattcgatcctcctgcatcagctagaactacagtagtttcagattttgaagtgtgtatgtgtatgtgtgatatgtatatattccgtgtgtatagaaatggagagtatcttatttgagttgttgttttcagtaatgctgtcaagtattgttagagggtgataaatgataacatttgtttttatttgagcttatgaagaatttcttgactttctagctaaatgatcagttcacttctcttagcctcaattttattgcgtctaaattccagaagttcttgattgctataagattccttcagctttaaatattaatatttgatattgattttgtttctgcccaaacacattgtttggtcaccgccggtaatgttagcaaagagaattttttttggccaacaaatgtctcataccacattcagtttttataagaaaaacttttatggtatgttgttattctgagttcattaaacattcgctttaccttatatccctgctgttctttaaagttacagagggagaatgtgggtgtgtcacttttgtttctgttgatttgtatcttaattatgccttggtactccttggtttcttggcaattgcagatttaaaaaaatttgctttagtggttatcttgagtctgaattgtcctacacattagggtgggtaggctgttttgaaaacctattggcagctcagacaaatcctttttcttgggttcacgttgaaatttattttatatatatatcgtgtctttgtttttgcacataaatttaaatctgagaatggagatagatgtttctctagaagcatacaaatagaattgtaaacctgtttctcgtcaaagagatgttagtggagtattggttctattaaaaaaaaaatgaaggctgagtgtggtggctcacacctgtagtcccagcactttgggaggctgaggtggacagatcacctgaggtcaggagtttgagaccagtctggccaacatggtgaaactccgtctctacaaaaattagccgggcgtgatggtgggcaactgtaatcccagctactcgagaggctgaggcaggagaatcgcttgaacccaggaggcagaggttgcagtgagccaagattgcgccattgcactccatactgggaaataagagtgaaactctgtctcaaaaaaaaaaacaacaaaaaaacaaacaaacaaacaaacaaaaaactgaaaatattggagcctttagatagtaggttacatgtctaaaatgggagttagcaaatgtataaatgtagaagtttttttttcagggagaaattgaaattgctcaaagactttatcaccttgaagaagcaagtatgtagtttatttatttttttgagacacagtcatgctgtcacccaggctggagtgtagtggcgcgatctcagctcacttcaaccacctcctcctgggttcaagcgattctcccacctcagcctcccgagtagctgggactacaggtgtgcaccaccatgcctgactactttttgtatttttattagagacgaggtttcaccatgtgggccaggctggtcttgaactcctgacctcaggtgatccgcccaccttggcctcccaaagtgctgggattacaggcgtgagccaccgtacccatcccctaatttattattttaggaatttggttcaaagttgtgattgaaatctattgcctttatttttgcctttgatatttttaaactgaagacatttttttttttgagacgaagtttcactcttgttgcccaggctggagtgcaatggcatgatctcggctcactgcaatctccgccttctgggttcaagcagttctcctgcctcagccttctgagtagctgggattacaggtgcgcaccaccaccccagctaatttttgtatttttagtagagatggggttttaccatgttggcccagctggtctcgaactcctgacctcaggtgatccacccgcctcagcctcccaaagtgctgggattacaggtgtgagccacggagcccggcctcagactgaggacttaaaaagtgaggtcagggtgggcatggtggctcacgcctgtaatcccagcactttgggaggctgaggcgggtggatcacctgagatgaggatttcaagaccagcctggccaacatggcaaaaccccgtctctactaaaaatacaaaaaattagctaggcatggtggcaggagcctgtaatctcagctatttgggaggctgaggcaggagaatcacttgaacccgggaggctgaggttgcagtgagctgagatcgccccattgcactctagcctgggcaacaagagcgaaactccctctcaagaaaaaaaaaaaccatcctggccgacatggtgaaaccccgtctctactaaaaatacaaaaattagctgggcgtggtggcaggctcgggaggttgaggcaggagaatcacttgaacccgggaggcggaggttgcagtgagccgagattgtgccactgcactccagccttgagacagagggagactccatctcaaaaaaaaaaaaaaaaagcggtcaatcttagaatgcaaagttaggtaagcaatacagcttgagaaaagtgtaattaaaaataacttttctatgtagtcatgtgatattaatgtattcaacttgttcacagttgatttaagttattgatatagtaggtattgttactatgctgggaattttagaaaatccttagcaaattgctatttgtctctttttgtctgtaattttggctgggcttggtggctaacacctgtaattctagcaagttgggaagccgagacaaaaggattgcttggggcccagagtttgaaactagactgggcaacatagtgagatcctgtctctacactcagttggttgtggtggtatgcctgtagtcccagctactcaggaggctgaggcagtagtaggatcacttgaggccagaagtttgagactgcagtgagccatgatcatgccactgcattccagcctaggcaacagagcaagatcctgtcaaaaaaaaaaaaaaaggagaaaattctcttggcagtgggtaagagtagttattagggttgtagatttcctgtctggaattagagaaagaagggtcatattttctgttattttgtgtatctacctctaagtggactgtttgcctcttgtcacgaattagtagcctcttcagtttaccatcatgtgctcttattttctctgcatacagtgaagtgattgtcattacaatttataatcctgacctggtacttttatatttaattgggctgatattttctaattcttcccagtgtacaaaggttttatgctttgttgttgttgttgagacaggctaggtgctttggatgtggagaattaaatgagcatggcattttcagaggatacttgttggagattgcttgggtaggatggatgtagtcaggtaatggggcctagaaattcagactgaagcatttggtattgatgtgatgggaactggcagcccttgagagattttagctgagaagtgatgtaaaatctgtttggaagactttgagtagaggagattagaggcaaggttaggatgtagggtatgttgcaatagtaattaagacttaagaatcggcccagtggcatgtacctgtagtcctagctactctggaggccgaggcaggaggatcacttgaggctgcaattagctgtgattgtgcctgtgaatagccactgcactccaacctaggcaatataatgagattctgtctcttaaaaaaaaaatgagcacagtgagtactctaaagaaagggggtaaatctaaaagattatttcaaagggagaaaattggcagctttttgggggctacctgatctggaggcagattggagtctggatttgaggaatggagagagatgaggcagatgatgtctaaggcttatagttttgctgcctgagacaaaaatgattcctcagaggttccttcctcttctctacccatcatcccacaattttctactccctccttagctatcttggaagaaaattgatctcttcacacctgaggttctgctctctctccgattccctcctggctgggtgaccttttttgtttgtttttgtttttgttttgagacagagtctcactctgtcacccaggctggagtgcagtggggcgatctcggctcactgcaacctctgcctcccaggttcaagcaattctctgcctcagcctctggagtagctgggattacaggcgcccgccaccgcaaccagctaatttttatatttttagtagagacggggtttcaccatcttggccaggctggtcttgaactcctgacctcgtgatccacccgccttggcctcccaaagtgctgggattacaggcgtgagccaccgcgcgcagcctttttttttttttttttttttttttttttaagatgaattcttgctctgttgcccaggctggagtgcagtggtgtgaccttagcccacggcaacctccatctcctgggttcaagagattcttgtgcctcagcctcccaagtagctgggattgcaggcgccctccaccatgcttggctaatttttgtatttttagcagagagaggtttcaccatgttggccaggctggtctcgaacccctaacctcaagtgatccacctgcttcagtctttcaaagtgctggaattacaggtgtgagccaccacgacctgcataccacttctcaaacagtccttttttgcgtccttgttctctttttcttcctctttctctgcagtctcattcactttcattgattctgctgctactccactctatgaaactctcttctgaactgacttcaaaccaacaaattctacttgtcaactaagctgctcctctaccttgtgttatattcacctaaaatgtaatattatttccttttttatttttcctttggacagggtctttctctgtcacccaggctgtagtgcagtggtgccatctcggctcactgcaacctctgccttctgggttcaagtgattctcctgcctcagcctcctaagtagctgggactacaggcgcccaccaccatgcctggctaatttttgtatttttagtagagacagggttttgccatgttggccatgctggtctcaaactcctgacctcaagtgatacgcctgcctctgcttcccaaagtgctgggattacaggcatgagccactgcgcccagcctattattttcattttgaacccatctcttttattgccaaacacgcatttacttctgtgttcatgatgacatcattatcctattcatctcaaagctggaaaccttgcagtcaatcatttaaatgattaaaatacatttgagtacctcttgagccaggcactgccagtataataaaaaataaaaaaattaaaaaaaggaaagagatagtttgcttttaaggaacttcactgtgtggcaaaaactagtgtaaacaatgacaatacagaatactaagtggtctggtaggtgttatgtatgcagtactttgggagtgtggaggaaggcatgcctagaataatcagggaggacttcacagagtggttatttatagtttaagcagagacataccagtaagagggaatagcatatgcaagtggccagaaatccttggctagctatctgggaggagtggggttgtcaggagataaaggtataaagataggcttatatgccgtgctgtatagttgaatgtttttactattacaaaattttacagatgccctcagtttctccctttattcatttttctatgacatctttattgttggtcttcatttagtctttccttccagtctatcctgtgtaaaattacttcctacttccaaaatgagaaatactgggtctctacttaaatttgtaacctaaatgcctcacacctcattttctgaacaaataaagcccaaattcagtgtcctttttgataggatcctgtcctgacctttccaaatctgatgctagagccttgtgtaccctgagttcagccaaactgaactcttaatggtcccttgctccatactctccccttgctcatgcctttattctcctggtctgattcatctttgcatcttaacagtgtatagcatggtgccttctttttactggggacatatcgagttaatgaatgaatgatgctattacagaggtacagtttgggaaggggagtgagtacattttagaaaggtgataagtggattgtcagccttcatcattttcaatggaccaaattactaaaactttacaggttggttggttttttttcttttttcatttcctcatgtactcaatttctaaggctttttgaatttgagcttcctaatatctcatgcattaatttttttctccattctcaactttcactcttttaattaaggataataatttttttttttgagatggagtcttgctctgttgcacaggttcgagggcagtggtgcgatcttggctcactgcaatctccgtctgccgtgttcaagcaattctcctgcctcagcctcctgagtagctgggattacaggtgcatgccaccacgcctggctaatttttgtatttttagaagagatggggtttcaccacgttggttaagctggtcttgaactcctgaccttatggtccgcctgcctcagcctcccaaagtgctgggattacaggcatgagccactgagcctggccaaggataataaattataatggttttaggttggacatctctgactgcatactgcactgtgtttactggaagaagtcccttaatgtctctaaggcccatttcctcagttctaaattacggctagtaccttcattggagggttgttaagtctatgatacaagataactttttttttttttttttttttgagacagagtctctatcgcccaggctggagtgcaaaatggcacgatcttggctcactgcaacctccacctcatgggttcaagttgattctcctgcctcagcctcccaagtagcttggattataggcatgcgccaccatgcccgactaattttgtgtttttagtagagatggggttcaccacgttggccaggctggtcgaactcctgacctcaggtgatcgacccacctcggcctcccaaagttgctaggattacaggtgtgagccatctctcctggccatgatacaagataatttatatgaagtaatacactgctggttctgaagtaggtgtgcagtaagtgatgcctactgctgcatgccaagagtcaaatgtatatttgaaagagttgtgaatttcaagaaagatatttttgagtttttttttttttctttctgagacagggtcttgtactgtttcccaggctagagtgcagtggcctgatcttggctcctggctgggcccaagtgatccaccgccctcagccttccaacgtattgggattacgggaatgagccactgcatttggctaagtttttgtttttttttttctctatttttccaaacttatttgattagtaagataaagacattaactgctgttgacagtttccatttttaattagtaatcaggagcatttgttgtatttttgtttgataatcagaataatttaatttgtgcaataggatcaatagctttctgtattccaactgttaagtggtgtaagtttattacattgttgctttttgcaggttgtcctttgttctagatagaaatgtttaatttattcttcctggttttcaggggagcccattgaaaggagatccagtctctgaaatttagtggtaggataataacaattgaacagttacttttgaatctaatttaaataatctcaattgtagccttttaaagcaattcctatgaacctttttgaatttagaaaagtaatacttggccgggcgcggtggttcacatctataatcccagcactttgggaggctgagggggtggattatctgaggtcaggagttcaagaccagcctggccaacgtagtgaaaccctgtctctactgaaaatacaaaaaaaaattagctgggtgtggtggcacgtgcctgtagtcccagctactcaggaggctgatgcaggaggatcgcttgaacccaggaggcagaggttgcagtaagctgggattgtgccactgcactccagcctgggtgacagagtgagactttgtctcaaaaaaaaaaaaaaaaaagtcaaacttaaaaatggaatataaaaatctcttgatttttgtcagttttcatatactccctcatttacactcttaatattctattagaaattgtctcttctctctacacacccctttttttcccttttggttaatatgttaagacatcttttcatatgagcatgtaacatgtaacaagattttttttttttttttggacagtgtctcgctctgttgctcaggctggagtctagtagtatgatcacaactcactgcagtttagacctcctgtgttaaagtgattctcctactttagcctcatgagtagttgggactacaggcccatgccaccacgcctggctaattaaagaaaaaattatttggtagagacagggtcttgctatgttgcccaggctggtcttgaatttctggcttcaggcaattctcctactctgcatgagccacctcagccgcgaatattttcttattatgaaatttttgtttagataaatgttgattcacatgcagttgtaacaaattccatggccaggctgggcgtggtggctcacgcctgtaatcccagcactttgggaggctgaggtggatcacctgaggttgggagtccaagaccagcctgaccaacatggagaaaccccgtctctactaaaaatacaaaattagccaggcgtgatggtgcgtgcttgtaatcccagctacttgggaggctgaggcagaagaatcacttgaacccgggaggcggaggttgtagtgagccaagatcgtgccattgcactccagcctgggctagaagagcgaaactccatctcaaaaaaaaaaaaaaaaaatcaggaaattccatgggctaggcacagtgacttatgcctgtaatcccagcgttttggaaggctgaggttggaggattgcttgagcccaggagtttgaggctacagtgaacactgactgtgccactgcactccagcctgggtgaccctgtctcttaaaaaaaaaaaagaatacagagaggtcccttgtatattttgcctggttttgcaatggtaatattttgcaaaaaatatctaataccacacaaccagaatattgatgttgatgtacttcaccaatcgttttttttttttttttttgagtcggagtctccatctgatgcccaggctagagtgcagtggctcaatctcggctcactgcaacctccacctcctgggttcaagcaattctcctgcctcagcctcctgagtagctgggactacaggcgtgtgctatgacgcccagctagtttttgtatttttagtagagacggtgtttcaccgtgttatccagggtggtctcaatctcccgaccttgtgatccgcccgcctcagcctcccaaagtgttgggattacaggcttgagccaccgcgtccagccagtcttacttaggcattgacgttcatgtaatttatccatcttattcagatgtccttaaattttatctttttccttaaaagaaatctgtatttctatcaggacattctggatgtccccagttttactggtagtctttcattgtgtgtatattaagttctttgtttttatcacctgtataggttagtatatccatgactcccgtcaactttctaaatgttcgctgggtgcagtggctcatgcctgtaatcccagcactttgggaggctgaggcggctggatcacctgaggtcagtagttcgagaccagtctggccaacatggtgaaaccccgtgtctactaaaaataaaaaaaaaattagctggatatggtgggtcatgcctgtaatcctagctactcgggaggctgaggttggagaatcgcttgaacccaggaggcggaagttgcagtgagctgagatcgcgccgctgcactctagcctgggtgacagagtatgtctctgtctcaaaaaaaaaaaaaaaagttgctaaacatttctaataccataaggatccctgctgttgccagccgttttaaaactacatccatcgtcttcttggcaaccttccatctctttttcgtatgtgacagcgtcttgctctgccgcccaggctggagtgcagtagttgcatctcagctcactgcaccctctgtgtcccaggcttaagcgatcctcccacctcagcctcctgattagctgcgactacaggcacttgccaccatgccccactaatttttgtatgtttttgtagagatggggttttaccatgttgctcaagctcgtcttgaactcgtgagctcaagcaatccgcctgccttggcctcccaaatggctgggattacaggcaggagccaccatgcctggcctagcccctccatctctagcctttgtcagttactaaactttttttcctgaagttttgtcatttcacaaatgttagataaacatgagtcatacagtatgcagccttttgggattgtctttttttcccttagcataatttccaggggattcatctaagttgttgactaaatcaatagttgttttttttgtttgttttttttttgagacggagtttcactcttgtggaccaggctggagtgcaatggcatgatcttggctcactgcaacctccgcctcccaggttcaagcgattctcctgcctcagcctcctgagcagttgggattataggcccctgccaccacacccagctaatttttgtatttttagtagagatggggtttcaccatgttggtcagggtagtcttgaactcctggcctcaagtgatctacctgcattggcctcccaaagtgctgggattacaggtgtgagccactgcgcacggccctagttttttcctttttatcactaagtaatattccatgatacaaatataccatggtttgcttgaccgttcacctgttgaaggacatctggggcaatgctagcttttggtaattaaggtaaaagtactatttatgttcatttatggggttttgtgtgactgtaagttttcacttctctgggataaataccagtagaacaattgcagtattatatggtaatggcatgttaagttttttttttttcctgagagggagtttcgatcttgttgcccaggctggagtgcaattgcgcgatcttggctcgctgcaacctctgcctcctgggttcaagcgattgtcctttctcagcctcgcatgtagctgggattataggtgtcaaccaccacacccagctcatttttgtatttttagtagagatggggtttcactgtgtttgccaggctggtcccaaactcttgaccccaggtgatccaccctcctcagcctcccaaagtgctgggattacaggcgtgagccacggcgccccgccaatgttcagttgtttttttgtttttttgagacaatctctctctgtcacccaggctggagggcagtggcgcgatcctggctcactgcaacctctgcctcccggattcaagcgattatcccgcctcaggctcctgagtagctgggaccacaggtgcacaccaccacaccaggctaatttttttatttttagtagagacggggtttcaccatgttgggtcaggctggtctcgaactcctgacctcaggtgatccacccacctcggcctcccgaagtgctgggattacaggtgtgagccaccacgcctggcccaatgttcagttttataagaaactaccaagctgttttccctagtgtctgtaccatttacattctcactagcagtatatgagtgatccagtttcttttattttttgttttttgagacggagtctcgccctgttgcccaggctgaagtgcagtggcacgatctcggctcactgcaacctctgcttcccggcttcaagtgattctcctgcatcagcctcccaagtagctgggattacaggcatgtgcaccatgcctggctaattttttgtatttttagtagagatagggtttcaccatgttggccaggctggtctcgaactcctgacctcaggtaatccacccatcttggcttcccaaagtcctgggatttcaggcatgagccattgcacctggccgagtgcttcagtttctatgcatcctcaccagcatttggtgtggtcactattttaattttagccattcgtgtagatatgtagtaatgtctcatctcattatgttttgtttttttttttgagacggaatgttgctcttgttgcccagactggagtgcagtgatgccatctcggttcactgcaacctccacctgctgagttcaagcaattctcgtgcgtcagcctctggagtagctgggattataggtgtgcatcaccacgcctggctaatttttgtattttttagtagacatggggtttcaccacgttggccaggctgttcttgaactcctgacctcaggtgagctgcccacctcggcctcccaaagtgctgggattacagttttgtatggtggattccatgcagagagagttttttctgtagtctagattagcagtccccagcctttttggcaccagggaccaaattcctgggaaacagtttttccacaggtgggagtgggatggtttggggatgaaacttttccaccttagattatcacgcattagttagaatctcataagaagcgcgcaacctagatcccttgcatttgcagttcacaatagggttcatgatcctctgagaatctaatgccacccctgatgtgacaggagtgggagctcaggcgataatgctcccttgtctgctgttcacctcctgctatgcagcccggttcctaacaggctgagaggaccagtaccattctgtggcctgggcgttggggacccctgttctagatgatccacattcttttaaatgcctatatacaaaccatactttctttatttcttttctttttttgagacagtcttactctgtcacccaggctagagtgcaattgcgtgatcttggcacactgcaacctctgcctcccaagttcaagtgattctcctgcctcagcctcccgagtagttaggactacaggtgtgtcccaccatgcctggctaattttttatatttgtattttttaatttttatttatttatttatttttttgagatggagtctcgctctgtcacgcaagctggaatgcaatggcacgatctcggctcactgcaacctccgcctcccgagctcaagcgattctcctgcctcagcctcctgtgtagctgggattacaggcacccgccacgacgcctggcttttttgtatttttgtagagacaggttttcactgtgttgtccgttctggtctcaaactcctgagttcagggaatccaccgccttggcctcccaaagtgctgggattacagtcgtgagccaccgcgccctgccacaaaccatactttgaaaacgttgcttccatttttagataatttgttaggaaaccaataaaatcatacatacttgtgattttcccttagtaaaacacaaattttagtgttttttgctgttattattaatacttctaaagttcctttcacattgctagtgaccttatataaaataccataatgctcttctagcaattgctggaaagataaaatctattttagagaatgaacaattatattttcacattagattaaattaaaagtaattactggttatgtgatattccctcacataccagagtgagtctgaaggtagtctttctttgtaaattatgaggctatatttcctgtgttatctctgatttctcttgatgctgtaattggagttgttgggtctccctggtgaaagtaggtgatgtgcaagttgtgtctatacccagtgaaaataacagacattaatgctacactaatttgtcattggaattttacattcaaaagcatttctttttaaaaatatgattgtaaattggtaatttatagttgtatataccaaaggcatttctttaacgttatagttggttcaactgaaaatacgttaagtctgtttttataattagtatattgaggaacagcacttccatcgtgtcacaatatattaagaattgccagcagggcacggtggctcacgcctataatcccagcactttgggaggcctaggcgggaggatcacctgaagccaggagtcgagaccagcctggctaacgtggccaaacccctatctactaaaaatacaaaaattagccaggtgtgatggcgggtgcctgtagtcccagctactcgggaggctgaggcaggagaatccagaattgaattgaacccaggagacggaggttgcagtgagccaagattgtgccattgcactccagcctggacaacacagcgagactcagtcttttttatttttatttttatttttgagacggagtttcgctcttgttgcccaggctggagtgcaatggcacagtctcggctccctgcaacttctgcctcccgggttcaagcgattcacctacctcagcctcccgactagctgggattacaggcatgtgccaccacgcccggctaatttttgtatttttagtagagatgggatttctccatgttggtcagacttgtctcggactcccaacctctggtgatctgcccgcctcggcttcccaaagtgctgggattacaggcatgagccaccgtgcgtgtccttttttttttttttatcttttgagacagggtctcactctgttggctaggctggagtgcagtgatgcagtcacaactcactgcagcctcaacctcccagtctcaagcaatacccccacctctgcccctttgagtaggctgggactacaggtgtgtgccttcatacctagctaattttttttgttttgttttttgagacagtcttgccccatcgcccaggctggagtgcagtggtgccatctcggctcactgaaagctccgcctcccgggttcacgccattctcctgcctcagcctcccgagtaactgggaccacaggtgcccgccaccacacccggctaattttttgtatttttagtagagacggggtttcaccatgttagccaggatagtctcgttctcctgacctcatgatccgcctgccttggcctcccaaagtgctgggattacaggtgtgagccactgcacctggccatgcccagctaatttttgtatttttttgtagggatgggatggcactatgttccctaggctagtctttaattcttgggttcaagtggtcctcctgcctcggcctcccaaagtgttgggattacaggtgtgagccactgtgccgagccaggttgtgtgtgtgtgtatgtatgtatgtatgtatgtatgtatgtatgtatgtatgtttgtatatatttatatttatttttttggaactgcatctcactttcatccaggcccgaatgcagtgacatgatctcagctcactgcaacttctgcctcctgggttcaagcgattctttttttttttttttttttttgagacggagtctccctctgtcgccaggttcactgcaagctctggctcccgggttcacgccattctcctgcctcagcctcccaagtagctgggactacagatgcccaccagcatgcctggctaattttttgtatttttagtagagatggggtttcactggggtttcaccatgttagccaggatggtcttgatctcctgaccttgtgatccgcccgcctctgcttcccaaagtgctgggattacaggcgtgagccactgcgcctggccatttcttttttttttttggcaagtgattcttgtgcctcagcctcccgagtagctgaaattataggcgtgtgccctcaacgcctgggtaatttttgtatttttagtagagacagggtttcaccatgttggacaggctggtctcaaactcctggcctcaagtgatccaccctcctcagcctcccaaagtgctgggataacagctgtgagccaccgtgcccttcccaggttttatatttattcttttttccttttaaattatgtttttatttaggtattgtacgtaaagtgcttttctaacagagctttggggcagaagtgttagggcaggtcattaaaccactgaaattagttctttggaggagaagataattgttagagttgtaagtgaagtcttgatagataccttatcaatttcatagtaatgtctgtggaatttctttttctgttttttttttttttaattatttcttgaggattaactgctgatagtggaatatcatatatatagttggctcttgatgtacttatttctggatggctttccaaaaggattttaccattttacacacagttctaaatagtatatgaatttagcatttgtcccacacttagatagcactgatttttttttttattaagtgggtgcaaaatgctactacaagattgctttaattactacagttttattgatgaaaatgatttctacttgtttactgtttgtatttttttctaggagttttgtgtctatattctttgctgatgtatctttttggatttaatgttttatacatattaaatttctgtctcattggatataaatattttcccaatctggttttcattttagttaatgattttctgtagttgtatagtcaaagtttcatttattatatagctagatctgtgttttcgagtgatttattgattcaaagcttattgtgcttctagatatttgataaactgactttagactcttgtaaaaatttgaagaactcatatctactacagtcttactgatttaataggggttttaatatccagtactatgctaataatttttatagtgtttttacgacaattttttgagaacataagtttttagagctgtggatggaatgttttctgctctatcagttatcccttctgcgtaacagacccctaagtgtagcagcttagaggagtaaatatttattatctcacattttgtaaggaatcatggagtggcttagctggatggtgctggctcagtctctctaatgaatttacagtcaagatgtctgccagggctgcggtctctgaaggctgtaggatccctgtccaagacggctcactcatatggatgctagctctttgtatgaggcctgttctttcccacttgcacttctccataggcctgcttactgtatggtagctggcttttcccggagtgagtgatccaagagacagggacagaccaagcaggaagatgcagtaactttttatgatgtgtattctattggctggccacacataccaagcagatagggaagggattacacaaaggcatgaataccatcaggctgggataattgggggccagcttggaatctggctaccatatccaaccaaataagaaattaatagttttaattaaaggaaaaggattatattaaatagacattcgttagtttttacttttaagctgacccaatcatttttcagattgaagttttgaatagatatatgattaaaaaatacatgaaaagttaaccagtgaagtgacctctgtgccatgtttgctcaggtaacgcacctccaattcttgtgctttcccggagaccaccttttttaagagaaaggtagtggactgtgcacacttggtcttcctttttcacataatggtgtatgttgaaatctttccattttagagcatagctttccctttttaattttattattattattatttttgagacagagtctccctctgtcgccccagctggaatgcaatggtgcgatctcggctcactgcaacctccagctcctgggttcaagtgattctcctgcctcagccacctgagtagctgggattacagtcgcctgccaccatgctcggctaatttttgtatttttagtagcgacggggtttcaccatgttggccaggctggtctcgaactcctgacctcaggttatccacctacctcagcctcccaaagtgctgggattacaggcgtgaggcaccgtgcccggcaattttttttttttgagtcagagtcttgttctgttgcccaagttggagtgcagtggtttgatctcggctcactgcaacctgtacctcctgggttcaagtgattctcctgcctcagcctcccgagtagctgggactacaggcatgccccaccatgcttggctaattttgtattttagtagagactaggtttctccatgttggtcaggctcgtgtcaaactccctacctcaggggatccgcccaccttggcctcccaaagtgctgggattatagacgttagccaccgcgcctggcctaatttttgtattttcagtagaaatttttgtatttcactgtattggtcaggctggtctggaactcctgagctcaggtgatccacccgcctcggcctcccaaagtgctgggataacaggagtgagccactaggtgtgacctaatttttgtatttttagtagagatgggatttcaccatgtcggctaagctggtctcgaactcctgacctcaggtgatctgcctgccttggcctcccaatgtgctgggattataggcataagccaccgcactggctttttttttttttttttttttaaacctggatggttttattttgcatgaatgtatagatatttcctgttcatacattctgaaagtgaacaactgtatatatgcaatttatttttattcttatttatttatttgtttattttttgagaccagagtctcactctgtcgcccaggctagagtgcaatgacacaatctcggttcactgcaacctctgcctcctgggttaagcaattcttctgcctcagcttccccagtagctgggattacaggtgtccgctaatttttgtatttttacaaaatacacccaggtaattttttgtaattttggtagagacaggtttcaccatgtcggccaggctggtctcgaactcctgacctcaggtgatatgcccgactcagcctcccaaagtgctgggattacaggtgtgagccactgcgtctggcctgcatggggattcttaatgaagattaattattgtagttgagggggaaaaggaataataaatatttattggaccctaaataccttcgaatatggaataccctaggtattctagggcatttagggaccaataaatatttattcctccgtactcttccctcgctcttttcagatttttttttttttttttttttttttgagatggagtcttgctctgtctccaggctggagtgcagtggcgcgatcttggctcactgcaacctctgcctcctgggttgaagtgattctcttgcctcagcctcctgagtggctgggactacaggtgcataccactatgcccagctaatttttgtattttttgtagagacaggctttcaccatgttggccaggatggtctcgttctttagacctcgtgatctgtcttcctcagcctcccaaagtgttggaattacaggcgtaagcctccgccgggccttttttagatttttaagagaatttttgttaaagcatgaacttaaaaaatcagacttggcttggagcggtggctcatggcctctagtcccaggactttgggtggctgaggcaagtggattgcttgagcccaggagttcaagacctgccttggcaataatatcaagaccccctcttcatgaaaaacaatcaagctaatacttgatactattttacataagaattttttatagtatgtcatgttttaatgtatattggttatatagttgcaaatttaaaggcatggtggtggctcatacctgtaatcccagcactttgggaggctggggcgggcagatcttctgaggtcaggagttcaagaccagcctggccaacatggtggaaccccgtcttaggctgaggcaggagaatagcttgtgcccaggaggcagaggttgctttgagctgagatcgcaccacggcattccagcctggaggacagagcgagactctgtctctaaataaataaataaataaataaatgtatactaactgcattagcaagactccgtctctaaataaataagtgaataaataaatgtatactaattgcattttaaaaatcaaagtataggccgggtacggtggctcacaactgtaatcctagcacttttggaggctgaggtggatggatcacctgaggtcaggagtttgagaccagcctgaccaacatggtgaaactttgtctctactaaaaatacaaaattagctggtgtggtggcgcatggctgtaatcccagctactcgggaggctgaggtaggagaattgcttgaacctgagaggtggaggttgtggtgagcggagatcgtgctgttgcactccagcctgggcaacaagagcgaaacttcgtctccaagaaaaaaaaaatatataattcacataagataaaattcaccctctttggccaggcgcagtggctcatgcctgtaatcccagcactttgggaggtagaggtgggcagatcacttgaggtcagggagtttgagaccagcctggccaacatggtgaaaccccatctctactaaaaatacaaaaattagcccggtgtggtggcatacacctgtaatccacctactcaggacgctgagtctgcactcagtccctgggctacagggtgaaactgtatctcaaaaataaagaataaaatgcagctacttaaagggtgtagagttgaacaactgttaccactgtctaattccagaacctttcatcaccccaaaagaaaacccattcccagcagtcatttcccattaagtctcctctagcccctcacaaccactaatctaattcatgtttctatgtatttgcctattctaggcgtttcatacaaatacagtcatataatttgtggcctttcgtgtctgacttgtttaacttagcataatgttttaaggcccatttatgttgttgtatgtatgcatacttcattccattttactgctgaatattgctttgtactgatgccactttttgtttgtcttttcatcacttgacggacattttgtttcttccactttgtggctgttacaggcagtgctactgtgaaaatttgtattaaagttttagcgtgaatatatgttttcagttctcttgggaaaatacctagaagtggtattgtcggatcatagggtcattctatgtttagcattttgaggaacagccagactgttttacatagtggttgcaccgttttacagtcctactttagcctatatgggttctaatttctttctttctttctttctttctttctttctttctttctttctttctttctttctttctttcttttctttcttttctttcttttctttcttttctttctttctttctttctttttttagaacagagtctccctctgtagcccaggctggagtgcagtggcatggtcttggctcactgcagcctccgcctctcgggttcaagcaattctctgcctcagcctcccaagtagcttggactacaggcgcccgccaccacgcctggctaatgtttgtatttttggtagtgacagggtttcaccacattggccaggttggtcttgaactcctgacctcaggtgattcacccacctcggcctcccaaagtgccgagattacaggcatgagccactgcatccgggcgtgggttctaaattcttaatattctcatcaacatttattgctgtctttttaattttagcctgtaatcccagctactagggcgactgaggtggtagcatcgcttgagcccaggaagctgaggctgcagtgagccaagattgcaccactgcactccaggctaggtgatgaagtgagacttcatctcaaaaaaaaaaaaaaggaagtaatggcaaaaactggaattattttgcaccaacttaaatatttagatctttaatacctttggaaagttttttatatatagtttgtgtgtgtgtgtgtgtatatatacacacatatatatatacacacacatatatacacacatatatatgaatgattttatatatatatatatatatatatatatgaatgatatatatatatatatatgaatgaatgaatgagatggagtctcactctgtcacccaggcaggagtgcagtggtgccattttggcttatggcagcctccgcctccggggttcaagtgattcttgtacctcagcctcccgagttgctgggattacaggcactcgccaccatgcccggattttttgtcttaattcatgaaggatgaattaagtctgcagttgttctttttccctttttctttccagtttttttttttgtttgtttgtttgtttttgagacacagtctcactcggttgtccaggctggagtgcggtggcagtatcttggctccctgtaacccatctccctggttcaagcgattccggtgcctcagcttcccaagtagctaggattacaggtgtgtgacaccacacctggttaatttttgtatttttagtagagacgaggtttcaccgcattggttaggttggtctcaaaactcctgacctcaggtgaaccgcccacctaagccttccaaagtgctgagattacatgcatgagccaccaagtctggcctaagtctgaatttttttttttttttttttgagacggagtttcgctcttgttgcccaggctggagtgcaatggtgcgatcttggctaaccgcaacctccgcctcccacgttcaagcaattctgcctcagcctcccgagtagctgggattgcaggcatataccaccacgcctggctaattttgtatttttgttagagatggggtttctccgtgttgagactggtctcgaactcctgacctcaggtgatccgcctgcctcggcctcccaaagtgctgggattacaggtgtgaaccactgcacccggccgaatatatttttttttttttaaatggagtctcgctctgtggcccaggctggaatgcagcggtgtgatcttagctcactgcaacctctgcctccctggctcaagcgattctcctgcttcagcctcctgagtacctgggaccacaggtgtgcaccaccatgcctgaataatttttttgtgtttttgtagagatggagtttcaccatgttggccaggctgatcttgaactactgacctcaggtgatgtgcctgcctccgccttcccaagtgctgggattacaggcatgagctactgtacccggctaagtgtacagtgttcttgtgatgtctttgtctggtgttggtatcagggtaatactgtcttcaagattacccttgaatgagctttacttcattttttaatgtgtttttttttcttttcttttgttttttgtttttgagacagagtttcactctgtcgcacaggctggaatccacactctaggctcgctgcagcctccacctcccaggttcaagagattctcctgtgtcagcctcttgagtagctggggttacaggcacgtgccacgacgcccggctgatttttttgtatttttagtagtgacgggctttcaccatgttggccaggctggtctcgaactcctgacatcaagtgacctgccttcctcagcctcccaaagtgttgggattacaggagtgagccactgtgccccgcctgcaattacttcttaagttctcaattaaaagagagtttatcaaggactttttttggtaattttgcattttgaaaattgctaacattaactgggacagcccttttatttatttatttgtcactcagttgtttttttgagttgcctactatgtcccaggcactggtaagataggagtatcattgtacctgaggcagggcaacatgtgcttgcttgagaggagcatgatctaggattataaggactgcaacctccccttcccaggttgaagcagttctcatgcctcagcctcccaagtagctgggactacagccatgagccaccacgcccagctaatttttgtgtttttagtagagatgaggtttccccatgttggccaggctagtctcaacttctggacctcaggtgatctgcccacttcagcctcccaaagtgctgaaattacaggagtaattttattctcccaaagctgctgctttgggagaataaaaagttgagtatgggccaggcatgggggctgatgcctgtgatcgcagcactttaggagactgaggtgggagtctagcttgagcccagtagtttgagacaagcctggggaacatagggagatccggcctctacaaaaaaaataaattagctgggtggagtggcatgtgcctgtggtcccagctacttgggtggttgaggtgggaagatatctgagctcaggagttccaggctgcagtgagctctgattatgcactccagcctgggtgacagagtgagatgctgtctcaaaaaaaaaaattcagtgtggcgtgattaggctgggagggtggggcaggaagggatgacattggaggggtaggcaaggtgtagatagacctttccctatattctcctatttttaaaaaatttttttctaaatagagatagggtcttactattttgcccaggctgggtctcaaactcctgggctcaagtaatccttccatctaggcctctattttttgtgcaaacgattgaaattatattttttttacctgaatttttcctgtgaacattgggttatttataaacctgttttctgtttctttctttctttttttttttttttgtttttgttttttgagatagagtccagcctggagtgctgtggcatgatcttggcacacttgcaacctctgcctcctgggttcaggtgattctcctcctctagcctcctccacgcctggctaatatttgtatttttagtagagatggggtttcaccatgttggccgggctgttcttgaactcctggtttcaacagatccacctgcctcagcctgccaaagtgctgagattacaggtgtgagccactgttctaggcacttgtttctgtttcttaattttggctgctactcagtgggaaaaagcacagattgaatctaattgaggccgggcgctgtggctcactcctgtaatttcagcactttgggaggctgaggtgggcagatcacctgagatccagagttcgagactagcctggccaacatggggaaacctcatctctactaaaaacacaaaaattagttgggcgtggtggctcatggctgtagtcccagctactcgggaggctgaggcatgagaattgcttcaacccgggaggtggaggttgcagtgagctgagatcaggacactgccctccaggttgggcaagagagtgagactcggtcttaaaaaaaaaaaaaaatctagttgaaaaatgtcatcgggtctttccaaatttttactaggaatttgttaaaattaaccaggctggaagtcattatagtttgtttgtttgtttgtttgagatgggggtctcactctgtcacgcaggctggagttcagtggtaggatctcggctcactgcaacctctgcatcccagattcaagcgatcctctcacctctgcctcatgagtagttggaaccacaggcatgtgtcaccatgcttttgtagagacagggtttctttcgccctattggctaggctggtctcaaacttgtgagctcaagcgatccgcccaccttggcctcccaaagtgctgggattacaggcatgagttaccttgccttgcccattatagcttttttgaggctgggtcttactctctgtcatgcaggctggactgcagtggtgtgatctaagctcactgcctcctgggctcaagcagtcctcccacctcagcctcctgagtagctggcacaggcgctacctcacccatctaattttttattttttttagagatggggttttgccatgtttgcccaggctggtctagaattcatgagctcaagtgatctacctgcctcggcctcccaatgtgctgggattacagacatgagccactatgttcagccatacctggctaatttttaaaaaatgttttcaagagacagggtctccctgtgttgcccaggttggtctcaagttcctgggattactgctggccttcaaaagtaaatgtgaaataattagttaatttctccctcagttgacaaataatgccaaaagtgataaagattaatgaaatgtctcttttttttttttttttttgagacggagtctcgttctgttgccaagtctggaatgcagtggcacgatctcggctcactgcaacgtccacctactgggttcaagtgattctcctgcctcagcctcccgagtagctgggactacaggcacgcatcaccatgcccggctaatttttgtatttttagtagagacggggtttcactatgttggccaggctggtcttgaactcctgacctcatgatccacccaccttggcctcccaaagtgctgggattacaggcatgagccaccgcgcccagccatgaaatttcttacgtagaaaggcagcttgggattgtagaaagaatgtaggctttggagttggacaggcctccatttgagaccatacttgagtcccgtgcttgccttagacaaagaacctctcaaccttagtttttaatctataaggtgttttgaaaattaattcctagttcagtacatggcacatggtaggtacctgctgctatccataattctcttagttaatatattcggtgccacatgccaggcagccaggatctgtactaagcacctaataagtattatctcatttaatcctcaaaagaaccccacctgagttgctagacagccattatttcagggttacacattaggaaattgaagcttagagagatttaagtggttagccaagtgatggtgctggtattccaactaaggtcatctgctttcagagcatttactttctgttaggctgcctctcctgttgcaaagtactaagaacacaactacataatgtatttttagtggattcttgtctttttgtaaatagaaggttaaaatgagaggaattttttttttgtttgggagacgtggtctcgctctgatgagagctagaaatttgattacttgtatttctggtctgcataaaaatttggcctaaaaacatcaatagaaaggcaagtgtcatctgcaaatctgtcccatcctgttcttcacaggaaaatgtaaccttttttttttttttttctttttttgagatggagtctagctctgttgcccaagctggagtgcaatggcatggtttcccgctcactgcaacctctgccttctgggttctagcagttctcctgcctcagcctcctgagtagctgggattacaggcgcctgccaccatgcctggctaatttttgtatttttagtagagacagggtttcaccatgttggccaggctggtctttaactcctgacctcaggtgatccgcctgcctcggcctcccaaagtgctgggatcacaggtgtgagccactgcgcccgggctcaaaatgtaacgtctgtctagtatgaggatttatttccttgtctgacttctgagttgtaatcgtttattaacaatcacattgtaagtttatctatgaagtaataaaatgttctttctgtatattatactggaaatgaatgcttcattcaaaaaatagttttatcttgggaaggtagccactttttaaaaattgaggtaaaacggccaggcacggtggctcacgcccataattccagcactttgggaggccaaggtgggtggagatcacctgaggtcagaagttcaagaccagcctggccaatatggtgaaactccatctctactaaaatacaaaaattagaccggcatggtggcaggtgcctgtaatcccagctactcaggaagctgaggcaggagaatcgcttgaacccaggaggtggaggttacagtgagccgagatcctgccgctgcattgaagcctgggtgagaagagcgaaactctgtctcattaaaaaaaaaaaaaaagaggtaaaatttaaataacttaaggctgattgtattggcttacacttgtaattccagcattttgggagaccaaggcaggaggatcacttgaactcagaagtttgagaccagcctggtcaacatagggaaacctcatctccacaaaaaataaaaaataaaatataaaaacttcaaaattaaataagttacagttcaccattgtaaccattttattttatcctatttattttgagacagtcttgttttgtcacccaggctggagtacagtggtgggatcacagctcactacagcctccaccttccaggttcaagtgattcttctgcctcagcctctgtaactgggattacaggtgcttgccaccacaccctgctaatttttgtattttgattagagacagggtttcaccatgttggcccgattggtctcgaactcctgagctcaagtgatctgcctgtcttggcctcccaaaatgagccaccgtgcctgtccccttagtctactttaaaattcaatttgccttttttttaaattgtaagaattccttatatattttggatattaaatccttaactagggatatgattcgcaaatttttttcccccattctgtttctgtaggctctttgacattctttttctttctctttttgagacaaggtcttactctgttgcccaggctagagtacagtggtgtgatcatagcttactacagcctcgacttccctgggctgaagcaatccttacctcccacctcagcctcccaggtaaccaggactacaggtgtacaccaccatgcctggcaaatcactgttgttgttgttgttgttgttatagccataggctcccactgtgttgcccaggctgggctcaagcaatcttctagccttctagccttggcctcccgaagtggtgggattatgcgcatgatcgctgctcccagcccacaatctttttttttttttttttgagatggagtctcgctctgtcacccaggctggagtgcggtggcgcaatctcggctcattgcaacctccgcctcccgggttcaagcgattctcctgcctcagcctcccgagtggctgggattacaggcacgtgctgccacgcccagctaatttttgtattttttttttagaagagatggggtttcaccatattggccaggatggtctcgaactcctgacctcatgatatgcccaccttggcctcccaaagtgccgggattacaggcatgagccaccgcgcctggcctcccagcccacattcttgataattttctttgcttctaaaagttttgcttttagggttgggcaaggtggcttatgcctataatcctagcactttgggaggctgaggtgggcggatctcttgagctcaggagttcaagaacaccctgagcaacatggaaaaaccgtgtctctacaaaaaatgcaaaaattagccaagtgtggtggcatgcacctgtagtctgagctactggggaggctgtgacaggaggatcacttgaattggactggaggctgcagtgagtgaaaatggtaccactgcactccagcctgggtaatagagtgagatgctgtctgaaaaaaaaaaaaagttttagtttttttgggtggggggattttaacttcacctat

SEQ ID NO: 6 - DNA, 156 bp, Homo sapiens
  source 1..156: mol_type = genomic DNA
tgaaaccttaaaatggaacagctcagaaagttccagtggaacaaacagcctcagagcagttagtggcagggcatgaggcgcccactacccgcccaatcacagcagggttagaactaacattgcatgcagtccgcccgagtgattggctgaacatct

SEQ ID NO: 7 - DNA, 20 bp, Homo sapiens
  source 1..20: mol_type = genomic DNA
ctggaggcatctgaaggcat

SEQ ID NO: 8 - DNA, 30 bp, Homo sapiens
  source 1..30: mol_type = genomic DNA
gcatctgaaggcatcaacacagagcactaa

SEQ ID NO: 9 - DNA, 23 bp, Homo sapiens
  source 1..23: mol_type = genomic DNA
cagtactgtacctttcttcttgc

SEQ ID NO: 10 - DNA, 20 bp, Homo sapiens
  source 1..20: mol_type = genomic DNA
taaggtttcactaagggaga

SEQ ID NO: 11 - DNA, 20 bp, Homo sapiens
  source 1..20: mol_type = genomic DNA
aagcacttacagatgttcag

SEQ ID NO: 12 - DNA, 15 bp, Homo sapiens
  source 1..15: mol_type = genomic DNA
ctgcactcatctgac

SEQ ID NO: 13 - DNA, 15 bp, Homo sapiens
  source 1..15: mol_type = genomic DNA
acaccctgcactcat

SEQ ID NO: 14 - DNA, 15 bp, Homo sapiens
  source 1..15: mol_type = genomic DNA
caagcacaccctgca

SEQ ID NO: 15 - DNA, 15 bp, Homo sapiens
  source 1..15: mol_type = genomic DNA
cgtaacaagcacacc

SEQ ID NO: 16 - DNA, 15 bp, Homo sapiens
  source 1..15: mol_type = genomic DNA
ctcctcgtaacaagc

SEQ ID NO: 17 - DNA, 15 bp, Homo sapiens
  source 1..15: mol_type = genomic DNA
tcagcctcctcgtaa

SEQ ID NO: 18 - DNA, 15 bp, Homo sapiens
  source 1..15: mol_type = genomic DNA
ccacctcagcctcct

SEQ ID NO: 19 - DNA, 15 bp, Homo sapiens
  source 1..15: mol_type = genomic DNA
tgtttccacctcagc

SEQ ID NO: 20 - DNA, 15 bp, Homo sapiens
  source 1..15: mol_type = genomic DNA
tcggctgtttccacc

SEQ ID NO: 21 - DNA, 15 bp, Homo sapiens
  source 1..15: mol_type = genomic DNA
agagctcggctgttt

SEQ ID NO: 22 - DNA, 15 bp, Homo sapiens
  source 1..15: mol_type = genomic DNA
gtgtgagagctcggc

SEQ ID NO: 23 - DNA, 15 bp, Homo sapiens
  source 1..15: mol_type = genomic DNA
acacggtgtgagagc

SEQ ID NO: 24 - DNA, 15 bp, Homo sapiens
  source 1..15: mol_type = genomic DNA
acaagacacggtgtg

SEQ ID NO: 25 - DNA, 15 bp, Homo sapiens
  source 1..15: mol_type = genomic DNA
cactaacaagacacg

SEQ ID NO: 26 - DNA, 15 bp, Homo sapiens
  source 1..15: mol_type = genomic DNA
cagagcactaacaag

SEQ ID NO: 27 - DNA, 15 bp, Homo sapiens
  source 1..15: mol_type = genomic DNA
caacacagagcacta

SEQ ID NO: 28 - DNA, 15 bp, Homo sapiens
  source 1..15: mol_type = genomic DNA
ggcatcaacacagag

SEQ ID NO: 29 - DNA, 15 bp, Homo sapiens
  source 1..15: mol_type = genomic DNA
ctgaaggcatcaaca

SEQ ID NO: 30 - DNA, 15 bp, Homo sapiens
  source 1..15: mol_type = genomic DNA
ggcatctgaaggcat

SEQ ID NO: 31 - DNA, 15 bp, Homo sapiens
  source 1..15: mol_type = genomic DNA
ctggaggcatctgaa

SEQ ID NO: 32 - DNA, 15 bp, Homo sapiens
  source 1..15: mol_type = genomic DNA
ctgggctggaggcat

SEQ ID NO: 33 - DNA, 15 bp, Homo sapiens
  source 1..15: mol_type = genomic DNA
agggactgggctgga

SEQ ID NO: 34 - DNA, 15 bp, Homo sapiens
  source 1..15: mol_type = genomic DNA
acaacagggactggg

SEQ ID NO: 35 - DNA, 15 bp, Homo sapiens
  source 1..15: mol_type = genomic DNA
gcaccacaacaggga

SEQ ID NO: 36 - DNA, 15 bp, Homo sapiens
  source 1..15: mol_type = genomic DNA
ttgcagcaccacaac

SEQ ID NO: 37 - DNA, 15 bp, Homo sapiens
  source 1..15: mol_type = genomic DNA
cagccttgcagcacc

SEQ ID NO: 38 - DNA, 15 bp, Homo sapiens
  source 1..15: mol_type = genomic DNA
cgtaccagccttgca

SEQ ID NO: 39 - DNA, 15 bp, Homo sapiens
  source 1..15: mol_type = genomic DNA
aggagcgtaccagcc

SEQ ID NO: 40 - DNA, 15 bp, Homo sapiens
  source 1..15: mol_type = genomic DNA
cttcgaggagcgtac

SEQ ID NO: 41 - DNA, 15 bp, Homo sapiens
  source 1..15: mol_type = genomic DNA
tgcttcgaggagcgt

SEQ ID NO: 42 - DNA, 15 bp, Homo sapiens
  source 1..15: mol_type = genomic DNA
catggtgcttcgagg

SEQ ID NO: 43 - DNA, 15 bp, Homo sapiens
  source 1..15: mol_type = genomic DNA
catgccatggtgctt

SEQ ID NO: 44 - DNA, 15 bp, Homo sapiens
  source 1..15: mol_type = genomic DNA
catctcatgccatgg

SEQ ID NO: 45 - DNA, 15 bp, Homo sapiens
  source 1..15: mol_type = genomic DNA
acctccatctcatgc

SEQ ID NO: 46 - DNA, 15 bp, Homo sapiens
  source 1..15: mol_type = genomic DNA
taggaacctccatct

SEQ ID NO: 47 - DNA, 15 bp, Homo sapiens
  source 1..15: mol_type = genomic DNA
gcttctaggaacctc

SEQ ID NO: 48 - DNA, 15 bp, Homo sapiens
  source 1..15: mol_type = genomic DNA
ttcttgcttctagga

SEQ ID NO: 49 - DNA, 15 bp, Homo sapiens
  source 1..15: mol_type = genomic DNA
ctttcttcttgcttc

SEQ ID NO: 50 - DNA, 15 bp, Homo sapiens
  source 1..15: mol_type = genomic DNA
tgtacctttcttctt

SEQ ID NO: 51 - DNA, 15 bp, Homo sapiens
  source 1..15: mol_type = genomic DNA
agtactgtacctttc

SEQ ID NO: 52 - DNA, 15 bp, Homo sapiens
  source 1..15: mol_type = genomic DNA
tggtcagtactgtac

SEQ ID NO: 53 - DNA, 15 bp, Homo sapiens
  source 1..15: mol_type = genomic DNA
ccaactggtcagtac

SEQ ID NO: 54 - DNA, 15 bp, Homo sapiens
  source 1..15: mol_type = genomic DNA
caaggccaactggtc

SEQ ID NO: 55 - DNA, 15 bp, Homo sapiens
  source 1..15: mol_type = genomic DNA
taaatcaaggccaac

SEQ ID NO: 56 - DNA, 15 bp, Homo sapiens
  source 1..15: mol_type = genomic DNA
atcagtaaatcaagg

SEQ ID NO: 57 - DNA, 15 bp, Homo sapiens
  source 1..15: mol_type = genomic DNA
aacacatcagtaaat

SEQ ID NO: 58 - DNA, 15 bp, Homo sapiens
  source 1..15: mol_type = genomic DNA
tttaaaacacatcag

SEQ ID NO: 59 - DNA, 15 bp, Homo sapiens
  source 1..15: mol_type = genomic DNA
cacagtttaaaacac

SEQ ID NO: 60 - DNA, 15 bp, Homo sapiens
  source 1..15: mol_type = genomic DNA
gcagacacagtttaa

SEQ ID NO: 61 - DNA, 15 bp, Homo sapiens
  source 1..15: mol_type = genomic DNA
aaatggcagacacag

SEQ ID NO: 62 - DNA, 15 bp, Homo sapiens
  source 1..15: mol_type = genomic DNA
atataaaatggcaga

SEQ ID NO: 63 - DNA, 15 bp, Homo sapiens
  source 1..15: mol_type = genomic DNA
tgcagatataaaatg

SEQ ID NO: 64 - DNA, 15 bp, Homo sapiens
  source 1..15: mol_type = genomic DNA
aacagtgcagatata

SEQ ID NO: 65 - DNA, 15 bp, Homo sapiens
  source 1..15: mol_type = genomic DNA
ctccaaacagtgcag

SEQ ID NO: 66 - DNA, 15 bp, Homo sapiens
  source 1..15: mol_type = genomic DNA
ccctcctccaaacag

SEQ ID NO: 67 - DNA, 15 bp, Homo sapiens
  source 1..15: mol_type = genomic DNA
cccgtccctcctcca

SEQ ID NO: 68 - DNA, 15 bp, Homo sapiens
  source 1..15: mol_type = genomic DNA
ccttccccgtccctc

SEQ ID NO: 69 - DNA, 15 bp, Homo sapiens
  source 1..15: mol_type = genomic DNA
ttccaccttccccgt

SEQ ID NO: 70 - DNA, 17 bp, Homo sapiens
  source 1..17: mol_type = genomic DNA
ccatggtgcttcgagga

SEQ ID NO: 71 - DNA, 18 bp, Homo sapiens
  source 1..18: mol_type = genomic DNA
tcatgccatggtgcttcg

SEQ ID NO: 72 - DNA, 18 bp, Homo sapiens
  source 1..18: mol_type = genomic DNA
gtcagtactgtacctttc

SEQ ID NO: 73 - DNA, 15 bp, Homo sapiens
  source 1..15: mol_type = genomic DNA
ggcattctattcaaa

SEQ ID NO: 74 - DNA, 15 bp, Homo sapiens
  source 1..15: mol_type = genomic DNA
tcttgggcattctat

SEQ ID NO: 75 - DNA, 15 bp, Homo sapiens
  source 1..15: mol_type = genomic DNA
gcccctcttgggcat

SEQ ID NO: 76 - DNA, 15 bp, Homo sapiens
  source 1..15: mol_type = genomic DNA
ataatgcccctcttg

SEQ ID NO: 77 - DNA, 15 bp, Homo sapiens
  source 1..15: mol_type = genomic DNA
cctaaataatgcccc

SEQ ID NO: 78 - DNA, 15 bp, Homo sapiens
  source 1..15: mol_type = genomic DNA
tgtttcctaaataat

SEQ ID NO: 79 - DNA, 15 bp, Homo sapiens
  source 1..15: mol_type = genomic DNA
atcagtgtttcctaa

SEQ ID NO: 80 - DNA, 15 bp, Homo sapiens
  source 1..15: mol_type = genomic DNA
ccaagatcagtgttt

SEQ ID NO: 81 - DNA, 15 bp, Homo sapiens
  source 1..15: mol_type = genomic DNA
tccttccaagatcag

SEQ ID NO: 82 - DNA, 15 bp, Homo sapiens
  source 1..15: mol_type = genomic DNA
atttgtccttccaag

SEQ ID NO: 83 - DNA, 15 bp, Homo sapiens
  source 1..15: mol_type = genomic DNA
tacttatttgtcctt

SEQ ID NO: 84 - DNA, 15 bp, Homo sapiens
  source 1..15: mol_type = genomic DNA
ttgattacttatttg

SEQ ID NO: 85 - DNA, 15 bp, Homo sapiens
  source 1..15: mol_type = genomic DNA
tttatttgattactt

SEQ ID NO: 86 - DNA, 15 bp, Homo sapiens
  source 1..15: mol_type = genomic DNA
ttaagtttatttgat

SEQ ID NO: 87 - DNA, 15 bp, Homo sapiens
  source 1..15: mol_type = genomic DNA
cattcttaagtttat

SEQ ID NO: 88 - DNA, 15 bp, Homo sapiens
  source 1..15: mol_type = genomic DNA
gaaaacattcttaag

SEQ ID NO: 89 - DNA, 15 bp, Homo sapiens
  source 1..15: mol_type = genomic DNA
ggagagaaaacattc

SEQ ID NO: 90 - DNA, 15 bp, Homo sapiens
  source 1..15: mol_type = genomic DNA
ctaagggagagaaaa

SEQ ID NO: 91 - DNA, 15 bp, Homo sapiens
  source 1..15: mol_type = genomic DNA
tttcactaagggaga

SEQ ID NO: 92 - DNA, 15 bp, Homo sapiens
  source 1..15: mol_type = genomic DNA
taaggtttcactaag

SEQ ID NO: 93 - DNA, 15 bp, Homo sapiens
  source 1..15: mol_type = genomic DNA
cattttaaggtttca

SEQ ID NO: 94 - DNA, 15 bp, Homo sapiens
  source 1..15: mol_type = genomic DNA
tgttccattttaagg

SEQ ID NO: 95 - DNA, 15 bp, Homo sapiens
  source 1..15: mol_type = genomic DNA
tgagctgttccattt

SEQ ID NO: 96 - DNA, 15 bp, Homo sapiens
  source 1..15: mol_type = genomic DNA
ctttctgagctgttc

SEQ ID NO: 97 - DNA, 15 bp, Homo sapiens
  source 1..15: mol_type = genomic DNA
tggaactttctgagc

SEQ ID NO: 98 - DNA, 15 bp, Homo sapiens
  source 1..15: mol_type = genomic DNA
tccactggaactttc

SEQ ID NO: 99 - DNA, 15 bp, Homo sapiens
  source 1..15: mol_type = genomic DNA
tttgttccactggaa

SEQ ID NO: 100 - DNA, 15 bp, Homo sapiens
  source 1..15: mol_type = genomic DNA
ggctgtttgttccac

SEQ ID NO: 101 - DNA, 15 bp, Homo sapiens
  source 1..15: mol_type = genomic DNA
aatgttagttctaac

SEQ ID NO: 102 - DNA, 15 bp, Homo sapiens
  source 1..15: mol_type = genomic DNA
catgcaatgttagtt

SEQ ID NO: 103 - DNA, 15 bp, Homo sapiens
  source 1..15: mol_type = genomic DNA
gactgcatgcaatgt

SEQ ID NO: 104 - DNA, 15 bp, Homo sapiens
  source 1..15: mol_type = genomic DNA
gggcggactgcatgc

SEQ ID NO: 105 - DNA, 15 bp, Homo sapiens
  source 1..15: mol_type = genomic DNA
cactcgggcggactg

SEQ ID NO: 106 - DNA, 15 bp, Homo sapiens
  source 1..15: mol_type = genomic DNA
ccaatcactcgggcg

SEQ ID NO: 107 - DNA, 15 bp, Homo sapiens
  source 1..15: mol_type = genomic DNA
ttcagccaatcactc

SEQ ID NO: 108 - DNA, 15 bp, Homo sapiens
  source 1..15: mol_type = genomic DNA
agatgttcagccaat

SEQ ID NO: 109 - DNA, 15 bp, Homo sapiens
  source 1..15: mol_type = genomic DNA
cttacagatgttcag

SEQ ID NO: 110 - DNA, 15 bp, Homo sapiens
  source 1..15: mol_type = genomic DNA
aagcacttacagatg

SEQ ID NO: 111 - DNA, 15 bp, Homo sapiens
  source 1..15: mol_type = genomic DNA
ccattaagcacttac

SEQ ID NO: 112 - DNA, 15 bp, Homo sapiens
  source 1..15: mol_type = genomic DNA
tctagccattaagca

SEQ ID NO: 113 - DNA, 15 bp, Homo sapiens
  source 1..15: mol_type = genomic DNA
atttgtctagccatt

SEQ ID NO: 114 - DNA, 15 bp, Homo sapiens
  source 1..15: mol_type = genomic DNA
ctgctatttgtctag

SEQ ID NO: 115 - DNA, 15 bp, Homo sapiens
  source 1..15: mol_type = genomic DNA
ctgggctgctatttg

SEQ ID NO: 116 - DNA, 15 bp, Homo sapiens
  source 1..15: mol_type = genomic DNA
tccctctgggctgct

SEQ ID NO: 117 - DNA, 15 bp, Homo sapiens
  source 1..15: mol_type = genomic DNA
ccccctccctctggg

SEQ ID NO: 118 - DNA, 15 bp, Homo sapiens
  source 1..15: mol_type = genomic DNA
tttgaccccctccct

SEQ ID NO: 119 - DNA, 15 bp, Homo sapiens
  source 1..15: mol_type = genomic DNA
ttccatttgaccccc

SEQ ID NO: 120 - DNA, 15 bp, Homo sapiens
  source 1..15: mol_type = genomic DNA
gtctcttccatttga

SEQ ID NO: 121 - DNA, 15 bp, Homo sapiens
  source 1..15: mol_type = genomic DNA
ttgatgtctcttcca

SEQ ID NO: 122 - DNA, 15 bp, Homo sapiens
  source 1..15: mol_type = genomic DNA
tattattgatgtctc

SEQ ID NO: 123 - DNA, 15 bp, Homo sapiens
  source 1..15: mol_type = genomic DNA
atctgtattattgat

SEQ ID NO: 124 - DNA, 15 bp, Homo sapiens
  source 1..15: mol_type = genomic DNA
cccacatctgtatta

SEQ ID NO: 125 - DNA, 15 bp, Homo sapiens
  source 1..15: mol_type = genomic DNA
aatgtcccacatctg

SEQ ID NO: 126 - DNA, 15 bp, Homo sapiens
  source 1..15: mol_type = genomic DNA
aaaataatgtcccac

SEQ ID NO: 127 - DNA, 15 bp, Homo sapiens
  source 1..15: mol_type = genomic DNA
aagaaaaaataatgt

SEQ ID NO: 128 - DNA, 15 bp, Homo sapiens
  source 1..15: mol_type = genomic DNA
ttgcaaagaaaaaat